(19) United States
(12) Patent Application Publication
(10) Pub. No.: US 2024/0382617 A1
(43) Pub. Date: Nov. 21, 2024

(54) VIRUS COMPOSITIONS TO EFFICIENTLY TARGET THE NERVOUS SYSTEM AND THE LUNGS

(71) Applicant: CAPSIDA BIOTHERAPEUTICS, INC., Thousand Oaks, CA (US)

(72) Inventors: Viviana Gradinaru, Pasadena, CA (US); Xinhong Chen, Pasadena, CA (US); Sripriya Ravindra Kumar, Chennai (IN)

(21) Appl. No.: 18/692,014

(22) PCT Filed: Sep. 20, 2022
(86) PCT No.: PCT/US2022/076730

§ 371 Date: Mar. 14, 2024

Related U.S. Application Data
(60) Provisional application No. 63/246,632, filed on Sep. 21, 2021.

(51) Int. Cl. A61K 48/00; A61K 9/00; C07K 14/005; C12N 7/00; C12N 15/86

(52) U.S. Cl. CPC A61K 48/0041; A61K 9/0019; A61K 48/0075; C07K 14/005; C12N 7/00; C12N 15/86; C12N 2750/14122; C12N 2750/14143

(57) ABSTRACT

Disclosed herein include compositions and kits comprising recombinant adeno-associated viruses (rAAVs) with tropisms to the central nervous system, the peripheral nervous system, and/or the lung, with increased specificity and transduction efficiency. Also described include methods of treating various diseases and conditions using the rAAVs.

### RELATED APPLICATIONS

This application claims the benefit under 35 U.S.C. § 119(e) of U.S. Provisional Application No. 63/246,632, filed Sep. 21, 2021, the content of this related application is incorporated herein by reference in its entirety for all purposes.

### STATEMENT REGARDING FEDERALLY SPONSORED R&D

This invention was made with government support under Grant No. NS111369 & MH117069 awarded by the National Institutes of Health. The government has certain rights in the invention.

### REFERENCE TO SEQUENCE LISTING

The present application is being filed along with a Sequence Listing in electronic format. The Sequence Listing is provided as a file entitled 30KJ_302454_WO_Sequence_Listing, created Sep. 15, 2022, which is 393 kilobytes in size. The information in the electronic format of the Sequence Listing is incorporated herein by reference in its entirety.

### BACKGROUND

### Field

The present disclosure relates generally to the field of adeno-associated virus (AAV) vectors, more specifically nervous system, and lung-tropic AAVs for genetic access to whole-body neurons and epithelial cells following non-invasive systemic delivery.

### Description of the Related Art

Gene delivery to the central and peripheral nervous systems (CNS and PNS) has greatly accelerated neuroscience research in the last decade, and has the potential to translate this research into novel therapies for neurological disorders. However, the lack of potent vectors enabling non-invasive gene delivery across species is a significant bottleneck that can hinder fast progress.

AAVs are single-stranded DNA viruses with relatively low immunogenic responses and stable transgene expression. The natural serotypes of AAV exist across different mammalian species, and their ability to target dividing and non-dividing mammalian cell types has become an attractive area of research among bioengineers to improve its usability as a safe gene delivery vector with improved efficiency and specificity to target a cell-type of scientific or therapeutic interest. With several ongoing gene therapy clinical trials that use recombinant AAVs (rAAVs) as a gene delivery vector, and with 3 gene therapies approved to date, engineering AAV capsids (outer protein shell) to alter or improve their tropism to suit scientific and therapeutic needs is an active area of research.

The success of gene delivery relies on a safe and efficient vector, and for this reason, most in vivo applications use AAV vectors. AAVs offer several advantages, including stable, long-term transgene expression and low immunogenicity. The natural serotypes of AAV have demonstrated considerable success in targeting different cell populations within the nervous system through direct routes of gene delivery, such as intracranial, intracerebroventricular (ICV), intrathecal, intraganglionic, intrasciatic and intracolonic. These direct delivery routes suffer from limitations, however, including the need for invasive surgery. In addition, anatomical barriers can restrict surgical access (such as for nodose ganglia (NG) or dorsal root ganglia (DRG)). Localized delivery can lead to incomplete coverage of a large complex system such as the enteric nervous system (ENS) or CNS, and multiple direct interventions can be needed to increase coverage.

An alternative, non-invasive, intravenous (IV) route circumvents these limitations. Some natural serotypes, including AAV9, can target the CNS or PNS systemically. However, lack of specificity towards the target and low efficiency, necessitating high vector load, both can lead to toxicity issues. To address the ongoing need for engineered capsids, an in vivo direct evolution method: CREATE (Cre-recombination based AAV Targeted Evolution) and its recent predecessor Multiplexed-CREATE (M-CREATE) were developed to address the challenge of developing novels capsids with enhanced ability to target the nervous system such as the PNS and the CNS by crossing the blood-brain barrier (BBB) through systemic delivery. The outcome led to AAV-PHP.B, AAV-PHP.eB, AAV-PHP.S, AAV-PHP.V1 and AAV-PHP.N that are now used for various gene delivery applications in neuroscience.

The first generation of engineered capsids can be further improved on their efficiency and specificity in addition to their translatability across species. Recent research has highlighted the heterogeneity of BBB across species, and hence identifying capsids with enhanced BBB crossing to target the CNS across non-human primates has remained a challenge to date. Similarly, the need for improved capsids remains to be addressed for PNS and other organs of therapeutic interest, such as lung. Years of capsid engineering efforts have now yielded a toolbox of improved CNS capsids for rodents. These include the potent vector AAV-PHP.eB (engineered using CREATE) for the CNS, but its application is restricted to selective mice strains. Unlike PHP.B/eB, the recently engineered AAV-F (engineered using iTransduce) and AAV-PHP.Cs (engineered using Multiplexed-CREATE or M-CREATE) for the CNS work across mouse strains. However, the heterogeneity of the BBB depending on genetic background has posed a significant challenge for developing capsids that have the potential to translate across species. This issue is particularly acute for non-human primates (NHPs), which are commonly used as pre-clinical research models for gene therapy. With several CNS and PNS-based therapies in the pipeline, there is a need for next-generation systemic AAV vectors with potent neurotropic behavior in order to achieve efficient and safe gene delivery for translational applications.

### SUMMARY

Disclosed herein include adeno-associated virus (AAV) targeting peptides. In some embodiments, the AAV targeting peptide comprises an amino acid sequence that comprises at least 4 contiguous amino acids from a sequence selected from PHEGSSR (SEQ ID NO: 169), LNNTKTT (SEQ ID NO: 237), SNLARNV (SEQ ID NO: 274) and TNNTKPL (SEQ ID NO: 390). For example, the targeting peptide can comprise at least 5 contiguous amino acids from the sequence of PHEGSSR (SEQ ID NO: 169), at least 6 contiguous amino acids from the sequence of PHEGSSR (SEQ ID NO: 169), or PHEGSSR (SEQ ID NO: 169). In some embodiments, the targeting peptide comprises at least 5 contiguous amino acids from the sequence of LNNTKTT (SEQ ID NO: 237). For example, the targeting peptide can comprise at least 6 contiguous amino acids from the sequence of LNNTKTT (SEQ ID NO: 237) (e.g., LNNTKTT (SEQ ID NO: 237)). In some embodiments, the targeting peptide comprises at least 5 contiguous amino acids from the sequence of SNLARNV (SEQ ID NO: 274). For example, the targeting peptide can comprise at least 6 contiguous amino acids from the sequence of SNLARNV (SEQ ID NO: 274) (e.g., SNLARNV (SEQ ID NO: 274)). In some embodiments, the targeting peptide comprises at least 5 contiguous amino acids from the sequence of TNNTKPL (SEQ ID NO: 390). In some embodiments, the targeting peptide comprises at least 6 contiguous amino acids from the sequence of TNNTKPL (SEQ ID NO: 390). In some embodiments, the targeting peptide comprises TNNTKPL (SEQ ID NO: 390).

In some embodiments, the AAV targeting peptide comprises an amino acid sequence of 7 contiguous amino acids X₁X₂X₃X₄X₅X₆X₇, wherein X₁ is G; X₂ is N; X₃ is A, C, D, E, F, G, H, I, K, L, M, P, Q, S, T, V, W, or Y; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is R; and X₇ is A, C, D, E, F, G, H, I, K, L, M, N, P, Q, R, S, T, V, W, or Y.

In some embodiments, the AAV targeting peptide comprises an amino acid sequence of 7 contiguous amino acids X₁X₂X₃X₄X₅X₆X₇, wherein X₁ is A, C, D, E, F, G, H, I, K, L, M, N, P, Q, R, T, V, W, or Y; X₂ is N; X₃ is R; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is A, C, D, E F, G, H, I, K, L, M, N, P, Q, R, S, T, V, W, or Y; and X₇ is T.

In some embodiments, the AAV targeting peptide comprises an amino acid sequence of 7 contiguous amino acids X₁X₂X₃X₄X₅X₆X₇, wherein X₁ is A, C, D, E, F, G, H, I, K, L, M, N, P, Q, R, S, T, V, W, or Y; X₂ is N; X₃ is R; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is D; and X₇ is A, C, D, E, F, G, H, I, K, L, M, N, P, Q, R, S, T, V, W, or Y.

In some embodiments, the AAV targeting peptide comprises an amino acid sequence of 7 contiguous amino acids X₁X₂X₃X₄X₅X₆X₇, wherein X₁ is S; X₂ is N; X₃ is R; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is A, C, D, E, F, G, H, I, K, L, M, N, P, Q, R, S, T, V, W, or Y; and X₇ is A, C, D, E, F, G, H, I, K, L, M, N, P, Q, R, S, T, V, W, or Y.

In some embodiments, the AAV targeting peptide comprises an amino acid sequence of 7 contiguous amino acids X₁X₂X₃X₄X₅X₆X₇, wherein X₁ is K, L, S, H, I, Y, or N; X₂ is N; X₃ is R; X₄ is K, A, T, D, M, H, or V; X₅ is A, R, I, M, D, Y, or K; X₆ is D; and X₇ is A, G, R, S, V, D, or M.

In some embodiments, the AAV targeting peptide comprises an amino acid sequence of 7 contiguous amino acids X₁X₂X₃X₄X₅X₆X₇, wherein X₁ is S; X₂ is N; X₃ is R; X₄ is R, V, P, T, E, F, or I; X₅ is A, T, P, G, S, R, or V; X₆ is F, H, P, I, L, T, or D; and X₇ is A, E, I, Q, Y, V, or M.

In some embodiments, the AAV targeting peptide comprises an amino acid sequence of 7 contiguous amino acids X₁X₂X₃X₄X₅X₆X₇, wherein X₁ is L, M, A, K, G, T, or E; X₂ is N; X₃ is R; X₄ is S, I, Q, N, A, G, or Y; X₅ is V, K, S, E, D, A, or N; X₆ is V, N, P, S, R, Q, or Y; and X₇ is T.

In some embodiments, the AAV targeting peptide comprises an amino acid sequence of 7 contiguous amino acids X₁X₂X₃X₄X₅X₆X₇, wherein X₁ is G; X₂ is N; X₃ is Q, Y, L, T, A, I, or G; X₄ is T, D, H, P, A, K, or Y; X₅ is P, A, I, E, N, S, or Q; X₆ is R; and X₇ is K, G, M, S, A, P, or H.

The targeting peptide disclosed herein can be a central nervous system (CNS) targeting peptide, a peripheral nervous system (PNS) targeting peptide, and/or a lung targeting peptide. In some embodiments, the targeting AAV peptide is part of an AAV. In some embodiments, the targeting peptide is part of a capsid protein of the AAV. In some embodiments, the targeting peptide is conjugated to a nanoparticle, a second molecule, a viral capsid protein, or a combination thereof.

Disclosed herein include AAV capsid proteins comprising an AAV targeting peptide provided herein. In some embodiments, the AAV capsid is derived from AAV9, or a variant thereof. In some embodiments, the AAV capsid is derived from AAV1, AAV2, AAV3, AAV3b, AAV4, AAV5, AAV6, AAV7, AAV8, AAV9, AAV10, human isolate hu.31, human isolate hu.32, rhesus isolate rh.8, or rhesus isolate rh.10.

Disclosed herein include nucleic acids. In some embodiments, the nucleic acid comprises a sequence encoding the AAV targeting peptide provided herein. In some embodiments, the nucleic acid comprises a sequence encoding the AAV capsid protein provided herein. Disclosed herein include recombinant adeno-associated viruses (rAAVs). In some embodiments, the rAAV comprises an AAV targeting peptide provided herein, or an AAV capsid protein provided herein.

Disclosed herein include rAAVs comprising an AAV capsid protein which comprises an AAV targeting peptide provided herein. In some embodiments, the AAV capsid protein comprises the AAV targeting peptide provided herein inserted between two adjacent amino acids in AA586-592 or functional equivalents thereof of the AAV capsid protein. In some embodiments, the two adjacent amino acids are AA588 and AA589. In some embodiments, the AAV capsid protein comprises, or consists thereof, SEQ ID NO: 391. In some embodiments, the rAAV comprises an rAAV vector genome.

Disclosed herein include compositions comprising an AAV targeting peptide provided herein, an AAV capsid protein provided herein, a nucleic acid provided herein, an rAAV provided herein, or a combination thereof. In some embodiments, the composition is a pharmaceutical composition comprising one or more pharmaceutically acceptable carriers.

Disclosed herein include compositions for use in the delivery of an agent to a target environment of a subject in need. The composition can comprise an AAV comprising (1) an AAV capsid protein provided herein and (2) an agent to be delivered to the target environment of the subject. The target environment can be the nervous system, the lung or a combination thereof. The nervous system can be the central nervous system (CNS), the PNS, or a combination thereof. In some embodiments, the target environment is a neuron, a glial cell, an oligodendrocyte, an ependymal cell, an astrocyte, a Schwann cell, a satellite cell, or an enteric glial cell in the CNS, a neuron or an astrocyte in the PNS, an endothelial cell in the lung, or a combination thereof. In some embodiments, the agent is delivered to neural tissue in the CNS, ganglia or nerve fibre in the PNS, or epithelial lining of the lung or a combination thereof of the subject.

The composition can, for example, comprise an AAV comprising (1) an AAV capsid protein comprising an AAV targeting peptide and (2) an agent to be delivered to the target environment of the subject. In some embodiments, the AAV targeting peptide comprises at least 4 contiguous amino acids from the sequence of PNASVNS (SEQ ID NO: 387). For example, the targeting peptide can comprise the sequence of PNAS (SEQ ID NO: 428), NASV (SEQ ID NO: 429), ASVN (SEQ ID NO: 430), or SVNS (SEQ ID NO: 431). In some embodiments, the AAV targeting peptide comprises at least 5 contiguous amino acids from the sequence of PNASVNS (SEQ ID NO: 387). For example, the targeting peptide can comprise the sequence of PNASV (SEQ ID NO: 432), NASVN (SEQ ID NO: 433), or ASVNS (SEQ ID NO: 434). In some embodiments, the AAV targeting peptide comprises at least 6 contiguous amino acids from the sequence of PNASVNS (SEQ ID NO: 387). For example, the targeting peptide can comprise the sequence of PNASVN (SEQ ID NO: 435), or NASVNS (SEQ ID NO: 436). In some embodiments, the AAV targeting peptide comprises the sequence of PNASVNS (SEQ ID NO: 387).

The target environment can be the PNS. For example, the target environment can be a neuron and/or a glial cell in the PNS. In some embodiments, the glial cell is an astrocyte. In some embodiments, the agent is delivered to ganglia or nerve fibre in the PNS, or a combination thereof of the subject. In some embodiments, the agent is delivered to dorsal root ganglia, nodose ganglia, enteric ganglia in the PNS, or a combination thereof of the subject.

In some embodiments, the composition comprises an AAV comprising (1) an AAV capsid protein comprising an AAV targeting peptide and (2) an agent to be delivered to the target environment of the subject. In some embodiments, the AAV targeting peptide comprises at least 4 contiguous amino acids from the sequence of LNTIRNV (SEQ ID NO: 388). For example, the targeting peptide can comprise the sequence of LNTI (SEQ ID NO: 437), NTIR (SEQ ID NO: 438), TIRN (SEQ ID NO: 439), or IRNV (SEQ ID NO: 440). In some embodiments, the AAV targeting peptide comprises at least 5 contiguous amino acids from the sequence of LNTIRNV (SEQ ID NO: 388). For example, the targeting peptide can comprise the sequence of LNTIR (SEQ ID NO: 441), NTIRN (SEQ ID NO: 442), or TIRNV (SEQ ID NO: 443). In some embodiments, the AAV targeting peptide comprises at least 6 contiguous amino acids from the sequence of LNTIRNV (SEQ ID NO: 388). For example, the targeting peptide can comprise the sequence of LNTIRN (SEQ ID NO: 444), or NTIRNV (SEQ ID NO: 445). In some embodiments, the AAV targeting peptide comprises the sequence of LNTIRNV (SEQ ID NO: 388).

The target environment can be the lung. In some embodiments, the target environment is an alveolar cell. In some embodiments, the composition is a pharmaceutical composition comprising one or more pharmaceutically acceptable carriers. In some embodiments, the agent to be delivered comprises a nucleic acid, a peptide, a small molecule, an aptamer, or a combination thereof. In some embodiments, the nucleic acid comprises one or more of: a) a DNA sequence that encodes a trophic factor, a growth factor, or a soluble protein; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

The subject in need can be a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, lysosomal storage disorders that involve cells within the CNS, bronchiectasis, bronchitis, bronchopulmonary dysplasia, interstitial lung disease, occupational lung disease, cystic fibrosis, acute respiratory distress syndrome (ARDS), severe acute respiratory syndrome (SARS), intermittent asthma, mild persistent asthma, moderate persistent asthma, severe persistent asthma, chronic bronchitis, chronic obstructive pulmonary disease (COPD), interstitial lung disease, sarcoidosis, asbestosis, aspergilloma, aspergillosis, lobar pneumonia, multilobar pneumonia, bronchial pneumonia, interstitial pneumonia, pulmonary fibrosis, pulmonary tuberculosis, rheumatoid lung disease, pulmonary embolism, and non-small-cell lung carcinoma, adenocarcinoma, squamous-cell lung carcinoma, large-cell lung carcinoma, or small-cell lung carcinoma. In some embodiments, the lysosomal storage disorder is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease. In some embodiments, the subject in need is a subject suffering from, at risk to develop, or has suffered from a stroke, traumatic brain injury, epilepsy, spinal cord injury, emphysema, lung reperfusion injury, ischemia-reperfusion injury of the lung, or ventilator-induced lung injury. The composition can be for intravenous administration and/or systemic administration. In some embodiments, the subject is an adult animal.

Disclosed herein include methods of delivering an agent to a nervous system or a lung of a subject. In some embodiments, the method comprises: providing an AAV vector comprising an AAV capsid protein provided herein; and administering the AAV vector to the subject. In some embodiments, the AAV vector further comprises an agent to be delivered to the nervous system, the lung, or a combination thereof. In some embodiments, the agent is delivered to the nervous system or the lung of the subject at least 1.5-fold, 2-fold, 3-fold, 4-fold, 5-fold, 6-fold, 7-fold, 8-fold, 9-fold, or 10-fold more efficiently than the delivery of the agent to other organs or tissues. In some embodiments, the agent is delivered to the nervous system or the lung of the subject with an enrichment score relative to other organs or tissues of at least 0.01, 0.05, 0.1, 0.15, 0.2, 0.25, 0.3, 0.4, 0.45, 0.5, 0.6, 0.7, 0.8, 0.9, 1, 1.5, or 2. In some embodiments, the agent is delivered to the nervous system or lung of the subject at least 1.5-fold, 2-fold, 5-fold, 10-fold, 20-fold, 30-fold, 40-fold, 50-fold, 100-fold, 150-fold, or 200-fold more efficiently than the agent is delivered to the nervous system or lung of the subject by an AAV vector that does not comprise the targeting peptide. In some embodiments, the nervous system is the CNS and/or the PNS. In some embodiments, the agent is delivered to a neuron or an astrocyte of the nervous system of the subject at least 1.5-fold, 2-fold, or 3-fold more efficiently than the delivery of the agent to other cells of the organs in which the PNS is embedded. In some embodiments, the agent is delivered to a neuron or an astrocyte of the nervous system of the subject with an enrichment score relative to other cells of the organs in which the PNS is embedded of at least 0.01, 0.05, 0.1, 0.15, 0.2, 0.25, 0.3, 0.4, 0.45, 0.5, 0.6, 0.7, 0.8, 0.9, 1, 1.5, or 2. In some embodiments, the agent is delivered to a neuron or an astrocyte of the nervous system of the subject at least 1.5-fold, 2-fold, 5-fold, 10-fold, 20-fold, 30-fold, 40-fold, 50-fold, 100-fold, 150-fold, or 200-fold more efficiently than the agent is delivered to a neuron or an astrocyte of the nervous system of the subject by an AAV vector that does not comprise the targeting peptide. In some embodiments, the agent is delivered to the PNS in the gastrointestinal tract of the subject at least 1.5-fold, 2-fold, or 3-fold more efficiently than the delivery of the agent to the nervous system or the lung than other organs or tissues. In some embodiments, the agent is delivered to the PNS in the gastrointestinal tract of the subject with an enrichment score relative to other organs or tissues of at least 0.01, 0.05, 0.1, 0.15, 0.2, 0.25, 0.3, 0.4, 0.45, 0.5, 0.6, 0.7, 0.8, 0.9, 1, 1.5, or 2. In some embodiments, the agent is delivered to the PNS in the gastrointestinal tract of the subject at least 1.5-fold, 2-fold, 5-fold, 10-fold, 20-fold, 30-fold, 40-fold, 50-fold, 100-fold, 150-fold, or 200-fold more efficiently than the agent is delivered to the PNS in the gastrointestinal tract of the subject by an AAV vector that does not comprise the targeting peptide.

Disclosed herein include methods of delivering an agent to a PNS of a subject. In some embodiments, the method comprises: providing an AAV vector comprising an AAV capsid protein, and administering the AAV vector to the subject. In some embodiments, the AAV capsid protein comprises an AAV targeting peptide that comprises at least 4 contiguous amino acids from the sequence of PNASVNS (SEQ ID NO: 387). In some embodiments, the AAV vector further comprises an agent to be delivered to the PNS. In some embodiments, the agent is delivered to a neuron or an astrocyte of the PNS of the subject at least 1.5-fold, 2-fold, or 3-fold more efficiently than the delivery of the agent to the cells other than the neurons and the astrocytes in the organs in which the PNS is embedded. In some embodiments, the agent is delivered to a neuron or an astrocyte of the PNS of the subject with an enrichment score relative to cells other than the neurons and the astrocytes in the organs in which the PNS is embedded of at least 0.01, 0.05, 0.1, 0.15, 0.2, 0.25, 0.3, 0.4, 0.45, 0.5, 0.6, 0.7, 0.8, 0.9, 1, 1.5, or 2. In some embodiments, the agent is delivered to the PNS in the gastrointestinal tract of the subject at least 1.5-fold, 2-fold, or 3-fold more efficiently than the delivery of the agent to the nervous system or the lung than other organs or tissues. In some embodiments, the agent is delivered to the PNS in the gastrointestinal tract of the subject with an enrichment score relative to other organs or tissues of at least 0.01, 0.05, 0.1, 0.15, 0.2, 0.25, 0.3, 0.4, 0.45, 0.5, 0.6, 0.7, 0.8, 0.9, 1, 1.5, or 2. In some embodiments, the agent is delivered to the PNS in the gastrointestinal tract of the subject at least 1.5-fold, 2-fold, 5-fold, 10-fold, 20-fold, 30-fold, 40-fold, 50-fold, 100-fold, 150-fold, or 200-fold more efficiently than the agent is delivered to the PNS in the gastrointestinal tract of the subject by an AAV vector that does not comprise the targeting peptide.

Disclosed herein include methods of delivering an agent to a lung of a subject. The method can comprise: providing an AAV vector comprising an AAV capsid protein; and administering the AAV vector to the subject. The AAV capsid protein can comprise an AAV targeting peptide that comprises at least 4 contiguous amino acids from the sequence of LNTIRNV (SEQ ID NO: 388). In some embodiments, the AAV vector further comprises an agent to be delivered to the lung. In some embodiments, the agent is delivered to alveolar cells in the lung of the subject at least 1.5-fold, 2-fold, 3-fold, 4-fold, 5-fold, 6-fold, 7-fold, 8-fold, 9-fold, or 10-fold more efficiently than the delivery of the agent to any cells in other organs or tissues. In some embodiments, the agent is delivered to the lung of the subject with an enrichment score relative to other organs or tissues of at least 0.01, 0.05, 0.1, 0.15, 0.2, 0.25, 0.3, 0.4, 0.45, 0.5, 0.6, 0.7, 0.8, 0.9, 1, 1.5, or 2 higher than the enrichment score in any cells in other organs or tissues. In some embodiments, the agent is delivered to the lung of the subject at least 1.5-fold, 2-fold, 5-fold, or 10-fold, more efficiently than the agent is delivered to the lung of the subject by an AAV vector that does not comprise the targeting peptide. The subject can be a primate. The administration can be a systemic administration and/or an intravenous administration.

Disclosed herein include methods of delivering an agent to a cell. The method can comprise: contacting an AAV vector comprising an AAV capsid protein provided herein with the cell. In some embodiments, the AAV vector further comprises an agent to be delivered to a nervous system, a lung, or a combination thereof. In some embodiments, the cell is a neuron, or an astrocyte in the nervous system, any cell in the lung or a combination thereof. The method can comprise: contacting an AAV vector comprising an AAV capsid protein with the cell. In some embodiments, the AAV capsid protein comprises an AAV targeting peptide that comprises at least 4 contiguous amino acids from the sequence of PNASVNS (SEQ ID NO: 387). In some embodiments, the AAV vector further comprises an agent to be delivered to a PNS. In some embodiments, the cell is a neuron, an astrocyte, or a combination thereof. The method can comprise: contacting an AAV vector comprising an AAV capsid protein with the cell. In some embodiments, the AAV capsid protein comprises an AAV targeting peptide that comprises at least 4 contiguous amino acids from the sequence of LNTIRNV (SEQ ID NO: 388). In some embodiments, the AAV vector further comprises an agent to be delivered to a lung. In some embodiments, the cell is any cell in the lung. In some embodiments, contacting the AAV vector with the cell occurs in vitro, in vivo or ex vivo. In some embodiments, the cell is present in a tissue, an organ, or a subject. In some embodiments, the agent to be delivered comprises a nucleic acid, a peptide, a small molecule, an aptamer or a combination thereof. In some embodiments, the nucleic acid encodes a therapeutic protein. In some embodiments, the nucleic acid comprises one or more of: a) a DNA sequence that encodes a trophic factor, a growth factor, or other soluble factors capable of being released from the transduced cells and affect the survival or function of that cell and/or surrounding cells; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene. The AAV vector can be an AAV9 vector, or a variant thereof. In some embodiments, the AAV vector is a vector derived from AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof. In some embodiments, the serotype of the AAV vector is different from the serotype of the AAV capsid.

### BRIEF DESCRIPTION OF THE DRAWINGS

FIG. 1A-FIG. 1F depict non-limiting exemplary embodiments and data related to multiplexed-CREATE selection for AAV capsids targeting the nervous system across species. FIG. 1A shows an overview of the capsid selection method, Multiplexed-CREATE, and characterization of selected capsids across species. The first panel of FIG. 1A depicts the evolution of the AAV9 capsid (PDB 3UX1) with a zoom-in (area in the square box) of a three-fold axis and the 7-mer-i library insertion site between residues 588-589 marked by “*”. The diagrams in the bottom half of the first panel of FIG. 1A depicts the arrangement of the acceptor vector in the absence (top) or presence (bottom) of Cre, with the corresponding orientations of the forward/reverse primers used for Cre+ selective recovery. The remaining panels of FIG. 1A depict the selection workflow involving four key steps: (1) generation of capsid library and intravenous (IV) delivery into transgenic mouse lines where Cre is restricted to cell types of interest (SNAP-Cre, GFAP-Cre, Tek-Cre, n=2 mice per Cre line). (2) Two weeks post-injection, viral DNA is recovered across cell-types/tissues using Cre-dependent PCR and Illumina next-generation sequencing (NGS), and (3) fed into synthetic pool library production. (4) This library then goes through a second round of in vivo selection. Following this selection process, identified variants are validated for (5) virus production and (6) in vivo transduction across species. FIG. 1B depicts heatmaps of capsid variants' mean enrichment by Cre-dependent recovery across tissues of interest and Cre-independent recovery across off-targets after two rounds of selection. Cre lines are plotted separately (the first chart in FIG. 1B, n=3 mice per organ) or grouped by organs (FIG. 1B). The y-axis represents capsids unique at the amino acid (AA) level, ranked by ‘neuron mean’, which is the mean of the enrichment of all targets of interest. FIG. 1C depicts UMAP cluster representation of ˜9,000 variants that were recovered after two rounds of selection (UMAP parameters: n_neighbors=15, min_dist=0.1, n_components=2, random_state=42, metric=‘correlation’, verbose=3). Three separable clusters are shown along with the positions of known capsids (AAV9, PHP.S, PHP.B, PHP.B4, PHP.C1) and new capsids (MaCPNS1 and MaCPNS2). Heatmaps (FIG. 1C) show enrichment of representative capsids from Clusters-1 and -2 across organs. FIG. 1D depicts heatmap of enrichment fold changes against parental AAV9 across organs. FIG. 1E depicts heatmap of enrichment fold changes against parental AAV9 of variants MaCPNS1 and MaCPNS2 and previously-engineered variant PHP.S across organs. FIG. 1F depicts identity of hepta-AA peptides inserted between positions 588-589 of AAV9 for PHP.S, MaCPNS1 and MaCPNS2 capsids.

FIG. 2A-FIG. 2K depict non-limiting exemplary embodiments and data showing engineered AAVs can efficiently target the peripheral nervous system in mice following systemic delivery. FIG. 2A shows an illustration demonstrating the IV administration of AAV capsids packaged with ssAAV:CAG-2xNLS-eGFP and ssAAV:hSyn-tdTomato genome in a mouse model (˜8 week-old young C57BL/6J adults). FIG. 2B depicts (Top) an illustration of the nodose ganglia (NG) (Left), and representative images of AAV9, PHP.S, MaCPNS1 and MaCPNS2 vector-mediated expression of nuclear-localized (NLS) eGFP (lighter puncta) in NG (Right). (Bottom) An illustration of the dorsal root ganglia (DRG) in the spinal cord (left) and (right) representative images of AAV vector-mediated expression of NLS-eGFP (lighter puncta) in DRG across segment T2 (Thoracic) of the spinal cord (n≥4 per group, ˜8 week-old C57BL/6J males, 3×10¹¹ vg IV dose per mouse, 3 weeks of expression). Cloudy fluorescent areas: αNeuN antibody staining for neurons. Images are matched in fluorescence intensity to the respective AAV9 control. Scale bar: 200 μm. FIG. 2C depicts the percentage of AAV-mediated eGFP expression overlapping with the αNeuN marker in NG. One-way ANOVA non-parametric Kruskal-Wallis test (exact P=0.0003), and follow-up multiple comparisons with uncorrected Dunn's test are reported (P=0.0499 for AAV9 versus MaCPNS1, P=0.0251 for AAV9 versus MaCPNS2, P=0.0094 for PHP.S versus MaCPNS1, P=0.0038 for PHP.S versus MaCPNS2). *P≤0.05, **P≤0.01 are shown, P>0.05 is not shown; n≥4 per group, same experimental parameters as FIG. 2B. Each data point represents 1-2 nodose ganglia per mouse comprising >700 cells, mean±s.e.m is plotted. FIG. 2D depicts the percentage of eGFP expression overlapping with the αNeuN marker in DRG (the first chart in FIG. 2D) where each data point shows the mean per mouse across select DRGs within thoracic and lumbar segments of the spinal cord. A one-way ANOVA, non-parametric Kruskal-Wallis test (exact P=0.0005), and follow-up multiple comparisons with uncorrected Dunn's test are reported (P=0.0018 for PHP.S versus MaCPNS1, P=0.0087 for PHP.S versus MaCPNS2). *P≤0.05, **P≤0.01 are shown, P>0.05 is not shown; n≥4 per group, same experimental parameters as FIG. 2B. Each data point shows mean±s.e.m of DRGs across different areas of each mouse, comprising a mean of 1-2 DRGs per area with >200 αNeuN+ cells per DRG. (FIG. 2D (Continued)) Percentage of eGFP expression overlapping with the αNeuN marker in DRG across spinal cord areas in individual mice. A two-way ANOVA and Tukey's multiple comparisons tests with adjusted P values are reported (*P≤0.05, **P≤0.01 and ***P≤0.001 are shown, P>0.05 is not shown). Each data point shows the mean±s.e.m of 1-2 DRG per mouse comprising >200 αNeuN+ cells per DRG. FIG. 2E depicts an illustration of the adult mouse gastrointestinal (GI) tract, highlighting the enteric ganglia (zoom-in) that are spread across the different segments of the GI tract. FIG. 2F depicts the percentage of cells expressing NLS-eGFP delivered by AAV vectors in the myenteric plexus across the GI tract: stomach, duodenum, jejunum, ileum, proximal colon and cecum (ssAAV:CAG-2xNLS-eGFP genome, n≥5 per group, ˜8 weeks old C57BL/6J males, 3×10¹¹ vg IV dose per mouse, 3 weeks of expression). A one-way ANOVA non-parametric Kruskal-Wallis test (approximate P=0.2985), and follow-up multiple comparisons using uncorrected Dunn's test are reported (individual P>0.05, n.s.). Each data point shows the mean±s.e.m of >100 enteric ganglia per intestinal segment per mouse). FIG. 2G. depicts the percentage of cells expressing NLS-eGFP (lighter dots in FIG. 2H) in the myenteric plexus of small intestinal segments: duodenum, jejunum and ileum delivered by AAV vectors: AAV9, PHP.S and MaCPNS2 (ssAAV:CAG-2xNLS-eGFP genome, n=3 per group, 1×10¹¹ vg IV dose per mouse, 3 weeks of expression). Two-way ANOVA, Tukey's multiple comparisons tests with adjusted P values are reported (*P≤0.05, **P≤0.01 and ***P≤0.001 are shown, P>0.05 is not shown). Each data point shows the mean±s.e.m of ≥2 images per mouse comprising >100 enteric ganglia. FIG. 2H shows representative images of AAV-mediated eGFP expression across the individual intestinal segments analyzed in FIG. 2G. Scale bar: 50 μm. The tissues were co-stained with αS100β (cloudy fluorescent areas) antibody for glia and αPGP9.5 (darker puncta) for neurons. The images are matched in fluorescence intensity to the respective AAV9 control. FIG. 2I depicts MaCPNS2 vector-mediated expression of tdTomato from ssAAV:hSyn-tdTomato in the proximal and distal segments of the colon at three different IV doses per mouse: 7×10¹¹ vg, 5×10¹¹ vg, 3×10¹¹ vg (3 weeks of expression, n=3 per group, scale bar: 200 μm, dotted white box inset represents a zoomed-in view of the indicated area in each image). Images in the distal and proximal colon are matched in fluorescence intensity. FIG. 2J depicts representative images of AAV vector-mediated expression of NLS-eGFP (light puncta) from ssAAV:CAG-2xNLS-eGFP in the liver (n=6 per group, 3×10¹¹ vg IV dose/mouse, 3 weeks of expression, darker staining: DAPI staining of nuclei, scale bar: 1 mm (left, full cross-sectional image), and 50 μm (right, higher magnification view). The images are matched in fluorescence intensity to the respective AAV9 control. FIG. 2K depicts the percentage of eGFP+ cells overlapping with the DAPI marker in the liver. A non-parametric Kruskal-Wallis test (approximate P=0.0168), and follow-up multiple comparisons with uncorrected Dunn's test (P=0.0035 for AAV9 versus MaCPNS1 and P=0.0189 for PHP.S versus MaCPNS1) are reported (*P≤0.05, **P≤0.01 are shown, P>0.05 is not shown; same experimental parameters as FIG. 2J, each data point shows the mean±s.e.m of 4 images per mouse comprising >780 DAPI+ cells per image). FIG. 2L depicts mean brightness of the eGFP+ cells quantified in FIG. 2K. A non-parametric Kruskal-Wallis test (approximate P=0.1698), and follow-up multiple comparisons with uncorrected Dunn's test (P=0.0433 for AAV9 versus MaCPNS1) is reported (*P≤0.05 is shown, P>0.05 is not shown; each data point shows the mean±s.e.m of 4 images per mouse comprising >780 DAPI+ cells per image).

FIG. 3A-FIG. 3G depict non-limiting exemplary embodiments and data related to the systemic delivery of GCaMP sensor and excitatory DREADD actuator using AAV-MaCPNS1 to enable functional characterization of DRG neurons in mouse models. FIG. 3A shows an illustration of IV administration of MaCPNS1 capsid packaged with ssAAV:CAG-jGCaMP8s genome in mice (˜8 week-old young adults, C57BL/6J males, 1×10¹² vg IV dose/mouse, n=4). Three weeks post-expression, the mice were anesthetized and subjected to in vivo calcium imaging in nodose ganglia (NG, top zoom-in), and glucose infusion and distension in the gut (bottom zoom-in). FIG. 3B depicts representative images of MaCPNS1 vector-mediated expression of jGCaMP8s in NG in vivo (left), and in post-hoc fixed tissue (right) (scale bar: 100 μm). FIG. 3C depicts single-cell activity response measured by calcium signal dynamics in the NG (data pooled from 4 experimental mice). Left panel: NG neuronal response to glucose infusion (white dotted line). Right panel: NG neuronal response to gut distension (white dotted line). FIG. 3D depicts an illustration of the pain induction experimental workflow. MaCPNS1 vector with ssAAV:hSyn-DIO-hM3D(Gq)-mCherry was intraperitoneally administered to a TrpV1-Cre mouse model (postnatal stage 1 (P1), males, 3×10¹¹ vg IV dose/mouse). After six weeks of expression, the mice (AAV injected or untreated) were habituated and subjected to intraplantar injection with the agonist CNO, after which the mice were monitored for nocifensive lifting/licking behaviors. FIG. 3E depicts representative images of DRG sections showing MaCPNS1 vector-mediated mCherry (intermediate fluorescence matching that in the panel labeled as mCherry) expression. The tissues were co-stained with αNF200 (lighter fluorescence matching that in the panel labeled as NF200), αTuj1 (dark fluorescence matching that in the panel labeled as Tuj1) and αCGRP (light fluorescence matching that in the panel labeled as CGRP) markers. FIG. 3F depicts total bouts of, and FIG. 3G total time spent lifting or licking the footpad within 15 minutes of injection in uninfected (n=6) and MaCPNS1-infected (n=10) mice treated with CNO. **P≤0.01 by unpaired t-test. Mean+/−sem are shown.

FIG. 4A-FIG. 4G depict non-limiting exemplary embodiments and data showing engineered AAVs can efficiently target the peripheral nervous system in adult rats following systemic administration. FIG. 4A depicts an illustration of IV administration of AAV capsids MaCPNS1 and MaCPNS2 packaged with ssAAV:hSyn-tdTomato genome in a rat model (young adults, Sprague Dawley, male, 2×10¹³ vg/kg per rat). The rat tissues were stained with αDsRed antibody against tdTomato. FIG. 4B depicts representative images of MaCPNS1 vector-mediated tdTomato expression in major pelvic ganglia (left), sympathetic chain ganglia (middle) and inferior mesenteric ganglia (right) in adult rats 3 weeks post expression (n>2 per group, scale bar: 100 μm). FIG. 4C depicts representative images of MaCPNS1 vector-mediated tdTomato (intermediate fluorescence matching that in the panel labeled as hSyn:tdT) expression in DRG and TG as indicated by labels. The tissues were co-stained with either αNF200 (light fluorescence matching that in the panel labeled as NF200), αCGRP (light fluorescent color matching that in the panel labeled as CGRP), or αTRPV1 (darker fluorescence matching that in the panel labeled as TRPV1) markers (scale bars: 100 μm). FIG. 4D depicts quantification of proportion of AAV-mediated tdTomato expressing cells that overlap with αNF200, αCGRP and αTRPV1 markers in TG (top) and DRG (bottom), and FIG. 4E depicts proportion of αNF200 marker+ cells that overlap with the AAV-mediated tdTomato expressing cells in DRG and TG (n≥2 per group, each data point represents the average of at least 3 images from each rat, mean±s.e.m is plotted for n>2, mean is plotted for n=2). FIG. 4F depicts representative images of MaCPNS1 and MaCPNS2 vector-mediated tdTomato expression across different segments of the GI tract: jejunum, ileum, proximal colon and distal colon (scale bar: 200 μm). FIG. 4G depicts representative images of MaCPNS1 vector-mediated tdTomato expression in the spinal cord (top, scale bar: 500 μm) with zoomed-in views of selected areas (white boxes, right, scale bar: 100 μm), and hindbrain (bottom, scale bar: 2 mm) with zoomed-in view of Sp5O region (scale bar: 100 μm). The tissues were co-stained with αNeuN (dark fluorescence matching that in the panel labeled as NeuN) antibodies.

FIG. 5A-FIG. 5F depict non-limiting exemplary embodiments and data showing engineered AAVs can efficiently transduce the central and peripheral nervous system in marmoset. FIG. 5A depicts an illustration of AAV vector delivery to adult marmoset to study transduction across the CNS and PNS after 3 weeks of expression. The capsids (AAV9/MaCPNS1/MaCPNS2) and their corresponding genomes (ssAAV:CAG-eGFP/tdTomato) are shown on the left. Two AAV vectors packaged with colored fluorescent reporters were mixed and intravenously delivered at a total dose of 7×10¹³ vg/kg per adult marmoset (16-month-old Callithrix jacchus, i.e. 3.5×10¹³ vg/kg per AAV). Representative images of marmoset FIG. 5B depicts DRGs (scale bar: 200 μm, left and 50 μm, right), FIG. 5C depicts small intestine (scale bar: 500 μm, left and 100 μm, right), FIG. 5D depicts fibers in the dorsal column of the spinal cord (scale bar: 500 μm), FIG. 5E depicts coronal brain sections of the midbrain (left) and hindbrain (right) (scale bar: 500 μm), and FIG. 5F depicts selective brain areas: cortex, thalamus, globus pallidus, cerebellum and brainstem (scale bar: 400 μm), showing AAV9 vector-mediated expression of eGFP or tdTomato, MaCPNS1-mediated expression of eGFP and MaCPNS2-mediated expression of tdTomato. The images are matched in fluorescence intensity to the respective AAV9 control. Zoomed-in views of selected areas (dotted white boxes) are shown on the right in FIG. 5B and FIG. 5C. In FIG. 5B, the zoomed-in view shows the overlap of MaCPNS1 and MaCPNS2-mediated expression with the neuronal marker Tuj1 (dark fluorescence matching that in the panel labeled as Tuj1).

FIG. 6A-FIG. 6F depict non-limiting exemplary embodiments and data showing engineered variants efficiently target the central and peripheral nervous system in macaque following systemic delivery. FIG. 6A depicts an illustration of AAV vector delivery to macaque to study transduction across the CNS and PNS after 4 weeks of expression. The capsids (AAV9/MaCPNS1/MaCPNS2) and their corresponding genomes (ssAAV:CAG-eGFP/tdTomato) are shown on the left. Two AAVs packaged with different fluorescent proteins were mixed and intravenously injected at a dose of 5×10¹³ vg/kg per macaque (Macaca mulatta, injected within 10 days of birth, female, i.e. 2.5×10¹³ vg/kg per AAV). FIG. 6B depicts representative images of macaque, including GI regions of esophagus (top) and colon (bottom) (scale bar: 500 μm, left and 200 μm, far right), FIG. 6C depicts DRGs (scale bar: 200 μm), FIG. 6D depicts spinal cord (scale bar: 500 μm, top and 200 μm, bottom), FIG. 6E depicts coronal sections of forebrain, hindbrain and cerebellum (scale bar: 2 mm), and FIG. 6F depicts selected brain areas: cortex, hippocampus, putamen, and brainstem (scale bar: 200 μm, top and 500 μm, bottom), showing AAV9 vector-mediated expression of eGFP, MaCPNS1-mediated expression of eGFP and MaCPNS2-mediated expression of tdTomato. The eGFP images of MaCPNS1 are matched in fluorescence intensity to the AAV9 control. The zoomed-in views in FIG. 6B (right panels) show the overlap of MaCPNS2-mediated expression of tdTomato (intermediate fluorescence matching MaCPNS2 panel) with the neuronal marker Tuj1 (lighter fluorescence). The zoomed-in views in FIG. 6D (bottom panels) shows AAV-mediated expression of eGFP (AAV9 and MaCPNS1) and tdTomato (MaCPNS2) in the fibers in the dorsal column (white dashed boxes).

FIG. 7A-FIG. 7G depict non-limiting exemplary embodiments and data related to multiplexed-CREATE selection for AAV capsids targeting lung. FIG. 7A depicts a heatmap of capsid variants' enrichment by Cre-independent recovery across organs post 2 rounds of selection (top panel, n=:3 mice per organ, mean is plotted, y-axis represents capsids unique at amino acid (AA) level that are ranked by enrichment score). FIG. 7B depicts heatmap of enrichment of parent AAV9, variant Lung1 and Lung2, and prior engineered variant PHP.S across cell-types or organs. FIG. 7C depicts a UMAP cluster representation of variants that were recovered post 2 rounds of selection (Umap parameter: n_neighbors=i5, min_dist=0.1, n_conponents=2, random_state=42, metric=‘correlation’, verbose=3). A distinguishable cluster (cluster 1) was circled out by a dotted box. FIG. 7D depicts a heatmap showing enrichment of representative capsids from Cluster 1 across organs. FIG. 7E depicts a heatmap of enrichment of parent AAV9, variant CNSLung2 and CNSLung3, and prior engineered variant PHP.S across cell-types or organs. FIG. 7F depicts the insertion of hepta-AA peptides between AA positions 588-589 of AAV9 for Lung1, Lung2, CNSLung3, CNSLung2 capsids. FIG. 7G depicts top 25 lung-enriched sequence in PNS library with the corresponding AA frequency logos (AA size represents prevalence and color encodes AA properties) (Top panel) and top 25 sequence in UMAP cluster1 in CNS library with the corresponding AA frequency logos (AA size represents prevalence and color encodes AA properties) (Bottom panel).

FIG. 8A-FIG. 8G depict non-limiting exemplary embodiments and data showing engineered AAVs efficiently target lung in mice and marmoset following systemic delivery. FIG. 8A depicts an illustration demonstrating the IV administration of AAV capsids packaged with ssAAV:CAG-2xNLS-eGFP a genome in a mouse model (˜8 weeks old, young adults C57BL/6J). FIG. 8B depicts representative images of AAV9, Lung1, CNSLung2 and CNSLung3 vectors mediated expression of NLS-eGFP in lung (n≥4 per group, ˜8 weeks old C57BL/6J males, 3 weeks of expression). Top panel shows expression with 3×10¹¹ vg IV dose per mouse; bottom panel shows expression with 1×10¹² vg IV dose per mouse. The images are matched in fluorescence intensity to the respective control. FIG. 8C depicts representative images of AAV9, Lung2, CNSLung2 and CNSLung3 vectors mediated expression of NLS-eGFP (light fluorescence, top row) in lung co-stained with cellular marker for AT2 (alveolar type 11 cells), anti-Pro-SPC (intermediate fluorescence, middle row). Bottom row depicts the overlay. FIG. 8D depicts the percentage of eGFP expression that overlapped with the Pro-SPC marker in lung. FIG. 8E depicts an illustration demonstrating the AAV vector delivery to marmoset to study transduction across organs after 3 weeks of expression. Lung1, Lung, CNSLung2, CNSLung3, CAPA4, PNS1, X₁ along with AAV9 and AAV5 as controls, packaging human frataxin (FXN) fused to an HA tag under the control of the ubiquitous CAG promoter, were pooled and intravenously injected into adult marmosets at doses of 7×10¹³ vg/kg (8×10¹² vg/kg per AAV) total FIG. 8F depicts NGS quantification within the lung of the unique DNA or RNA barcode associated with each virus. FIG. 8G depicts NGS quantification within the liver of the unique DNA or RNA barcode associated with each virus.

FIG. 9A-FIG. 9B depict non-limiting exemplary embodiments and data related to AAV capsid library outcome post round-2 in vivo selection. FIG. 9A depicts heatmaps of Cluster 1 (left cluster in FIG. 1C) and Cluster 2 (middle, and right clusters in FIG. 1C) variants' enrichments across different cell-types/organs where the mean enrichment score across areas of interest (horizontal label with lighter font shade) is >0.0 (left and middle clusters in FIG. 1C) and >0.1 (right cluster in FIG. 1C) (log 10 scale). FIG. 9B depicts UMAP embedding of 601 positively enriched variants based on tropism, enrichments of 3 known AAV capsids for CNS (PHP.B, PHP.B4, PHP.C1) and AAV9 are added to the dataset. (Umap parameter: n_neighbors=5, min_dist=0.1, n_components=2, random_state=42, metric=‘correlation’, verbose=3). Positions of known capsids (AAV9, PUPS, PHP.B, PHP.B4, PHP.C1) and new capsids (PHP.PNS1 and PHP.PNS2) are shown.

FIG. 10A-FIG. 10J depict non-limiting exemplary embodiments and data related to characterization of AAV variants across different organs in mice following systemic delivery. Related to FIG. 2A-FIG. 2K. FIG. 10A depicts an illustration of IV administration of AAV9/PHP.S/MaCPNS1 capsid packaged with either ssAAV:CAG-eGFP or ssAAV:CAG-NLS-eGFP genome in mice (˜8 weeks old, C57BL/6J males) at 1×10¹² vg dose/mouse. FIG. 10B depicts vector (AAV9/PHP.S/MaCPNS1)-mediated expression of eGFP (light puncta) in DRG after 3 weeks of expression in vivo (n=3 per group). FIG. 10C depicts vector-mediated expression of NLS-eGFP (light puncta) in DRG after 3 weeks of expression, with imaging parameters matched across samples. Quantification of eGFP+ cells in DRG is shown at right (n=2-3 per group, mean±s.e.m is plotted for n>2, mean is plotted for n=2). FIG. 10D depicts MaCPNS2 vector-mediated expression of tdTomato from ssAAV:hSyn-tdTomato in the proximal and distal segments of the colon at three different IV doses per mouse: 7×10¹¹ vg, 5×10¹¹ vg, 3×10¹¹ vg (3 weeks of expression, n=3 per group, white boxes show zoomed-in views of selected areas). FIG. 10E depicts vector (AAV9, PUPS, MaCPNS1 and MaCPNS2)-mediated expression of NLS-eGFP in organs after 3 weeks of expression. Top panels show expression in heart. Middle panels show expression in the brain with zoomed-in views of the cortex. Bottom panels show expression in the spinal cord. FIG. 10F depicts MaCPNS2 vector-mediated expression of tdTomato (light fluorescence) from ssAAV:hSyn4dTomato in the brain at three different IV doses per mouse: 7×10¹¹ vg, 5×10¹¹ vg, 3×10¹¹ vg (3 weeks of expression, n=3 per group). Bottom panel shows PHP.eB vector-mediated expression of tdTomato using ssAAVhSyn-tdTomato in the brain at an IV dose per mouse of 3×10¹¹ (3 weeks of expression, n=3 per group). The tissues were co-stained with the nuclear stain DAPI (darker fluorescence). Imaging parameters in FIG. 10B-FIG. 10F were matched across samples to the respective control in the experiment or the area. FIG. 10G depicts an illustration of intraperitoneal administration of MaCPNS1 vector packaged with ssAAV:hSyn-tdTomato in a C57BL/6J mouse model (postnatal stage 1 (P1), males, 3×10¹¹ vg IV dose/mouse). After six weeks of expression, the DRG were harvested. FIG. 10H depicts representative images of DRG sections showing MaCPNS1 vector-mediated tdTomato (intermediate fluorescence matching the panel labeled as tdT) expression. The tissues were co-stained with αNF200 (light fluorescence matching the panel labeled as NF200), a-furl (dark fluorescent color matching the panel labeled as Tuj1) and αCGRP (light fluorescence matching the panel labeled as CGRP) markers. FIG. 10I depicts quantification of the proportion of αNF200 and αCGRP marker+ cells that overlap with the MV-mediated tdTomato-expressing cells in DRG. FIG. 10J depicts proportion of AAV-mediated tdTomato-expressing cells that overlap with αTuj1, αNF200 and αCGRP markers in DRG (n=4 per group, unpaired West. Mean+/−sem are shown).

FIG. 11A-FIG. 11F depict non-limiting exemplary embodiments and data showing novel variants transduce peripheral ganglia and CNS in rats. Related to FIG. 3A-FIG. 3G. AAV capsids (MaCPNS1 and MaCPNS2 packaged with ssAAV:hSyn-tdTomato genome) were intravenously administered in a rat model (young adults, Sprague Dawley, male, 2×10¹³ vg/kg per rat). The tissues were stained with αDsRed (light fluorescence) antibody against tdTomato. FIG. 11A depicts representative image of MaCPNS1 vector-mediated tdTomato expression in inferior mesenteric ganglion with inferior mesenteric artery (scale bar, 500 μm). FIG. 11B depicts representative images of MaCPNS2 vector-mediated tdTomato expression in major pelvic ganglia (left), sympathetic chain ganglia (middle) and inferior mesenteric ganglia (right) in adult rats after 3 weeks of expression (n≥2 per group, scale bar: 200 μm). FIG. 11C depicts representative images of MaCPNS1 (left) and MaCPNS2 (right) vector-mediated tdTomato expression in the jejunum and distal colon at the submucosal plexus layer (top) and proximal colon and distal colon at the myenteric plexus layer (bottom). (scale bar: 200 μm). FIG. 11D depicts representative images of MaCPNS1 vector-mediated tdTomato expression in the brain including forebrain (top left, scale bar 2 mm) with zoomed-in view of the cortex (white box, scale bar 100 μm), thalamus (bottom left, scale bar 100 μm) and hippocampus (bottom right, scale bar 200 μm). FIG. 11E depicts representative images of MaCPNS1 and MaCPNS2 vector-mediated tdTomato expression in the liver. The tissues were co-stained with DAPI (dark nuclei) (scale bar: 100 μm). FIG. 11F depicts representative images of vector-mediated tdTomato expression in the forebrain (top left, scale bar: 2 mm) with zoom-in views of cortex (white box, scale bar: 100 μm) and brainstem (top right, scale bar: 1 mm) with zoom-in view (scale bar: 100 pun). The tissues were stained with αDsRed antibodies. Representative images show PNS1 (e.g., MaCPNS1) and PNS2 (e.g., MaCPNS2) vector-mediated tdTomato expression in the forebrain (bottom left, scale bar: 2 mm), brainstem (bottom middle, scale bar: 1 mm) and hindbrain (bottom right, 2 mm).

FIG. 12 depicts non-limiting exemplary embodiments and data showing novel variants efficiently transduce macaque CNS while maintaining similar transduction in liver compared to AAV9. Related to FIG. 6A-FIG. 6F. AAV capsids (AAV9/MaCPNS1/MaCPNS2) and their corresponding genomes (ssAAV:CAG-eGFP/tdTomato) were delivered to macaque to study transduction across the CNS and PNS after 3 weeks of expression. Two AAVs packaged with different fluorescent proteins were mixed and intravenously injected at a dose of 5×10¹³ vg/kg per macaque (Macaca mulatta, female, injected within 10 days of birth, 2.5×10¹³ vg/kg per AAV). Representative images of the brain (left) and liver (right) are shown (scale bars 2 mm).

FIG. 13A-FIG. 13D depict non-limiting exemplary embodiments and data related to cell-type profiles of engineered AAV9 in marmoset brain. Related to FIG. 5A-FIG. 5F. FIG. 13A depicts representative images of AAV9, MaCPNS1 and MaCPNS2 vector-mediated fluorescent protein expression in the marmoset cortex and thalamus (scale bar 50 μm). Slices were co-stained with NeuN (top 2 rows, white) or S100β (bottom 2 rows, white). FIG. 13B, and FIG. 13C depict quantification of the fold change of Fluorescent+/marker over mean AAV9 in cortex and thalamus. Each data point is a slice. FIG. 13D depicts representative images of MaCPNS1 and MaCPNS2 vector-mediated fluorescent protein expression in the marmoset cortex (scale bar 50 μm). Slices were co-stained with Parvalbumin (white).

FIG. 14A-FIG. 14F depict non-limiting exemplary embodiments and data showing that engineered vectors transduce spinal cord and DRG in macaque efficiently. Related to FIG. 6A-FIG. 6F. FIG. 14A depicts representative images of AAV9, MaCPNS1 and MaCPNS2 vector-mediated fluorescent protein expression in the macaque lumbar spinal cord (scale bar 500 μm) with zoomed-in views of each channel (scale bar 100 μm). Slices were co-stained with NeuN (white). FIG. 14B depicts quantification of the fold change of Fluorescent+/marker over mean AAV9 in lumbar spinal cord. Each data point is a slice. FIG. 14C depicts representative images of MaCPNS1 and MaCPNS2 vector-mediated fluorescent protein expression in the macaque thoracic spinal cord and coccygeal spinal cord (scale bar 500 μm). FIG. 14D depicts quantification of the fold change of Fluorescent+/marker over mean AAV9 in thoracic spinal cord. Each data point is a slice. FIG. 14E and FIG. 14F (Top Panel) depict representative images of AAV9, MaCPNS1 and MaCPNS2 vector-mediated fluorescent protein expression in the macaque lumbar DRG and thoracic DRG, respectively (scale bars: 50 μm). Slices were co-stained with Tuj1 (dark fluorescence). FIG. 14E and FIG. 14F (Bottom Panel) show quantification of the fold change of Fluorescent+/marker over mean AAV9 in DRGs and percentage of Tuj1+ within the fluorescent+ population. Each data point is a slice.

FIG. 15A-FIG. 15D depict non-limiting exemplary embodiments and data related to cell-type profiles of engineered AAVs in macaque brain. Related to FIG. 6A-FIG. 6F. FIG. 15A depicts representative images of AAV9, MaCPNS1 and MaCPNS2 vector-mediated fluorescent protein expression in the macaque cortex and thalamus (scale bar 50 μm). Slices were co-stained with NeuN (first row, white), S100β (second row, white), Olig2 (third row, white) or GLUT1 (fourth row, white). FIG. 15B and FIG. 15C depict quantification of the fold change of Fluorescent+/marker over mean AAV9 in cortex and thalamus. Each data point is a slice. FIG. 15D depicts representative images of MaCPNSI and MaCPNS2 vector-mediated fluorescent protein expression in the macaque cortex (scale bar: 50 μm). Slices were co-stained with Parvalbumin (white).

FIG. 16A-FIG. 16B depict non-limiting exemplary embodiments and data related to AAV capsid library outcome post round-2 in vivo selection. Related to FIG. 1A-FIG. 1F. FIG. 16A depicts a heatmap of 6300 capsid variants which showed a bias towards one or more of the neuronal tissues. Heatmap shows mean enrichment by Cre-dependent recovery across tissues of interest (text in lighter color) and Cre-independent recovery across off-targets (text in black) after two rounds of selection. Cre lines are grouped by organs. The y-axis represents capsids unique at the amino acid (AA) level, ranked by ‘neuron mean’, which is the mean of the enrichment of all targets of interest. FIG. 16B depicts AAV vector yields from an established laboratory protocol. One-way analysis of variance (ANOVA) non-parametric Kruskal-Wallis test (approximate P=0.6407, n.s.), and follow-up multiple comparisons with uncorrected Dunn's test are reported (individual P>0.05, n.s.; n≥4 per group, each data point is the mean of 3 technical replicates, mean±s.e.m is plotted).

FIG. 17A-FIG. 17C depict non-limiting exemplary embodiments and data related to multiplexed-CREATE selection for capsids targeting the nervous system across species. FIG. 17A depicts a heatmap of capsid variants' enrichment by Cre-dependent recovery across tissues of interest and Cre-independent recovery across off-targets post 2 rounds of selection (in the first chart of FIG. 17A, n=3 mice per organ, mean is plotted, y-axis represents capsids unique at amino acid (AA) level that are ranked by enrichment score). Capsids with a positive enrichment score in at least one of the Cre-dependent selection (black box) are plotted separately as shown in IG. 17A (Continued). FIG. 17B depicts a U-MAP cluster representation of ˜9000 variants that were recovered post 2 rounds of selection (Umap parameter: n_neighbors=15, min_dist=0.1, n_components=:2, random_state=42, metric=‘correlation’, verbose===3), Two separable clusters: Cluster-1 (dots in the left cluster with the same color as the label “Cluster 1”) and -2 (dots in the right cluster with the same color as the label “Cluster 2”) with positions of known capsids (AAV9, PHP.S, PHP.B, PHP.B4, PHP.C1) and new capsids (PHP.PNS1 and PHP.PNS2) are shown. Heatmaps show enrichment of representative capsids from Cluster-1 and -2 across organs. FIG. 17C depicts a heatmap of enrichment of parent AAV9, variant PHP.PNS1 and PNS2, and prior engineered variant PHP.S across cell-types or organs.

FIG. 18 depicts non-limiting exemplary embodiments and data related to marmoset brain injected with MaCPNS1 and MaCPNS2. Related to FIG. 5A-FIG. 5F. MaCPNS1 packaging ssAAV:CAG-eGFP and MaCPNS2 packaging ssAAV:CAG-tdTomato were mixed and intravenously delivered at a total dose of 7×10¹³ vg/kg per adult marmoset (16-month-old Callithrix jacchus, i.e. 3.5×10¹³ vg/kg per AAV). Representative overview of marmoset midbrain.

FIG. 19 depicts non-limiting exemplary embodiments and data related to macaque midbrain injected with MaCPNS1 and MaCPNS2. Related to FIG. 6A-FIG. 6F. MaCPNS1 packaging ssAAV:CAG-eGFP and MaCPNS2 packaging ssAAV:CAG-tdTomato were mixed and intravenously injected at a dose of 5×10¹³ vg/kg per macaque (Macaca mulatta, injected within 10 days of birth, female, i.e. 2.5×10¹³ vg/kg per AAV). Representative overview of macaque midbrain.

FIG. 20 depicts non-limiting exemplary embodiments and data related to macaque hindbrain injected with MaCPNS1 and MaCPNS2, related to FIG. 6A-FIG. 6F. MaCPNS1 packaging ssAAV:CAG-eGFP and MaCPNS2 packaging ssAAV:CAG-tdTomato were mixed and intravenously injected at a dose of 5×10¹³ vg/kg per macaque (Macaca mulatta, injected within 10 days of birth, female, i.e. 2.5×10¹³ vg/kg per AAV). Representative overview of macaque hindbrain.

FIG. 21 depicts non-limiting exemplary embodiments and data related to macaque cerebellum injected with MaCPNS1 and MaCPNS2, related to FIG. 6A-FIG. 6F. MaCPNS1 packaging ssAAV:CAG-eGFP and MaCPNS2 packaging ssAAV:CAG-tdTomato were mixed and intravenously injected at a dose of 5×10¹³ vg/kg per macaque (Macaca mulatta, injected within 10 days of birth, female, i.e. 2.5×10¹³ vg/kg per AAV). Representative overview of macaque cerebellum.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein and made part of the disclosure herein.

All patents, published patent applications, other publications, and sequences from GenBank, and other databases referred to herein are incorporated by reference in their entirety with respect to the related technology.

Disclosed herein include AAV targeting peptides. In some embodiments, the AAV targeting peptide comprises an amino acid sequence that comprises at least 4 contiguous amino acids from a sequence selected from PHEGSSR (SEQ ID NO: 169), LNNTKTT (SEQ ID NO: 237), SNLARNV (SEQ ID NO: 274) and TNNTKPL (SEQ ID NO: 390). In some embodiments, the AAV targeting peptide comprises an amino acid sequence of 7 contiguous amino acids X₁X₂X₃X₄X₅X₆X₇, wherein X₁ is G; X₂ is N; X₃ is A, C, D, E, F, G, H, I, K, L, M, P, Q, S, T, V, W, or Y; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is R; and X₇ is A, C, D, E, F, G, H, I, K, L, M, N, P, Q, R, S, T, V, W, or Y. In some embodiments, the AAV targeting peptide comprises an amino acid sequence of 7 contiguous amino acids X₁X₂X₃X₄X₅X₆X₇, wherein X₁ is A, C, D, E, F, G, H, I, K, L, M, N, P, Q, R, T, V, W, or Y; X₂ is N; X₃ is R; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is A, C, D, E F, G, H, I, K, L, M, N, P, Q, R, S, T, V, W, or Y; and X₇ is T. In some embodiments, the AAV targeting peptide comprises an amino acid sequence of 7 contiguous amino acids X₁X₂X₃X₄X₅X₆X₇, wherein X₁ is A, C, D, E, F, G, H, I, K, L, M, N, P, Q, R, S, T, V, W, or Y; X₂ is N; X₃ is R; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is D; and X₇ is A, C, D, E, F, G, H, I, K, L, M, N, P, Q, R, S, T, V, W, or Y. In some embodiments, the AAV targeting peptide comprises an amino acid sequence of 7 contiguous amino acids X₁X₂X₃X₄X₅X₆X₇, wherein X₁ is S; X₂ is N; X₃ is R; X₄ is A, C, D, E, F, G, H, I, K, L, M, N, P, Q, R, S, T, V, W, or Y; X₅ is A, C, D, E, F, G, H, I, K, L, M, N, P, Q, R, S, T, V, W, or Y; X₆ is A, C, D, E, F, G, H, I, K, L, M, N, P, Q, R, S, T, V, W, or Y; and X₇ is A, C, D, E, F, G, H, I, K, L, M, N, P, Q, R, S, T, V, W, or Y. In some embodiments, the AAV targeting peptide comprises an amino acid sequence of 7 contiguous amino acids X₁X₂X₃X₄X₅X₆X₇, wherein X₁ is K, L, S, H, I, Y, or N; X₂ is N; X₃ is R; X₄ is K, A, T, D, M, H, or V; X₅ is A, R, I, M, D, Y, or K; X₆ is D; and X₇ is A, G, R, S, V, D, or M. In some embodiments, the AAV targeting peptide comprises an amino acid sequence of 7 contiguous amino acids X₁X₂X₃X₄X₅X₆X₇, wherein X₁ is S; X₂ is N; X₃ is R; X₄ is R, V, P, T, E, F, or I; X₅ is A, T, P, G, S, R, or V; X₆ is F, H, P, I, L, T, or D; and X₇ is A, E, I, Q, Y, V, or M. In some embodiments, the AAV targeting peptide comprises an amino acid sequence of 7 contiguous amino acids X₁X₂X₃X₄X₅X₆X₇, wherein X₁ is L, M, A, K, G, T, or E; X₂ is N; X₃ is R; X₄ is S, I, Q, N, A, G, or Y; X₅ is V, K, S, E, D, A, or N; X₆ is V, N, P, S, R, Q, or Y; and X₇ is T. In some embodiments, the AAV targeting peptide comprises an amino acid sequence of 7 contiguous amino acids X₁X₂X₃X₄X₅X₆X₇, wherein X₁ is G; X₂ is N; X₃ is Q, Y, L, T, A, I, or G; X₄ is T, D, H, P, A, K, or Y; X₅ is P, A, I, E, N, S, or Q; X₆ is R; and X₇ is K, G, M, S, A, P, or H.

Disclosed herein include AAV capsid proteins comprising one or more of the AAV targeting peptide provided herein. There are also provided nucleic acids comprising a sequence encoding the AAV targeting peptide and/or AAV capsid protein provided herein.

Disclosed herein include recombinant rAAVs comprising one or more of the AAV targeting peptides provided herein, and/or an AAV capsid protein provided herein. In some embodiments, the AAV capsid protein comprises the AAV targeting peptide provided herein inserted between two adjacent amino acids in AA586-592 or functional equivalents thereof of the AAV capsid protein.

Disclosed herein include compositions comprising one or more of the AAV targeting peptides disclosed herein, one or more of the AAV capsid proteins disclosed herein, one or more of the nucleic acids disclosed herein, one or more of the rAAVs disclosed herein, or a combination thereof. Disclosed herein include compositions for use in the delivery of an agent to a target environment of a subject in need. The composition can comprise an AAV comprising (1) an AAV capsid protein provided herein and (2) an agent to be delivered to the target environment of the subject. The target environment can be the nervous system, the lung or a combination thereof.

The composition can, for example, comprise an AAV comprising (1) an AAV capsid protein comprising an AAV targeting peptide disclosed herein and (2) an agent to be delivered to the target environment of the subject. The target environment can be the PNS. In some embodiments, the composition comprises an AAV comprising (1) an AAV capsid protein comprising an AAV targeting peptide disclosed herein and (2) an agent to be delivered to the target environment of the subject. The target environment can be the lung. In some embodiments, the agent to be delivered comprises a nucleic acid, a peptide, a small molecule, an aptamer, or a combination thereof.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, the terms “nucleic acid” and “polynucleotide” are interchangeable and can refer to any nucleic acid, whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, bridged phosphoramidate, bridged phosphoramidate, bridged methylene phosphonate, phosphorothioate, methylphosphonate, phosphorodithioate, bridged phosphorothioate or sultone linkages, and combinations of such linkages. The terms “nucleic acid” and “polynucleotide” also specifically include nucleic acids composed of bases other than the five biologically occurring bases (adenine, guanine, thymine, cytosine and uracil).

The term “vector” as used herein, can refer to a vehicle for carrying or transferring a nucleic acid. Non-limiting examples of vectors include plasmids and viruses (for example, AAV viruses).

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “plasmid” can refer to a nucleic acid that can be used to replicate recombinant DNA sequences within a host organism. The sequence can be a double stranded DNA.

The term “virus genome” refers to a nucleic acid sequence that is flanked by cis acting nucleic acid sequences that mediate the packaging of the nucleic acid into a viral capsid. For AAVs and parvoviruses, for example it is known that the “inverted terminal repeats” (ITRs) that are located at the 5′ and 3′ end of the viral genome have this function and that the ITRs can mediate the packaging of heterologous, for example, non-wildtype virus genomes, into a viral capsid.

The term “element” can refer to a separate or distinct part of something, for example, a nucleic acid sequence with a separate function within a longer nucleic acid sequence. The term “regulatory element” and “expression control element” are used interchangeably herein and refer to nucleic acid molecules that can influence the expression of an operably linked coding sequence in a particular host organism. These terms are used broadly to and cover all elements that promote or regulate transcription, including promoters, core elements required for basic interaction of RNA polymerase and transcription factors, upstream elements, enhancers, and response elements (see e.g., Lewin, “Genes V” (Oxford University Press, Oxford) pages 847-873). Exemplary regulatory elements in prokaryotes include promoters, operator sequences and a ribosome binding site. Regulatory elements that are used in eukaryotic cells can include, without limitation, transcriptional and translational control sequences, such as promoters, enhancers, splicing signals, polyadenylation signals, terminators, protein degradation signals, internal ribosome-entry element (IRES), 2A sequences, and the like, that provide for and/or regulate expression of a coding sequence and/or production of an encoded polypeptide in a host cell.

As used herein, the term “promoter” is a nucleotide sequence that permits binding of RNA polymerase and directs the transcription of a gene. Typically, a promoter is located in the 5′ non-coding region of a gene, proximal to the transcriptional start site of the gene. Sequence elements within promoters that function in the initiation of transcription are often characterized by consensus nucleotide sequences. Examples of promoters include, but are not limited to, promoters from bacteria, yeast, plants, viruses, and mammals (including humans). A promoter can be inducible, repressible, and/or constitutive. Inducible promoters initiate increased levels of transcription from DNA under their control in response to some change in culture conditions, such as a change in temperature.

As used herein, the term “enhancer” refers to a type of regulatory element that can increase the efficiency of transcription, regardless of the distance or orientation of the enhancer relative to the start site of transcription.

As used herein, the term “operably linked” is used to describe the connection between regulatory elements and a gene or its coding region. Typically, gene expression is placed under the control of one or more regulatory elements, for example, without limitation, constitutive or inducible promoters, tissue-specific regulatory elements, and enhancers. A gene or coding region is said to be “operably linked to” or “operatively linked to” or “operably associated with” the regulatory elements, meaning that the gene or coding region is controlled or influenced by the regulatory element. For instance, a promoter is operably linked to a coding sequence if the promoter effects transcription or expression of the coding sequence.

The term “construct,” as used herein, can refer to a recombinant nucleic acid that has been generated for the purpose of the expression of a specific nucleotide sequence(s), or that is to be used in the construction of other recombinant nucleotide sequences.

As used herein, the term “variant” can refer to a polynucleotide or polypeptide having a sequence substantially similar to a reference polynucleotide or polypeptide. In the case of a polynucleotide, a variant can have deletions, substitutions, additions of one or more nucleotides at the 5′ end, 3′ end, and/or one or more internal sites in comparison to the reference polynucleotide. Similarities and/or differences in sequences between a variant and the reference polynucleotide can be detected using conventional techniques known in the art, for example polymerase chain reaction (PCR) and hybridization techniques. Variant polynucleotides also include synthetically derived polynucleotides, such as those generated, for example, by using site-directed mutagenesis. Generally, a variant of a polynucleotide, including, but not limited to, a DNA, can have at least about 50%, about 55%, about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polynucleotide as determined by sequence alignment programs known by skilled artisans. In the case of a polypeptide, a variant can have deletions, substitutions, additions of one or more amino acids in comparison to the reference polypeptide. Similarities and/or differences in sequences between a variant and the reference polypeptide can be detected using conventional techniques known in the art, for example Western blot. Generally, a variant of a polypeptide, can have at least about 60%, about 65%, about 70%, about 75%, about 80%, about 85%, about 90%, about 91%, about 92%, about 93%, about 94%, about 95%, about 96%, about 97%, about 98%, about 99% or more sequence identity to the reference polypeptide as determined by sequence alignment programs known by skilled artisans.

The term “AAV” or “adeno-associated virus” refers to a Dependoparvovirus within the Parvoviridae genus of viruses. For example, the AAV can be an AAV derived from a naturally occurring “wild-type” virus, an AAV derived from an rAAV genome packaged into a capsid derived from capsid proteins encoded by a naturally occurring cap gene and/or an rAAV genome packaged into a capsid derived from capsid proteins encoded by a non-natural capsid cap gene, for example, AAV capsid protein comprising sequence of SEQ ID NOs: 385 and/or 386. Non-limited examples of AAV include AAV type 1 (AAV1), AAV type 2 (AAV2), AAV type 3 (AAV3), AAV type 4 (AAV4), AAV type 5 (AAV5), AAV type 6 (AAV6), AAV type 7 (AAV7), AAV type 8 (AAV8), AAV type 9 (AAV9), AAV type 10 (AAV10), AAV type 11 (AAV11), AAV type 12 (AAV12), AAV type DJ (AAV-DJ), avian AAV, bovine AAV, canine AAV, equine AAV, primate AAV, non-primate AAV, and ovine AAV. In some instances, the AAV is described as a “Primate AAV,” which refers to AAV that infects primates. Likewise, an AAV may infect bovine animals (e.g., “bovine AAV”, and the like). In some instances, the AAV is wild type, or naturally occurring. In some instances, the AAV is recombinant.

The term “AAV capsid” as used herein refers to a capsid protein or peptide of an adeno-associated virus. In some instances, the AAV capsid protein is configured to encapsidate genetic information (e.g., a heterologous nucleic acid, a transgene, therapeutic nucleic acid, viral genome). In some instances, the AAV capsid of the instant disclosure is a variant AAV capsid, which means in some instances that it is a parental or wild-type AAV capsid that has been modified in an amino acid sequence of the parental AAV capsid protein.

The term “AAV genome” as used herein can refer to nucleic acid polynucleotide encoding genetic information related to the virus. The genome, in some instances, comprises a nucleic acid sequence flanked by AAV inverted terminal repeat (ITR) sequences. The AAV genome can be an rAAV genome generated using recombinatorial genetics methods, and which can include a heterologous nucleic acid (e.g., transgene) that comprises and/or is flanked by the ITR sequences.

The term “rAAV” refers to a “recombinant AAV”. In some embodiments, a recombinant AAV has an AAV genome in which part or all of the rep and cap genes have been replaced with heterologous sequences. The term “AAV particle”, “AAV nanoparticle”, or an “AAV vector” as used interchangeably herein refers to an AAV virus or virion comprising an AAV capsid within which is packaged a heterologous DNA polynucleotide, or “genome”, comprising nucleic acid sequence flanked by AAV (ITR sequences. In some cases, the AAV particle is modified relative to a parental AAV particle.

The term “cap gene” refers to the nucleic acid sequences that encode capsid proteins that form, or contribute to the formation of, the capsid, or protein shell, of the virus. In the case of AAV, the capsid protein may be VP1, VP2, or VP3. For other parvoviruses, the names and numbers of the capsid proteins can differ.

The term “rep gene” refers to the nucleic acid sequences that encode the non-structural proteins (rep78, rep68, rep52 and rep40) required for the replication and production of virus.

The terms “native” and “wild type” are used interchangeably herein, and can refer to the form of a polynucleotide, gene or polypeptide as found in nature with its own regulatory sequences, if present.

As used herein, “endogenous” refers to the native form of a polynucleotide, gene or polypeptide in its natural location in the organism or in the genome of an organism. “Endogenous polynucleotide” includes a native polynucleotide in its natural location in the genome of an organism. “Endogenous gene” includes a native gene in its natural location in the genome of an organism. “Endogenous polypeptide” includes a native polypeptide in its natural location in the organism.

As used herein, “heterologous” refers to a polynucleotide, gene or polypeptide not normally found in the host organism but that is introduced into the host organism. “Heterologous polynucleotide” includes a native coding region, or portion thereof, that is reintroduced into the source organism in a form that is different from the corresponding native polynucleotide. “Heterologous gene” includes a native coding region, or portion thereof, that is reintroduced into the source organism in a form that is different from the corresponding native gene. For example, a heterologous gene may include a native coding region that is a portion of a chimeric gene including non-native regulatory regions that is reintroduced into the native host. “Heterologous polypeptide” includes a native polypeptide that is reintroduced into the source organism in a form that is different from the corresponding native polypeptide. The subject genes and proteins can be fused to other genes and proteins to produce chimeric or fusion proteins. The genes and proteins useful in accordance with embodiments of the subject disclosure include not only the specifically exemplified full-length sequences, but also portions, segments and/or fragments (including contiguous fragments and internal and/or terminal deletions compared to the full-length molecules) of these sequences, variants, mutants, chimerics, and fusions thereof.

The term “exogenous” gene as used herein is meant to encompass all genes that do not naturally occur within the genome of an individual. For example, a miRNA could be introduced exogenously by a virus, e.g. an AAV nanoparticle.

As used herein, a “subject” refers to an animal that is the object of treatment, observation or experiment. “Animal” includes cold- and warm-blooded vertebrates and invertebrates such as fish, shellfish, reptiles, and in particular, mammals. “Mammal,” as used herein, refers to an individual belonging to the class Mammalia and includes, but not limited to, humans, domestic and farm animals, zoo animals, sports and pet animals. Non-limiting examples of mammals include mice; rats; rabbits; guinea pigs; dogs; cats; sheep; goats; cows; horses; primates, such as monkeys, chimpanzees and apes, and, in particular, humans. In some embodiments, the mammal is a human. However, in some embodiments, the mammal is not a human. In some embodiments, the subject is a rodent (e.g., rat or mouse). In some embodiments, the subject is a primate (e.g., human or money).

As used herein, the term “treatment” refers to an intervention made in response to a disease, disorder or physiological condition manifested by a patient. The aim of treatment may include, but is not limited to, one or more of the alleviation or prevention of symptoms, slowing or stopping the progression or worsening of a disease, disorder, or condition and the remission of the disease, disorder or condition. The term “treat” and “treatment” includes, for example, therapeutic treatments, prophylactic treatments, and applications in which one reduces the risk that a subject will develop a disorder or other risk factor. Treatment does not require the complete curing of a disorder and encompasses embodiments in which one reduces symptoms or underlying risk factors. In some embodiments, “treatment” refers to both therapeutic treatment and prophylactic or preventative measures. Those in need of treatment include those already affected by a disease or disorder or undesired physiological condition as well as those in which the disease or disorder or undesired physiological condition is to be prevented. As used herein, the term “prevention” refers to any activity that reduces the burden of the individual later expressing those symptoms. This can take place at primary, secondary and/or tertiary prevention levels, wherein: a) primary prevention avoids the development of symptoms/disorder/condition; b) secondary prevention activities are aimed at early stages of the condition/disorder/symptom treatment, thereby increasing opportunities for interventions to prevent progression of the condition/disorder/symptom and emergence of symptoms; and c) tertiary prevention reduces the negative impact of an already established condition/disorder/symptom by, for example, restoring function and/or reducing any condition/disorder/symptom or related complications. The term “prevent” does not require the 100% elimination of the possibility of an event. Rather, it denotes that the likelihood of the occurrence of the event has been reduced in the presence of the compound or method.

As used herein, the term “effective amount” refers to an amount sufficient to effect beneficial or desirable biological and/or clinical results.

As used herein, the term “pharmaceutically acceptable” carriers are ones which are nontoxic to the cell or mammal being exposed thereto at the dosages and concentrations employed. “Pharmaceutically acceptable” carriers can be, but not limited to, organic or inorganic, solid or liquid excipients which is suitable for the selected mode of application such as oral application or injection, and administered in the form of a conventional pharmaceutical preparation, such as solid such as tablets, granules, powders, capsules, and liquid such as solution, emulsion, suspension and the like. Often the physiologically acceptable carrier is an aqueous pH buffered solution such as phosphate buffer or citrate buffer. The physiologically acceptable carrier may also comprise one or more of the following: antioxidants including ascorbic acid, low molecular weight (less than about 10 residues) polypeptides, proteins, such as serum albumin, gelatin, immunoglobulins; hydrophilic polymers such as polyvinylpyrrolidone, amino acids, carbohydrates including glucose, mannose, or dextrins, chelating agents such as EDTA, sugar alcohols such as mannitol or sorbitol, salt-forming counterions such as sodium, and nonionic surfactants such as Tween™, polyethylene glycol (PEG), and Pluronics™. Auxiliary, stabilizer, emulsifier, lubricant, binder, pH adjustor controller, isotonic agent and other conventional additives may also be added to the carriers.

Gene therapy offers great promise in addressing neuropathologies associated with the central and peripheral nervous systems (CNS and PNS). However, genetic access remains difficult, reflecting the critical need for development of effective and non-invasive gene delivery vectors across species. As described herein, adeno-associated virus serotype 9 (AAV9) capsid was evolved in mice, and two capsids, AAV-MaCPNS1 and AAV-MaCPNS2 (also referred to herein as PSN1 or PNS2), were validated across rodent species (mice and rats) and non-human primate (NIP) species (marmosets and rhesus macaques). Intravenous administration of either AAV efficiently transduced the PNS in rodents, and both the PNS and CNS in NHPs. AAV-MaCPNS1 was used in mice to systemically deliver: (1) the neuronal sensor jGCaMP8s to record calcium signal dynamics in nodose ganglia, and (2) the neuronal actuator DREADD to dorsal root ganglia to mediate pain. This conclusively demonstrates the translatability of these two systemic AAVs across four species, and their functional utility through proof-of-concept studies in mice.

M-CREATE method can be used as described herein for selecting improved PNS-targeting capsids outperforming the prior engineered variant, AAV-PHP.S, which requires a high dose to exhibit its potent PNS tropism via IV delivery. Compared to the CNS, the PNS is a more challenging AAV engineering target. Cell populations are sparser, and there is no strong source of selection pressure across targets (akin to the BBB for the CNS). M-CREATE is uniquely well suited to this problem as it capitalizes on deep recovery of capsid libraries across cell-types/organs to select capsids enriched in areas of interest, and a customized analysis pipeline that incorporates positive and negative selections to identify variants with desired properties.

As described herein, M-CREATE was used to identify a family of 7-mer containing AAV9 capsids that were to be biased towards PNS areas, and the properties of two such selected AAVs, AAV-MaCPNS1 and AAV-MaCPNS2, in rodents and NHPs are described herein. In vivo validation of their tropism was provided in mice. Their applications with proof-of-concept studies for functional readout and modulation of sensory ganglia were demonstrated. In addition to finding improved PNS-targeting capsids, the fundamental question of the translatability of capsids selected in mouse models was addressed. In the present disclosure, these capsids were examined across four species commonly used in basic through pre-clinical applications: mice, rats, marmosets, and rhesus macaques. The variants discussed in the present disclosure showed improved efficiency and specificity towards the PNS, and translated their potent behavior across mammalian species. Due to the heterogeneity of the BBB, these variants also show efficient crossing of the BBB to infect the CNS in NHPs (Table 1).

TABLE 1
MAIN FEATURES OF NOVEL AAVS ACROSS SPECIES FOLLOWING
SYSTEMIC DELIVERY

| | Mouse | Rat | Marmoset | Macaque |
|---|---|---|---|---|
| MaCPNS1 | efficient | transduction of sensory | enhanced | enhanced |
| | transduction | ganglia (DRG/TG), | transduction of | transduction of |
| | of sensory | sympathetic chain | DRG, SI and | DRG |
| | ganglia | ganglia, mixed | ascending fiber | transduction of |
| | (NG/DRG), | sympathetic- | tracts in dorsal | sensory nerve |
| | demonstrated | parasympathetic ganglia | column of the | fibers entering |
| | functional | and enteric ganglia | SC, robust | SC and |
| | readout and | across SI and LI, | transduction of | ascending |
| | modulation | transduction of sensory | neurons and | afferent tracts in |
| | of sensory | nerve fibers entering SC | astrocytes, more | dorsal column, |
| | ganglia | and ascending afferent | biased to | robust |
| | | tracts in dorsal column, | neurons in the | transduction of |
| | | observed labeling of | brain | neurons and |
| | | neurons across brain | | non-neuronal |
| | | | | cells in brain |
| | | | | and SC, more |
| | | | | biased to |
| | | | | neurons |
| MaCPNS2 | efficient | transduction of sensory | enhanced | enhanced |
| | transduction | ganglia (DRG/TG), | transduction of | transduction of |
| | of sensory | sympathetic chain | DRG, SI and | DRG and GI |
| | ganglia | ganglia, mixed | ascending fiber | tract, including |
| | (NG/DRG), | sympathetic- | tracts in dorsal | esophagus, |
| | efficient | parasympathetic ganglia | column of the | colon and SI, |
| | transduction | and enteric ganglia | SC, robust | transduction of |
| | of ENS, | across SI and LI, | transduction of | sensory nerve |
| | especially SI | transduction of sensory | neurons and | fibers entering |
| | | nerve fibers entering SC | astrocytes in the | SC and |
| | | and ascending afferent | brain | ascending |
| | | tracts in dorsal column, | | afferent tracts in |
| | | observed labeling of | | dorsal column, |
| | | neurons across brain | | robust |
| | | | | transduction of |
| | | | | neurons and |
| | | | | non-neuronal |
| | | | | cells in brain |
| | | | | and SC |

M-CREATE has been extended further to select capsids that can efficiently target the nervous system across species and the cell populations of the lung. The cap gene of the natural AAV9 can be modified between amino acid (AA) positions 588 and 589 by a randomized combination of 7-11 AA long peptides. The produced AAV9 library was subjected to in vivo selections across Cre-transgenic mice lines to selectively recover the mutated capsids that were enriched in the Cre expressing cell types. The cell types of interest include neurons, astrocytes and endothelial cells across different organs or tissues that constituted the CNS, PNS and lung cell population. Multiplexed-CREATE has identified a large pool of targeting peptides that when inserted into an AAV vector can enable enhanced targeting of distinct cell types of the PNS, CNS and lung through systemic administration (Table 2-Table 5).

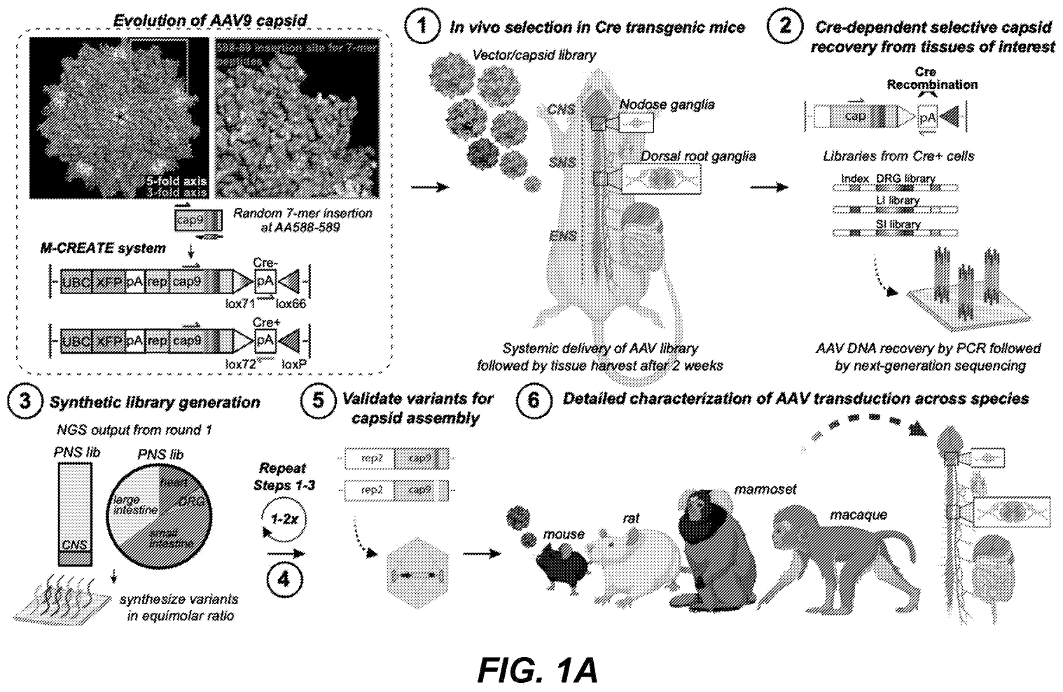

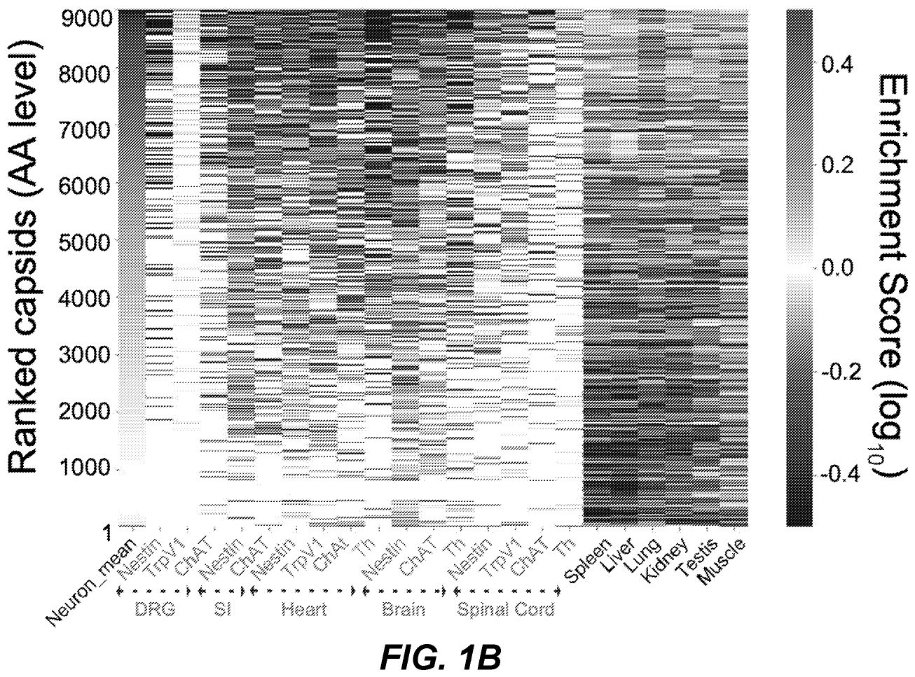

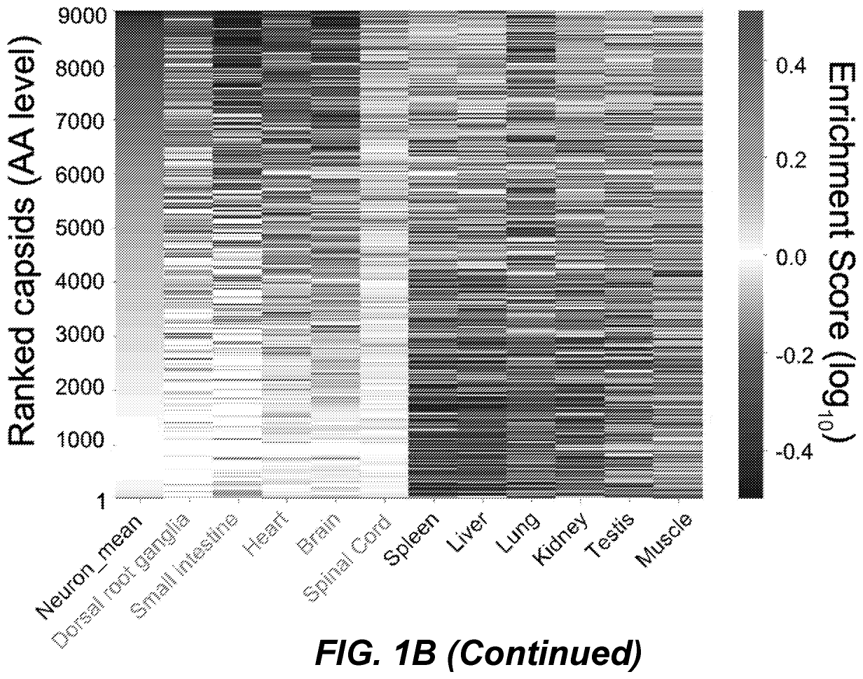

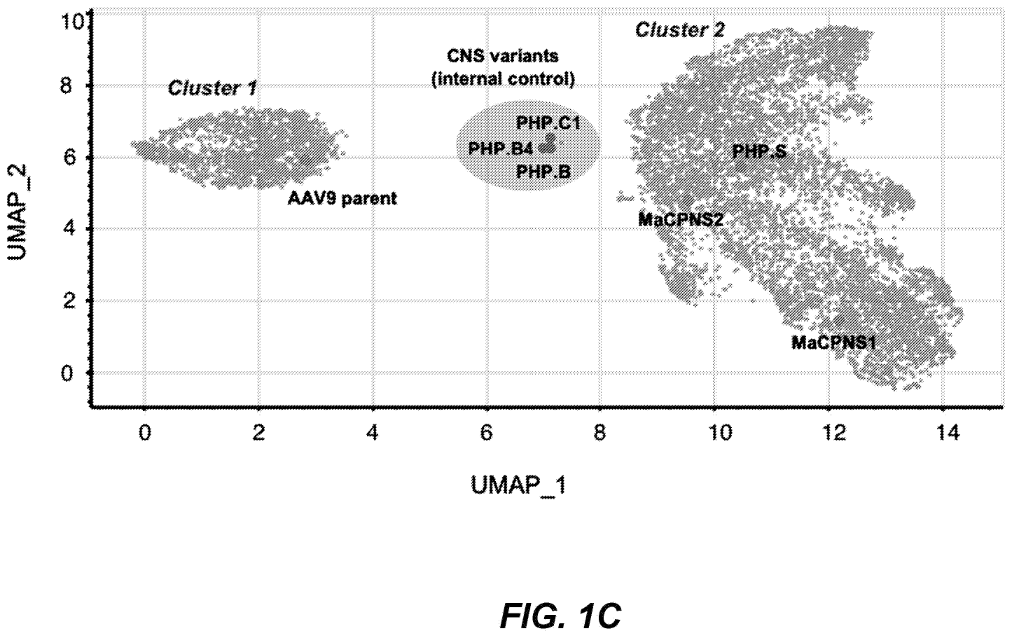

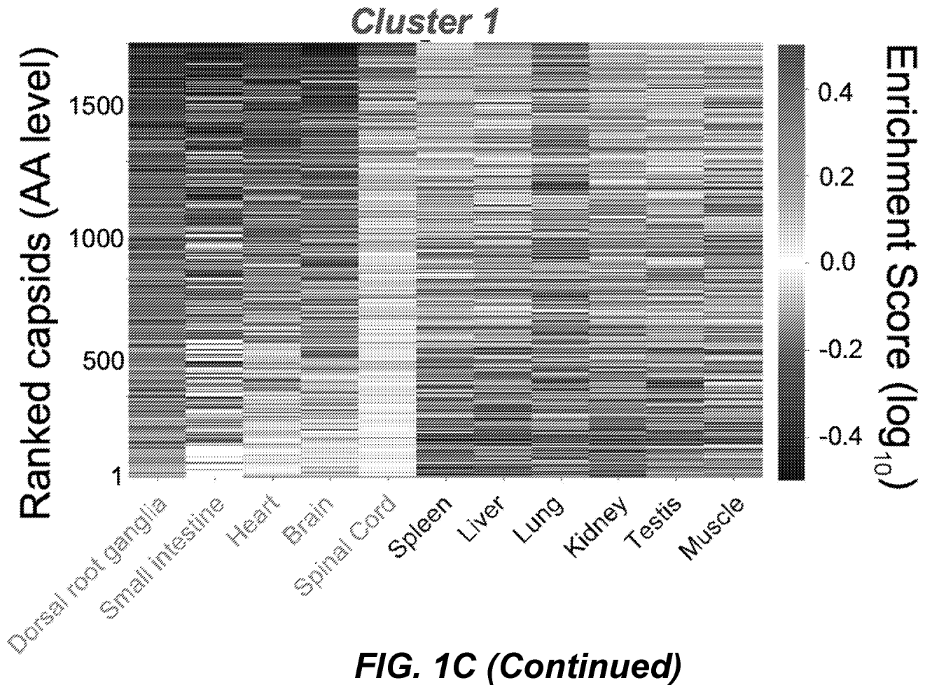

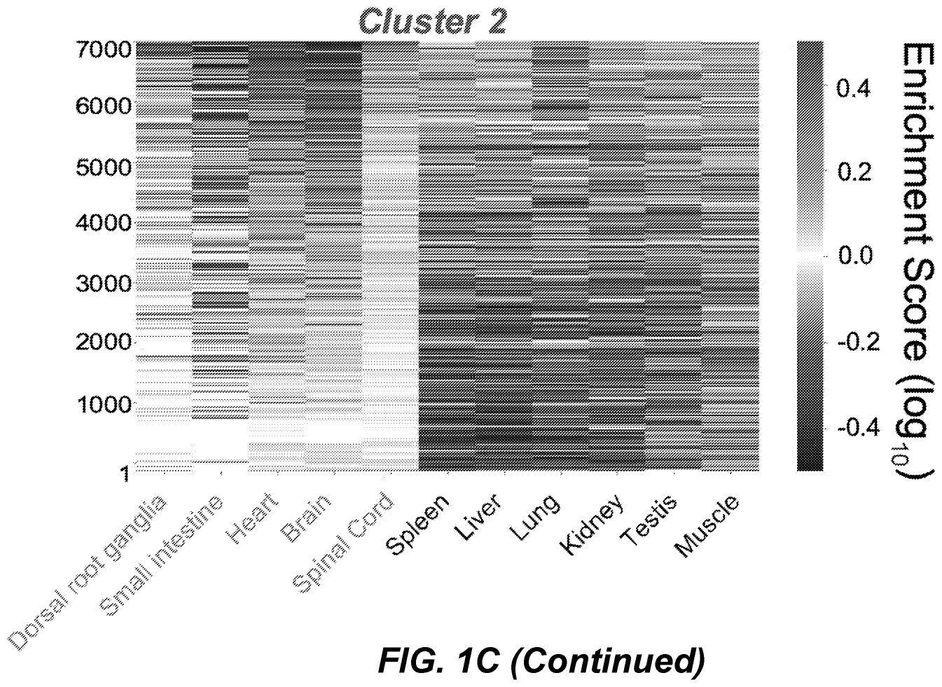

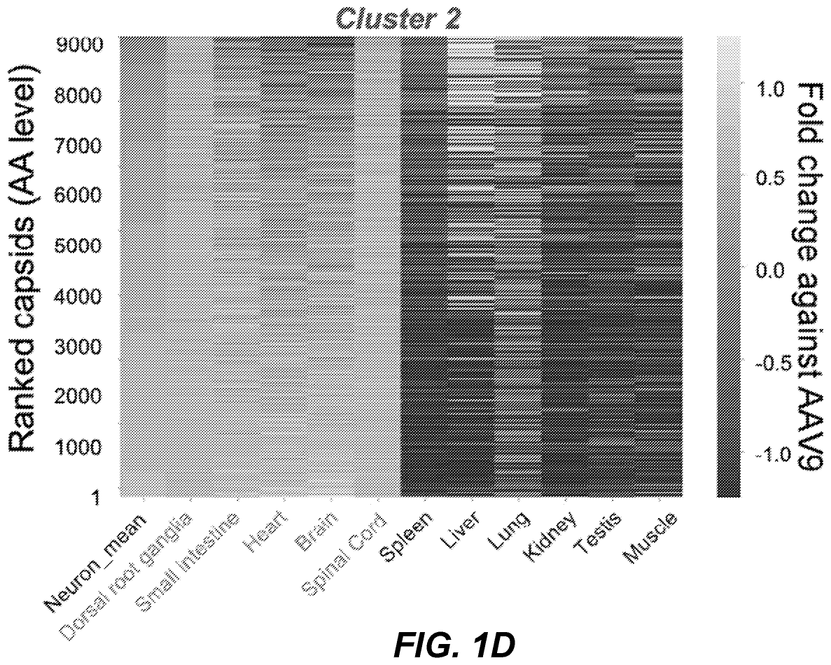

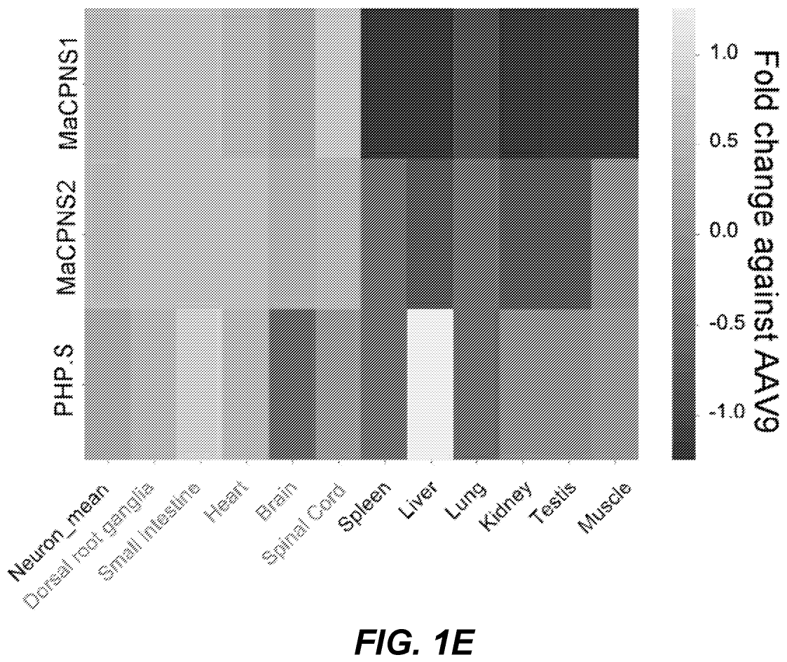

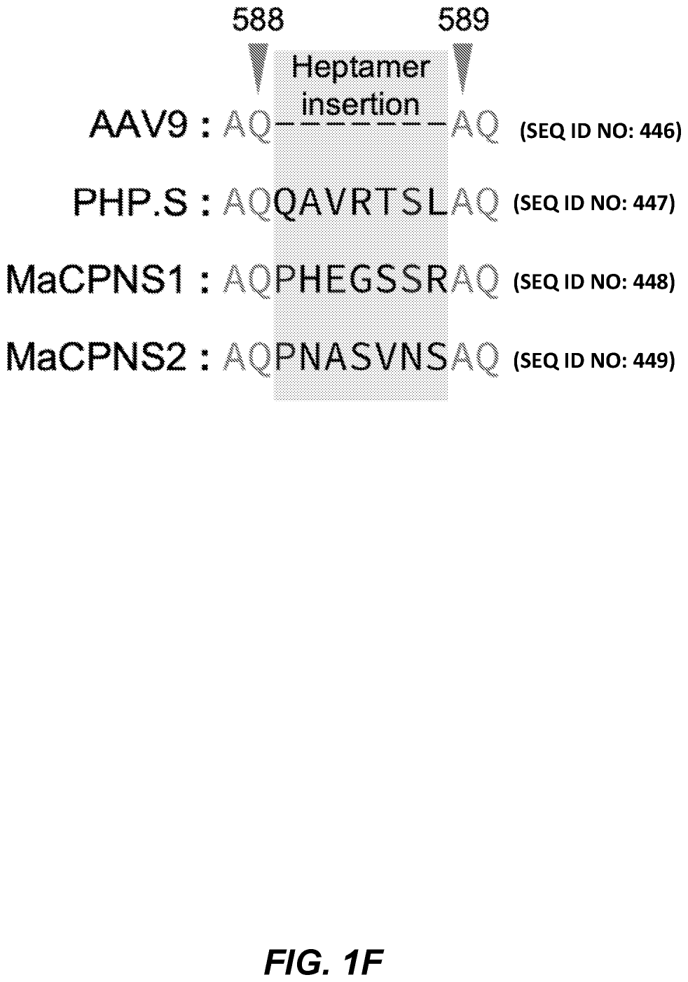

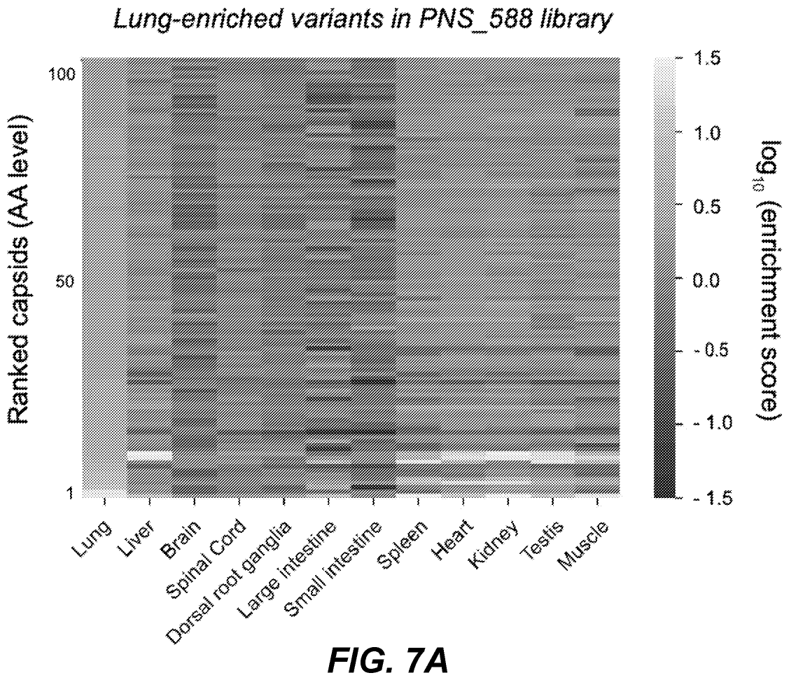

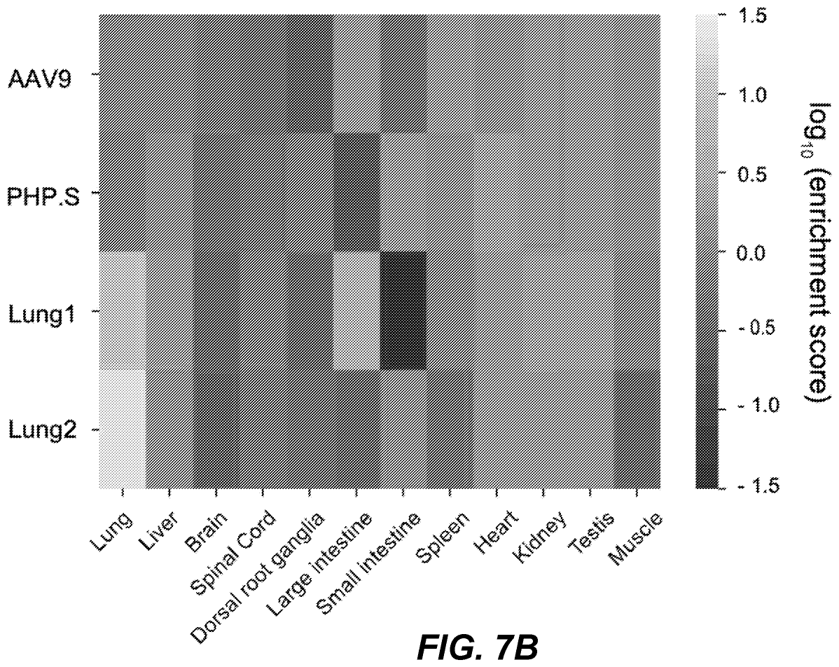

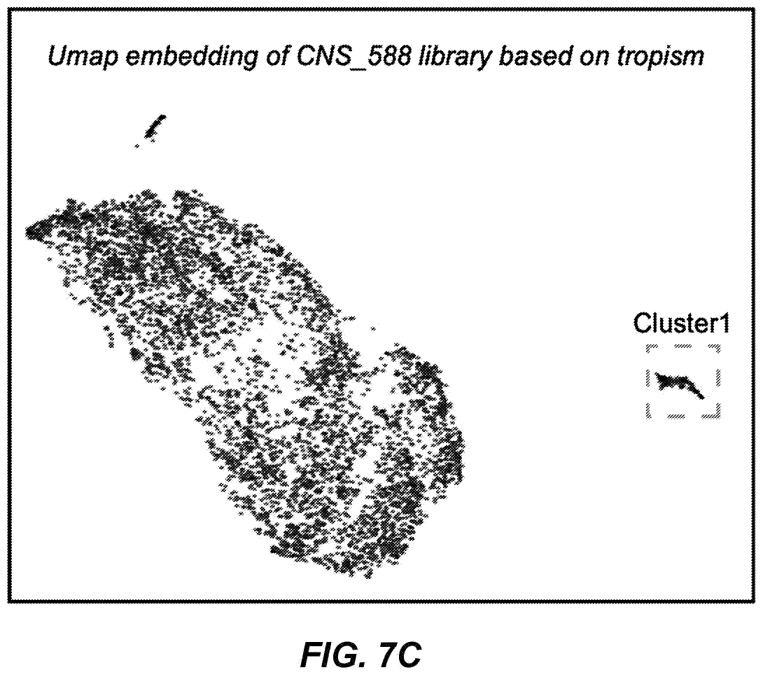

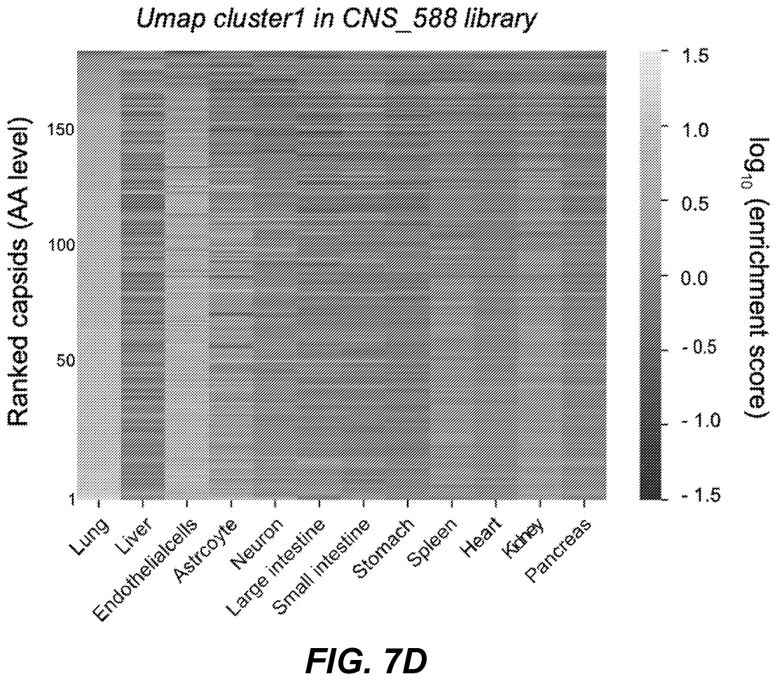

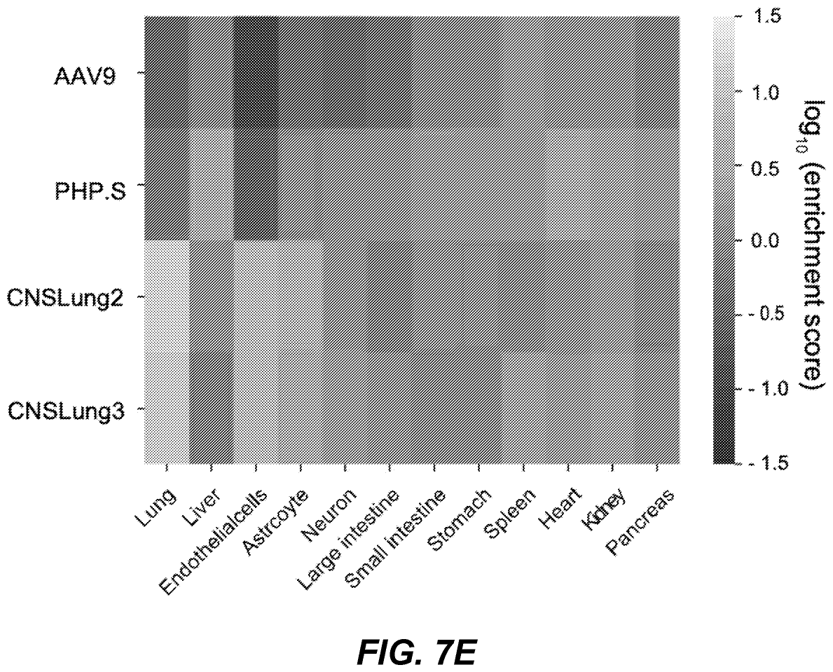

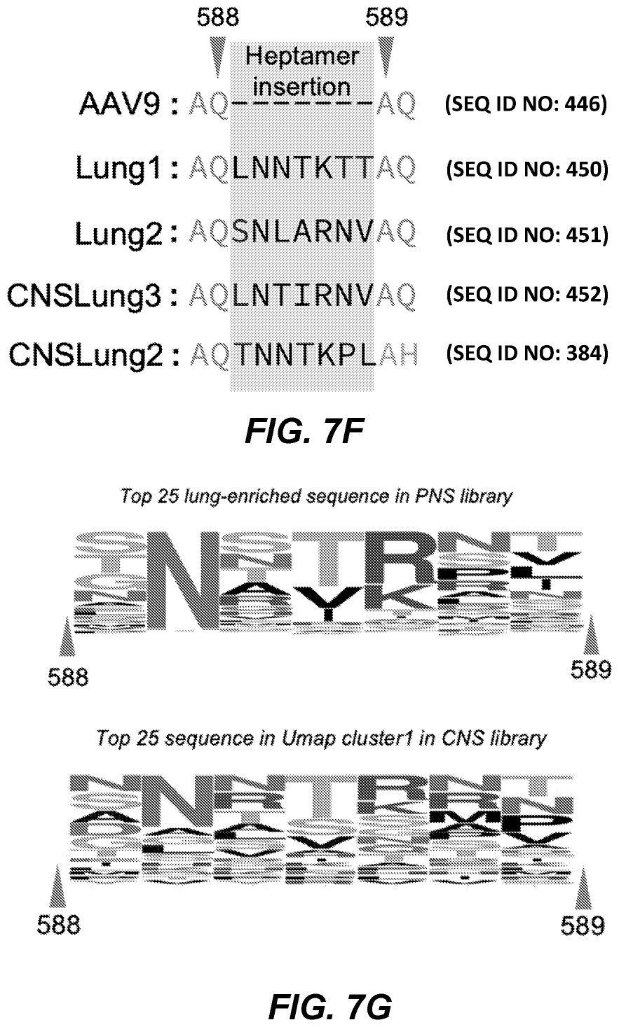

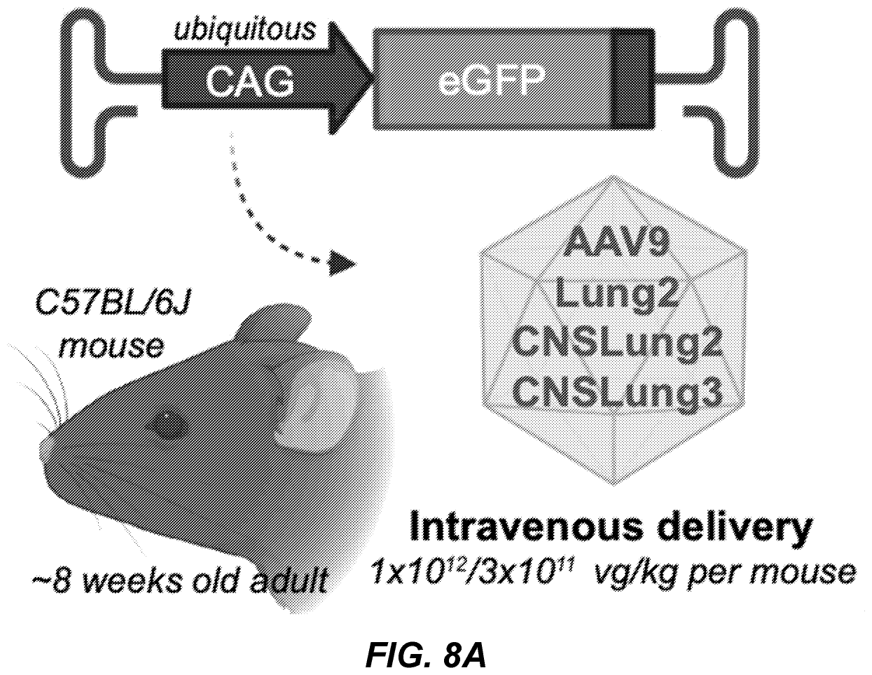

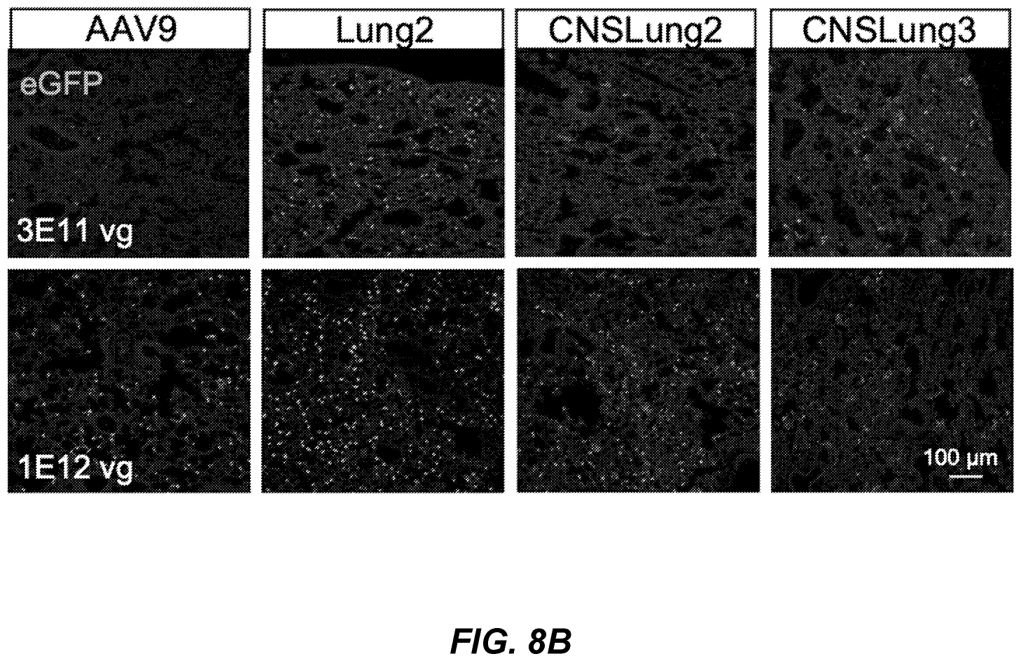

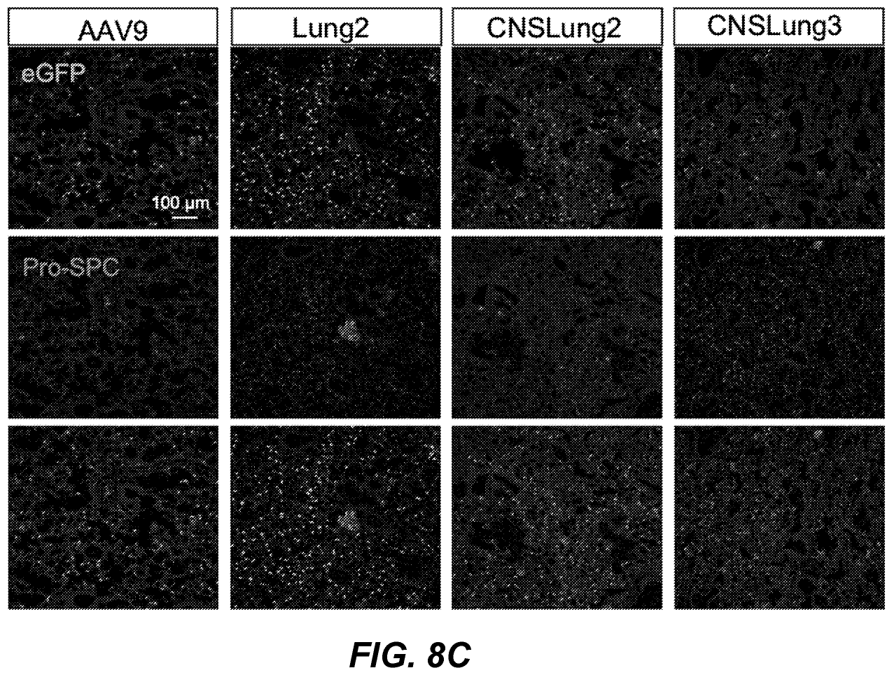

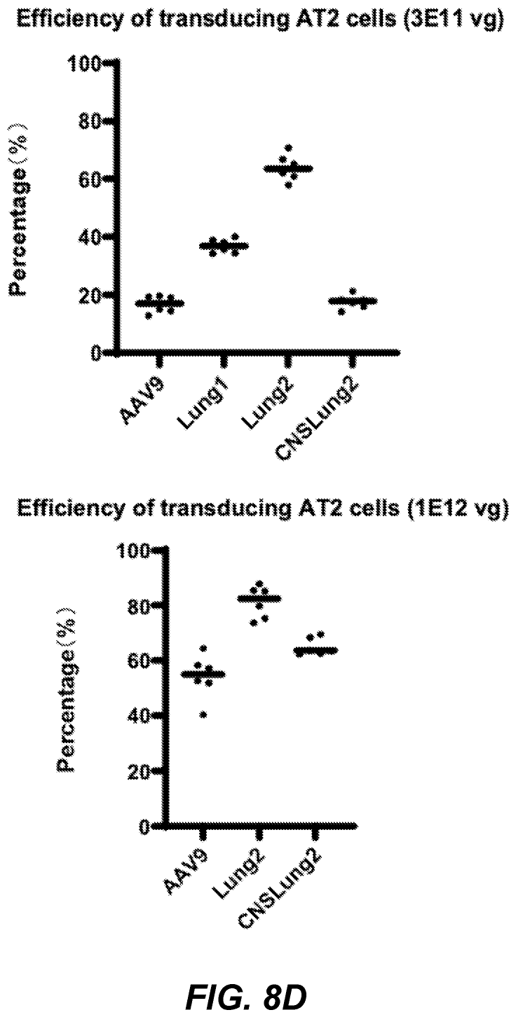

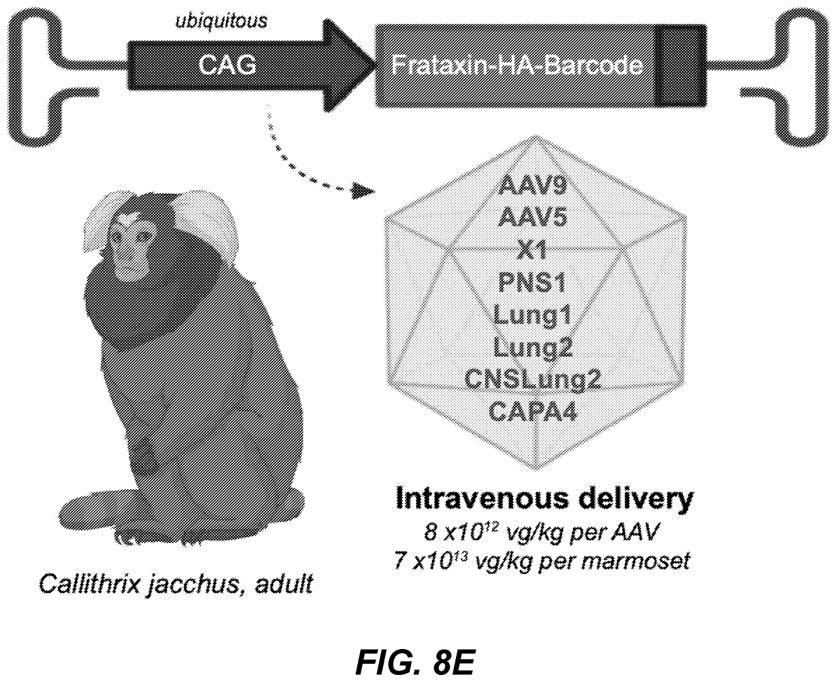

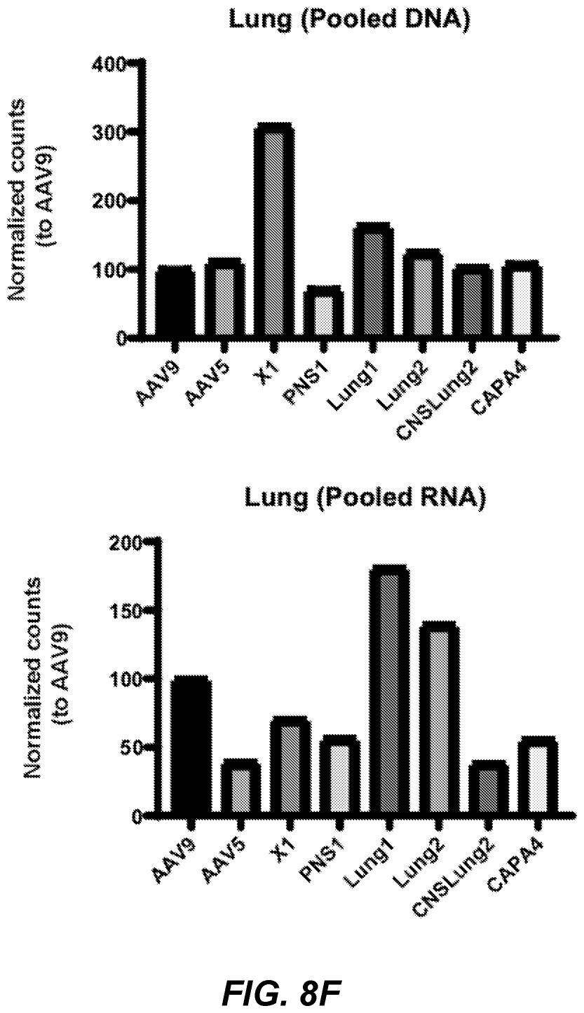

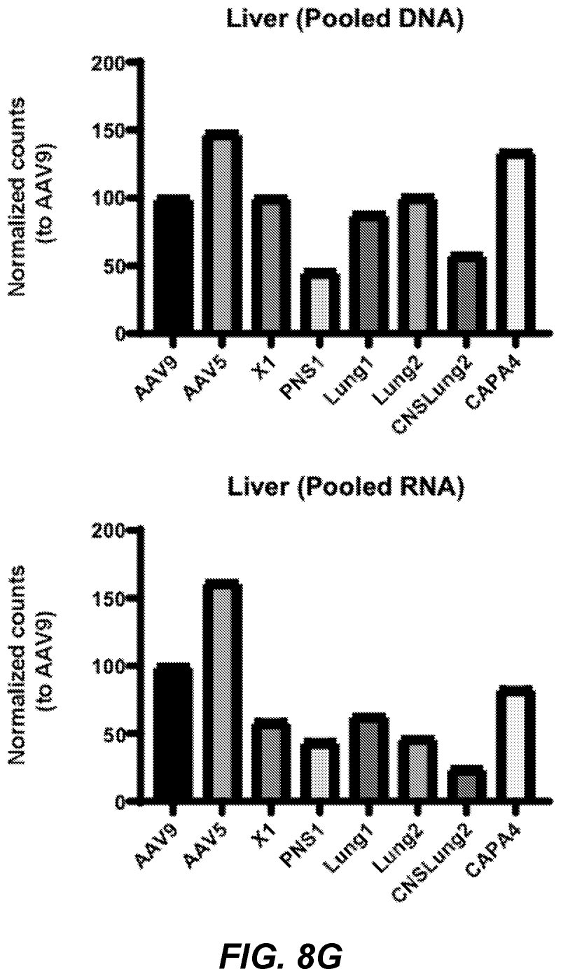

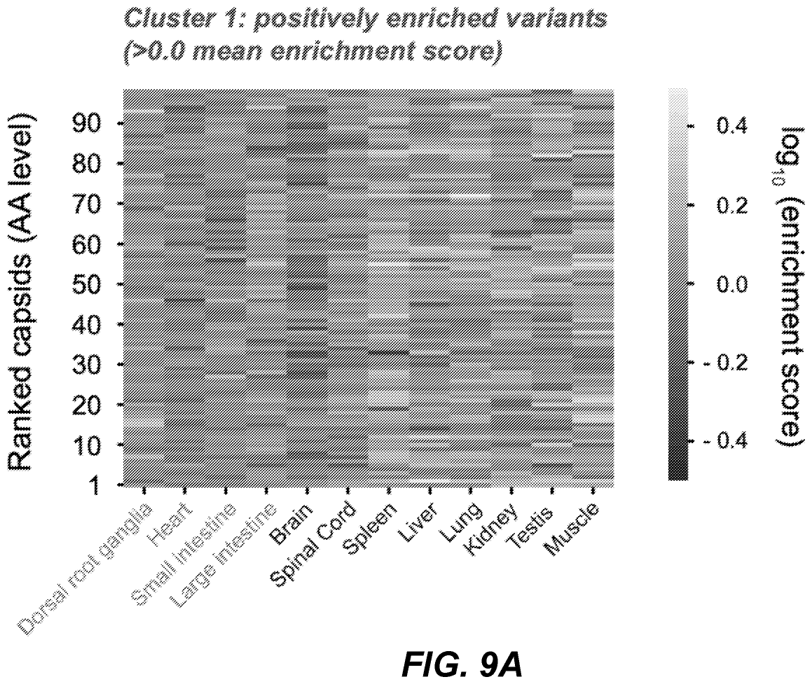

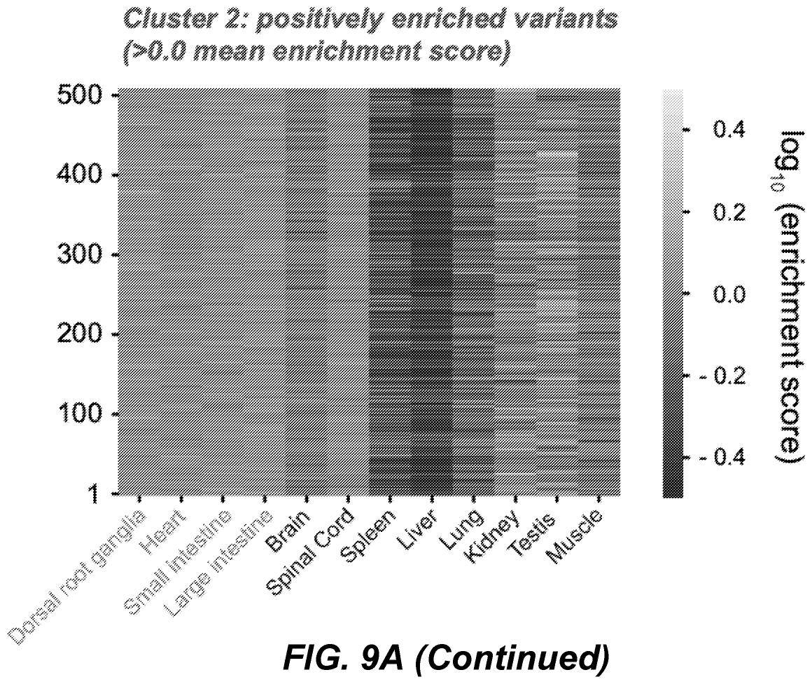

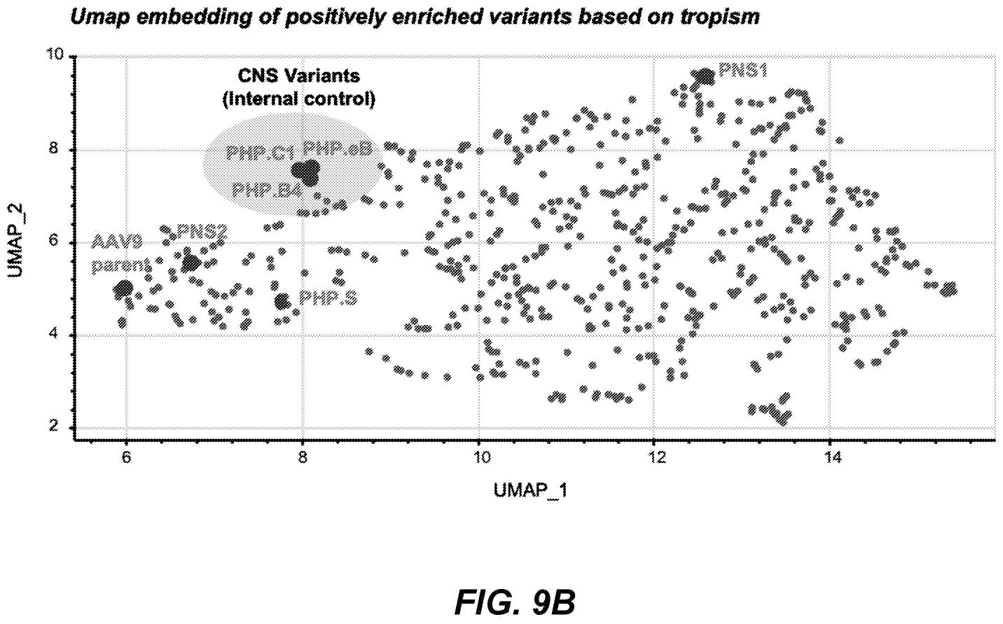

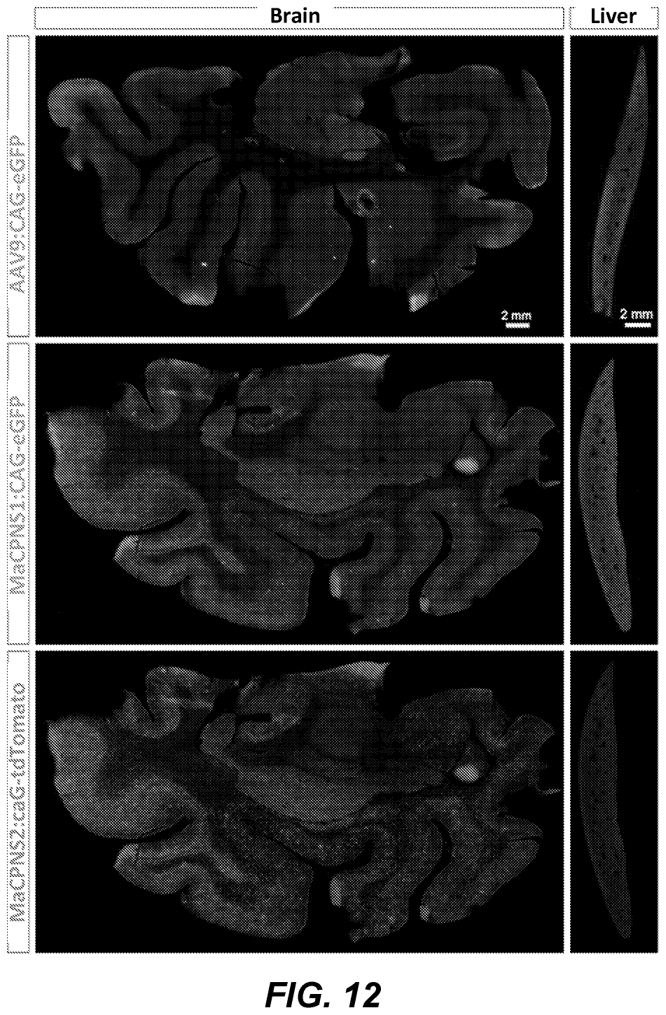

The peptides disclosed herein can direct vectors to different environments such as the CNS and PNS across species (Table 2-Table 5, FIG. 1A-FIG. 6F, FIG. 9A-FIG. 11E, FIG. 12). The disclosed peptides can direct vectors to cell types of the lung across species (Table 3-Table 5, FIG. 7A-FIG. 8G).

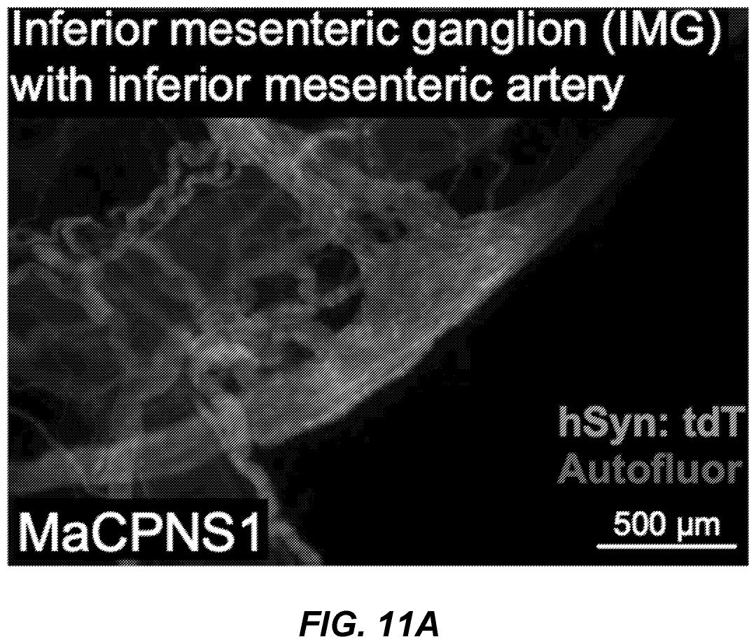

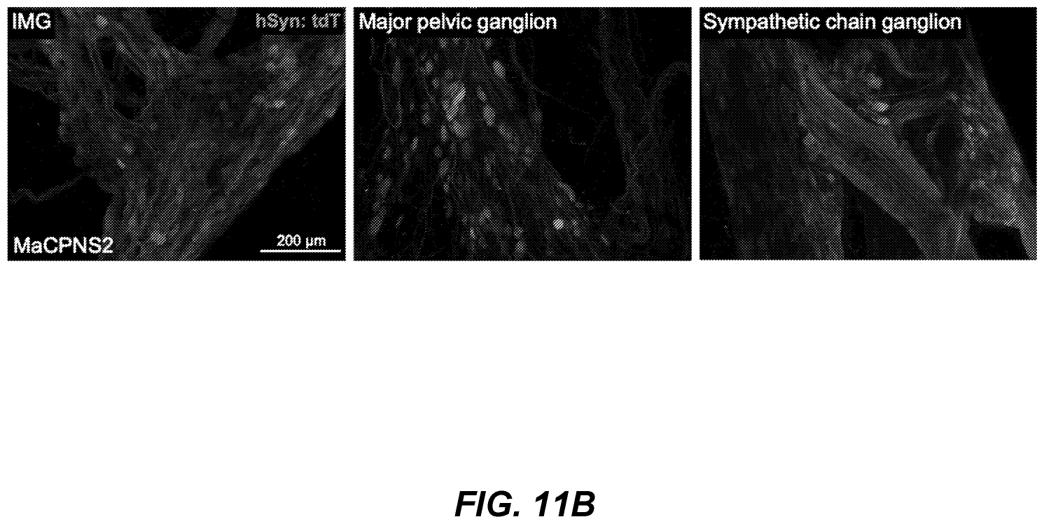

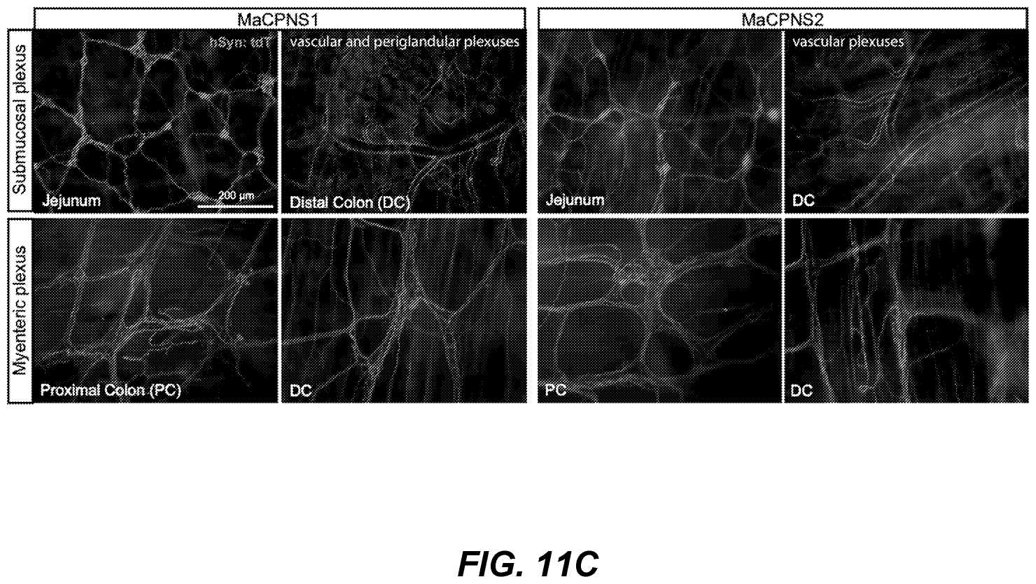

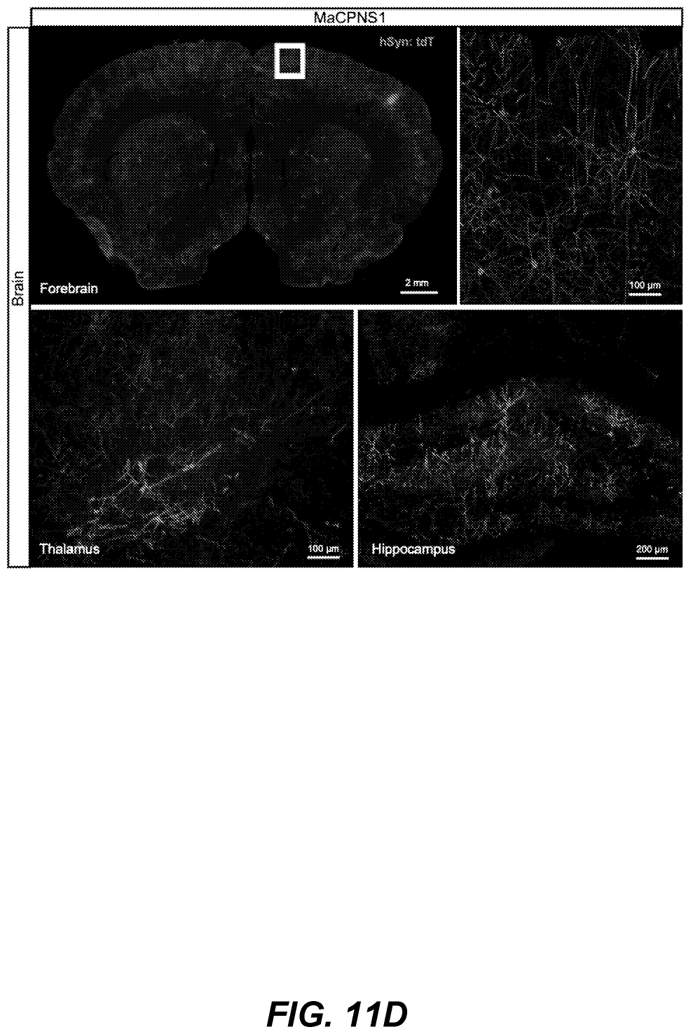

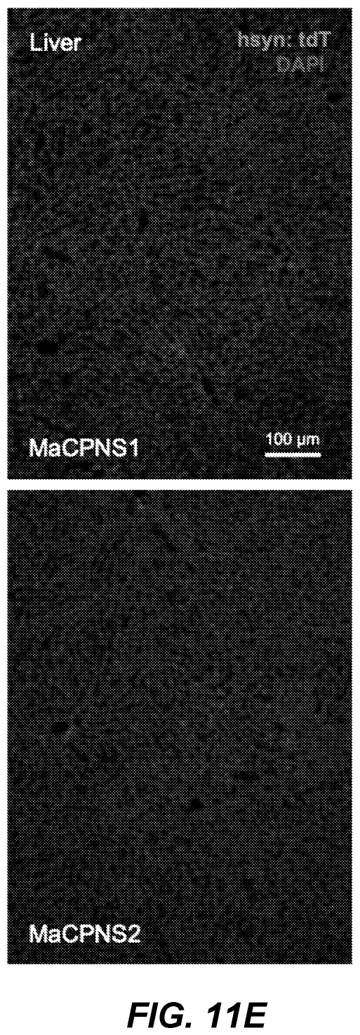

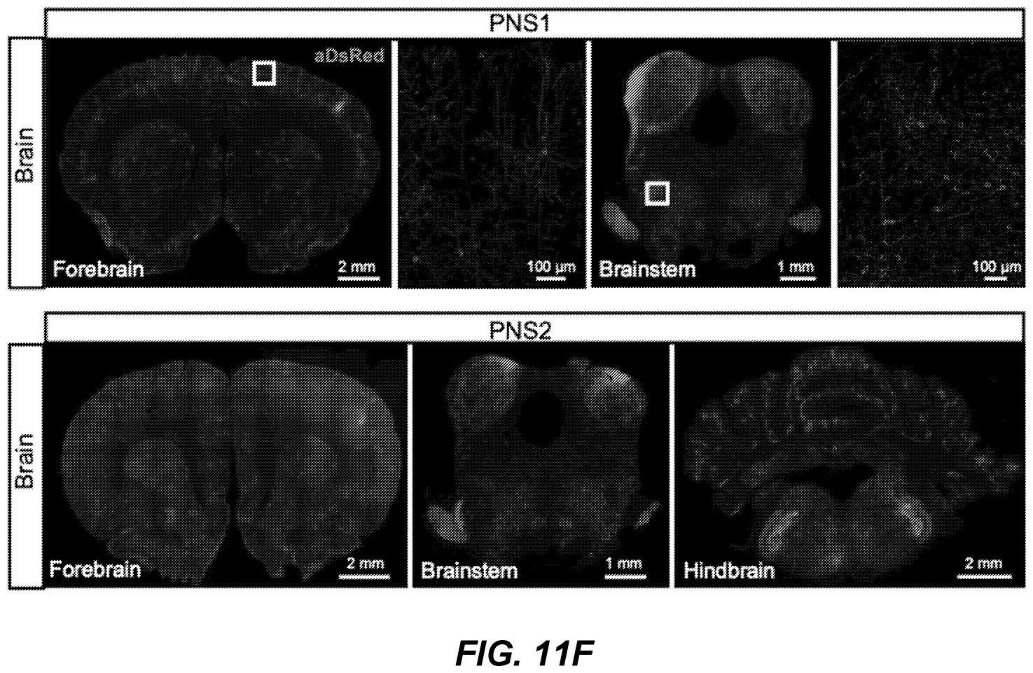

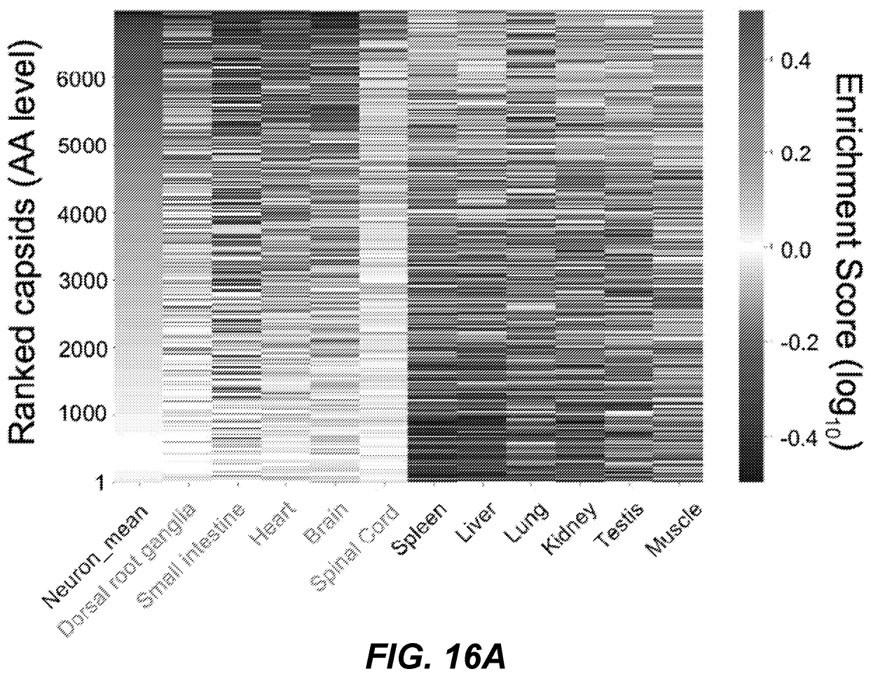

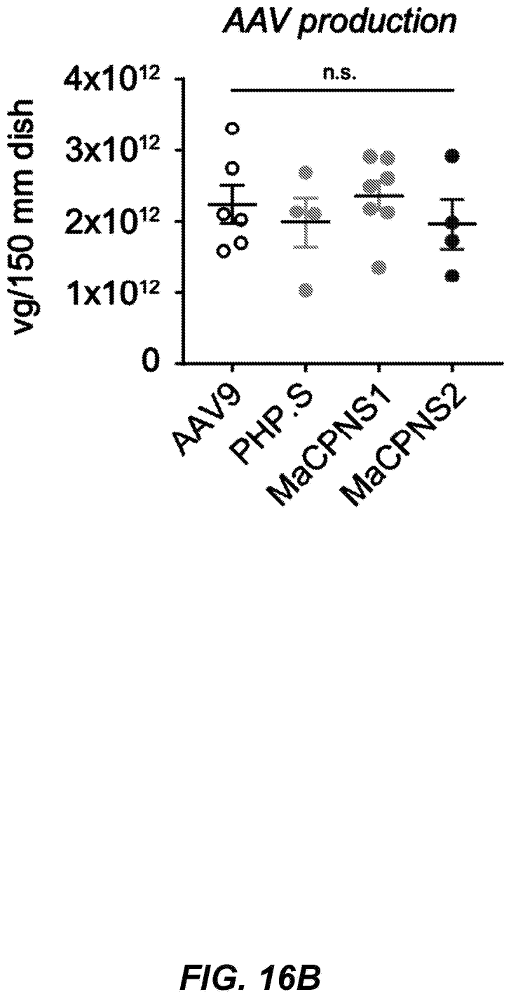

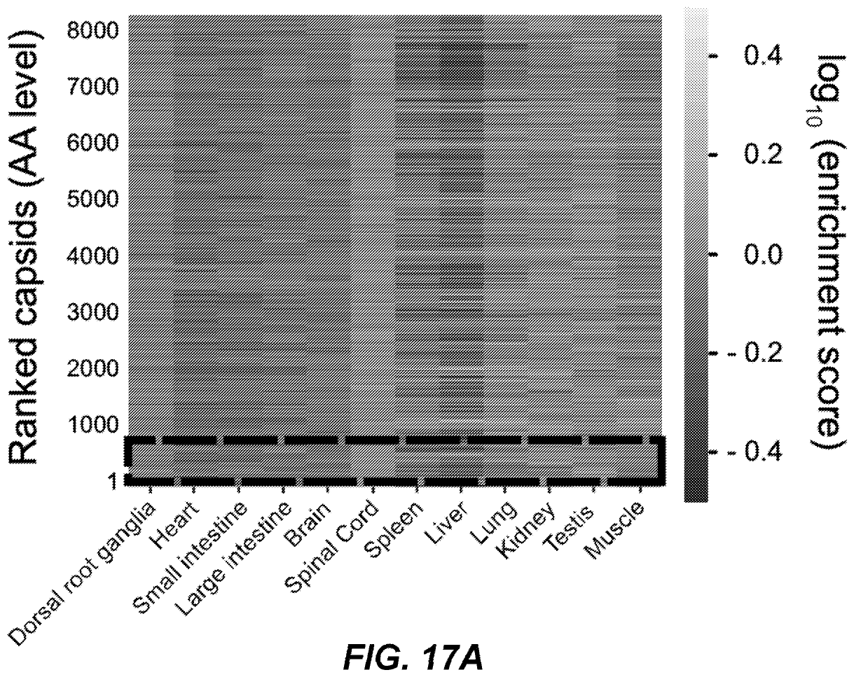

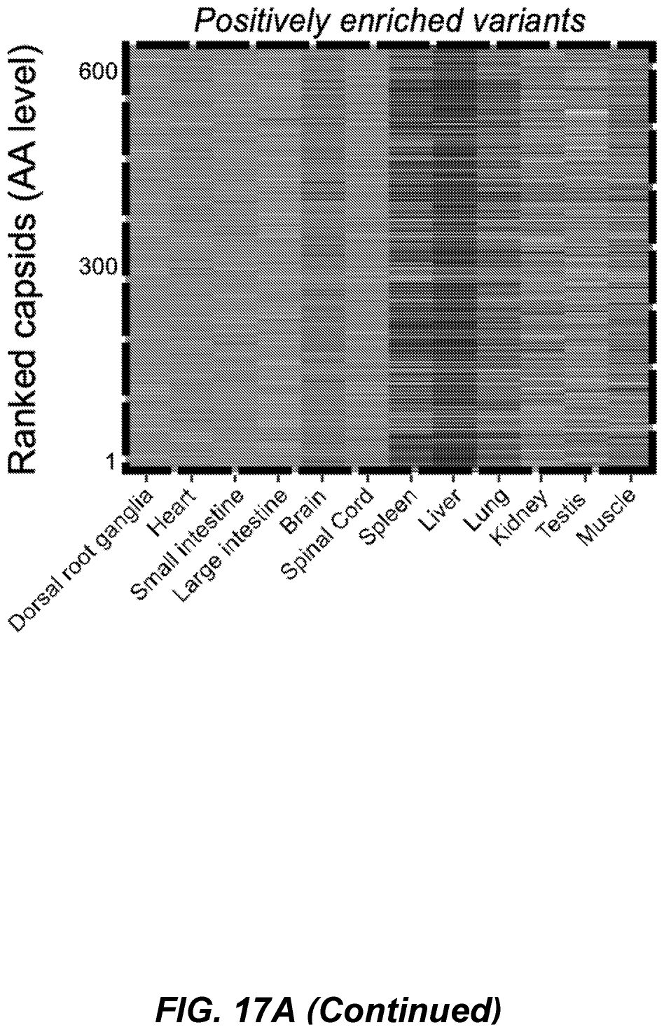

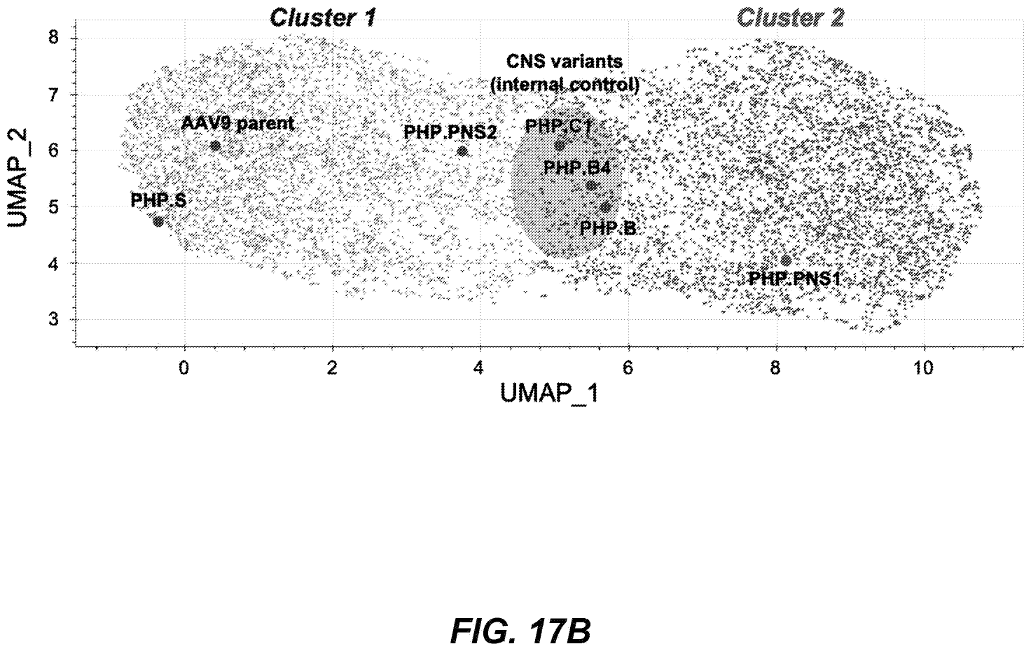

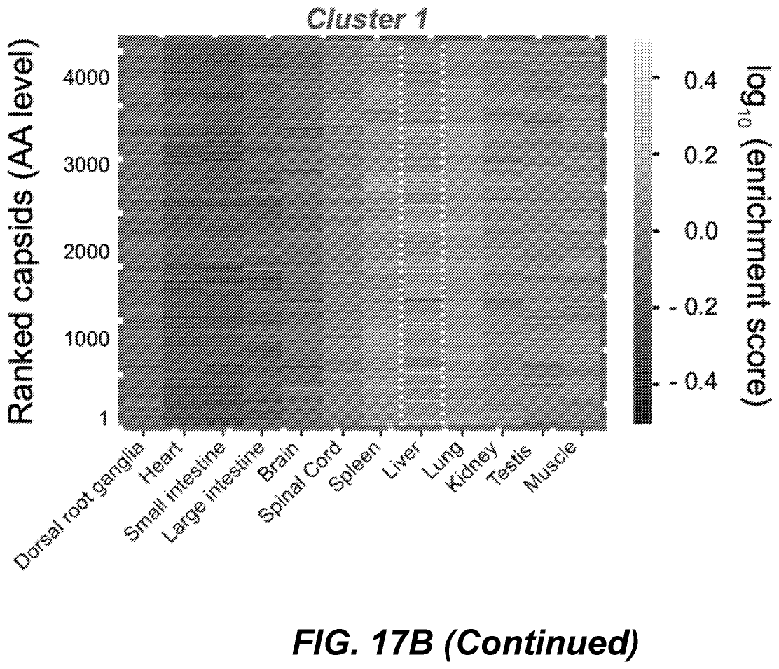

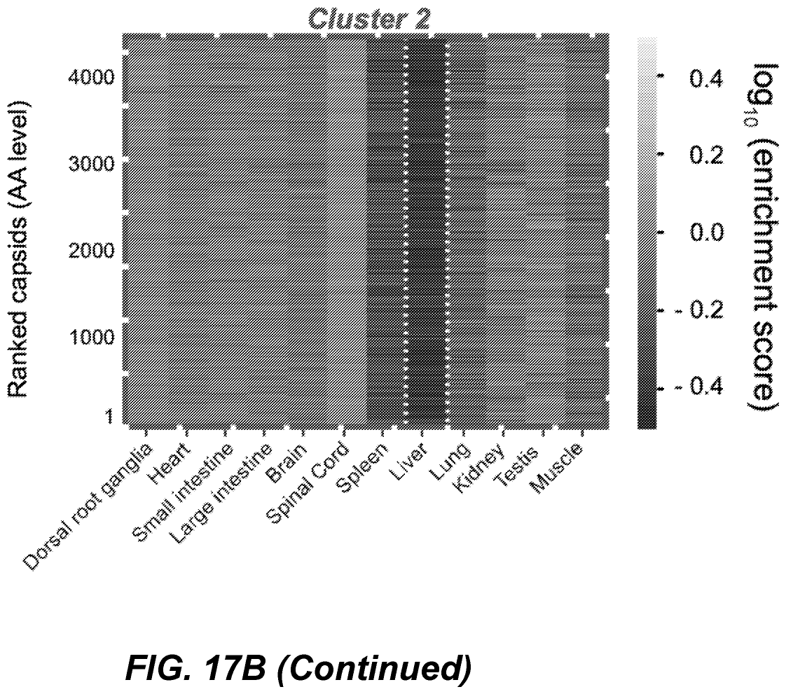

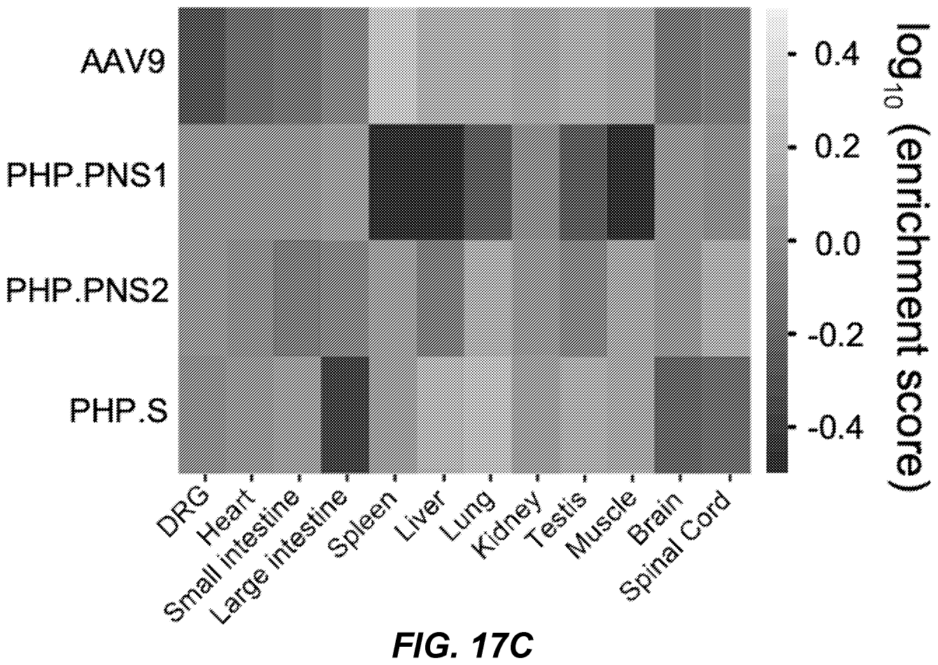

Disclosed herein include amino acid sequences combinations described below in addition to those in Table 2-Table 4, alone or part of a vector (e.g., AAV capsid). The 7 amino acid peptides can be inserted between AA588-589 of AAV9 capsid, and the 11 amino acid peptides were replaced between AA587-590 of AAV9 capsid (FIG. 7F). PHEGSSR (SEQ ID NO:169) insertion at AA position 588/589 of AAV9 VP1, which is listed in Table 2, can target the central and peripheral nervous system across species (FIG. 1A, FIG. 1F-FIG. 6F, FIG. 9A-FIG. 11C, FIG. 11F-FIG. 12, FIG. 16B-FIG. 17C). LNNTKTT (SEQ ID NO: 237) insertion at AA position 588/589 of AAV9 VP1, which is listed in Table 3, can target the cell types of the lung across species. (FIG. 7A-FIG. 8G) SNLARNV (SEQ ID NO: 274) insertion at AA position 588/589 of AAV9 VP1, which is listed in Table 3, can target the cell types of the lung across species. (FIG. 7A-FIG. 8G)

The peptides in Table 2 (200 sequences, SEQ ID NOs: 1-200) can target the central and peripheral nervous system across species. The peptides in Table 3 (101 sequences, SEQ ID NOs: 201-301) can target the cell types of the lung across species. The peptides in Table 4 (78 sequences, SEQ ID NOs: 302-379) can target the cell types of the lung across species. The peptide AQTNNTKPLAH (SEQ ID NO: 384) replacing AA position 587-590 of AAV9 VP1 (is listed in Table 5 (FIG. 7A-FIG. 8G)) can target the lung across species. The peptides in Table 5 (5 sequences, SEQ ID NOs: 380-384) can target the cell types of the lung across species.

Some embodiments provide the use of AAV capsids made by these methods described herein for delivery of DNA molecules, therapeutic proteins, small therapeutic molecules, or other biologics in vitro and in vivo.

The peptide sequence “PHEGSSR” (SEQ ID NO: 169) can be positioned between AA 588-589 of AAV9 capsid (shown below as SEQ ID NO: 385).

(SEQ ID NO: 385)
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
IWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSRTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
PHEGSSRAQAQTGWVQNQGILPGMVWQDRDVYLQGPIWAKIPHTDGNFH
PSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIG
TRYLTRNL

The peptide sequence “AQTNNTKPLAH” (SEQ ID NO: 384) can be positioned by replacing of AA 587-590 of AAV9 capsid (shown below as SEQ ID NO: 386)).

(SEQ ID NO: 386)
MAADGYLPDWLEDNLSEGIREWWALKPGAPQPKANQQHQDNARGLVLPG
YKYLGPGNGLDKGEPVNAADAAALEHDKAYDQQLKAGDNPYLKYNHADA
EFQERLKEDTSFGGNLGRAVFQAKKRLLEPLGLVEEAAKTAPGKKRPVE
QSPQEPDSSAGIGKSGAQPAKKRLNFGQTGDTESVPDPQPIGEPPAAPS
GVGSLTMASGGGAPVADNNEGADGVGSSSGNWHCDSQWLGDRVITTSTR
TWALPTYNNHLYKQISNSTSGGSSNDNAYFGYSTPWGYFDFNRFHCHFS
PRDWQRLINNNWGFRPKRLNFKLFNIQVKEVTDNNGVKTIANNLTSTVQ
VFTDSDYQLPYVLGSAHEGCLPPFPADVFMIPQYGYLTLNDGSQAVGRS
SFYCLEYFPSQMLRTGNNFQFSYEFENVPFHSSYAHSQSLDRLMNPLID
QYLYYLSRTINGSGQNQQTLKFSVAGPSNMAVQGRNYIPGPSYRQQRVS
TTVTQNNNSEFAWPGASSWALNGRNSLMNPGPAMASHKEGEDRFFPLSG
SLIFGKQGTGRDNVDADKVMITNEEEIKTTNPVATESYGQVATNHQSAQ
TNNTKPLAHAQTGWVQNQGILPGMVWQDRDVYLQGPIWAKIPHTDGNFH
PSPLMGGFGMKHPPPQILIKNTPVPADPPTAFNKDKLNSFITQYSTGQV
SVEIEWELQKENSKRWNPEIQYTSNYYKSNNVEFAVNTEGVYSEPRPIG
TRYLTRNL

TABLE 2A
PNS SEQUENCE 7 MER

| SEQ ID | | | | Small | Large | | Spinal |
|---|---|---|---|---|---|---|---|
| NO | Amino Acid | DRG | Heart | Intestine | Intestine | Brain | Cord |
| 1 | TPGNRTP | 0.09 | 0.04 | 0.08 | 0.09 | 0.03 | −0.13 |
| 2 | QIGSGGD | 0.00 | 0.06 | 0.16 | 0.06 | −0.07 | 0.00 |
| 3 | SYTDGPK | 0.22 | 0.06 | 0.00 | 0.00 | −0.10 | −0.01 |
| 4 | MHNNPAA | −0.09 | 0.04 | 0.12 | 0.20 | −0.31 | 0.07 |
| 5 | AVTLHTE | 0.09 | −0.15 | 0.09 | 0.23 | −0.20 | 0.10 |
| 6 | QLTNNLG | 0.18 | −0.19 | 0.17 | 0.10 | 0.02 | −0.17 |
| 7 | RVNDPIL | 0.10 | 0.08 | −0.03 | 0.10 | −0.35 | −0.05 |
| 8 | NYGTMNE | 0.00 | 0.10 | 0.10 | 0.04 | −0.27 | −0.10 |
| 9 | HGTSITN | 0.18 | 0.01 | 0.00 | 0.05 | 0.01 | −0.09 |
| 10 | WSAAAQE | 0.07 | 0.00 | 0.00 | 0.17 | −0.04 | 0.00 |
| 11 | TSSQNVE | 0.11 | 0.02 | 0.00 | 0.09 | 0.02 | 0.04 |
| 12 | NQPAVTA | 0.00 | 0.00 | 0.03 | 0.19 | −0.07 | 0.07 |
| 13 | TMLASGE | 0.14 | −0.04 | 0.12 | 0.00 | 0.05 | 0.04 |
| 14 | HDSNISS | 0.22 | −0.07 | 0.00 | 0.07 | −0.05 | 0.12 |
| 15 | GDIIYTK | 0.28 | −0.07 | 0.00 | 0.00 | −0.21 | 0.00 |
| 16 | LPETSNV | 0.16 | −0.09 | 0.14 | −0.01 | −0.10 | 0.03 |
| 17 | DNTFLST | 0.00 | 0.10 | 0.00 | 0.10 | 0.05 | 0.00 |
| 18 | YLEKMSA | 0.00 | 0.01 | 0.16 | 0.03 | 0.07 | 0.08 |
| 19 | YDASQLA | 0.07 | −0.04 | 0.11 | 0.06 | −0.06 | −0.07 |
| 20 | LDATYGS | 0.07 | 0.00 | 0.00 | 0.12 | −0.07 | −0.07 |
| 21 | TGHNQLA | 0.08 | −0.05 | 0.04 | 0.12 | 0.02 | 0.18 |
| 22 | STAAPRL | 0.08 | 0.11 | −0.10 | 0.11 | −0.18 | 0.09 |
| 23 | NITAIPN | 0.12 | 0.09 | 0.00 | −0.02 | −0.07 | 0.01 |
| 24 | TGTSIHP | 0.13 | 0.07 | 0.00 | −0.01 | 0.07 | 0.02 |
| 25 | MSVASQH | 0.07 | 0.07 | −0.10 | 0.14 | −0.06 | 0.07 |
| 26 | LPKPVIS | 0.00 | 0.09 | 0.00 | 0.09 | 0.11 | 0.19 |
| 27 | DPRTSLD | 0.17 | 0.01 | 0.00 | 0.00 | −0.34 | 0.09 |
| 28 | ANINKEN | 0.00 | 0.13 | 0.00 | 0.05 | −0.03 | −0.03 |
| 29 | ASNSPAL | 0.00 | 0.00 | 0.00 | 0.17 | −0.11 | −0.07 |
| 30 | SNLMSDF | 0.00 | 0.00 | 0.00 | 0.17 | −0.01 | 0.00 |
| 31 | VAASVSS | 0.00 | 0.05 | −0.02 | 0.14 | −0.13 | 0.11 |
| 32 | QRIEFSS | −0.10 | 0.01 | 0.00 | 0.25 | −0.19 | 0.03 |
| 33 | SMTPTSV | 0.00 | 0.10 | −0.11 | 0.18 | −0.30 | 0.07 |
| 34 | TTSTVTS | 0.00 | 0.00 | 0.07 | 0.09 | −0.21 | 0.06 |
| 35 | HDNFGSA | 0.13 | −0.07 | 0.07 | 0.04 | −0.11 | 0.02 |
| 36 | AEAKHPP | 0.05 | 0.04 | −0.01 | 0.08 | 0.03 | −0.07 |
| 37 | LKAQLPD | 0.08 | 0.01 | 0.00 | 0.07 | 0.05 | −0.14 |
| 38 | DINPTYS | 0.00 | 0.00 | 0.03 | 0.13 | 0.01 | 0.05 |
| 39 | SSASLLA | 0.09 | 0.02 | 0.06 | −0.01 | −0.03 | 0.03 |
| 40 | GNQTPSV | 0.00 | 0.00 | 0.00 | 0.16 | −0.26 | 0.07 |
| 41 | IGPTVRM | −0.15 | 0.17 | −0.09 | 0.22 | 0.01 | 0.05 |
| 42 | GIPTRFS | 0.16 | 0.00 | −0.10 | 0.10 | −0.22 | 0.03 |
| 43 | RESLPGH | 0.00 | 0.07 | 0.00 | 0.09 | −0.07 | 0.02 |
| 44 | GDTLREC | 0.10 | 0.06 | 0.00 | 0.00 | −0.02 | 0.01 |
| 45 | VPSMTVE | 0.00 | 0.00 | 0.05 | 0.11 | −0.02 | −0.04 |
| 46 | TDQAFRS | 0.15 | 0.00 | 0.00 | 0.01 | 0.06 | 10.09 |
| 47 | GVNDHVH | 0.25 | 0.00 | −0.12 | 0.02 | −0.16 | −0.03 |
| 48 | LPANQGT | 0.00 | 0.04 | 0.00 | 0.11 | −0.06 | 10.05 |
| 49 | TLLPRSN | 0.00 | −0.12 | 0.12 | 0.16 | −0.02 | −0.02 |
| 50 | GSGLAGN | −0.08 | 0.03 | 0.11 | 0.09 | −0.09 | 0.09 |
| 51 | HMPSANY | 0.00 | 0.00 | 0.07 | 0.08 | −0.12 | 0.00 |
| 52 | LSGSGSA | 0.00 | −0.07 | −0.07 | 0.29 | −0.09 | 0.17 |
| 53 | VFDLSRT | 0.00 | 0.00 | 0.10 | 0.05 | −0.06 | 0.03 |
| 54 | TALTGPT | 0.00 | 0.00 | 0.11 | 0.04 | 0.00 | 0.02 |
| 55 | TLNPKSP | 0.24 | −0.10 | 0.00 | 0.01 | 0.08 | 0.08 |
| 56 | TGPTMSL | 0.06 | 0.00 | 0.00 | 0.08 | −0.12 | 0.03 |
| 57 | RAKILAS | 0.07 | 0.07 | 0.00 | 0.00 | −0.16 | 0.01 |
| 58 | LAIASAP | 0.00 | 0.00 | 0.00 | 0.14 | 0.12 | 0.13 |
| 59 | SKNDAVA | 0.00 | 0.07 | 0.01 | 0.07 | −0.15 | 0.14 |
| 60 | VPATDDK | 0.00 | 0.00 | 0.00 | 0.14 | −0.03 | 0.11 |
| 61 | LSISLGD | −0.11 | 0.06 | 0.11 | 0.09 | −0.25 | 0.09 |
| 62 | QASNLEM | 0.00 | 0.00 | 0.11 | 0.03 | −0.05 | −0.03 |
| 63 | DLLSNIA | 0.00 | 0.09 | 0.00 | 0.05 | −0.09 | 0.06 |
| 64 | LSSALPK | 0.04 | 0.13 | −0.01 | −0.02 | −0.03 | −0.07 |
| 65 | HTTLGIN | 0.00 | 0.00 | 0.00 | 0.13 | 0.03 | 0.07 |
| 66 | LHVNSTN | 0.12 | −0.05 | 0.00 | 0.07 | 0.02 | 0.03 |
| 67 | PVTQLSR | 0.06 | 0.07 | 0.00 | 0.00 | −0.03 | 0.06 |
| 68 | IALNSEP | 0.13 | 0.00 | −0.10 | 0.11 | −0.07 | −0.10 |
| 69 | QEIPRLN | −0.06 | 0.00 | 0.00 | 0.20 | 0.04 | 0.02 |
| 70 | INSTLQF | 0.00 | 0.05 | 0.19 | −0.10 | −0.07 | −0.10 |
| 71 | AVPAKER | 0.07 | 0.00 | 0.16 | −0.10 | −0.11 | 0.06 |
| 72 | GQNKEPG | 0.05 | 0.07 | 0.01 | 0.00 | 0.01 | −0.01 |
| 73 | DPQNSGL | 0.00 | 0.01 | 0.00 | 0.12 | 0.24 | 0.09 |
| 74 | DELQSRS | 0.13 | 0.00 | 0.00 | 0.00 | −0.10 | 0.00 |
| 75 | MQTTERE | 0.00 | −0.08 | 0.11 | 0.11 | 0.27 | 0.10 |
| 76 | ELSHQLA | 0.00 | 0.00 | 0.09 | 0.04 | −0.11 | 0.09 |
| 77 | TSAQGTQ | 0.05 | −0.08 | 0.05 | 0.11 | −0.11 | 0.03 |
| 78 | LNKESSW | 0.19 | −0.01 | 0.00 | −0.06 | −0.06 | −0.05 |
| 79 | ESSPHRT | −0.01 | 0.09 | 0.04 | 0.02 | −0.17 | 0.19 |
| 80 | RSLSDFS | 0.14 | 0.00 | 0.00 | −0.01 | −0.02 | 0.04 |
| 81 | TLIDSAA | 0.00 | 0.00 | 0.00 | 0.13 | −0.13 | 0.00 |
| 82 | ADTMSFR | 0.00 | 0.00 | 0.00 | 0.13 | −0.12 | 0.02 |
| 83 | DVAGRGF | 0.05 | 0.03 | 0.00 | 0.04 | −0.02 | −0.10 |
| 84 | LLGAEPP | 0.00 | 0.00 | 0.12 | 0.01 | −0.08 | 0.01 |
| 85 | QLSDAMA | 0.00 | 0.00 | 0.07 | 0.05 | −0.08 | 0.07 |
| 86 | GSEIYRG | 0.07 | 0.00 | −0.04 | 0.09 | −0.08 | 0.03 |
| 87 | DGVSHTL | −0.11 | 0.00 | 0.10 | 0.13 | −0.19 | −0.02 |
| 88 | VVHEASL | 0.00 | 0.06 | 0.00 | 0.06 | 0.04 | −0.08 |
| 89 | ILQSHPL | 0.16 | −0.07 | 0.00 | 0.03 | −0.06 | 0.02 |
| 90 | ATYVTST | 0.00 | 0.05 | 0.08 | −0.01 | 0.09 | −0.08 |
| 91 | TAMQLGH | 0.00 | 0.02 | 0.04 | 0.10 | −0.03 | 0.18 |
| 92 | PVNSTGA | 0.01 | 0.04 | 0.00 | 0.07 | −0.03 | 0.09 |
| 93 | RPVSPRA | 0.00 | −0.01 | 0.00 | 0.13 | −0.13 | 0.00 |
| 94 | IPASPFT | 0.08 | 0.00 | 0.12 | −0.08 | −0.12 | 0.04 |
| 95 | ANDYRYD | 0.00 | 0.06 | 0.00 | 0.06 | −0.14 | −0.03 |
| 96 | YNSEPRI | 10.00 | 0.05 | −0.11 | 10.28 | −0.14 | 0.07 |
| 97 | AIETRNV | 0.00 | 0.08 | 0.07 | 0.12 | −0.01 | 0.12 |
| 98 | PASQGLS | 0.00 | −0.06 | 0.10 | 0.08 | 0.07 | 0.05 |
| 99 | RDSDALR | 0.00 | 0.00 | 0.00 | 0.12 | −0.18 | 0.00 |
| 100 | CQAPKAD | 0.08 | 0.00 | 0.00 | 0.03 | −0.25 | 0.00 |
| 101 | ALSGAVT | 0.06 | 0.00 | 0.00 | 0.05 | −0.03 | −0.03 |
| 102 | VQPIFPL | 0.00 | 0.00 | 0.00 | 0.11 | −0.03 | 0.00 |
| 103 | SVLGNAS | 0.08 | −0.07 | 0.07 | 0.03 | −0.23 | 0.03 |
| 104 | APRLDAA | 0.00 | −0.07 | 0.12 | 0.07 | 0.03 | 0.04 |
| 105 | SASSIVN | 0.00 | 0.08 | 0.00 | 0.03 | 0.04 | −0.03 |
| 106 | GVHVKPN | 0.21 | −0.14 | 0.10 | −0.05 | −0.16 | −0.01 |
| 107 | LQVSEPR | 0.11 | 0.02 | 0.00 | −0.02 | −0.08 | 0.06 |
| 108 | AGAHVPS | 0.00 | 0.00 | 0.00 | 0.11 | 0.01 | 0.15 |
| 109 | PLMGTAS | 0.00 | 0.00 | 0.00 | 0.11 | −0.14 | −0.02 |
| 110 | QVPTWNS | 0.00 | 0.11 | 0.00 | 0.00 | 0.02 | 0.07 |
| 111 | DHRTTYK | 0.00 | 0.00 | 0.00 | 0.11 | −0.25 | 0.04 |
| 112 | HPEKEAA | 0.00 | 0.00 | 0.14 | −0.03 | −0.02 | −0.07 |
| 113 | AGPGLGL | 0.09 | −0.05 | 0.00 | 0.07 | −0.09 | 0.07 |
| 114 | ITPTPSS | 0.00 | −0.06 | 0.00 | 0.17 | −0.10 | 0.07 |
| 115 | TGMSVVH | 0.00 | 0.00 | 0.00 | 0.11 | −0.06 | 0.03 |
| 116 | LMDASGT | 0.00 | 0.00 | 0.00 | 0.11 | −0.08 | 0.08 |
| 117 | SPYPNTS | 0.00 | 0.00 | 0.00 | 0.11 | −0.02 | −0.04 |
| 118 | SNHAPVS | 0.06 | 0.01 | −0.14 | 0.18 | −0.27 | 0.07 |
| 119 | ASGLALA | 0.00 | −0.04 | 0.08 | 0.06 | −0.14 | 0.03 |
| 120 | PTAATGT | 0.07 | 0.00 | 0.00 | 0.04 | 0.14 | −0.06 |
| 121 | DFVHVAN | −0.02 | 0.00 | 0.00 | 0.13 | 0.05 | 0.00 |
| 122 | LTNSRFM | 0.12 | 0.08 | −0.11 | 0.02 | −0.30 | −0.03 |
| 123 | LIIPSLP | 0.00 | 0.00 | 0.10 | 0.00 | −0.01 | 0.11 |
| 124 | GTLTMQT | 0.12 | −0.07 | 0.13 | −0.08 | −0.13 | 0.12 |
| 125 | GTDHYTA | 0.00 | 0.08 | 0.05 | 0.14 | −0.11 | 0.07 |
| 126 | ALPKGTT | 0.33 | −0.15 | −0.05 | −0.03 | −0.20 | 0.09 |
| 127 | FLAPVQS | 0.00 | 0.00 | 0.00 | 0.10 | 0.03 | 0.04 |
| 128 | HDAPSIS | 0.00 | 0.00 | 0.09 | 0.01 | −0.09 | 0.04 |
| 129 | VTIGPQR | 0.00 | 0.10 | 0.00 | 0.00 | −0.04 | −0.07 |
| 130 | VDHGTMA | 0.00 | 0.02 | 0.00 | 0.08 | −0.22 | 0.01 |
| 131 | TDSVSTT | 0.14 | −0.08 | 0.00 | 0.04 | −0.03 | −0.14 |
| 132 | STVAIVP | 0.00 | 0.05 | 0.00 | 0.04 | −0.09 | −0.07 |
| 133 | ALQADRA | 0.00 | 0.00 | 0.09 | 0.01 | 0.01 | 0.02 |
| 134 | DPRLSVE | 0.06 | 0.00 | 0.00 | 0.04 | −0.09 | 0.02 |
| 135 | NTQTPAS | 0.00 | 0.00 | −0.03 | 0.13 | −0.01 | 0.09 |
| 136 | SGNGGMH | 0.00 | 0.06 | 0.01 | 0.03 | −0.17 | 0.00 |
| 137 | KNLLESI | 0.00 | 0.10 | 0.00 | 0.00 | −0.08 | −0.02 |
| 138 | NSSDSAY | 0.12 | 0.00 | 0.00 | −0.03 | 0.00 | 0.22 |
| 139 | LGYATAE | 0.00 | 0.04 | 0.00 | 0.05 | −0.14 | 0.03 |
| 140 | VLDHSAD | 0.00 | 0.00 | 0.00 | 0.09 | −0.18 | 0.00 |
| 141 | LLGALAP | 0.11 | −0.05 | 0.00 | 0.04 | −0.08 | 0.07 |
| 142 | HIPDRQH | 0.00 | 0.00 | 0.00 | 0.09 | −0.15 | −0.09 |
| 143 | LPASSGL | 0.00 | 0.01 | −0.12 | 0.22 | −0.10 | 0.06 |
| 144 | PVTTSTS | 0.00 | 0.00 | 0.00 | 0.09 | −0.06 | 0.05 |
| 145 | AHNANMA | 0.04 | −0.23 | 0.00 | 0.28 | −0.14 | −0.01 |
| 146 | LLQNLSL | 0.00 | 0.00 | 0.00 | 0.09 | −0.10 | 0.00 |
| 147 | TVTMPAS | 0.00 | 0.00 | 0.00 | 0.09 | 0.03 | −0.02 |
| 148 | LSLSHNQ | 0.00 | 0.00 | 0.00 | 0.09 | −0.05 | 0.09 |
| 149 | SHPPSLP | 0.12 | 0.00 | −0.03 | −0.01 | −0.01 | 0.01 |
| 150 | PEPNGTP | 0.00 | 0.00 | 0.00 | 0.09 | −0.04 | 0.00 |
| 151 | TDQVMLY | 0.00 | 0.00 | 0.09 | 0.00 | −0.06 | 0.02 |
| 152 | EKMLVGA | 0.01 | 0.00 | 0.09 | 0.01 | −0.20 | −0.01 |
| 153 | PQSHSRQ | 0.09 | 0.00 | 0.00 | 0.00 | −0.11 | 0.00 |
| 154 | LQAISLS | 0.14 | 0.14 | 0.00 | 0.10 | 0.01 | 0.03 |
| 155 | IGPTDRM | 0.08 | −0.06 | 0.00 | 0.07 | 0.07 | 0.03 |
| 156 | IVNDSVG | 0.09 | 0.00 | 0.00 | 0.00 | −0.04 | 0.10 |
| 157 | TNGAAGW | 0.00 | 0.00 | −0.06 | 0.15 | −0.10 | 0.00 |
| 158 | TGIAVQS | 0.06 | 0.00 | 0.03 | 0.00 | −0.08 | 0.05 |
| 159 | SSGTPSW | 0.00 | 0.09 | −0.07 | 0.07 | −0.08 | 0.11 |
| 160 | LLTLAPS | 0.09 | 0.00 | 0.00 | 0.00 | −0.08 | −0.07 |
| 161 | SWSERPV | 0.13 | −0.08 | 0.00 | 0.04 | −0.13 | 0.04 |
| 162 | NCPMKSQ | 0.09 | 0.00 | 0.00 | 0.00 | 0.00 | 0.00 |
| 163 | KLSSGLD | 0.07 | −0.07 | −0.10 | 0.19 | −0.04 | −0.10 |
| 164 | TTGMIST | 0.00 | 0.08 | 0.00 | 0.01 | 0.01 | 0.05 |
| 165 | MAHDTQS | 0.13 | −0.06 | 0.00 | 0.01 | −0.09 | 0.00 |
| 166 | ILSLAES | 0.00 | 0.00 | 0.00 | 0.09 | −0.02 | 0.02 |
| 167 | GGDVLTL | 0.00 | 0.00 | 0.00 | 0.08 | −0.14 | −0.07 |
| 168 | LSQGTQT | 0.10 | 0.06 | −0.05 | −0.04 | −0.12 | 0.05 |
| 169 | PHEGSSR | 0.00 | 0.00 | 0.00 | 0.08 | 0.03 | 0.00 |
| 170 | QIVNNTY | 0.00 | 0.01 | 0.00 | 0.07 | −0.14 | −0.02 |
| 171 | GLLATNA | 0.08 | 0.06 | 0.08 | −0.14 | −0.12 | −0.02 |
| 172 | QGDTSGL | 0.00 | 0.00 | 0.02 | 0.06 | −0.11 | 0.00 |
| 173 | KVSTPLR | 0.22 | 0.00 | −0.17 | 0.03 | 0.00 | 0.00 |
| 174 | SNAMSST | 0.00 | 0.03 | 0.00 | 0.05 | 0.00 | 0.03 |
| 175 | LTALEMM | 0.00 | 0.00 | 0.08 | 0.00 | −0.02 | 0.00 |
| 176 | ILSIDQS | 0.00 | 0.00 | 0.00 | 0.08 | −0.09 | −0.07 |
| 177 | PLTLAPK | 0.10 | 0.00 | −0.01 | 0.00 | −0.30 | 0.05 |
| 178 | YVAEAAV | 0.00 | 0.08 | 0.00 | 0.00 | −0.12 | −0.02 |
| 179 | GKQSWAD | 0.00 | 0.04 | 0.00 | 0.04 | −0.10 | 0.06 |
| 180 | NLEDKST | 0.00 | 0.00 | 0.00 | 0.08 | −0.15 | 0.00 |
| 181 | YSNSQHH | 0.07 | −0.15 | 0.16 | −0.01 | −0.04 | 0.08 |
| 182 | KVTPSTP | 0.10 | 0.14 | −0.24 | 0.08 | −0.12 | 0.09 |
| 183 | KTNVPGD | 0.00 | −0.06 | 0.07 | 0.07 | −0.09 | 0.09 |
| 184 | NRTTVDN | 0.23 | 0.29 | 0.23 | −0.09 | −0.10 | 0.21 |
| 185 | LIPEPPK | 0.09 | 0.00 | 0.00 | −0.01 | −0.02 | −0.02 |
| 186 | HSPMAIN | 0.00 | 0.00 | 0.00 | 0.08 | 0.02 | 0.03 |
| 187 | YAPQMPF | 0.00 | 0.05 | 0.00 | 0.02 | −0.12 | −0.04 |
| 188 | DTLQYPR | 0.00 | −0.02 | 0.01 | 0.09 | 0.00 | −0.03 |
| 189 | IIVSECS | 0.00 | 0.07 | 0.00 | 0.00 | −0.06 | 0.00 |
| 190 | WLDKLSK | 0.08 | 0.00 | 0.00 | 0.00 | −0.31 | −0.02 |
| 191 | PVQHVSN | 0.00 | 0.00 | 0.00 | 0.07 | 0.02 | −0.15 |
| 192 | AVENTTP | 0.06 | 0.00 | −0.08 | 0.10 | −0.10 | 0.00 |
| 193 | IDTTSCC | 0.00 | 0.00 | 0.00 | 0.07 | 0.00 | 0.00 |
| 194 | GQLVTNL | 0.00 | 0.00 | 0.00 | 0.07 | −0.07 | −0.02 |
| 195 | NGTGSTT | 0.05 | 0.05 | 0.00 | −0.03 | −0.07 | 0.07 |
| 196 | STPTVEP | 0.00 | −0.07 | 0.00 | 0.14 | −0.11 | 0.00 |
| 197 | MNGQLTC | 0.04 | −0.04 | 0.00 | 0.06 | −0.11 | 0.06 |
| 198 | GNFLRAD | 0.00 | 0.02 | 0.00 | 0.05 | −0.09 | 0.01 |
| 199 | TGLPHNE | 0.00 | 0.00 | 0.00 | 0.07 | −0.05 | 0.03 |
| 200 | TSGEDAL | 0.00 | 0.00 | 0.00 | 0.07 | −0.08 | 0.00 |

log10 enrichment scores for the indicated tissue or organ are shown

TABLE 2B
PNS SEQUENCE 7 MER (CONTINUED)

| SEQ | | | | | | | | |
|---|---|---|---|---|---|---|---|---|
| ID | | | | | | | | PNS |
| NO | Eye | Spleen | Liver | Lung | Kidney | Testis | Muscle | average |
| 1 | 0.57 | 0.24 | 0.38 | 0.27 | 0.16 | 0.15 | 0.01 | 0.07 |
| 2 | 0.00 | 0.09 | −0.19 | −0.09 | −0.05 | 0.25 | 0.04 | 0.07 |
| 3 | 0.10 | −0.42 | −0.73 | −0.13 | 0.01 | −0.06 | −0.42 | 0.07 |
| 4 | 0.17 | 0.13 | 0.09 | 0.15 | 0.12 | 0.12 | 0.13 | 0.07 |
| 5 | 0.05 | −0.08 | −0.53 | −0.09 | −0.01 | 0.01 | −0.18 | 0.07 |
| 6 | −0.18 | 0.04 | −0.01 | 0.02 | 0.07 | −0.14 | −0.06 | 0.06 |
| 7 | 0.00 | −0.33 | −0.65 | −0.20 | −0.04 | −0.25 | 0.28 | 0.06 |
| 8 | 0.07 | −0.01 | −0.13 | −0.05 | 0.01 | 0.05 | 0.08 | 0.06 |
| 9 | 0.10 | 0.22 | 0.06 | 0.07 | −0.02 | 0.06 | 0.11 | 0.06 |
| 10 | 0.17 | −0.32 | −0.76 | −0.33 | −0.10 | 0.07 | −0.16 | 0.06 |
| 11 | −0.16 | −0.11 | −0.42 | −0.12 | 0.02 | 0.02 | −0.29 | 0.06 |
| 12 | 0.27 | 0.11 | 0.02 | −0.01 | 0.21 | 0.03 | 0.07 | 0.05 |
| 13 | 0.00 | −0.05 | −0.30 | −0.20 | −0.14 | −0.17 | −0.03 | 0.05 |
| 14 | 0.00 | −0.32 | −0.33 | −0.25 | 0.10 | 0.14 | 0.05 | 0.05 |
| 15 | 0.00 | −0.79 | −0.74 | −0.49 | −0.18 | −0.17 | −0.33 | 0.05 |
| 16 | 0.00 | −0.10 | −0.66 | −0.16 | −0.12 | 0.16 | −0.12 | 0.05 |
| 17 | 0.00 | −0.03 | −0.21 | 0.05 | −0.10 | 0.03 | −0.08 | 0.05 |
| 18 | 0.13 | 0.02 | −0.25 | −0.01 | 0.01 | −0.18 | 0.15 | 0.05 |
| 19 | 0.06 | −0.47 | −0.73 | −0.15 | 0.04 | 0.02 | −0.06 | 0.05 |
| 20 | 0.17 | −0.04 | −0.59 | −0.13 | −0.06 | −0.01 | −0.20 | 0.05 |
| 21 | 0.12 | 0.20 | 0.07 | 0.02 | 0.18 | 0.16 | 0.20 | 0.05 |
| 22 | 0.02 | −0.25 | −0.22 | −0.06 | 0.06 | 0.05 | −0.12 | 0.05 |
| 23 | 0.00 | −0.19 | −0.21 | −0.12 | −0.26 | −0.21 | −0.05 | 0.05 |
| 24 | 0.09 | 0.18 | 0.05 | 0.20 | −0.05 | 0.07 | 0.33 | 0.05 |
| 25 | 0.20 | −0.05 | −0.01 | 0.05 | −0.13 | 0.15 | −0.03 | 0.05 |
| 26 | 0.18 | 0.08 | 0.25 | 0.31 | 0.14 | 0.01 | −0.02 | 0.05 |
| 27 | 0.00 | −0.22 | −0.70 | −0.48 | −0.06 | 0.15 | 0.03 | 0.04 |
| 28 | 0.13 | 0.01 | −0.23 | 0.08 | −0.01 | 0.14 | −0.15 | 0.04 |
| 29 | 0.00 | −0.18 | −0.30 | 0.16 | 0.02 | −0.14 | 0.02 | 0.04 |
| 30 | 0.00 | −0.06 | −0.08 | −0.12 | 0.02 | 0.00 | 0.00 | 0.04 |
| 31 | 0.12 | 0.19 | −0.12 | 0.09 | −0.09 | −0.02 | −0.11 | 0.04 |
| 32 | 0.19 | −0.12 | −0.32 | −0.17 | −0.03 | −0.07 | −0.11 | 0.04 |
| 33 | −0.25 | −0.29 | −0.46 | −0.21 | −0.10 | −0.23 | −0.03 | 0.04 |
| 34 | 0.18 | 0.27 | 0.14 | 0.22 | 0.11 | 0.03 | 0.06 | 0.04 |
| 35 | 0.01 | −0.09 | −0.49 | −0.13 | −0.19 | −0.10 | −0.23 | 0.04 |
| 36 | 0.19 | 0.10 | 0.06 | 0.21 | 0.15 | −0.16 | 0.29 | 0.04 |
| 37 | 0.05 | 0.20 | 0.00 | 0.24 | −0.05 | 0.22 | 0.15 | 0.04 |
| 38 | 0.08 | −0.60 | 0.81 | −0.40 | 0.03 | −0.23 | −0.16 | 0.04 |
| 39 | 0.06 | 0.13 | 0.05 | 0.04 | 0.07 | −0.12 | 0.05 | 0.04 |
| 40 | 0.00 | −0.55 | −0.87 | −0.20 | 0.16 | −0.03 | −0.26 | 0.04 |
| 41 | 0.00 | 0.08 | 0.17 | 0.19 | 0.10 | 0.04 | 0.28 | 0.04 |
| 42 | 0.00 | −0.22 | −0.03 | −0.11 | −0.08 | 0.22 | −0.50 | 0.04 |
| 43 | 0.08 | −0.36 | −0.49 | −0.17 | 0.02 | 0.03 | −0.24 | 0.04 |
| 44 | 0.00 | −0.21 | −0.59 | −0.21 | 0.10 | −0.07 | −0.17 | 0.04 |
| 45 | 0.09 | −0.17 | −0.35 | −0.10 | −0.15 | −0.34 | −0.18 | 0.04 |
| 46 | 0.13 | 0.23 | −0.02 | 0.00 | −0.16 | 0.31 | 0.22 | 0.04 |
| 47 | −0.19 | −0.49 | −0.97 | −0.25 | −0.06 | −0.19 | −0.30 | 0.04 |
| 48 | 0.09 | −0.06 | −0.41 | −0.20 | 0.05 | −0.15 | −0.07 | 0.04 |
| 49 | 0.00 | −0.13 | 0.04 | 0.12 | 0.13 | 0.08 | 0.06 | 0.04 |
| 50 | −0.03 | 0.04 | −0.28 | −0.10 | −0.14 | 0.13 | −0.17 | 0.04 |
| 51 | 0.00 | −0.25 | −0.27 | −0.02 | −0.11 | 0.07 | 0.02 | 0.04 |
| 52 | 0.10 | 0.45 | 0.29 | 0.19 | 0.20 | 0.11 | 0.39 | 0.04 |
| 53 | 0.16 | −0.54 | −0.47 | −0.17 | −0.02 | −0.02 | −0.25 | 0.04 |
| 54 | 0.00 | −0.28 | −0.37 | −0.09 | −0.09 | 0.03 | −0.21 | 0.04 |
| 55 | −0.49 | 0.14 | 0.14 | 0.16 | −0.06 | −0.09 | 0.36 | 0.04 |
| 56 | 0.00 | −0.09 | −0.34 | −0.11 | −0.11 | 0.05 | 0.01 | 0.04 |
| 57 | 0.00 | −0.54 | −0.13 | −0.17 | −0.01 | 0.24 | −0.21 | 0.04 |
| 58 | 0.00 | 0.26 | −0.03 | 0.01 | 0.08 | −0.04 | 0.19 | 0.04 |
| 59 | 0.21 | −0.03 | −0.17 | 0.20 | 0.04 | 0.05 | −0.06 | 0.04 |
| 60 | 0.00 | −0.72 | −0.84 | −0.51 | −0.28 | −0.05 | −0.18 | 0.04 |
| 61 | 0.09 | −0.32 | −0.64 | −0.14 | 0.17 | −0.35 | −0.15 | 0.04 |
| 62 | 0.00 | −0.46 | −0.98 | −0.32 | −0.08 | −0.14 | −0.25 | 0.03 |
| 63 | 0.00 | −0.24 | −0.92 | 0.02 | 0.00 | −0.18 | −0.08 | 0.03 |
| 64 | −0.39 | −0.43 | −0.66 | −0.15 | 0.05 | −0.17 | −0.27 | 0.03 |
| 65 | 0.17 | −0.02 | −0.14 | 0.11 | −0.12 | −0.10 | 0.21 | 0.03 |
| 66 | 0.00 | −0.16 | 0.01 | −0.06 | −0.01 | 0.17 | 0.09 | 0.03 |
| 67 | −0.04 | −0.49 | −0.67 | −0.12 | −0.27 | 0.07 | −0.51 | 0.03 |
| 68 | 0.00 | −0.19 | −0.75 | −0.17 | 0.11 | −0.09 | 0.04 | 0.03 |
| 69 | 0.00 | 0.00 | −0.27 | 0.02 | 0.27 | 0.10 | 0.17 | 0.03 |
| 70 | 0.07 | −0.34 | −0.53 | −0.16 | −0.36 | −0.12 | −0.47 | 0.03 |
| 71 | 0.20 | −0.38 | −0.37 | −0.15 | 0.04 | −0.34 | −0.10 | 0.03 |
| 72 | 0.00 | 0.09 | −0.13 | 0.05 | 0.14 | −0.07 | −0.05 | 0.03 |
| 73 | 0.16 | −0.13 | −0.83 | −0.13 | −0.05 | −0.15 | −0.11 | 0.03 |
| 74 | 0.00 | −0.26 | −0.48 | −0.26 | 0.00 | −0.10 | −0.17 | 0.03 |
| 75 | 0.00 | −0.23 | −0.18 | 0.03 | −0.09 | 0.12 | 0.16 | 0.03 |
| 76 | 0.00 | −0.14 | −0.45 | −0.18 | 0.12 | −0.09 | −0.02 | 0.03 |
| 77 | 0.24 | 0.09 | −0.03 | 0.00 | 0.03 | −0.12 | 0.21 | 0.03 |
| 78 | −0.41 | −0.34 | −0.71 | 0.05 | −0.12 | 0.13 | −0.22 | 0.03 |
| 79 | 0.45 | 0.05 | 0.11 | 0.07 | −0.03 | 0.17 | 0.19 | 0.03 |
| 80 | 0.20 | −0.21 | −0.37 | −0.23 | −0.05 | 0.09 | −0.01 | 0.03 |
| 81 | 0.00 | −0.12 | −0.49 | −0.58 | 0.08 | 0.03 | −0.01 | 0.03 |
| 82 | 0.00 | −0.30 | −0.57 | −0.13 | 0.04 | −0.04 | 0.02 | 0.03 |
| 83 | 0.29 | 0.20 | 0.10 | 0.23 | 0.08 | 0.10 | 0.14 | 0.03 |
| 84 | 0.00 | −0.30 | −0.68 | −0.36 | −0.02 | −0.12 | −0.23 | 0.03 |
| 85 | 0.00 | −0.37 | −0.72 | 0.06 | 0.12 | −0.04 | −0.11 | 0.03 |
| 86 | 0.06 | −0.12 | −0.45 | −0.19 | −0.17 | −0.06 | −0.13 | 0.03 |
| 87 | 0.00 | −0.46 | −0.69 | −0.49 | −0.09 | −0.19 | −0.17 | 0.03 |
| 88 | 0.00 | −0.22 | −0.49 | −0.09 | 0.02 | −0.14 | −0.05 | 0.03 |
| 89 | 0.10 | −0.20 | −0.44 | −0.13 | 0.08 | 0.09 | −0.28 | 0.03 |
| 90 | 0.38 | −0.03 | −0.17 | 0.21 | −0.11 | 0.10 | 0.18 | 0.03 |
| 91 | 0.17 | −0.23 | −0.44 | −0.10 | −0.08 | −0.15 | −0.24 | 0.03 |
| 92 | 0.08 | 0.23 | −0.02 | 0.03 | 0.18 | −0.11 | −0.02 | 0.03 |
| 93 | 0.00 | −0.76 | −0.45 | −0.57 | −0.09 | −0.29 | −0.09 | 0.03 |
| 94 | 0.00 | 0.01 | −0.16 | 0.11 | 0.07 | −0.35 | −0.24 | 0.03 |
| 95 | 0.18 | −0.31 | −0.62 | −0.35 | 0.07 | −0.04 | −0.27 | 0.03 |
| 96 | −0.13 | −0.46 | −0.57 | −0.22 | 0.05 | −0.17 | −0.10 | 0.03 |
| 97 | 0.03 | −0.01 | −0.18 | −0.15 | 0.07 | −0.04 | −0.06 | 0.03 |
| 98 | 0.00 | −0.03 | 0.07 | −0.04 | 0.12 | 0.15 | 0.02 | 0.03 |
| 99 | 0.00 | −0.40 | −0.61 | −0.06 | −0.02 | 0.08 | 0.02 | 0.03 |
| 100 | 0.00 | −0.52 | −0.54 | −0.23 | 0.17 | 0.02 | 0.05 | 0.03 |
| 101 | −0.27 | 0.05 | −0.21 | 0.02 | −0.05 | −0.39 | −0.09 | 0.03 |
| 102 | 0.00 | −0.42 | −0.88 | −0.21 | −0.06 | 0.00 | 0.00 | 0.03 |
| 103 | −0.02 | −0.22 | −0.49 | −0.07 | −0.01 | −0.25 | −0.03 | 0.03 |
| 104 | 0.11 | 0.27 | 0.07 | 0.01 | −0.01 | 0.04 | 0.13 | 0.03 |
| 105 | 0.18 | 0.05 | −0.03 | −0.02 | 0.01 | −0.03 | 0.02 | 0.03 |
| 106 | 0.09 | −0.06 | 0.24 | 0.23 | −0.08 | 0.03 | −0.19 | 0.03 |
| 107 | 0.11 | −0.32 | −0.48 | −0.08 | −0.22 | −0.12 | 0.07 | 0.03 |
| 108 | −0.12 | 0.14 | −0.05 | −0.05 | 0.25 | 0.14 | 0.18 | 0.03 |
| 109 | 0.09 | −0.02 | −0.29 | −0.07 | 0.11 | −0.02 | 0.17 | 0.03 |
| 110 | 0.00 | −0.05 | −0.16 | −0.36 | 0.22 | 0.02 | 0.16 | 0.03 |
| 111 | −0.27 | −0.61 | −0.44 | −0.23 | −0.32 | −0.32 | −0.11 | 0.03 |
| 112 | 0.00 | −0.44 | −1.08 | −0.52 | 0.00 | 0.03 | −0.08 | 0.03 |
| 113 | 0.00 | −0.05 | −0.56 | −0.12 | −0.07 | −0.21 | −0.13 | 0.03 |
| 114 | 0.00 | −0.20 | −0.49 | −0.19 | −0.14 | 0.31 | −0.23 | 0.03 |
| 115 | 0.14 | −0.64 | −0.60 | 0.30 | −0.13 | −0.16 | 0.02 | 0.03 |
| 116 | 0.00 | −0.19 | −0.87 | −0.05 | −0.17 | 0.00 | −0.08 | 0.03 |
| 117 | 0.07 | −0.10 | −0.25 | −0.18 | −0.12 | 0.19 | 0.06 | 0.03 |
| 118 | 0.13 | 0.27 | 0.00 | 0.06 | 0.21 | 0.02 | −0.03 | 0.03 |
| 119 | −0.04 | −0.03 | −0.26 | −0.11 | 0.07 | −0.06 | 0.11 | 0.03 |
| 120 | 0.19 | 0.00 | −0.11 | −0.03 | 0.19 | 0.06 | 0.33 | 0.03 |
| 121 | 0.00 | −0.02 | −0.20 | −0.06 | 0.00 | 0.00 | −0.04 | 0.03 |
| 122 | 0.11 | −0.15 | −0.24 | −0.27 | −0.06 | 0.21 | −0.58 | 0.03 |
| 123 | 0.19 | −0.12 | −0.69 | −0.22 | −0.02 | 0.00 | −0.02 | 0.03 |
| 124 | 0.15 | −0.10 | −0.46 | −0.14 | −0.05 | −0.02 | 0.08 | 0.03 |
| 125 | 0.00 | −0.24 | −0.75 | −0.03 | 0.03 | 0.10 | −0.13 | 0.03 |
| 126 | 0.30 | 0.05 | 0.05 | 0.04 | 0.04 | 0.11 | 0.15 | 0.03 |
| 127 | 0.14 | −0.62 | −0.48 | −0.26 | 0.02 | 0.01 | 0.11 | 0.03 |
| 128 | 0.01 | −0.28 | −0.70 | −0.24 | −0.01 | 0.06 | −0.29 | 0.03 |
| 129 | 0.00 | −0.21 | −0.53 | −0.09 | −0.14 | 0.24 | −0.03 | 0.02 |
| 130 | 0.00 | −0.39 | −0.62 | −0.31 | −0.24 | 0.03 | −0.13 | 0.02 |
| 131 | 0.00 | −0.08 | −0.72 | −0.14 | −0.27 | 0.24 | −0.12 | 0.02 |
| 132 | 0.00 | −0.02 | −0.15 | −0.02 | −0.01 | 0.16 | 0.08 | 0.02 |
| 133 | 0.00 | −0.31 | −0.50 | −0.09 | 0.03 | −0.15 | 0.01 | 0.02 |
| 134 | 0.00 | −0.41 | −0.63 | −0.43 | −0.11 | −0.24 | −0.32 | 0.02 |
| 135 | 0.21 | 0.16 | 0.06 | 0.09 | 0.20 | 0.13 | 0.12 | 0.02 |
| 136 | 0.00 | −0.53 | −0.56 | −0.21 | −0.25 | −0.01 | −0.22 | 0.02 |
| 137 | 0.00 | −0.72 | −0.78 | −0.61 | −0.23 | −0.09 | −0.30 | 0.02 |
| 138 | 0.00 | −0.26 | −0.66 | −0.30 | −0.19 | 0.19 | 0.11 | 0.02 |
| 139 | 0.00 | −0.36 | −0.56 | −0.36 | 0.04 | −0.06 | −0.06 | 0.02 |
| 140 | 0.00 | −0.17 | −0.97 | −0.02 | 0.04 | 0.12 | −0.04 | 0.02 |
| 141 | 0.00 | 0.03 | −0.07 | −0.14 | −0.18 | 0.05 | −0.09 | 0.02 |
| 142 | −0.11 | −0.83 | −0.67 | −0.32 | −0.12 | −0.31 | −0.13 | 0.02 |
| 143 | 0.09 | −0.07 | −0.19 | −0.16 | −0.16 | 0.06 | −0.08 | 0.02 |
| 144 | 0.00 | 0.02 | −0.18 | −0.05 | 0.06 | −0.07 | −0.31 | 0.02 |
| 145 | −0.06 | 0.17 | 0.14 | 0.32 | 0.12 | 0.17 | −0.17 | 0.02 |
| 146 | −0.23 | −0.54 | −0.63 | −0.27 | −0.28 | −0.44 | −0.44 | 0.02 |
| 147 | 0.00 | −0.13 | −0.39 | −0.17 | 0.10 | 0.07 | 0.06 | 0.02 |
| 148 | 0.00 | 0.14 | −0.05 | 0.26 | 0.14 | 0.32 | 0.29 | 0.02 |
| 149 | 0.08 | −0.10 | −0.18 | 0.07 | 0.14 | 0.29 | 0.07 | 0.02 |
| 150 | 0.21 | −0.34 | −0.53 | −0.26 | 0.04 | 0.06 | 0.07 | 0.02 |
| 151 | 0.00 | −0.33 | −0.49 | −0.18 | −0.25 | 0.21 | −0.19 | 0.02 |
| 152 | −0.02 | −0.25 | −0.56 | 0.06 | −0.01 | 0.04 | −0.45 | 0.02 |
| 153 | 0.14 | −0.49 | −0.26 | −0.18 | −0.08 | 0.30 | −0.19 | 0.02 |
| 154 | −0.05 | 0.02 | −0.18 | 0.02 | −0.17 | −0.08 | −0.16 | 0.02 |
| 155 | 0.00 | 0.03 | 0.00 | 0.00 | 0.00 | 0.06 | 0.01 | 0.02 |
| 156 | 0.00 | −0.42 | −0.52 | −0.06 | 0.04 | −0.17 | −0.04 | 0.02 |
| 157 | 0.11 | −0.32 | −0.36 | −0.29 | 0.20 | 0.06 | 0.06 | 0.02 |
| 158 | 0.09 | −0.01 | −0.32 | 0.15 | −0.13 | −0.07 | 0.00 | 0.02 |
| 159 | 0.00 | −0.40 | −0.58 | −0.37 | −0.02 | 0.17 | −0.33 | 0.02 |
| 160 | 0.00 | −0.18 | −0.41 | −0.11 | 0.06 | −0.27 | −0.02 | 0.02 |
| 161 | 0.00 | −0.38 | −0.56 | −0.18 | 0.23 | 0.02 | −0.17 | 0.02 |
| 162 | 0.17 | −0.56 | −0.17 | −0.20 | −0.10 | 0.00 | 0.00 | 0.02 |
| 163 | 0.06 | 0.14 | 0.07 | 0.09 | 0.01 | 0.04 | 0.02 | 0.02 |
| 164 | 0.21 | 0.06 | −0.20 | −0.06 | 0.39 | 0.03 | 0.09 | 0.02 |
| 165 | 0.19 | −0.23 | −0.73 | −0.04 | 0.02 | −0.12 | −0.32 | 0.02 |
| 166 | 0.00 | −0.45 | −0.66 | −0.30 | −0.15 | −0.08 | −0.10 | 0.02 |
| 167 | 0.00 | −0.57 | −0.76 | −0.30 | −0.15 | −0.03 | −0.20 | 0.02 |
| 168 | −0.19 | 0.23 | 0.15 | 0.03 | 0.19 | 0.26 | 0.01 | 0.02 |
| 169 | 0.13 | −0.46 | −0.74 | −0.25 | −0.02 | −0.27 | −0.51 | 0.02 |
| 170 | 0.00 | −0.51 | −0.62 | −0.29 | −0.39 | 0.12 | −0.17 | 0.02 |
| 171 | 0.16 | 0.05 | −0.20 | 0.05 | −0.22 | −0.04 | 0.16 | 0.02 |
| 172 | 0.00 | −0.31 | −0.83 | −0.24 | −0.18 | −0.35 | −0.04 | 0.02 |
| 173 | 0.00 | −0.76 | −0.87 | −0.63 | −0.39 | −0.11 | −0.33 | 0.02 |
| 174 | 0.00 | −0.29 | −0.24 | −0.21 | 0.02 | 0.00 | 0.00 | 0.02 |
| 175 | 0.00 | −0.60 | −0.91 | −0.43 | −0.08 | −0.14 | −0.13 | 0.02 |
| 176 | 0.22 | −0.61 | −0.84 | −0.36 | −0.25 | −0.15 | −0.06 | 0.02 |
| 177 | 0.00 | −0.41 | −0.96 | −0.22 | −0.16 | −0.20 | −0.29 | 0.02 |
| 178 | 0.00 | −0.40 | 1.07 | −0.36 | 0.06 | −0.13 | −0.27 | 0.02 |
| 179 | 0.09 | −0.37 | −0.26 | −0.22 | −0.10 | 0.30 | 0.00 | 0.02 |
| 180 | 0.00 | −0.49 | −0.62 | −0.49 | −0.18 | −0.04 | −0.17 | 0.02 |
| 181 | −0.83 | −0.22 | −0.23 | −0.07 | −0.10 | 0.11 | −0.41 | 0.02 |
| 182 | 0.19 | 0.00 | 0.04 | 0.04 | 0.03 | −0.23 | −0.18 | 0.02 |
| 183 | −0.24 | −0.20 | −0.41 | 0.08 | −0.38 | −0.17 | −0.17 | 0.02 |
| 184 | 0.21 | 0.17 | 0.15 | 0.03 | −0.04 | 0.08 | 0.11 | 0.02 |
| 185 | 0.00 | −0.59 | −0.88 | −0.07 | 0.07 | −0.05 | −0.26 | 0.02 |
| 186 | 0.00 | −0.54 | −0.59 | −0.30 | −0.27 | 0.00 | −0.13 | 0.02 |
| 187 | 0.00 | −0.61 | −0.47 | −0.05 | −0.12 | 0.20 | −0.17 | 0.02 |
| 188 | 0.00 | −0.47 | −0.18 | −0.25 | 0.07 | −0.02 | −0.04 | 0.02 |
| 189 | 0.00 | −0.51 | −0.95 | −0.19 | 0.10 | −0.07 | −0.12 | 0.02 |
| 190 | 0.00 | −0.50 | −1.21 | −0.71 | −0.11 | −0.23 | −0.27 | 0.02 |
| 191 | −0.08 | −0.06 | −0.23 | 0.15 | −0.15 | −0.01 | 0.04 | 0.02 |
| 192 | 0.00 | −0.45 | −0.65 | −0.27 | −0.08 | 0.02 | −0.15 | 0.02 |
| 193 | 0.06 | −0.49 | −0.51 | −0.16 | −0.16 | 0.13 | −0.26 | 0.02 |
| 194 | 0.00 | −0.09 | −0.79 | −0.11 | −0.05 | −0.06 | −0.05 | 0.02 |
| 195 | 0.07 | −0.01 | −0.34 | −0.10 | 0.12 | 0.02 | −0.08 | 0.02 |
| 196 | 0.00 | −0.40 | −0.67 | −0.35 | 0.01 | 0.09 | −0.07 | 0.02 |
| 197 | 0.00 | −0.38 | −0.70 | −0.76 | −0.20 | −0.31 | 0.10 | 0.02 |
| 198 | 0.00 | −0.55 | −0.11 | −0.53 | −0.07 | 0.26 | −0.23 | 0.02 |
| 199 | 0.00 | −0.70 | −0.89 | −0.13 | −0.21 | −0.15 | −0.10 | 0.02 |
| 200 | 0.00 | −0.45 | −0.97 | −0.49 | −0.07 | −0.35 | −0.17 | 0.02 |

log10 enrichment scores for the indicated tissue or organ are shown

TABLE 3A
PNS LUNG SEQUENCE 7 MER

| SEQ ID | | | | | | |
|---|---|---|---|---|---|---|
| NO | Amino Acid | Brain | Cord | DRG | Spleen | Liver |
| 201 | AAVSSIP | −0.13 | 0.00 | 0.13 | 0.89 | 0.09 |
| 202 | ANYTVGK | −0.09 | −0.03 | −0.03 | −0.53 | −0.22 |
| 203 | AQNSTRA | −0.41 | −0.36 | −0.23 | 0.04 | 0.10 |
| 204 | ASARVNV | −0.55 | 0.04 | −0.21 | −0.01 | 0.12 |
| 205 | DNRSKMI | −0.63 | −0.31 | 0.00 | −0.33 | 0.10 |
| 206 | DNTTVMR | 0.19 | 0.00 | −0.01 | −0.51 | −0.64 |
| 207 | DNTVRHN | −0.54 | 0.09 | 0.00 | −0.52 | 0.05 |
| 208 | DTAPTRS | −0.02 | 0.27 | 0.25 | 0.72 | 1.77 |
| 209 | EGTKSMS | −0.35 | 0.06 | −0.23 | 0.05 | 0.03 |
| 210 | EIRSSSY | −0.49 | −0.01 | −0.53 | 0.04 | −0.06 |
| 211 | ELRRSAN | −0.07 | −0.15 | −0.14 | −0.17 | 0.35 |
| 212 | FRTTGNA | −0.60 | −0.34 | −0.40 | 0.27 | 0.18 |
| 213 | GADLRNP | 0.06 | −0.06 | 0.00 | 0.96 | −0.04 |
| 214 | GFHTTSG | −0.15 | 0.03 | −0.14 | 0.13 | 0.04 |
| 215 | GGTGSRI | −0.23 | −0.09 | −0.31 | 0.03 | 0.12 |
| 216 | GLKTSGL | 0.07 | −0.35 | −0.28 | 0.15 | −0.01 |
| 217 | GMTSKAT | −0.66 | −0.22 | −0.43 | 0.15 | 0.06 |
| 218 | GNSVFPD | −0.08 | 0.07 | 0.00 | 0.56 | 0.39 |
| 219 | GNVNNRV | −0.37 | −0.11 | −0.32 | 0.00 | −0.01 |
| 220 | GQGHTSP | −0.23 | −0.01 | −0.10 | 0.25 | 0.02 |
| 221 | GQNPLRQ | −0.16 | 0.07 | −0.25 | −0.07 | 0.10 |
| 222 | GTNSRPN | −0.31 | −0.06 | −0.25 | 0.28 | 0.25 |
| 223 | HENFTRS | 0.00 | −0.14 | −0.23 | −0.29 | −0.05 |
| 224 | HSASGAA | −0.02 | −0.13 | −0.26 | 0.22 | 0.18 |
| 225 | IATIGSK | −0.52 | 0.04 | −0.09 | −0.05 | −0.25 |
| 226 | IGGSASQ | 0.00 | 0.01 | 0.00 | 0.09 | 0.01 |
| 227 | IGPTGRM | 0.11 | 0.05 | −0.22 | 0.70 | 1.37 |
| 228 | INSARDT | −0.26 | −0.31 | −0.10 | 0.22 | 0.18 |
| 229 | IRSLERD | −0.64 | −0.12 | −0.39 | 0.12 | 0.04 |
| 230 | KMASTQS | −0.40 | 0.04 | −0.16 | 0.13 | 0.00 |
| 231 | KNEAITC | −0.09 | 0.00 | 0.00 | 0.36 | 0.04 |
| 232 | KNGLAQN | −0.29 | −0.15 | −0.61 | 0.08 | −0.11 |
| 233 | KQLTASA | 0.41 | 0.01 | −0.25 | 0.11 | 0.02 |
| 234 | KTPSVAS | −0.24 | −0.04 | −0.28 | 0.19 | 0.13 |
| 235 | KVTLTSN | −0.13 | 0.02 | −0.13 | 0.03 | −0.02 |
| 236 | LNGVSSR | −0.03 | −0.12 | −0.13 | 0.01 | −0.15 |
| 237 | LNNTKTT | −0.58 | −0.13 | −0.51 | −0.09 | 0.20 |
| 238 | LNSVRRD | −0.21 | −0.22 | 0.00 | −0.44 | −0.09 |
| 239 | LQSKLNS | −0.38 | 0.02 | 0.00 | 0.13 | 0.14 |
| 240 | LRTGELT | −0.70 | −0.21 | −0.12 | 0.02 | 0.02 |
| 241 | LSTRQTT | 0.40 | −0.15 | −0.50 | 0.13 | 0.25 |
| 242 | LTGVGSQ | 0.34 | −0.04 | 0.00 | 0.13 | −0.03 |
| 243 | LTKTVSA | 0.64 | −0.14 | −0.31 | 0.17 | 0.10 |
| 244 | LINGSNY | −0.13 | −0.15 | −0.02 | 0.21 | 0.02 |
| 245 | LTYATVG | 0.36 | 0.02 | 0.00 | 0.02 | 0.08 |
| 246 | LVQNNTM | 0.25 | 0.03 | 0.00 | 0.04 | −0.29 |
| 247 | MNRTLST | −0.49 | 0.07 | −0.31 | −0.10 | 0.18 |
| 248 | MNVRQSA | −0.64 | −0.22 | −0.41 | 0.14 | 0.19 |
| 249 | MRLERAG | 0.68 | −0.13 | −0.45 | 0.09 | 0.10 |
| 250 | MTRSSGY | 0.01 | 0.00 | −0.09 | 0.19 | 0.24 |
| 251 | NKTSNTP | −0.60 | 0.12 | −0.14 | 0.14 | 0.07 |
| 252 | NLKTGAT | 0.51 | −0.23 | −0.15 | 0.04 | 0.08 |
| 253 | NSHQNTI | −0.11 | −0.10 | −0.09 | 0.07 | −0.10 |
| 254 | NSLKNVA | 0.29 | 0.01 | −0.18 | 0.14 | 0.06 |
| 255 | NSVKNVL | 0.28 | 0.14 | −0.19 | −0.41 | 0.09 |
| 256 | NSVSRDI | 0.14 | 0.12 | −0.16 | −0.44 | −0.19 |
| 257 | NTKNSSG | 0.68 | −0.03 | −0.24 | 0.14 | 0.20 |
| 258 | NVGKLQP | 0.36 | −0.18 | −0.53 | 0.03 | −0.02 |
| 259 | PAPGRVS | 0.29 | −0.04 | −0.22 | 0.21 | 0.17 |
| 260 | PNGYATV | 0.00 | −0.02 | 0.11 | 0.08 | −0.25 |
| 261 | PNTQVRA | 0.61 | −0.03 | 0.14 | 0.05 | 0.04 |
| 262 | PPSGQAL | 0.02 | 0.00 | −0.09 | 0.09 | 0.11 |
| 263 | PRTDKGS | 0.40 | −0.16 | −0.03 | 0.14 | 0.03 |
| 264 | PSPQLRS | 0.21 | −0.11 | 0.03 | −0.06 | 0.06 |
| 265 | PVNTQRV | 0.22 | 0.27 | 0.31 | 1.50 | 0.96 |
| 266 | QKSQQHG | 0.44 | −0.16 | −0.08 | −0.03 | 0.13 |
| 267 | QRESVKV | 0.58 | −0.23 | −0.32 | 0.09 | 0.10 |
| 268 | RHTVNPS | 0.10 | 0.02 | −0.28 | −0.29 | −0.05 |
| 269 | RSQHTTS | 0.37 | −0.08 | 0.14 | −0.03 | 0.18 |
| 270 | SAVTIKA | 0.33 | −0.18 | −0.30 | 0.13 | 0.02 |
| 271 | SGKTPHT | 0.23 | 0.01 | −0.17 | −0.11 | 0.10 |
| 272 | SLGKVEN | 0.15 | −0.07 | −0.10 | 0.02 | −0.04 |
| 273 | SNATHNT | 0.17 | −0.05 | −0.22 | 0.06 | 0.02 |
| 274 | SNLARNV | −0.60 | −0.30 | −0.36 | −0.35 | 0.01 |
| 275 | SRLNTPG | −0.16 | 0.03 | −0.14 | 0.11 | 0.06 |
| 276 | SSNVARG | −0.45 | −0.03 | −0.34 | 0.12 | 0.25 |
| 277 | SSRSAQA | −0.21 | 0.02 | −0.15 | 0.28 | 0.31 |
| 278 | STISTRP | −0.37 | −0.24 | −0.27 | 0.15 | 0.10 |
| 279 | SYLASAS | −0.37 | −0.03 | 0.00 | 0.00 | −0.04 |
| 280 | TFSQTRE | −0.51 | −0.09 | 0.02 | 0.01 | 0.09 |
| 281 | TGHNQMA | 0.07 | 0.21 | −0.10 | 1.39 | 0.01 |
| 282 | TGNSSGT | −0.06 | −0.16 | −0.29 | 0.22 | −0.03 |
| 283 | TKISPGS | −0.22 | −0.13 | −0.29 | 0.19 | 0.15 |
| 284 | TMNLAKA | −0.48 | −0.14 | −0.28 | 0.10 | 0.09 |
| 285 | TQRLPST | −0.65 | 0.11 | −0.26 | 0.08 | 0.16 |
| 286 | TQRSNYE | −0.18 | −0.16 | −0.35 | 0.22 | 0.14 |
| 287 | TRGSYPP | −0.42 | −0.09 | −0.14 | 0.04 | 0.10 |
| 288 | TRMDRGQ | −0.19 | −0.05 | −0.18 | −0.08 | 0.27 |
| 289 | TRSSLSG | 0.15 | 0.00 | −0.23 | 0.05 | 0.25 |
| 290 | TRTVTSA | 0.79 | −0.08 | −0.19 | 0.19 | 0.20 |
| 291 | TSQSLIG | −0.26 | −0.09 | −0.20 | 0.24 | 0.12 |
| 292 | TSTRQST | −0.72 | −0.09 | −0.15 | 0.09 | 10.22 |
| 293 | VNHVARP | −0.36 | −0.07 | −0.02 | 0.01 | 0.06 |
| 294 | VNTAKPF | 0.53 | −0.06 | −0.42 | 0.08 | 10.07 |
| 295 | VRTSQSS | −0.68 | −0.11 | −0.23 | 0.17 | 0.27 |
| 296 | VVITGRS | 0.27 | −0.04 | 0.00 | 0.11 | 0.12 |
| 297 | VVTKSLA | 0.68 | −0.15 | −0.31 | 0.13 | −0.03 |
| 298 | WSGSSLS | 0.22 | 0.02 | −0.21 | 0.09 | 0.00 |
| 299 | YNSASRN | 0.41 | −0.04 | −0.31 | 0.17 | 0.13 |
| 300 | YNTTRER | 0.27 | −0.06 | 0.00 | −0.49 | −0.19 |
| 301 | YRLSNDA | 0.07 | −0.19 | −0.15 | 0.16 | −0.02 |

log10 enrichment scores for the indicated tissue or organ are shown

TABLE 3B
PNS LUNG SEQUENCE 7 MER (CONTINUED

| SEQ ID NO | Amino Acid | Heart | Lung | Kidney | Testis | Muscle |
|---|---|---|---|---|---|---|
| 201 | AAVSSIP | 0.46 | 0.90 | 0.89 | 0.45 | 0.48 |
| 202 | ANYTVGK | 0.18 | 0.58 | −0.14 | −0.16 | −0.65 |
| 203 | AQNSTRA | 0.12 | 0.44 | 0.13 | 0.25 | 0.09 |
| 204 | ASARVNV | 0.15 | 0.38 | 0.11 | 0.16 | 0.12 |
| 205 | DNRSKMI | −0.05 | 0.77 | 0.16 | 0.01 | −0.34 |
| 206 | DNTTVMR | −0.23 | 0.43 | −0.41 | −0.26 | −0.13 |
| 207 | DNTVRHN | −0.20 | 0.61 | −0.09 | −0.28 | −0.85 |
| 208 | DTAPTRS | 1.18 | 0.62 | 1.93 | 1.30 | 0.74 |
| 209 | EGTKSMS | −0.04 | 0.31 | 0.09 | 0.08 | 0.02 |
| 210 | EIRSSSY | −0.04 | 0.33 | −0.06 | 0.02 | 0.08 |
| 211 | ELRRSAN | 0.01 | 0.49 | −0.09 | −0.28 | −0.17 |
| 212 | FRTTGNA | 0.25 | 0.34 | 0.34 | 0.14 | 0.14 |
| 213 | GADLRNP | 1.27 | 0.93 | 1.28 | 0.76 | 0.54 |
| 214 | GFHTTSG | 0.08 | 0.33 | 0.12 | −0.03 | 0.17 |
| 215 | GGTGSRI | 0.14 | 0.33 | 0.12 | 0.11 | −0.14 |
| 216 | GLKTSGL | 0.13 | 0.36 | 0.08 | 0.04 | 0.11 |
| 217 | GMTSKAT | 0.15 | 0.34 | 0.12 | 0.16 | 0.24 |
| 218 | GNSVFPD | 0.49 | 0.52 | 0.49 | 0.53 | 0.03 |
| 219 | GNVNNRV | 0.16 | 0.30 | 0.16 | 0.13 | 10.02 |
| 220 | GQGHTSP | 0.14 | 0.52 | 0.10 | −0.01 | 0.12 |
| 221 | GQNPLRQ | 0.05 | 10.33 | 0.05 | 0.12 | −0.06 |
| 222 | GTNSRPN | 0.24 | 0.46 | 0.33 | 0.07 | 0.22 |
| 223 | HENFTRS | −0.07 | 0.32 | −0.13 | −0.11 | −0.05 |
| 224 | HSASGAA | 0.17 | 0.45 | 0.37 | 0.16 | −0.06 |
| 225 | IATIGSK | 0.13 | 0.33 | 0.02 | −0.03 | 0.11 |
| 226 | IGGSASQ | −0.02 | 10.35 | −0.01 | 0.04 | 0.11 |
| 227 | IGPTGRM | 1.31 | 0.65 | 1.42 | 1.30 | 1.22 |
| 228 | INSARDT | 0.10 | 0.32 | 0.30 | 0.24 | 0.25 |
| 229 | IRSLERD | 0.15 | 0.34 | 0.13 | 0.14 | 0.26 |
| 230 | KMASTQS | 0.13 | 0.35 | 0.11 | −0.03 | 0.20 |
| 231 | KNEAITC | 0.41 | 0.39 | 0.40 | −0.14 | 0.43 |
| 232 | KNGLAQN | 0.14 | 0.32 | 0.04 | 0.10 | 0.28 |
| 233 | KQLTASA | 0.19 | 0.41 | 0.14 | 0.04 | 0.14 |
| 234 | KTPSVAS | 0.15 | 0.35 | 0.27 | 0.06 | 0.24 |
| 235 | KVTLTSN | 0.20 | 0.34 | 0.13 | −0.02 | 0.17 |
| 236 | LNGVSSR | 0.10 | 0.31 | 0.05 | 0.16 | −0.40 |
| 237 | LNNTKTT | 0.13 | 0.98 | 0.25 | 0.28 | −0.08 |
| 238 | LNSVRRD | −0.39 | 0.40 | −0.33 | −0.47 | −0.65 |
| 239 | LQSKLNS | 0.15 | 0.34 | 0.24 | 0.26 | 0.20 |
| 240 | LRTGELT | 0.01 | 0.45 | 0.02 | −0.02 | 0.10 |
| 241 | LSTRQTT | 0.24 | 0.34 | 0.26 | 0.13 | 0.19 |
| 242 | LTGVGSQ | −0.01 | 0.34 | 0.11 | −0.10 | 0.07 |
| 243 | LTKTVSA | 0.22 | 0.35 | 0.32 | 0.33 | −0.05 |
| 244 | LINGSNY | 0.02 | 0.32 | 0.16 | 0.02 | −0.07 |
| 245 | LTYATVG | 0.10 | 0.35 | 0.11 | 0.02 | 0.11 |
| 246 | LVQNNTM | −0.09 | 0.42 | −0.03 | −0.08 | 0.09 |
| 247 | MNRTLST | 0.15 | 10.58 | 0.14 | 0.29 | −0.06 |
| 248 | MNVRQSA | 0.23 | 0.34 | 0.21 | 0.04 | 0.06 |
| 249 | MRLERAG | 0.08 | 0.39 | 0.09 | −0.05 | −0.20 |
| 250 | MTRSSGY | 0.20 | 0.40 | −0.06 | −0.09 | 0.03 |
| 251 | NKTSNTP | 0.21 | 0.53 | 0.19 | 0.26 | 0.23 |
| 252 | NLKTGAT | 0.15 | 0.60 | 0.17 | −0.06 | 0.28 |
| 253 | NSHQNTI | −0.07 | 0.35 | −0.02 | −0.24 | −0.06 |
| 254 | NSLKNVA | 0.21 | 0.36 | 0.05 | −0.05 | 0.17 |
| 255 | NSVKNVL | −0.09 | 0.46 | 0.08 | −0.10 | −0.54 |
| 256 | NSVSRDI | −0.03 | 0.40 | −0.17 | −0.01 | −0.66 |
| 257 | NTKNSSG | 0.15 | 0.35 | 0.29 | 0.17 | 0.21 |
| 258 | NVGKLQP | 0.23 | 0.39 | 0.13 | 0.20 | 0.02 |
| 259 | PAPGRVS | 0.21 | 0.38 | 0.34 | 0.03 | 0.22 |
| 260 | PNGYATV | 0.10 | 0.31 | −0.10 | −0.05 | −0.15 |
| 261 | PNTQVRA | 0.24 | 0.31 | 0.14 | 0.23 | 0.34 |
| 262 | PPSGQAL | 0.04 | 0.34 | 0.12 | −0.07 | −0.43 |
| 263 | PRTDKGS | 0.19 | 0.41 | 0.14 | 0.12 | 0.23 |
| 264 | PSPQLRS | 0.12 | 0.33 | 0.11 | 0.06 | −0.44 |
| 265 | PVNTQRV | 1.07 | 1.40 | 1.54 | 1.06 | 1.37 |
| 266 | QKSQQHG | 0.11 | 0.32 | 0.05 | 0.20 | 0.09 |
| 267 | QRESVKV | 0.23 | 0.30 | 0.23 | 0.18 | 0.21 |
| 268 | RHTVNPS | 0.10 | 0.38 | −0.08 | −0.14 | −0.19 |
| 269 | RSQHTTS | 0.24 | 0.31 | −0.02 | 0.07 | −0.07 |
| 270 | SAVTIKA | 0.14 | 0.32 | 0.13 | 0.05 | 0.02 |
| 271 | SGKTPHT | 0.16 | 0.35 | −0.05 | −0.01 | 0.15 |
| 272 | SLGKVEN | −0.05 | 0.31 | −0.06 | 0.02 | 0.24 |
| 273 | SNATHNT | 0.09 | 0.52 | 0.11 | 0.30 | 0.01 |
| 274 | SNLARNV | 0.19 | 1.32 | 0.22 | 0.14 | −0.48 |
| 275 | SRLNTPG | 0.10 | 0.31 | 0.19 | 0.02 | 0.03 |
| 276 | SSNVARG | 0.21 | 0.60 | 0.22 | 0.19 | 0.19 |
| 277 | SSRSAQA | 0.28 | 0.38 | 0.37 | 0.25 | 0.18 |
| 278 | STISTRP | 0.24 | 0.37 | 0.22 | 0.11 | 0.20 |
| 279 | SYLASAS | 0.12 | 0.36 | −0.07 | 0.10 | −0.08 |
| 280 | TFSQTRE | 0.02 | 10.34 | 0.01 | −0.29 | −0.06 |
| 281 | TGHNQMA | 0.67 | 0.67 | 0.03 | 1.34 | 0.81 |
| 282 | TGNSSGT | 0.01 | 0.31 | 0.08 | 0.11 | 0.06 |
| 283 | TKISPGS | 0.21 | 0.32 | 0.23 | 0.13 | 0.28 |
| 284 | TMNLAKA | 0.15 | 0.34 | 0.16 | 0.03 | 0.12 |
| 285 | TQRLPST | 0.31 | 0.36 | 0.26 | 0.12 | 0.00 |
| 286 | TQRSNYE | 0.17 | 0.35 | 0.32 | 0.18 | 0.16 |
| 287 | TRGSYPP | 0.10 | 0.36 | 0.17 | 0.04 | 0.03 |
| 288 | TRMDRGQ | 0.12 | 0.33 | −0.04 | −0.02 | −0.27 |
| 289 | TRSSLSG | 0.12 | 0.38 | 0.14 | −0.12 | 0.03 |
| 290 | TRTVTSA | 0.27 | 0.41 | 0.22 | 0.28 | −0.03 |
| 291 | TSQSLIG | 0.19 | 0.38 | 0.21 | 0.08 | 0.25 |
| 292 | TSTRQST | 0.14 | 0.31 | 0.16 | 0.15 | 0.06 |
| 293 | VNHVARP | 0.09 | 0.31 | 0.16 | 0.05 | −0.03 |
| 294 | VNTAKPF | 0.05 | 0.35 | 0.13 | −0.11 | 0.02 |
| 295 | VRTSQSS | 0.18 | 0.40 | 0.27 | 0.21 | 0.16 |
| 296 | VVITGRS | 0.10 | 0.39 | 0.22 | −0.23 | 0.11 |
| 297 | VVTKSLA | 0.22 | 0.31 | 0.24 | 0.12 | 0.19 |
| 298 | WSGSSLS | 0.16 | 0.32 | 0.05 | −0.03 | −0.08 |
| 299 | YNSASRN | 0.07 | 0.40 | 0.27 | 0.06 | 0.11 |
| 300 | YNTTRER | −0.09 | 0.61 | −0.26 | 0.08 | −0.33 |
| 301 | YRLSNDA | 0.19 | 0.30 | 0.24 | 0.01 | 0.12 |

log10 enrichment scores for the indicated tissue or organ are shown

TABLE 4
CNS LUNG SEQUENCE 7 MER

| SEQ | Amino | Endothelial | | | Small | Large | |
|---|---|---|---|---|---|---|---|
| ID NO | Acid | Cells | Astrocytes | Neurons | Intestine | Intestine | Stomach |
| 302 | ANNSRPF | 0.45 | 0.35 | 0.03 | 0.05 | 0.09 | 0.13 |
| 303 | ANRIRDN | 0.20 | −0.14 | −0.33 | −0.31 | −0.41 | −0.49 |
| 304 | ANRTKDY | 0.33 | 0.09 | −0.15 | −0.41 | −0.18 | −0.34 |
| 305 | ANTIRNV | 0.74 | 0.52 | 0.09 | 0.07 | 0.25 | 0.18 |
| 306 | DNHVMAR | 0.42 | 0.36 | 0.33 | 0.05 | 0.18 | −0.07 |
| 307 | DNTTRLK | 0.03 | −0.31 | −0.78 | −0.36 | −0.29 | −0.59 |
| 308 | DRNTTVR | −0.11 | −0.46 | 0.19 | −0.31 | −0.28 | −0.27 |
| 309 | ENRTVPT | 0.02 | −0.17 | 0.03 | 0.15 | −0.07 | −0.03 |
| 310 | ENRVKSA | 0.22 | −0.15 | −0.41 | −0.18 | −0.10 | −0.47 |
| 311 | ENTTRIA | 0.11 | 0.04 | −0.32 | 0.20 | 0.14 | −0.39 |
| 312 | GNATPRS | 0.31 | 0.34 | −0.05 | 0.10 | −0.19 | −0.03 |
| 313 | GNAVRST | 0.41 | 0.07 | −0.08 | −0.27 | −0.17 | −0.33 |
| 314 | GNDIRSR | 0.23 | −0.22 | −0.32 | −0.59 | −0.94 | −0.68 |
| 315 | GNHARNL | 0.11 | −0.39 | −0.57 | −0.55 | −0.49 | −0.53 |
| 316 | GNMIKVA | 0.30 | −0.17 | −0.25 | 0.20 | 0.13 | −0.01 |
| 317 | GNMTKFT | 0.52 | −0.01 | 0.00 | −0.23 | −0.28 | −0.35 |
| 318 | GNNSRNM | 0.49 | 0.15 | −0.10 | −0.31 | −0.28 | −0.35 |
| 319 | GNNTAKL | −0.01 | −0.22 | 0.09 | 0.08 | −0.05 | 0.06 |
| 320 | GNQILRL | −0.12 | −0.13 | −0.09 | −0.25 | −0.26 | −0.39 |
| 321 | GNQTNRS | 0.31 | 0.24 | −0.32 | −0.08 | −0.22 | −0.12 |
| 322 | GNRISNT | 0.45 | 0.26 | −0.06 | −0.32 | −0.27 | −0.43 |
| 323 | GNRTTSS | 0.15 | −0.14 | −0.17 | 0.15 | 0.06 | −0.17 |
| 324 | GNSVRLI | 0.44 | −0.11 | −0.08 | −0.35 | −0.43 | −0.58 |
| 325 | GNWVKDI | 0.39 | −0.14 | −0.18 | −0.33 | −0.46 | −0.44 |
| 326 | GNYVSRP | −0.20 | −0.35 | −0.36 | 0.02 | −0.20 | −0.39 |
| 327 | HNLTRNL | 0.08 | −0.43 | −0.33 | −0.51 | −0.46 | −0.60 |
| 328 | LNAVKNV | 0.39 | 0.25 | 0.09 | −0.08 | −0.20 | −0.17 |
| 329 | LNSTRNT | 1.02 | 0.00 | −0.16 | −0.37 | 0.03 | −0.04 |
| 330 | LYSNKSN | 0.35 | 0.08 | −0.37 | −0.17 | −0.28 | −0.31 |
| 331 | MNSTKNV | 0.92 | 0.31 | 0.19 | −0.18 | 0.03 | −0.06 |
| 332 | NHIRAMQ | −0.05 | −0.42 | −0.29 | −0.22 | −0.35 | −0.50 |
| 333 | NHTRMNE | 0.30 | 0.22 | −0.06 | 0.21 | −0.21 | −0.02 |
| 334 | NNATRYT | 0.27 | −0.18 | −0.54 | −0.19 | −0.54 | −0.30 |
| 335 | NNLRPVY | 0.35 | −0.07 | −0.32 | −0.30 | −0.60 | −0.35 |
| 336 | NNLTRVT | −0.01 | −0.38 | −0.38 | −0.01 | −0.15 | −0.47 |
| 337 | NNNTKMN | 0.32 | 0.17 | −0.18 | 0.05 | 0.02 | −0.02 |
| 338 | NNRTTNN | 0.42 | 0.11 | −0.08 | 0.00 | 0.06 | 0.06 |
| 339 | NNSLRSN | 0.29 | −0.07 | −0.09 | 0.05 | −0.19 | −0.03 |
| 340 | NNTARSV | 0.12 | −0.13 | −0.13 | 0.16 | −0.07 | −0.07 |
| 341 | NNTVMRS | 0.12 | 0.00 | −0.26 | 0.30 | 0.18 | −0.05 |
| 342 | NSTRIIV | 0.34 | −0.07 | −0.20 | −0.12 | −0.24 | −0.21 |
| 343 | NSVRQSL | 0.43 | 0.26 | −0.23 | −0.12 | −0.59 | −0.03 |
| 344 | NTVRSST | 0.14 | 0.27 | 0.16 | 0.18 | −0.12 | 0.15 |
| 345 | QNLTKGQ | 0.44 | 0.29 | 0.17 | 0.25 | 0.05 | 0.13 |
| 346 | QNQSRFT | −0.22 | 0.09 | −0.30 | −0.20 | −0.28 | −0.14 |
| 347 | SANATKL | 0.14 | 0.12 | −0.23 | 0.13 | −0.07 | −0.01 |
| 348 | SNAIRPV | 0.47 | 0.39 | −0.18 | 0.02 | 0.36 | 0.07 |
| 349 | SNATMRF | 0.51 | 0.41 | 0.17 | 0.04 | 0.21 | 0.00 |
| 350 | SNHINRV | −0.21 | −0.32 | −0.60 | −0.32 | −0.81 | −0.59 |
| 351 | SNHTRSY | 0.21 | −0.32 | −0.33 | −0.45 | −0.44 | −0.41 |
| 352 | SNNSRPN | 0.18 | −0.01 | −0.61 | 0.11 | 0.07 | 0.02 |
| 353 | SNNTRPT | 0.32 | 0.38 | −0.12 | −0.18 | −0.21 | −0.27 |
| 354 | SNQIRDS | 0.31 | 0.07 | −0.36 | 0.34 | 0.26 | 0.04 |
| 355 | SNRIVPV | 0.13 | −0.09 | −0.18 | −0.06 | −0.39 | −0.20 |
| 356 | SNRKIDD | 0.47 | 0.31 | 0.00 | 0.18 | 0.03 | −0.09 |
| 357 | SNRTVRE | 0.46 | 0.26 | −0.20 | −0.17 | −0.15 | −0.21 |
| 358 | SNRVRDL | 0.24 | −0.26 | −0.23 | −0.34 | −0.40 | −0.40 |
| 359 | SNSTKAI | 0.82 | 0.87 | 0.34 | −0.30 | −0.14 | −0.10 |
| 360 | SNSTKPN | 0.64 | −0.63 | −0.20 | −0.17 | −0.32 | −0.19 |
| 361 | SNSTSRA | 0.45 | 0.07 | −0.27 | −0.07 | −0.01 | 0.00 |
| 362 | SNYTKII | 0.52 | 0.27 | 0.16 | −0.09 | −0.09 | −0.37 |
| 363 | TNFTKSG | −0.25 | −0.04 | −0.16 | 0.16 | −0.11 | −0.12 |
| 364 | TNHTRNT | 0.28 | −0.16 | −0.37 | −0.46 | −0.64 | −0.70 |
| 365 | TNHVKFT | 0.34 | −0.19 | −0.07 | −0.39 | −0.61 | −0.41 |
| 366 | TNNTRNT | 0.90 | −0.15 | 0.04 | 0.00 | −0.31 | −0.36 |
| 367 | TNQSKNF | 0.10 | −0.18 | −0.30 | 0.24 | 0.01 | 0.03 |
| 368 | TNRTIQT | 0.61 | 0.27 | 0.04 | 0.17 | 0.24 | 0.13 |
| 369 | TNRTQAH | −0.34 | −0.70 | −0.30 | −0.32 | −0.20 | −0.22 |
| 370 | TNRTQLY | −0.02 | −0.05 | −0.05 | 0.08 | −0.03 | −0.11 |
| 371 | TNSTLRQ | 0.51 | 0.12 | −0.06 | 0.08 | 0.15 | −0.02 |
| 372 | TNSTTKS | 0.14 | −0.61 | −0.42 | 0.24 | 0.00 | −0.03 |
| 373 | TNTTRAM | 0.40 | 0.21 | −0.06 | −0.15 | −0.19 | −0.14 |
| 374 | TNVTRST | 0.15 | 0.12 | 0.31 | −0.14 | −0.25 | −0.03 |
| 375 | VNKVRDV | 0.37 | −0.18 | −0.12 | 0.03 | 0.14 | −0.20 |
| 376 | VNSVRMT | 0.28 | 0.00 | 0.20 | −0.06 | 0.00 | −0.06 |
| 377 | VNTTRFS | 0.13 | −0.34 | −0.46 | 0.19 | −0.33 | 0.00 |
| 378 | VQSNRTN | 0.23 | −0.57 | −0.36 | 0.03 | 0.06 | −0.10 |
| 379 | WGNRIQI | −0.30 | 0.03 | −0.20 | 0.04 | 0.11 | −0.06 |

| SEQ | Amino | | | | | | | |
|---|---|---|---|---|---|---|---|---|
| ID NO | Acid | Liver | Lung | Heart | Kidney | Pancreas | Spleen | Eye |
| 302 | ANNSRPF | 0.30 | 0.80 | 0.01 | 0.17 | 0.10 | 0.04 | 0.14 |
| 303 | ANRIRDN | −0.12 | 0.51 | −0.30 | −0.24 | −0.41 | −0.18 | 0.01 |
| 304 | ANRTKDY | −0.12 | 0.59 | −0.20 | −0.13 | −0.27 | −0.13 | 0.05 |
| 305 | ANTIRNV | −0.22 | 0.99 | 0.05 | 0.10 | −0.03 | 0.13 | −0.02 |
| 306 | DNHVMAR | −0.06 | 0.74 | −0.24 | 0.02 | −0.01 | −0.29 | 0.05 |
| 307 | DNTTRLK | −0.19 | 0.25 | −0.41 | −0.35 | −0.54 | −0.45 | −0.08 |
| 308 | DRNTTVR | −0.30 | 0.24 | −0.45 | −0.31 | −0.37 | −0.38 | −0.04 |
| 309 | ENRTVPT | 0.05 | 0.28 | −0.09 | 0.02 | 0.09 | −0.07 | −0.21 |
| 310 | ENRVKSA | 0.22 | 0.57 | −0.15 | 0.00 | −0.25 | −0.03 | 0.14 |
| 311 | ENTTRIA | 0.00 | 0.43 | −0.20 | 0.02 | 0.00 | −0.14 | −0.16 |
| 312 | GNATPRS | 0.29 | 0.77 | −0.08 | 0.16 | −0.06 | −0.09 | 0.04 |
| 313 | GNAVRST | −0.24 | 0.90 | −0.02 | 0.06 | −0.12 | 0.04 | 0.27 |
| 314 | GNDIRSR | −0.39 | 0.54 | −0.43 | −0.36 | −0.51 | −0.50 | −0.35 |
| 315 | GNHARNL | −0.43 | 0.40 | −0.49 | −0.44 | −0.60 | −0.44 | −0.28 |
| 316 | GNMIKVA | 0.08 | 0.63 | −0.01 | 0.23 | −0.07 | 0.12 | 0.29 |
| 317 | GNMTKFT | −0.30 | 0.70 | −0.18 | −0.02 | −0.27 | −0.04 | 0.15 |
| 318 | GNNSRNM | −0.17 | 0.72 | −0.22 | −0.10 | −0.30 | −0.14 | 0.11 |
| 319 | GNNTAKL | 0.11 | 0.16 | −0.03 | 0.06 | −0.05 | −0.11 | −0.37 |
| 320 | GNQILRL | −0.38 | 0.10 | −0.42 | −0.40 | −0.41 | −0.35 | −0.06 |
| 321 | GNQTNRS | 0.21 | 0.76 | −0.28 | 0.05 | −0.11 | −0.22 | −0.03 |
| 322 | GNRISNT | −0.24 | 0.80 | −0.12 | −0.03 | −0.22 | −0.08 | 0.12 |
| 323 | GNRTTSS | 0.42 | 0.69 | −0.02 | 0.19 | 0.03 | 0.00 | 0.30 |
| 324 | GNSVRLI | −0.42 | 0.73 | −0.17 | −0.10 | −0.33 | −0.06 | 0.09 |
| 325 | GNWVKDI | −0.26 | 0.62 | −0.26 | −0.11 | −0.33 | −0.07 | −0.04 |
| 326 | GNYVSRP | 0.09 | 0.28 | −0.12 | −0.01 | −0.27 | 0.21 | 0.20 |
| 327 | HNLTRNL | −0.39 | 0.33 | −0.46 | −0.38 | −0.56 | −0.50 | −0.21 |
| 328 | LNAVKNV | −0.25 | 0.83 | 0.05 | 0.17 | −0.04 | 0.26 | 0.15 |
| 329 | LNSTRNT | −0.26 | 1.01 | −0.11 | 0.26 | 0.10 | 0.23 | 0.13 |
| 330 | LYSNKSN | 0.02 | 0.39 | −0.09 | −0.10 | −0.18 | 0.07 | 0.17 |
| 331 | MNSTKNV | −0.26 | 1.00 | 0.12 | 0.26 | 0.11 | 0.25 | 0.24 |
| 332 | NHIRAMQ | −0.21 | 0.25 | −0.37 | −0.35 | −0.51 | −0.26 | −0.12 |
| 333 | NHTRMNE | 0.25 | 0.63 | 0.00 | 0.23 | −0.01 | 0.08 | 0.08 |
| 334 | NNATRYT | −0.09 | 0.32 | −0.23 | −0.19 | −0.34 | −0.11 | 0.01 |
| 335 | NNLRPVY | −0.21 | 0.55 | −0.31 | −0.22 | −0.42 | −0.19 | 0.01 |
| 336 | NNLTRVT | −0.07 | 0.25 | −0.26 | −0.12 | −0.27 | −0.17 | 0.06 |
| 337 | NNNTKMN | 0.03 | 0.62 | −0.15 | 0.15 | −0.10 | −0.18 | 0.01 |
| 338 | NNRTTNN | 0.29 | 0.77 | −0.10 | 0.19 | 0.01 | −0.05 | 0.30 |
| 339 | NNSLRSN | 0.19 | 0.72 | −0.10 | 0.14 | −0.11 | −0.09 | 0.24 |
| 340 | NNTARSV | 0.31 | 0.41 | −0.03 | 0.13 | −0.06 | −0.04 | 0.09 |
| 341 | NNTVMRS | 0.29 | 0.55 | 0.04 | 0.28 | −0.07 | 0.10 | 0.23 |
| 342 | NSTRIIV | 0.06 | 0.58 | −0.21 | −0.01 | −0.20 | −0.05 | −0.12 |
| 343 | NSVRQSL | 0.13 | 0.77 | −0.11 | 0.05 | −0.25 | −0.13 | 0.02 |
| 344 | NTVRSST | 0.41 | 0.51 | 0.10 | 0.25 | −0.03 | 0.02 | −0.12 |
| 345 | QNLTKGQ | 0.18 | 0.68 | 0.03 | 0.21 | 0.12 | 0.04 | 0.12 |
| 346 | QNQSRFT | −0.10 | −0.15 | −0.26 | −0.29 | −0.30 | −0.29 | −0.30 |
| 347 | SANATKL | 0.16 | 0.16 | 0.04 | 0.11 | 0.04 | 0.02 | 0.00 |
| 348 | SNAIRPV | −0.46 | 0.76 | −0.15 | −0.27 | −0.03 | −0.23 | 0.00 |
| 349 | SNATMRF | 0.14 | 0.90 | 0.02 | 0.12 | 0.09 | 0.06 | 0.23 |
| 350 | SNHINRV | 0.03 | 0.03 | −0.42 | −0.16 | −0.60 | −0.28 | −0.25 |
| 351 | SNHTRSY | −0.32 | 0.51 | −0.34 | −0.27 | −0.37 | −0.25 | −0.31 |
| 352 | SNNSRPN | 0.32 | 0.58 | 0.01 | 0.27 | 0.03 | 0.00 | 0.17 |
| 353 | SNNTRPT | −0.36 | 0.62 | −0.24 | −0.31 | −0.05 | −0.25 | 0.01 |
| 354 | SNQIRDS | 0.11 | 0.65 | −0.07 | 0.25 | 0.18 | −0.08 | 0.26 |
| 355 | SNRIVPV | 0.18 | 0.49 | −0.24 | 0.08 | −0.23 | −0.11 | 0.03 |
| 356 | SNRKIDD | 0.08 | 0.86 | −0.09 | 0.32 | −0.04 | −0.04 | 0.23 |
| 357 | SNRTVRE | 0.06 | 0.77 | −0.06 | 0.11 | −0.14 | 0.07 | 0.20 |
| 358 | SNRVRDL | −0.07 | 0.56 | −0.25 | −0.19 | −0.32 | −0.16 | −0.05 |
| 359 | SNSTKAI | −0.25 | 0.80 | −0.17 | 0.02 | −0.31 | 0.06 | −0.04 |
| 360 | SNSTKPN | −0.32 | 0.83 | −0.13 | 0.04 | −0.01 | 0.07 | 0.27 |
| 361 | SNSTSRA | 0.26 | 0.83 | −0.06 | 0.25 | −0.04 | 0.14 | 0.24 |
| 362 | SNYTKII | −0.32 | 0.80 | 0.01 | 0.08 | −0.10 | 0.21 | 0.34 |
| 363 | TNFTKSG | −0.03 | 0.62 | −0.06 | 0.13 | −0.18 | 0.11 | 10.38 |
| 364 | TNHTRNT | −0.46 | 0.45 | −0.39 | −0.27 | −0.53 | −0.32 | −0.16 |
| 365 | TNHVKFT | −0.23 | 0.59 | −0.35 | −0.30 | −0.41 | −0.25 | −0.17 |
| 366 | TNNTRNT | −0.26 | 0.86 | −0.04 | 0.06 | −0.31 | 0.08 | 0.34 |
| 367 | TNQSKNF | 0.19 | 0.37 | −0.02 | 0.17 | −0.06 | −0.08 | 10.28 |
| 368 | TNRTIQT | 0.37 | 1.08 | 0.12 | 0.37 | 0.22 | 0.18 | 0.50 |
| 369 | TNRTQAH | 0.33 | 0.17 | −0.09 | −0.22 | −0.30 | −0.28 | −0.16 |
| 370 | TNRTQLY | 0.30 | 0.21 | 0.04 | 0.02 | −0.11 | −0.07 | −0.02 |
| 371 | TNSTLRQ | 0.23 | 0.91 | 0.02 | 0.30 | −0.01 | 0.10 | 0.31 |
| 372 | TNSTTKS | 0.21 | 0.54 | −0.04 | 0.29 | −0.10 | 0.02 | 0.22 |
| 373 | TNTTRAM | 0.14 | 0.84 | −0.04 | 0.18 | −0.16 | 0.09 | 0.16 |
| 374 | TNVTRST | 0.12 | 0.76 | −0.17 | 0.00 | −0.15 | 0.01 | −0.21 |
| 375 | VNKVRDV | 0.20 | 0.52 | −0.05 | 0.02 | 0.10 | 0.04 | 0.25 |
| 376 | VNSVRMT | −0.22 | 0.32 | −0.34 | −0.07 | 0.05 | −0.08 | 0.05 |
| 377 | VNTTRFS | 0.16 | 0.30 | 0.03 | 0.04 | −0.04 | 0.06 | 0.32 |
| 378 | VQSNRTN | 0.32 | 0.29 | −0.05 | 0.13 | −0.01 | 0.02 | 0.25 |
| 379 | WGNRIQI | 0.04 | 0.25 | −0.28 | −0.20 | −0.46 | −0.21 | 0.08 |

log10 enrichment scores for the indicated cell, tissue or organ are shown
log10 enrichment scores for the indicated tissue or organ are shown

TABLE 5
CNS LUNG SEQUENCE 11 MER

| SEQ | Amino | Endothelial | | | Small | Large | |
|---|---|---|---|---|---|---|---|
| ID NO | Acid | Cells | Astrocytes | Neurons | Intestine | Intestine | Stomach |
| 380 | AQGNNTRPTAH | 0.11 | −0.46 | 0.01 | −0.30 | 0.00 | −0.21 |
| 381 | AQGNRTTSSEQ | 0.17 | 0.58 | −0.03 | 0.02 | 0.32 | 0.02 |
| 382 | AQSNAVRANSQ | 0.46 | −0.57 | −0.45 | −0.48 | −0.33 | −0.06 |
| 383 | AQTNDTRRISQ | 0.36 | −0.41 | −0.18 | −0.19 | −0.22 | 0.00 |
| 384 | AQTNNTKPLAH | 0.72 | 0.46 | 0.07 | 0.03 | −0.16 | −0.03 |

| SEQ | Amino | | | | | | | |
|---|---|---|---|---|---|---|---|---|
| ID NO | Acid | Liver | Lung | Heart | Kidney | Pancreas | Spleen | Eye |
| 380 | AQGNNTRPTAH | −0.35 | 0.78 | −0.36 | −0.22 | −0.38 | −0.40 | 0.08 |
| 381 | AQGNRTTSSEQ | 0.08 | 0.67 | −0.26 | 0.19 | −0.22 | −0.24 | 0.36 |
| 382 | AQSNAVRANSQ | −0.14 | 0.59 | −0.34 | −0.07 | −0.10 | −0.22 | −0.01 |
| 383 | AQTNDTRRISQ | −0.42 | 0.43 | −0.33 | −0.27 | −0.39 | −0.45 | 0.07 |
| 384 | AQTNNTKPLAH | 0.04 | 1.16 | 0.02 | 0.14 | −0.10 | −0.02 | 0.11 |

log10 enrichment scores for the indicated cell, tissue or organ are shown
log10 enrichment scores for the indicated tissue or organ are shown

### Targeting Peptides and Capsid Proteins

Disclosed herein include AAV targeting peptides. The AAV targeting peptide can comprise an amino acid sequence that comprises at least 4 contiguous amino acids from the sequence of PHEGSSR (SEQ ID NO: 169). For example, the targeting peptide can comprise the sequence of PHEG (SEQ ID NO: 392), HEGS (SEQ ID NO: 393), EGSS (SEQ ID NO: 394) or GSSR (SEQ ID NO: 395). The targeting peptide can comprise at least 5 contiguous amino acids from the sequence of PHEGSSR (SEQ ID NO: 169). For example, the targeting peptide can comprise the sequence of PHEGS (SEQ ID NO: 396), HEGSS (SEQ ID NO: 397), or EGSSR (SEQ ID NO: 398). The targeting peptide can comprise at least 6 contiguous amino acids from the sequence of PHEGSSR (SEQ ID NO: 169). For example, the targeting peptide can comprise the sequence of PHEGSS (SEQ ID NO: 399) or HEGSSR (SEQ ID NO: 400). The targeting peptide can comprise PHEGSSR (SEQ ID NO: 169).

In some embodiments, the AAV targeting peptide comprises an amino acid sequence that comprises at least 4 contiguous amino acids from the sequence of LNNTKTT (SEQ ID NO: 237). For example, the targeting peptide can comprise the sequence of LNNT (SEQ ID NO: 401), NNTK (SEQ ID NO: 402), NTKT (SEQ ID NO: 403), or TKTT (SEQ ID NO: 404). The targeting peptide can comprise at least 5 contiguous amino acids from the sequence of LNNTKTT (SEQ ID NO: 237). For example, the targeting peptide can comprise the sequence of LNNTK (SEQ ID NO: 405), NNTKT (SEQ ID NO: 406), or NTKTT (SEQ ID NO: 407). The targeting peptide can comprise at least 6 contiguous amino acids from the sequence of LNNTKTT (SEQ ID NO: 237). For example, the targeting peptide can comprise the sequence of LNNTKT (SEQ ID NO: 408), or NNTKTT (SEQ ID NO: 409). The targeting peptide can comprise GNNTRDT LNNTKTT (SEQ ID NO: 237).

In some embodiments, the AAV targeting peptide comprises an amino acid sequence that comprises at least 4 contiguous amino acids from the sequence of SNLARNV (SEQ ID NO: 274). For example, the targeting peptide can comprise the sequence of SNLA (SEQ ID NO: 410), NLAR (SEQ ID NO: 411), LARN (SEQ ID NO: 412), or ARNV (SEQ ID NO: 413). The targeting peptide can comprise at least 5 contiguous amino acids from the sequence of SNLARNV (SEQ ID NO: 274). For example, the targeting peptide can comprise the sequence of SNLAR (SEQ ID NO: 414), NLARN (SEQ ID NO: 415), or LARNV (SEQ ID NO: 416). The targeting peptide can comprise at least 6 contiguous amino acids from the sequence of SNLARNV (SEQ ID NO: 274). For example, the targeting peptide can comprise the sequence of SNLARN (SEQ ID NO: 417) or NLARNV (SEQ ID NO: 418). The targeting peptide can comprise SNLARNV (SEQ ID NO: 274).

In some embodiments, the AAV targeting peptide comprises an amino acid sequence that comprises at least 4 contiguous amino acids from the sequence of TNNTKPL (SEQ ID NO: 390). For example, the targeting peptide can comprise the sequence of TNNT (SEQ ID NO: 419), NNTK (SEQ ID NO: 420), NTKP (SEQ ID NO: 421), or TKPL (SEQ ID NO: 422). The targeting peptide can comprise at least 5 contiguous amino acids from the sequence of TNNTKPL (SEQ ID NO: 390). For example, the targeting peptide can comprise the sequence of TNNTK (SEQ ID NO: 423), NNTKP (SEQ ID NO: 424), or NTKPL (SEQ ID NO: 425). The targeting peptide can comprise at least 6 contiguous amino acids from the sequence of TNNTKPL (SEQ ID NO: 390). For example, the targeting peptide can comprise the sequence of TNNTKP (SEQ ID NO: 426) or NNTKPL (SEQ ID NO: 427). The targeting peptide can comprise TNNTKPL (SEQ ID NO: 390).

In some embodiments, the AAV targeting peptide comprises at least 4 contiguous amino acids from the sequence of PNASVNS (SEQ ID NO: 387). For example, the targeting peptide can comprise the sequence of PNAS (SEQ ID NO: 428), NASV (SEQ ID NO: 429), ASVN (SEQ ID NO: 430), or SVNS (SEQ ID NO: 431). In some embodiments, the AAV targeting peptide comprises at least 5 contiguous amino acids from the sequence of PNASVNS (SEQ ID NO: 387). For example, the targeting peptide can comprise the sequence of PNASV (SEQ ID NO: 432), NASVN (SEQ ID NO: 433), or ASVNS (SEQ ID NO: 434). In some embodiments, the AAV targeting peptide comprises at least 6 contiguous amino acids from the sequence of PNASVNS (SEQ ID NO: 387). For example, the targeting peptide can comprise the sequence of PNASVN (SEQ ID NO: 435), or NASVNS (SEQ ID NO: 436). In some embodiments, the AAV targeting peptide comprises the sequence of PNASVNS (SEQ ID NO: 387).

In some embodiments, the AAV targeting peptide comprises at least 4 contiguous amino acids from the sequence of LNTIRNV (SEQ ID NO: 388). For example, the targeting peptide can comprise the sequence of LNTI (SEQ ID NO: 437), NTIR (SEQ ID NO: 438), TIRN (SEQ ID NO: 439), or IRNV (SEQ ID NO: 440). The AAV targeting peptide can comprise at least 5 contiguous amino acids from the sequence of LNTIRNV (SEQ ID NO: 388). For example, the targeting peptide can comprise the sequence of LNTIR (SEQ ID NO: 441), NTIRN (SEQ ID NO: 442), or TIRNV (SEQ ID NO: 443). The AAV targeting peptide can comprise at least 6 contiguous amino acids from the sequence of LNTIRNV (SEQ ID NO: 388). For example, the targeting peptide can comprise the sequence of LNTIRN (SEQ ID NO: 444), or NTIRNV (SEQ ID NO: 445). The AAV targeting peptide can comprise the sequence of LNTIRNV (SEQ ID NO: 388).

The targeting peptide can be a CNS targeting peptide, a PNS targeting peptide, and/or a lung targeting peptide. The targeting AAV peptide can be part of an AAV. The targeting peptide can be part of a capsid protein of the AAV. The targeting peptide can be conjugated to a nanoparticle, a second molecule, a viral capsid protein, or a combination thereof.

Disclosed herein include AAV capsid proteins comprising one or more AAV targeting peptides provided herein. The AAV serotype used to derive the AAV capsid protein can vary. The AAV capsid can be derived from AAV9, or a variant thereof. The AAV capsid can be derived from an AAV selected from AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10. The AAV capsid protein can be derived from an AAV serotype selected from AAV9, AAV9 K449R (or K449R AAV9), AAV1, AAVrhl0, AAV-DJ, AAV-DJ8, AAV5, AAVPHP.B (PHP.B), AAVPHP.A (PHP.A), AAVG2B-26, AAVG2B-13, AAVTH1.1-32, AAVTH1.1-35, AAVPHP.B2 (PHP.B2), AAVPHP.B 3 (PHP.B 3), AAVPHP.N/PHP.B-DGT, AAVPHP.B-EST, AAVPHP.B-GGT, AAVPHP.B-ATP, AAVPHP.B-ATT-T, AAVPHP.B-DGT-T, AAVPHP.B-GGT-T, AAVPHP.B-SGS, AAVPHP.B-AQP, AAVPHP.B-QQP, AAVPHP.B-SNP(3), AAVPHP.B-SNP, AAVPHP.B-QGT, AAVPHP.B-NQT, AAVPHP.B-EGS, AAVPHP.B-SGN, AAVPHP.B-EGT, AAVPHP.B-DST, AAVPHP.B-DST, AAVPHP.B-STP, AAVPHP.B-PQP, AAVPHP.B-SQP, AAVPHP.B-QLP, AAVPHP.B-TM1P, AAVPHP.B-TTP, AAVPHP.S/G2A12, AAVG2 Al 5/G2A3 (G2A3), AAVG2B4 (G2B4), AAVG2B5 (G2B5), PHP.S, AAV2, AAV2G9, AAV3, AAV3a, AAV3b, AAV3-3, AAV4, AAV4-4, AAV6, AAV6.1, AAV6.2, AAV6.1.2, AAV7, AAV7.2, AAV8, AAV9.11, AAV9.13, AAV9.16, AAV9.24, AAV9.45, AAV9.47, AAV9.61, AAV9.68, AAV9.84, AAV9.9, AAV10, AAV11, AAV 12, AAV16.3, AAV24.1, AAV27.3, AAV42.12, AAV42-1b, AAV42-2, AAV42-3a, AAV42-3b, AAV42-4, AAV42-5a, AAV42-5b, AAV42-6b, AAV42-8, AAV42-10, AAV42-11, AAV42-12, AAV42-13, AAV42-15, AAV42-aa, AAV43-1, AAV43-12, AAV43-20, AAV43-21, AAV43-23, AAV43-25, AAV43-5, AAV44.1, AAV44.2, AAV44.5, AAV223.1, AAV223.2, AAV223.4, AAV223.5, AAV223.6, AAV223.7, AAVl-7/rh.48, AAVl-8/rh.49, AAV2-15/rh.62, AAV2-3/rh.61, AAV2-4/rh.50, AAV2-5/rh.51, AAV3.1/hu.6, AAV3.1/hu.9, AAV3-9/rh.52, AAV3-1 1/rh.53, AAV4-8/rl 1.64, AAV4-9/rh.54, AAV4-19/rh.55, AAV5-3/rh.57, AAV5-22/rh.58, AAV7.3/hu.7, AAV16.8/hu.10, AAV16.12/hu.1 1, AAV29.3/bb.1, AAV29.5/bb.2, AAV106.1/hu.37, AAV1 14.3/hu.40, AAV127.2/hu.41, AAV127.5/hu.42, AAV128.3/hu.44, AAV130.4/hu.48, AAV145.1/hu.53, AAV145.5/hu.54, AAV145.6/hu.55, AAV161.10/hu.60, AAV161.6/hu.61, AAV33.12/hu.17, AAV33.4/hu.15, AAV33.8/hu.16, AAV52/hu.19, AAV52.1/hu.20, AAV58.2/hu.25, AAVA3.3, AAVA3.4, AAVA3.5, AAVA3.7, AAVC1, AAVC2, AAVC5, AAVF3, AAVF5, AAVH2, AAVrh.72, AAVhu.8, AAVrh.68, AAVrh.70, AAVpi.1, AAVpi.3, AAVpi.2, AAVrh.60, AAVrh.44, AAVrh.65, AAVrh.55, AAVrh.47, AAVrh.69, AAVrh.45, AAVrh.59, AAVhu. 12, AAVH6, AAVH-1/hu.1, AAVH-5/hu.3, AAVLG-10/rh.40, AAVLG-4/rh.38, AAVLG-9/hu.39, AAVN721-8/rh.43, AAVCh.5, AAVCh.SRi, AAVcy.2, AAVcy.3, AAVcy.4, AAVcy.5, AAVCy.SR1, AAVCy.5R2, AAVCy.5R3, AAVCy.5R4, AAVcy.6, AAVhu. 1, AAVhu.2, AAVhu.3, AAVhu.4, AAVhu.5, AAVhu.6, AAVhu.7, AAVhu.9, AAVhu.10, AAVhu. 11, AAVhu. 13, AAVhu.15, AAVhu.16, AAVhu. 1 7, AAVhu.1 8, AAVhu.20, AAVhu.21, AAVhu.22, AAVhu.23.2, AAVhu.24, AAVhu.25, AAVhu.27, AAVhu.28, AAVhu.29, AAVhu.29R, AAVhu.31, AAVhu.32, AAVhu.34, AAVhu.35, AAVhu.37, AAVhu.39, AAVhu.40, AAVhu.41, AAVhu.42, AAVhu.43, AAVhu.44, AAVhu.44R1, AAVhu.44R2, AAVhu.44R3, AAVhu.45, AAVhu.46, AAVhu.47, AAVhu.48, AAVhu.48R1, AAVhu.48R2, AAVhu.48R3, AAVhu.49, AAVhu.51, AAVhu.52, AAVhu.54, AAVhu.55, AAVhu.56, AAVhu.57, AAVhu.58, AAVhu.60, AAVhu.61, AAVhu.63, AAVhu.64, AAVhu.66, AAVhu.67, AAVhu. 1 4/9, AAVhu.t 19, AAVrh.2, AAVrh.2R, AAVrh.8, AAVrh.8R, AAVrh. 10, AAVrh.12, AAVrh. 13, AAVrh.13R, AAVrh. 14, AAVrh.17, AAVrh. 18, AAVrh.19, AAVrh.20, AAVrh.21, AAVrh.22, AAVrh.23, AAVrh.24, AAVrh.25, AAVrh.3 1, AAVrh.32, AAVrh.33, AAVrh.34, AAVrh.35, AAVrh.36, AAVrh.37, AAVrh.37R2, AAVrh.38, AAVrh.39, AAVrh.40, AAVrh.46, AAVrh.48, AAVrh.48.1, AAVrh.48.1.2, AAVrh.48.2, AAVrh.49, AAVrh.51, AAVrh.52, AAVrh.53, AAVrh.54, AAVrh.56, AAVrh.57, AAVrh.58, AAVrh.61, AAVrh.64, AAVrh.64R1, AAVrh.64R2, AAVrh.67, AAVrh.73, AAVrh.74, AAVrh8R, AAVrh8R A586R mutant, AAVrh8R R533 A mutant, AAAV, BAAV, caprine AAV, bovine AAV, AAVhEl.1, AAVhErl.5, AAVhER1.14, AAVhErl.8, AAVhErl.16, AAVhErl.18, AAVhErl.35, AAVhErl.7, AAVhErl.36, AAVhEr2.29, AAVhEr2.4, AAVhEr2.16, AAVhEr2.30, AAVhEr2.3 1, AAVhEr2.36, AAVhER1.23, AAVhEr3.1, AAV2.5T, AAV-PAEC, AAV-LK01, AAV-LK02, AAV-LK03, AAV-LK04, AAV-LK05, AAV-LK06, AAV-LK07, AAV-LK08, AAV-LK09, AAV-LK10, AAV-LK 11, AAV-LK12, AAV-LK13, AAV-LK14, AAV-LK15, AAV-LK16, AAV-LK17, AAV-LK18, AAV-LK19, AAV-PAEC2, AAV-PAEC4, AAV-PAEC6, AAV-PAEC7, AAV-PAEC8, AAV-PAEC11, AAV-PAEC12, AAV-2-pre-miRNA-101, AAV-8h, AAV-8b, AAV-h, AAV-b, AAV SM 10-2, AAV Shuffle 100-1, AAV Shuffle 100-3, AAV Shuffle 100-7, AAV Shuffle 10-2, AAV Shuffle 10-6, AAV Shuffle 10-8, AAV Shuffle 100-2, AAV SM 10-1, AAV SM 10-8, AAV SM 100-3, AAV SM 100-10, BNP61 AAV, BNP62 AAV, BNP63 AAV, AAVrh.50, AAVrh.43, AAVrh.62, AAVrh.48, AAVhu.19, AAVhu.11, AAVhu.53, AAV4-8/rh.64, AAVLG-9/hu.39, AAV54.5/hu.23, AAV54.2/hu.22, AAV54.7/hu.24, AAV54.1/hu.21, AAV54.4R/hu.27, AAV46.2/hu.28, AAV46.6/hu.29, AAV128.1/hu.43, true type AAV (ttAAV), EGRENN AAV 10, Japanese AAV 10 serotypes, AAV CBr-7.1, AAV CBr-7.10, AAV CBr-7.2, AAV CBr-7.3, AAV CBr-7.4, AAV CBr-7.5, AAV CBr-7.7, AAV CBr-7.8, AAV CBr-B7.3, AAV CBr-B7.4, AAV CBr-E1, AAV CBr-E2, AAV CBr-E3, AAV CBr-E4, AAV CBr-E5, AAV CBr-e5, AAV CBr-E6, AAV CBr-E7, AAV CBr-E8, AAV CHt-1, AAV CHt-2, AAV CHt-3, AAV CHt-6.1, AAV CHt-6.1O, AAV CHt-6.5, AAV CHt-6.6, AAV CHt-6.7, AAV CHt-6.8, AAV CHt-P1, AAV CHt-P2, AAV CHt-P5, AAV CHt-P6, AAV CHt-P8, AAV CHt-P9, AAV CKd-1, AAV CKd-10, AAV CKd-2, AAV CKd-3, AAV CKd-4, AAV CKd-6, AAV CKd-7, AAV CKd-8, AAV CKd-B1, AAV CKd-B2, AAV CKd-B3, AAV CKd-B4, AAV CKd-B5, AAV CKd-B6, AAV CKd-B7, AAV CKd-B8, AAV CKd-H1, AAV CKd-H2, AAV CKd-H3, AAV CKd-H4, AAV CKd-H5, AAV CKd-H6, AAV CKd-N3, AAV CKd-N4, AAV CKd-N9, AAV CLg-F1, AAV CLg-F2, AAV CLg-F3, AAV CLg-F4, AAV CLg-F5, AAV CLg-F6, AAV CLg-F7, AAV CLg-F8, AAV CLv-1, AAV CLvl-1, AAV Clvl-10, AAV CLvl-2, AAV CLv-12, AAV CLvl-3, AAV CLv-1 3, AAV CLvl-4, AAV Clvl-7, AAV Clvl-8, AAV Clvl-9, AAV CLv-2, AAV CLv-3, AAV CLv-4, AAV CLv-6, AAV CLv-8, AAV CLv-D1, AAV CLv-D2, AAV CLv-D3, AAV CLv-D4, AAV CLv-D5, AAV CLv-D6, AAV CLv-D7, AAV CLv-D8, AAV CLv-E1, AAV CLv-K1, AAV CLv-K3, AAV CLv-K6, AAV CLv-L4, AAV CLv-L5, AAV CLv-L6, AAV CLv-M1, AAV CLv-M1 1, AAV CLv-M2, AAV CLv-M5, AAV CLv-M6, AAV CLv-M7, AAV CLv-M8, AAV CLv-M9, AAV CLv-R1, AAV CLv-R2, AAV CLv-R3, AAV CLv-R4, AAV CLv-R5, AAV CLv-R6, AAV CLv-R7, AAV CLv-R8, AAV CLv-R9, AAV CSp-1, AAV CSp-1O, AAV CSp-1 1, AAV CSp-2, AAV CSp-3, AAV CSp-4, AAV CSp-6, AAV CSp-7, AAV CSp-8, AAV CSp-8.10, AAV CSp-8.2, AAV CSp-8.4, AAV CSp-8.5, AAV CSp-8.6, AAV CSp-8.7, AAV CSp-8.8, AAV CSp-8.9, AAV CSp-9, AAV.hu.48R3, AAV.VR-355, AAV3B, AAV4, AAV5, AAVF1/HSC1, AAVF11/HSC11, AAVF12/HSC12, AAVF13/HSC13, AAVF14/HSC14, AAVF15/HSC15, AAVF16/HSC16, AAVF17/HSC17, AAVF2/HSC2, AAVF3/HSC3, AAVF4/HSC4, AAVF5/HSC5, AAVF6/HSC6, AAVF7/HSC7, AAVF8/HSC8, AAVF9/HSC9, variants thereof, a hybrid or chimera of any of the foregoing AAV serotypes, or a combination thereof. The rAAV disclosed herein can have a capsid from a different serotype of AAV than the rAAV genome. The AAV capsid can be derived from AAV1, AAV2, AAV3, AAV3b, AAV4, AAV5, AAV6, AAV7, AAV8, AAV9, AAV10, human isolate hu.31, human isolate hu.32, rhesus isolate rh.8, or rhesus isolate rh.10.

The engineered AAV capsid proteins described herein can have, in some cases, an insertion or substitution of an amino acid that is heterologous to the wild-type AAV capsid protein at the amino acid position of the insertion or substitution. In some embodiments, the amino acid is not endogenous to the wild-type AAV capsid protein at the amino acid position of the insertion or substitution. The amino acid can be a naturally occurring amino acid in the same or equivalent amino acid position as the insertion of the substitution in a different AAV capsid protein.

The rAAV can comprise a chimeric AAV capsid. A “chimeric” AAV capsid refers to a capsid that has an exogenous amino acid or amino acid sequence. The rAAV can comprise a mosaic AAV capsid. A “mosaic” AAV capsid refers to a capsid that is made up of two or more capsid proteins or polypeptides, each derived from a different AAV serotype. The rAAV can be a result of transcapsidation, which, in some cases, refers to the packaging of an inverted terminal repeat (ITR) from a first serotype into a capsid of a second serotype, wherein the first and second serotypes are not the same. In some cases, the capsid genes of the parental AAV serotype can be pseudotyped, which means that the ITRs from a first AAV serotype (e.g., AAV1) can be used in a capsid from a second AAV serotype (e.g., AAV9), wherein the first and second AAV serotypes are not the same. As a non-limiting example, a pseudotyped AAV serotype comprising the AAV1 ITRs and AAV9 capsid protein can be indicated AAV1/9. The rAAV can additionally, or alternatively, comprise a capsid that has been engineered to express an exogenous ligand binding moiety (e.g., receptor), or a native receptor that is modified.

The rAAV capsid proteins can comprise a substitution or insertion of one or more amino acids in an amino acid sequence of an AAV capsid protein. The AAV capsid protein from which the engineered AAV capsid protein of the present disclosure is produced can be referred to as a “parental” or “wild-type” AAV capsid protein, or a “corresponding unmodified capsid protein.” In some cases, the parental AAV capsid protein has a serotype selected from AAV1, AAV2, rAAV3, AAV4, AAV5, AAV6, AAV7, AAV8, AAV9, AAV10, AAV 11, and AAV12. The complete genome of AAV-1 is provided in GenBank Accession No. NC_002077; the complete genome of AAV-2 is provided in GenBank Accession No. NC_001401 and Srivastava et al., J. Virol., 45: 555-564 (1983); the complete genome of AAV-3 is provided in GenBank Accession No. NC_1829; the complete genome of AAV-4 is provided in GenBank Accession No. NC_001829; the AAV-5 genome is provided in GenBank Accession No. AF085716; the complete genome of AAV-6 is provided in GenBank Accession No. NC_00 1862; at least portions of AAV-7 and AAV-8 genomes are provided in GenBank Accession Nos. AX753246 and AX753249, respectively; the AAV-9 genome is provided in Gao et al., J. Virol., 78: 6381-6388 (2004); the AAV-10 genome is provided in Mol. Ther., 13(1): 67-76 (2006); the AAV-11 genome is provided in Virology, 330(2): 375-383 (2004); portions of the AAV-12 genome are provided in Genbank Accession No. DQ813647; portions of the AAV-13 genome are provided in Genbank Accession No. EU285562. At least portions of the AAV-DJ genome are provided in Grimm, D. et al. J. Virol. 82, 5887-5911 (2008).

### Nucleic Acids Encoding the Targeting Peptides and Capsid Proteins

Disclosed herein include nucleic acids. The nucleic acid can comprise a sequence encoding any of the AAV targeting peptides provided herein. For example, there are provided herein plasmid vectors encoding the variant capsid proteins of the present disclosure (e.g., comprising targeting peptides). Also disclosed are nucleic acids encoding the rAAV capsids comprising variant AAV capsid proteins (e.g., comprising targeting peptides) of the present disclosure. Heterologous nucleic acids and transgenes of the present embodiment can also include plasmid vectors. Plasmid vectors are useful for the generation of the rAAV particles described herein. An AAV vector can comprise a genome of a helper virus. Helper virus proteins are required for the assembly of a recombinant rAAV, and packaging of a transgene containing a heterologous nucleic acid into the rAAV. The helper virus genes are adenovirus genes E4, E2a and VA, that when expressed in the cell, assist with AAV replication. An AAV vector can comprise E2, E4, and/or VA. The AAV vector can comprise one of helper virus proteins, or any combination thereof. In some instances, the plasmid vector is bacterial. In some instances, the plasmid vector is derived from Escherichia coli. In some instances, the nucleic acid sequence comprises, in a 5′ to 3′ direction: (1) a 5′ ITR sequence, (2) a Replication (Rep) gene, (3) a Capsid (Cap) gene, and (4) a 3′ ITR, wherein the Cap gene encodes the variant AAV capsid protein described herein. In some instances, the plasmid vector encodes a pseudotyped AAV capsid protein.

### Adeno-Associated Virus (AAV) Vectors and Recombinant AAVs

AAV is a replication-deficient parvovirus, the single-stranded DNA genome of which is about 4.7 kb in length including 145 nucleotide ITRs. The ITRs play a role in integration of the AAV DNA into the host cell genome. When AAV infects a host cell, the viral genome integrates into the host's chromosome resulting in latent infection of the cell. In a natural system, a helper virus (e.g., adenovirus or herpesvirus) provides genes that allow for production of AAV virus in the infected cell. In the case of adenovirus, genes E1A, E1B, E2A, E4 and VA provide helper functions. Upon infection with a helper virus, the AAV provirus is rescued and amplified, and both AAV and adenovirus are produced. In the instances of rAAV vectors having no Rep and/or Cap genes, the AAV can be non-integrating.

Disclosed herein include rAAVs comprising an AAV targeting peptide provided herein, or an AAV capsid protein provided herein. In some embodiments, the rAAV comprises an AAV capsid protein which can comprise the AAV targeting peptide provided herein.

The AAV vector can comprise coding regions of one or more proteins of interest. The AAV vector can include a 5′ ITR of AAV, a 3′ AAV ITR, a promoter, and a restriction site downstream of the promoter to allow insertion of a polynucleotide encoding one or more proteins of interest. The promoter and the restriction site can be located downstream of the 5′ AAV ITR and upstream of the 3′ AAV ITR. The AAV vector can include a posttranscriptional regulator-element downstream of the restriction site and upstream of the 3′ AAV ITR. The AAV vectors disclosed herein can be used as AAV transfer vectors carrying a transgene encoding a protein of interest for producing rAAV viruses that can express the protein of interest in a host cell.

Generation of the viral vector can be accomplished using any suitable genetic engineering techniques well known in the art, including, without limitation, the standard techniques of restriction endonuclease digestion, ligation, transformation, plasmid purification, and DNA sequencing, for example as described in Sambrook el al. (Molecular Cloning: A Laboratory Manual. Cold Spring Harbor Laboratory Press, N.Y. (1989)). The viral vector can incorporate sequences from the genome of any known organism. The sequences can be incorporated in their native form or can be modified in any way to obtain a desired activity. For example, the sequences can comprise insertions, deletions or substitutions.

The viral vectors can include additional sequences that make the vectors suitable for replication and integration in eukaryotes. The viral vectors disclosed herein can include a shuttle element that makes the vectors suitable for replication and integration in both prokaryotes and eukaryotes. The viral vectors can include additional transcription and translation initiation sequences, such as promoters and enhancers; and additional transcription and translation terminators, such as polyadenylation signals. Various regulatory elements that can be included in an AAV vector have been described in US2012/0232133 which is hereby incorporated by reference in its entirety.

Vectors comprising a nucleic acid sequence encoding the modified AAV capsid proteins of the present disclosure are also provided herein. For example, the vectors of the present disclosure can comprise a nucleic acid sequence encoding the two AAV viral genes, Rep (Replication), and a Cap (Capsid) gene, wherein the Cap gene, encoding viral capsid proteins VP1, VP2, and VP3 is modified to produce the modified AAV capsid proteins of the present disclosure.

Disclosed herein are methods of producing an rAAV. In some embodiments, all elements that are required for AAV production in target cell (e.g., HEK293 cells) are transiently transfected into the target cell using suitable methods known in the art. For example, the rAAV of the present disclosure can be produced by co-transfecting three plasmid vectors, a first vector with ITR-containing plasmid carrying the transgene (e.g., a DNA sequence that encodes a trophic factor, a growth factor, or other soluble factors), a second vector that carries the AAV Rep and Cap genes (e.g., one or more variant capsid proteins provided herein); and (3), a third vector that provides the helper genes isolated from adenovirus. In some cases, rAAVs of the present disclosure are generated using the methods described in Challis et al. Systemic AAV vectors for widespread and targeted gene delivery in rodents. Nat. Protoc. 14, 379 (2019), which is hereby incorporated by reference in its entirety. Briefly, triple transfection of HEK293T cells using polyethylenimine (PEI) is performed, viruses are collected after 120 hours from both cell lysates and media and purified over iodixanol.

Disclosed herein, are methods of manufacturing comprising: (a) introducing into a cell a nucleic acid comprising: (i) a first nucleic acid sequence (heterologous nucleic acid) encoding, e.g., a protein, enclosed by a 5′ and a 3′ inverted terminal repeat (ITR) sequence; (ii) a second nucleic acid sequence encoding a viral genome comprising a 5′ ITR sequence, a Replication (Rep) gene, one or more (Cap) genes, and a 3′ ITR, wherein the one or more Cap genes encodes a variant AAV capsid protein described herein; and (iii) a third nucleic acid sequence encoding a first helper virus protein selected from E4orf6, E2a, and VA RNA, and optionally, a second helper virus protein comprising E1a or E1b55k; (b) expressing in the cell the AAV capsid protein described herein; (c) assembling an AAV particle comprising the AAV capsid proteins disclosed herein; and (d) packaging the first nucleic acid sequence in the AAV particle. In some instances, the methods further comprise packing the first nucleic acid sequence encoding the therapeutic gene expression product such that it becomes encapsidated by the rAAV capsid protein. The rAAV particles can be isolated, concentrated, and purified using suitable viral purification methods, such as those described herein.

The rAAVs can be generated by triple transfection of precursor cells (e.g., HEK293T) cells using a standard transfection protocol (e.g., with PEI). Viral particles can be harvested from the media after a period of time (e.g., 72 h post transfection) and from the cells and media at a later point in time (e.g., 120 h post transfection). Virus present in the media can be concentrated by precipitation with 8% poly(ethylene glycol) and 500 mM sodium chloride and the precipitated virus is added to the lysates prepared from the collected cells. The viruses can be purified over iodixanol (Optiprep, Sigma) step gradients (15%, 25%, 40% and 60%). Viruses can be concentrated and formulated in PBS. Virus titers can be determined by measuring the number of DNaseI-resistant vector genome copies (VGs) using qPCR and the linearized genome plasmid as a control.

The Rep protein can be Rep78, Rep68, Rep52, and Rep40. The genome of the AAV helper virus can comprise an AAV helper gene selected from E2, E4, and VA. The first nucleic acid sequence and the second nucleic acid sequence can be in trans. In some instances, the first nucleic acid sequence and the second nucleic acid sequence are in cis. In some instances, the first nucleic acid sequence, the second nucleic acid sequence and the third nucleic acid sequence, are in trans.

The cell can be a cell from a human, a primate, a murine, a feline, a canine, a porcine, an ovine, a bovine, an equine, a caprine and a lupine host cell. The cell can be a progenitor or precursor cell, such as a stem cell. In some instances, the stem cell is a mesenchymal cell, embryonic stem cell, induced pluripotent stem cell (iPSC), fibroblast or other tissue specific stem cell. The cell can be immortalized. In some instances, the embryonic stem cell is a human embryonic stem cell. In some instances, the human embryonic stem cell is a human embryonic kidney 293 (HEK-293) cell. In some instances, the cell is a differentiated cell. Based on the disclosure provided, it is expected that this system can be used in conjunction with any transgenic line expressing a recombinase in the target cell type of interest to develop AAV capsids that more efficiently transduce that target cell population.

The rAAV capsid proteins can be isolated and purified. The AAV can be isolated and purified by methods standard in the art such as by column chromatography or cesium chloride gradients. Methods for purifying AAV from helper virus can be known in the art and can include methods disclosed in, for example, Clark et al., Hum. Gene Ther., 10(6): 1031-1039 (1999); Schenpp and Clark, Methods Mol. Med., 69: 427-443 (2002); U.S. Pat. No. 6,566,118 and WO 98/09657.

The rAAV capsid and/or rAAV capsid protein can be conjugated to a nanoparticle, a second molecule, or a viral capsid protein. In some cases, the nanoparticle or viral capsid protein would encapsidate the therapeutic nucleic acid described herein. In some instances, the second molecule is a therapeutic agent, e.g., a small molecule, antibody, antigen-binding fragment, peptide, or protein, such as those described herein. In some instances, the second molecule is a detectable moiety. For example, the modified AAV capsid and/or rAAV capsid protein conjugated to a detectable moiety can be used for in vitro, ex vivo, or in vivo biomedical research applications, the detectable moiety used to visualize the modified capsid protein. The modified AAV capsid and/or rAAV capsid protein conjugated to a detectable moiety can also be used for diagnostic purposes.

One or more insertions, substitutions, or point mutations can be employed in a single system (e.g., in a single AAV vector, a single AAV capsid protein, or a single rAAV). For example, one can employ one or more targeting sequences and also modify other sites to reduce the recognition of the AAVs by the pre-existing antibodies present in a subject, such as a human. The AAV vector can include a capsid, which influences the tropism/targeting, speed of expression and possible immune response. The vector can also include the rAAV, which genome carries the transgene/therapeutic aspects (e.g., sequences) along with regulatory sequences. The vector can include the targeting sequence within/on a substrate that is or transports the desired molecule (e.g., therapeutic molecule, diagnostic molecule).

The location of the targeting peptide within the capsid protein can vary. The AAV capsid protein can comprise the AAV targeting peptide provided herein inserted between two adjacent amino acids in AA586-592 (e.g., between AA586 and AA587, AA587 and AA588, AA588 and AA589, AA589 and AA590, AA590 and AA591, AA591 and AA592) or functional equivalents thereof of the AAV capsid protein. The two adjacent amino acids can be AA588 and AA589. The AAV capsid protein can comprise, or consist thereof, SEQ ID NO: 391 (e.g., the VP1 protein of AAV9).

The rAAV can comprise an rAAV vector genome which comprises genetic information (e.g., heterologous nucleic acid) that are assembled into a rAAV via viral packaging. In some instances, the viral genome is from an AAV serotype selected from AAV1, AAV2, AAV3, AAV4, AAV5, AAV6, AAV7, AAV8, AAV9, AAV10, AAV 11, and AAV12.

A viral genome, such as those described herein, can comprise a transgene, which in some cases encodes a heterologous gene expression product (e.g., therapeutic gene expression product, recombinant capsid protein, and the like). The transgene is in cis with two ITRs flanking the transgene. The transgene may comprise a therapeutic nucleic acid encoding a therapeutic gene expression product

The viral genome, in some cases, is a single stranded viral DNA comprising the transgene. The AAV vector can be episomal. In some instances, the viral genome is a concatemer. An episomal viral genome can develop chromatin-like organization in the target cell that does not integrate into the genome of the target cell. When infected into non-dividing cells, the stability of the episomal viral genome in the target cell enable the long-term transgene expression. Alternatively, the AAV vector can integrate the transgene into the genome of the target cell predominantly at a specific site (e.g., AAVS 1 on human chromosome 19).

The rAAV genome can, for example, comprise at least one inverted terminal repeat configured to allow packaging into a vector and a cap gene. It can further include a sequence within a rep gene required for expression and splicing of the cap gene. The genome can further include a sequence capable of expressing a capsid protein provided herein. The AAV genome contains both the full rep and cap sequence that have been modified so as to not prevent the replication of the virus under conditions in which it could normally replicate (co-infection of a mammalian cell along with a helper virus such as adenovirus). A pseudo wild-type (“wt”) genome can be one that has an engineered cap gene within a “wt” AAV genome. The “pseudo-wild type” AAV genome can contain the viral replication gene (rep) and capsid gene (cap) flanked by ITRs. The rAAV genome can contain the cap gene and only those sequences within the rep gene required for the expression and splicing of the cap gene products. A capsid gene recombinase recognition sequence can be provided with inverted terminal repeats flanking these sequences.

Uses of AAV Vectors and rAAVs

Disclosed herein include compositions. The composition can comprise an AAV targeting peptide provided herein, an AAV capsid protein provided herein, a nucleic acid provided herein, an rAAV provided herein, or a combination thereof. The composition can be a pharmaceutical composition comprising one or more pharmaceutically acceptable carriers. Disclosed herein include compositions for use in the delivery of an agent to a target environment of a subject in need. The composition can comprise an AAV comprising (1) an AAV capsid protein provided herein and (2) an agent to be delivered to the target environment of the subject. In some embodiments, the target environment is the nervous system, the lung or a combination thereof.

The nervous system can be the CNS, the PNS, or a combination thereof. The target environment can be a neuron, a glial cell, an oligodendrocyte, an ependymal cell, an astrocyte, a Schwann cell, a satellite cell, or an enteric glial cell in the CNS, a neuron or an astrocyte in the PNS, an endothelial cell in the lung, or a combination thereof. The agent can be delivered to neural tissue in the CNS, ganglia or nerve fibre in the PNS, or epithelial lining of the lung or a combination thereof of the subject.

Disclosed herein include compositions for use in the delivery of an agent to a target environment of a subject in need. The composition can comprise an AAV comprising (1) an AAV capsid protein comprising an AAV targeting peptide and (2) an agent to be delivered to the target environment of the subject. The AAV targeting peptide can comprise at least 4 contiguous amino acids from the sequence of PNASVNS (SEQ ID NO: 387). The target environment can be the PNS.

The target environment can be a neuron or a glial cell in the PNS, or a combination thereof. The glial cell can be an astrocyte. The agent can be delivered to ganglia or nerve fibre in the PNS, or a combination thereof of the subject. The agent can be delivered to dorsal root ganglia, nodose ganglia, enteric ganglia in the PNS, or a combination thereof of the subject.

The composition can comprise an AAV comprising (1) an AAV capsid protein comprising an AAV targeting peptide and (2) an agent to be delivered to the target environment of the subject. In some embodiments, the AAV targeting peptide can comprise at least 4 contiguous amino acids from the sequence of LNTIRNV (SEQ ID NO: 388). The target environment can be the lung. The target environment can be an alveolar cell.

The composition can be a pharmaceutical composition comprising one or more pharmaceutically acceptable carriers. The agent to be delivered can comprise a nucleic acid, a peptide, a small molecule, an aptamer, or a combination thereof. The AAV vectors disclosed herein can be effectively transduced to a target environment (e.g., the CNS, the PNS and the lung), for example, for delivering nucleic acids. A method of delivering a nucleic acid sequence to the nervous system is provided. The protein can be part of a capsid of an AAV. The AAV can comprise a nucleic acid sequence to be delivered to a nervous system. One can then administer the AAV to the subject.

The nucleic acid sequence to be delivered to a target environment (e.g., nervous system) can comprise one or more sequences that would be of some use or benefit to the nervous system and/or the local of delivery or surrounding tissue or environment. It can be a nucleic acid that encodes a protein of interest. The nucleic acid can comprise one or more of: a) a DNA sequence that encodes a trophic factor, a growth factor, or a soluble protein; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

The vector can also comprise regulatory control elements known to one of skill in the art to influence the expression of the RNA and/or protein products encoded by the polynucleotide within desired cells of the subject.

Functionally, expression of the polynucleotide can be at least in part controllable by the operably linked regulatory elements such that the element(s) modulates transcription of the polynucleotide, transport, processing and stability of the RNA encoded by the polynucleotide and, as appropriate, translation of the transcript. A specific example of an expression control element is a promoter, which is usually located 5′ of the transcribed sequence. Another example of an expression control element is an enhancer, which can be located 5′ or 3′ of the transcribed sequence, or within the transcribed sequence. Another example of a regulatory element is a recognition sequence for a microRNA. Another example of a regulatory element is an intron and the splice donor and splice acceptor sequences that regulate the splicing of said intron. Another example of a regulatory element is a transcription termination signal and/or a polyadenylation sequence.

Expression control elements and promoters include those active in a particular tissue or cell type, referred to herein as a “tissue-specific expression control elements/promoters.” Tissue-specific expression control elements are typically active in a specific cell or tissue (for example in the liver, brain, central nervous system, spinal cord, eye, retina or lung). Expression control elements are typically active in these cells, tissues or organs because they are recognized by transcriptional activator proteins, or other regulators of transcription, that are unique to a specific cell, tissue or organ type.

Expression control elements also include ubiquitous or promiscuous promoters/enhancers which are capable of driving expression of a polynucleotide in many different cell types. Such elements include, but are not limited to the cytomegalovirus (CMV) immediate early promoter/enhancer sequences, the Rous sarcoma virus (RSV) promoter/enhancer sequences, the CMV, chicken β-actin, rabbit β-globin (CAG) promoter/enhancer sequences, and the other viral promoters/enhancers active in a variety of mammalian cell types; promoter/enhancer sequences from ubiquitously or promiscuously expressed mammalian genes including, but not limited to, beta actin, ubiquitin or EF1 alpha; or synthetic elements that are not present in nature.

Expression control elements also can confer expression in a manner that is regulatable, that is, a signal or stimuli increases or decreases expression of the operably linked polynucleotide. A regulatable element that increases expression of the operably linked polynucleotide in response to a signal or stimuli is also referred to as an “inducible element” (that is, it is induced by a signal). Particular examples include, but are not limited to, a hormone (e.g., steroid) inducible promoter. A regulatable element that decreases expression of the operably linked polynucleotide in response to a signal or stimuli is referred to as a “repressible element” (that is, the signal decreases expression such that when the signal, is removed or absent, expression is increased). Typically, the amount of increase or decrease conferred by such elements is proportional to the amount of signal or stimuli present: the greater the amount of signal or stimuli, the greater the increase or decrease in expression.

The heterologous nucleic acid can comprise a 5′ ITR and a 3′ ITR. The agent can comprise a DNA sequence encoding a protein (e.g., a trophic factor, a growth factor, or a soluble protein). The heterologous nucleic acid can comprise a promoter operably linked to the polynucleotide encoding, e.g., a protein or an RNA agent. The promoter can be capable of inducing the transcription of the polynucleotide. Transcription of the polynucleotide can generate a transcript. The heterologous nucleic acid can comprise one or more of a 5′ UTR, 3′ UTR, a minipromoter, an enhancer, a splicing signal, a polyadenylation signal, a terminator, one or more silencer effector binding sequences, a protein degradation signal, and an internal ribosome-entry element (IRES). The silencer effector can comprise a microRNA (miRNA), a precursor microRNA (pre-miRNA), a small interfering RNA (siRNA), a short-hairpin RNA (shRNA), precursors thereof, derivatives thereof, or a combination thereof. The silencer effector can be capable of binding the one or more silencer effector binding sequences, thereby reducing the stability of the transcript and/or reducing the translation of the transcript. The silencing effector can comprise one or more miRNA binding sites (e.g., miR-122 binding sites). miRNA binding sites are operably linked regulatory elements that are typically located in the 3′UTR of the transcribed sequence. Binding of miRNAs to the target transcript (in complex with the RNA-Induced Silencing Complex, RISC) can reduce expression of the target transcript via translation inhibition and/or transcript degradation.

The polynucleotide further can comprise a transcript stabilization element. The transcript stabilization element can comprise woodchuck hepatitis post-translational regulatory element (WPRE), bovine growth hormone polyadenylation (bGH-polyA) signal sequence, human growth hormone polyadenylation (hGH-polyA) signal sequence, or any combination thereof. The nucleic acid can be or can encode an RNA agent. The RNA agent can comprise one or more of dsRNA, siRNA, shRNA, pre-miRNA, pri-miRNA, miRNA, stRNA, lncRNA, piRNA, and snoRNA. The RNA agent inhibits or suppresses the expression of a gene of interest in a cell. The gene of interest can be SOD1, MAPT, APOE, HTT, C90RF72, TDP-43, APP, BACE, SNCA, ATXN1, ATXN2, ATXN3, ATXN7, SCN1A-SCN5A, or SCN8A-SCN11A. The heterologous nucleic acid further can comprise a polynucleotide encoding one or more secondary proteins, and the protein and the one or more secondary proteins can comprise a synthetic protein circuit. The heterologous nucleic acid can comprise a single-stranded AAV (ssAAV) vector or a self-complementary AAV (scAAV) vector.

The promoter can comprise a ubiquitous promoter, for example a cytomegalovirus (CMV) immediate early promoter, a CMV promoter, a viral simian virus 40 (SV40) (e.g., early or late), a Moloney murine leukemia virus (MoMLV) LTR promoter, a Rous sarcoma virus (RSV) LTR, an RSV promoter, a herpes simplex virus (HSV) (thymidine kinase) promoter, H5, P7.5, and P11 promoters from vaccinia virus, an elongation factor 1-alpha (EF1a) promoter, early growth response 1 (EGR1), ferritin H (FerH), ferritin L (FerL), Glyceraldehyde 3-phosphate dehydrogenase (GAPDH), eukaryotic translation initiation factor 4A1 (EIF4A1), heat shock 70 kDa protein 5 (HSPA5), heat shock protein 90 kDa beta, member 1 (HSP90B1), heat shock protein 70 kDa (HSP70), β-kinesin (β-KIN), the human ROSA 26 locus, a Ubiquitin C promoter (UBC), a phosphoglycerate kinase-1 (PGK) promoter, 3-phosphoglycerate kinase promoter, a cytomegalovirus enhancer, human β-actin (HBA) promoter, chicken β-actin (CBA) promoter, a CAG promoter, a CBH promoter, or any combination thereof.

The promoter can be an inducible promoter, for example a tetracycline responsive promoter, a TRE promoter, a Tre3G promoter, an ecdysone responsive promoter, a cumate responsive promoter, a glucocorticoid responsive promoter, and estrogen responsive promoter, a PPAR-γ promoter, an RU-486 responsive promoter, or a combination thereof.

The promoter can comprise a tissue-specific promoter and/or a lineage-specific promoter. The tissue specific promoter can be a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a a-myosin heavy chain (a-MHC) promoter, or a cardiac Troponin T (cTnT) promoter. The tissue specific promoter can be a neuron-specific promoter, for example a synapsin-1 (Syn) promoter, a CaMKIIa promoter, a calcium/calmodulin-dependent protein kinase II a promoter, a tubulin alpha I promoter, a neuron-specific enolase promoter, a platelet-derived growth factor beta chain promoter, TRPV1 promoter, a Nav1.7 promoter, a Nav1.8 promoter, a Nav1.9 promoter, or an Advillin promoter. The tissue specific promoter can be a muscle-specific promoter. The muscle-specific promoter can comprise a MCK promoter.

The promoter can comprise an intronic sequence. The promoter can comprise a bidirectional promoter and/or an enhancer. In some embodiments, the enhancer can be a CMV enhancer. One or more cells of a subject can comprise an endogenous version of a nucleic acid sequence (e.g., a gene), and the promoter can comprise or can be derived from the promoter of the endogenous version. One or more cells of a subject can comprise an endogenous version of the nucleic acid sequence, and the sequence is not truncated relative to the endogenous version.

The promoter can vary in length, for example be less than 1 kb. The promoter can be greater than 1 kb. The promoter can have a length of 200, 210, 220, 230, 240, 250, 260, 270, 280, 290, 300, 310, 320, 330, 340, 350, 360, 370, 380, 390, 400, 410, 420, 430, 440, 450, 460, 470, 480, 490, 500, 510, 520, 530, 540, 550, 560, 570, 580, 590, 600, 610, 620, 630, 640, 650, 660, 670, 680, 690, 700, 710, 720, 730, 740, 750, 760, 770, 780, 790, 800 bp, or a number or a range between any two of these values, or more than 800 bp. The promoter can provide expression of the therapeutic gene expression product for a period of time in targeted tissues such as, but not limited to, the CNS. Expression of the therapeutic gene expression product can be for a period of 1 hour, 2, hours, 3 hours, 4 hours, 5 hours, 6 hours, 7 hours, 8 hours, 9 hours, 10 hours, 11 hours, 12 hours, 13 hours, 14 hours, 15 hours, 16 hours, 1 hours, 18 hours, 19 hours, 20 hours, 21 hours, 22 hours, 23 hours, 1 day, 2 days, 3 days, 4 days, 5 days, 6 days, 1 week, 8 days, 9 days, 10 days, 11 days, 12 days, 13 days, 2 weeks, 15 days, 16 days, 17 days, 18 days, 19 days, 20 days, 3 weeks, 22 days, 23 days, 24 days, 25 days, 26 days, 27 days, 28 days, 29 days, 30 days, 31 days, 1 month, 2 months, 3 months, 4 months, 5 months, 6 months, 7 months, 8 months, 9 months, 10 months, 11 months, 1 year, 13 months, 14 months, 15 months, 16 months, 17 months, 18 months, 19 months, 20 months, 21 months, 22 months, 23 months, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, 20 years, 21 years, 22 years, 23 years, 24 years, 25 years, 26 years, 27 years, 28 years, 29 years, 30 years, 31 years, 32 years, 33 years, 34 years, 35 years, 36 years, 37 years, 38 years, 39 years, 40 years, 41 years, 42 years, 43 years, 44 years, 45 years, 46 years, 47 years, 48 years, 49 years, 50 years, 55 years, 60 years, 65 years, or a number or a range between any two of these values, or more than 65 years.

As used herein, a “protein of interest” can be any protein, including naturally-occurring and non-naturally occurring proteins. A polynucleotide encoding one or more proteins of interest can be present in one of the AAV vectors disclosed herein, wherein the polynucleotide is operably linked with a promoter. In some instances, the promoter can drive the expression of the protein(s) of interest in a host cell (e.g., a neuron). The protein of interest can be an anti-tau antibody, an anti-AB antibody, and/or ApoE isoform.

The protein can comprise aromatic L-amino acid decarboxylase (AADC), survival motor neuron 1 (SMN1), frataxin (FXN), Cystic Fibrosis Transmembrane Conductance Regulator (CFTR), Factor X (FIX), RPE65, Retinoid Isomerohydrolase (RPE65), Sarcoglycan Alpha (SGCA), and sarco/endoplasmic reticulum Ca2+-ATPase (SERCA2a), ApoE2, GBA1, GRN, ASP A, CLN2, GLB1, SGSH, NAGLU, IDS, NPC1, GAN, CFTR, GDE, OTOF, DYSF, MYO7A, ABCA4, F8, CEP290, CDH23, DMD, ALMS1, or any combination thereof.

The protein can comprise a disease-associated protein. The level of expression of the disease-associated protein can correlate with the occurrence and/or progression of the disease. The protein can comprise methyl CpG binding protein 2 (MeCP2), DRK1A, KAT6A, NIPBL, HDAC4, UBE3A, EHMT1, one or more genes encoded on chromosome 9q34.3, NPHP1, LIMK1 one or more genes encoded on chromosome 7q11.23, P53, TPI1, FGFR1 and related genes, RA1, SHANK3, CLN3, NF-1, TP53, PFK, CD40L, CYP19A1, PGRN, CHRNA7, PMP22, CD40LG, derivatives thereof, or any combination thereof.

The nucleic acid can comprise a cDNA that encodes a protein to control or monitor the activity or state of a cell, and/or for assessing the state of a cell. The protein can comprise fluorescence activity, polymerase activity, protease activity, phosphatase activity, kinase activity, SUMOylating activity, deSUMOylating activity, ribosylation activity, deribosylation activity, myristoylation activity demyristoylation activity, or any combination thereof. The protein can comprise nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity, glycosylase activity, acetyltransferase activity, deacetylase activity, adenylation activity, deadenylation activity, or any combination thereof. The protein can comprise a nuclear localization signal (NLS) or a nuclear export signal (NES).

The protein can comprise a CRE recombinase, GCaMP, a cell therapy component, a knock-down gene therapy component, a cell-surface exposed epitope, or any combination thereof. The protein can comprise a chimeric antigen receptor. The protein can comprise a diagnostic agent (e.g., green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), TagRFP, Dronpa, Padron, mApple, mCherry, mruby3, rsCherry, rsCherryRev, derivatives thereof, or any combination thereof).

The nucleic acid can comprise a cDNA that encodes a protein for gene editing, or a guide RNA; or a DNA sequence for genome editing via homologous recombination. The protein can comprise a programmable nuclease. The programmable nuclease is SpCas9 or a derivative thereof, VRER, VQR, EQR SpCas9; xCas9-3.7; eSpCas9; Cas9-HF1; HypaCas9; evoCas9; HiFi Cas9; ScCas9; StCas9; NmCas9; SaCas9; CjCas9; CasX; Cas9 H940A nickase; Cas12 and derivatives thereof; dcas9-APOBEC1 fusion, BE3, and dcas9-deaminase fusions; dcas9-Krab, dCas9-VP64, dCas9-Tet1, and dcas9-transcriptional regulator fusions; Dcas9-fluorescent protein fusions; Cas13-fluorescent protein fusions; RCas9-fluorescent protein fusions; or Cas13-adenosine deaminase fusions. The programmable nuclease can comprise a zinc finger nuclease (ZFN) and/or transcription activator-like effector nuclease (TALEN). The programmable nuclease can comprise Streptococcus pyogenes Cas9 (SpCas9), Staphylococcus aureus Cas9 (SaCas9), a zinc finger nuclease, TAL effector nuclease, meganuclease, MegaTAL, Tev-m TALEN, MegaTev, homing endonuclease, Cas1, Cas1B, Cas2, Cas3, Cas4, Cas5, Cas6, Cas7, Cas8, Cas9, Cas100, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Cs×10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13c, derivatives thereof, or any combination thereof. The heterologous nucleic acid and/or rAAV can comprise a polynucleotide encoding (i) a targeting molecule and/or (ii) a donor nucleic acid. The targeting molecule can be capable of associating with the programmable nuclease. The targeting molecule can comprise single strand DNA or single strand RNA. The targeting molecule can comprise a single guide RNA (sgRNA).

The rAAV disclosed herein can comprise one or more of the heterologous nucleic acids disclosed herein. The heterologous nucleic acid can comprise a polynucleotide encoding a protein. The nucleic acid can be or can encode an RNA agent. The heterologous nucleic acid can comprise a promoter operably linked to the polynucleotide encoding a protein. As disclosed herein, the gene is operatively linked with appropriate regulatory elements in some embodiments. The one or more genes of the heterologous nucleic acid can comprise an siRNA, an shRNA, an antisense RNA oligonucleotide, an antisense miRNA, a trans-splicing RNA, a guide RNA, single-guide RNA, crRNA, a tracrRNA, a trans-splicing RNA, a pre-mRNA, a mRNA, or any combination thereof. The one or more genes of the heterologous nucleic acid can comprise one or more synthetic protein circuit components. The one or more genes of the heterologous nucleic acid can comprise can entire synthetic protein circuit comprising one or more synthetic protein circuit components. The one or more genes of the heterologous nucleic acid can comprise two or more synthetic protein circuits.

The protein can be any protein, including naturally-occurring and non-naturally occurring proteins. Examples include, but are not limited to, luciferases; fluorescent proteins (e.g., GFP); growth hormones (GHs) and variants thereof, insulin-like growth factors (IGFs) and variants thereof; granulocyte colony-stimulating factors (G-CSFs) and variants thereof; erythropoietin (EPO) and variants thereof, insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof; clotting factors and variants thereof, cystic fibrosis transmembrane conductance regulator (CFTR) and variants thereof; and interferons and variants thereof.

Examples of protein of interest include, but are not limited to, luciferases; fluorescent proteins (e.g., GFP); growth hormones (GHs) and variants thereof, insulin-like growth factors (IGFs) and variants thereof; granulocyte colony-stimulating factors (G-CSFs) and variants thereof; erythropoietin (EPO) and variants thereof, insulin, such as proinsulin, preproinsulin, insulin, insulin analogs, and the like; antibodies and variants thereof, such as hybrid antibodies, chimeric antibodies, humanized antibodies, monoclonal antibodies; antigen binding fragments of an antibody (Fab fragments), single-chain variable fragments of an antibody (scFV fragments); dystrophin and variants thereof, clotting factors and variants thereof, CFTR and variants thereof, and interferons and variants thereof.

The protein of interest can be a therapeutic protein or variant thereof. Non-limiting examples of therapeutic proteins include growth factors, such as keratinocyte growth factor (GF), stem cell factor (SCF), fibroblast growth factor (FGF, such as basic FGF and acidic FGF), hepatocyte growth factor (HGF), insulin-like growth factors (IGFs), bone morphogenetic protein (BMP), epidermal growth factor (EGF), growth differentiation factor-9 (GDF-9), hepatoma derived growth factor (HDGF), myostatin (GDF-8), nerve growth factor (NGF), neurotrophins, platelet-derived growth factor (PDGF), thrombopoietin (TPO), transforming growth factor alpha (TGF-a), transforming growth factor beta (TGF-β), and the like; soluble receptors, such as soluble TNF-α receptors, soluble VEGF receptors, soluble interleukm receptors (e.g., soluble IL-1 receptors and soluble type II IL-1 receptors), soluble γ/δ T cell receptors, ligand-binding fragments of a soluble receptor, and the like; enzymes, such as -glucosidase, imiglucarase, β-glucocerebrosidase, and alglucerase; enzyme activators, such as tissue plasminogen activator; chemokines, such as IP-10, monokine induced by interferon-gamma (Mig), Groa/IL-S, RANTES, MlP-1 a, MIP-I β, MCP-1, PF-4, and the like; angiogenic agents, such as vascular endothelial growth factors (VEGFs, e.g., VEGF121, VEGF165, VEGF-C, VEGF-2), transforming growth factor-beta, basic fibroblast growth factor, glioma-derived growth factor, angiogenin, angiogenin-2; and the like; anti-angiogenic agents, such as a soluble VEGF receptor; protein vaccine; neuroactive peptides, such as nerve growth factor (NGF), bradykinin, cholecystokinin, gastin, secretin, oxytocin, gonadotropin-releasing hormone, beta-endorphin, enkephalin, substance P, somatostatin, prolactin, galanin, growth hormone-releasing hormone, bombesin, dynorphin, warfarin, neurotensin, motilin, thyrotropin, neuropeptide Y, luteinizing hormone, calcitonin, insulin, glucagons, vasopressin, angiotensin II, thyrotropin-releasing hormone, vasoactive intestinal peptide, a sleep peptide, and the like; thrombolytic agents; atrial natriuretic peptide; relaxin; glial fibrillary acidic protein; follicle stimulating hormone (FSH); human alpha-1 antitrypsin; leukemia inhibitory factor (LIF); transforming growth factors (TGFs); tissue factors, luteinizing hormone; macrophage activating factors; tumor necrosis factor (TNF); neutrophil chemotactic factor (NCF); nerve growth factor; tissue inhibitors of nietalloproteinases; vasoactive intestinal peptide; angiogenin; angiotropin; fibrin; hirudin; IL-1 receptor antagonists; and the like. Some other non-limiting examples of protein of interest include ciliary neurotrophic factor (CNTF); brain-derived neurotrophic factor (BDNF); neurotrophins 3 and 4/5 (NT-3 and 4/5); glial cell derived neurotrophic factor (GDNF); aromatic amino acid decarboxylase (AADC); hemophilia related clotting proteins, such as Factor IX or Factor X; dystrophin or nini-dystrophm; lysosomal acid lipase; phenylalanine hydroxylase (PAH); glycogen storage disease-related enzymes, such as glucose-6-phosphatase, acid maltase, glycogen debranching enzyme, muscle glycogen phosphorylase, liver glycogen phosphorylase, muscle phosphofructokinase, phosphorylase kinase (e.g., PHKA2), glucose transporter (e.g., GLUT2), aldolase A, O-enolase, and glycogen synthase; lysosomal enzymes (e.g., beta-N-acetylhexosaminidase A); and any variants thereof.

The protein of interest can be, for example, an active fragment of a protein, such as any of the aforementioned proteins, a fusion protein comprising some or all of two or more proteins, or a fusion protein comprising all or a portion of any of the aforementioned proteins.

The viral vector can comprise a polynucleotide comprising coding regions for two or more proteins of interest, The two or more proteins of interest can be the same or different from each other. The two or more proteins of interest can be related polypeptides, for example light chain(s) and heavy chain(s) of the same antibody.

The protein of interest can be a multi-subunit protein. For example, the protein of interest can comprise two or more subunits, or two or more independent polypeptide chains. The protein of interest can be an antibody, including, but are not limited to, antibodies of various isotypes (for example, IgG1, IgG2, IgG3, IgG, IgA, IgD, IgE, and IgM); monoclonal antibodies produced by any means known to those skilled in the art, including an antigen-binding fragment of a monoclonal antibody; humanized antibodies; chimeric antibodies; single-chain antibodies; antibody fragments such as Fv, F(ab′)2, Fab′, Fab, Facb, scFv and the like; provided that the antibody is capable of binding to antigen. The antibody can be a full-length antibody. The protein of interest is not an immunoadhesin.

The resulting targeting molecules can be employed in methods and/or therapies relating to in vivo gene transfer applications to long-lived cell populations. These can be applied to any rAAV-based gene therapy, including, for example: spinal muscular atrophy (SMA), amyotrophic lateral sclerosis (ALS), Parkinson's disease, Friedreich's ataxia, Pompe disease, Huntington's disease, Alzheimer's disease, Battens disease, lysosomal storage disorders, glioblastoma multiforme, Rett syndrome, Leber's congenital amaurosis, chronic pain, stroke, spinal cord injury, traumatic brain injury and lysosomal storage disorders. In addition, rAAVs can also be employed for in vivo delivery of transgenes for non-therapeutic scientific studies such as optogenetics, gene overexpression, gene knock-down with shRNA or miRNAs, modulation of endogenous miRNAs using miRNA sponges or decoys, recombinase delivery for conditional gene deletion, conditional (recombinase-dependent) expression, or gene editing with CRISPRs, TALENs, and zinc finger nucleases.

The gene can encode immunogenic material capable of stimulating an immune response (e.g., an adaptive immune response) such as, for example, antigenic peptides or proteins from a pathogen. The expression of the antigen can stimulate the body's adaptive immune system to provide an adaptive immune response. Thus, it is contemplated that the heterologous nucleic acids provided herein can be employed as vaccines for the prophylaxis or treatment of infectious diseases (e.g., as vaccines).

As described herein, the nucleotide sequence encoding the protein can be modified to improve expression efficiency of the protein. The methods that can be used to improve the transcription and/or translation of a gene herein are not particularly limited. For example, the nucleotide sequence can be modified to better reflect host codon usage to increase gene expression (e.g., protein production) in the host (e.g., a mammal).

The degree of gene expression in the target cell can vary. The amount of the protein expressed in the subject (e.g., the CNS of the subject) can vary. For example, the protein can be expressed in the subject in the amount of at least about 9 μg/ml, at least about 10 μg/ml, at least about 50 μg/ml, at least about 100 μg/ml, at least about 200 μg/ml, at least about 300 μg/ml, at least about 400 μg/ml, at least about 500 μg/ml, at least about 600 μg/ml, at least about 700 g/ml, at least about 800 μg/ml, at least about 900 μg/ml, or at least about 1000 μg/ml. The protein can be expressed in the subject in the amount of about 9 μg/ml, about 10 μg/ml, about 50 μg/ml, about 100 μg/ml, about 200 μg/ml, about 300 μg/ml, about 400 μg/ml, about 500 μg/ml, about 600 μg/ml, about 700 μg/ml, about 800 μg/ml, about 900 μg/ml, about 1000 μg/ml, about 1500 g/ml, about 2000 μg/ml, about 2500 μg/ml, or a range between any two of these values. A skilled artisan will understand that the expression level in which a protein is needed for the method to be effective can vary depending on non-limiting factors such as the particular protein and the subject receiving the treatment, and an effective amount of the protein can be readily determined by a skilled artisan using conventional methods known in the art without undue experimentation.

The agent can be an inducer of cell death. The agent can induce cell death by a non-endogenous cell death pathway (e.g., a bacterial pore-forming toxin). The agent (e.g., a protein encoded by a nucleic acid) can be a pro-survival protein. The agent can be a modulator of the immune system. The agent can activate an adaptive immune response, and innate immune response, or both. The nucleic acid can encode immunogenic material capable of stimulating an immune response (e.g., an adaptive immune response) such as, for example, antigenic peptides or proteins from a pathogen. The expression of the antigen can stimulate the body's adaptive immune system to provide an adaptive immune response. Thus, it is contemplated that the compositions provided herein can be employed as vaccines for the prophylaxis or treatment of infectious diseases (e.g., as vaccines). The protein can comprise a CRE recombinase, GCaMP, a cell therapy component, a knock-down gene therapy component, a cell-surface exposed epitope, or any combination thereof. The protein can comprise CFTR, GDE, OTOF, DYSF, MYO7A, ABCA4, F8, CEP290, CDH23, DMD, and ALMS1.

The agent can comprise a non-protein coding gene, such as an RNA agent, e.g., sequences encoding antisense RNAs, RNAi, shRNAs and micro RNAs (miRNAs), miRNA sponges or decoys, recombinase delivery for conditional gene deletion, conditional (recombinase-dependent) expression, includes those required for the gene editing components described herein. A non-protein coding gene can also encode a tRNA, rRNA, tmRNA, piRNA, double stranded RNA, snRNA, snoRNA, and/or long non-coding RNA (lncRNA). The RNA agent can comprise non-natural or modified nucleotides (e.g., pseudouridine). The non-protein coding gene can modulate the expression or the activity of a target gene or gene expression product. For example, the RNAs described herein can be used to inhibit gene expression in a target cell, for example, a cell in the CNS. Inhibition of gene expression can refer to an inhibition by at least about 20%, 30%, 40%, 50%, 60%, 70%, 80%, 85%, 90%, 95% and 100%. In some cases, the protein product of the targeted gene is inhibited by at least about 20%, 30%, 40%, 50%, 60%, 70%, 80%, 85%, 90%, 95% and 100%. The gene can be either a wild-type gene or a gene with at least one mutation. The targeted protein can be a wild-type protein, or a protein with at least one mutation.

Examples of genes encoding therapeutic proteins include a sequence associated with a signaling biochemical pathway, e.g., a signaling biochemical pathway-associated gene or polynucleotide (e.g., a signal transducer). The methods and compositions disclosed herein can comprise knockdown of an endogenous signal transducer accompanied by tuned expression of a protein comprising an appropriate version of signal transducer. Examples of DNA or RNA sequences contemplated herein include sequences for a disease-associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It can be a gene that becomes expressed at an abnormally high level; it can be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products can be known or unknown, and can be at a normal or abnormal level. Signal transducers can be can be associated with one or more diseases or disorders. A disease or disorder can be characterized by an aberrant signaling of one or more signal transducers disclosed herein. The activation level of the signal transducer can correlate with the occurrence and/or progression of a disease or disorder. The activation level of the signal transducer can be directly responsible or indirectly responsible for the etiology of the disease or disorder.

The rAAV having a capsid protein comprising one or more targeting peptides disclosed herein can be used to deliver genes to specific cell types in the target environment of a subject. For example, the rAAV can be used for delivering genes to neurons and glia in the nervous system (including PNS, CNS, or both) of a subject (e.g., a mammal). The compositions and methods disclosed herein can be used in, for example, (i) reducing the expression of mutant Huntingtin in patients with Huntington's Disease by, for example, incorporating a Huntingtin-specific microRNA expression cassette within a rAAV genome and packaging the rAAV genome into a variant rAAV (e.g., MaCPNS1) for delivery through, for example the vasculature, (ii) delivering a functional copy of the Frataxin gene to patients with Friedreich's ataxia, (iii) restoring expression of an enzyme critical for normal lysosomal function in patients lacking expression of the enzyme due to genetic mutation (e.g., patients with Neimann-Pick disease, mucopolysaccharidosis III, and/or Gaucher's disease), (iv) using the rAAV (e.g., MaCPNS1) to generate animal models of disease, or a combination thereof.

The subject in need can be a subject suffering from any disease, disorder, or injury related to the CNS, PNS, and/or the lung. The subject in need can be a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, lysosomal storage disorders that involve cells within the CNS, bronchiectasis, bronchitis, bronchopulmonary dysplasia, interstitial lung disease, occupational lung disease, cystic fibrosis, acute respiratory distress syndrome (ARDS), severe acute respiratory syndrome (SARS), intermittent asthma, mild persistent asthma, moderate persistent asthma, severe persistent asthma, chronic bronchitis, chronic obstructive pulmonary disease (COPD), interstitial lung disease, sarcoidosis, asbestosis, aspergilloma, aspergillosis, lobar pneumonia, multilobar pneumonia, bronchial pneumonia, interstitial pneumonia, pulmonary fibrosis, pulmonary tuberculosis, rheumatoid lung disease, pulmonary embolism, and non-small-cell lung carcinoma, adenocarcinoma, squamous-cell lung carcinoma, large-cell lung carcinoma, or small-cell lung carcinoma. The lysosomal storage disorder can be Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease. The subject in need can be a subject suffering from, at risk to develop, or can have suffered from a stroke, traumatic brain injury, epilepsy, spinal cord injury, emphysema, lung reperfusion injury, ischemia-reperfusion injury of the lung, or ventilator-induced lung injury.

### Pharmaceutical Compositions and Methods of Administration

Also disclosed herein are pharmaceutical compositions comprising one or more of the rAAV viruses disclosed herein and one or more pharmaceutically acceptable carriers. The compositions can also comprise additional ingredients such as diluents, stabilizers, excipients, and adjuvants. As used herein, “pharmaceutically acceptable” carriers, excipients, diluents, adjuvants, or stabilizers are nontoxic to the cell or subject being exposed thereto (preferably inert) at the dosages and concentrations employed or that have an acceptable level of toxicity as determined by the skilled practitioners. The carriers, diluents and adjuvants can include buffers such as phosphate, citrate, or other organic acids: antioxidants such as ascorbic acid; low molecular weight polypeptides (e.g., less than about 10 residues); proteins such as serum albumin, gelatin or immunoglobulins; hydrophilic polymers such as polyvinylpyrrolidone; amino acids such as glycine, glutamine, asparagine, arginine, or lysine; monosaccharides, di saccharides, and other carbohydrates including glucose, mannose, or dextrins; chelating agents such as EDTA; sugar alcohols such as mannitol or sorbitol; salt-forming counterions such as sodium; and/or nonionic surfactants such as Tween™, Pluronics™ or polyethylene glycol (PEG). The physiologically acceptable carrier can be an aqueous pH buffered solution.

Disclosed herein include methods of delivering an agent to a nervous system or a lung of a subject. The method can comprise: providing an AAV vector comprising an AAV capsid protein provided herein; and administering the AAV vector to the subject. The AAV vector can further comprise an agent to be delivered to the nervous system, the lung, or a combination thereof. The subject can be an adult animal.

Titers of the rAAV to be administered can vary depending, for example, on the particular rAAV, the mode of administration, the treatment goal, the individual, and the cell type(s) being targeted, and can be determined by methods standard in the art. As will be readily apparent to one skilled in the art, the useful in vivo dosage of the recombinant virus to be administered and the particular mode of administration can vary depending upon the age, weight, the severity of the affliction, and animal species treated, the particular recombinant virus expressing the protein of interest that is used, and the specific use for which the recombinant virus is employed. The determination of effective dosage levels, that is the dosage levels necessary to achieve the desired result, can be accomplished by one skilled in the art using routine pharmacological methods. Typically, human clinical applications of products are commenced at lower dosage levels, with dosage level being increased until the desired effect is achieved. Alternatively, acceptable in vitro studies can be used to establish useful doses and routes of administration of the compositions identified by the present methods using established pharmacological methods.

The exact dosage can be determined on a drug-by-drug basis, in most cases, some generalizations regarding the dosage can be made. The rAAV for delivery of an agent to the nervous system (e.g., CNS) of a subject can be administered, for example via injection, to a subject at a dose of between 1×10¹⁰ viral genome (vg) of the recombinant virus per kg of the subject and 2×10¹⁴ vg per kg, for example between 5×10¹¹ vg/kg and 5×10¹² vg/kg. The dose of the rAAV administered to the subject can be no more than 2×10¹⁴ vg per kg. The dose of the rAAV administered to the subject can be no more than 5×10¹² vg per kg. The dose of the rAAV administered to the subject can be no more than 5×10¹¹ vg per kg.

An effective dose and dosage of pharmaceutical compositions to prevent or treat the disease or condition disclosed herein is defined by an observed beneficial response related to the disease or condition, or symptom of the disease or condition. Beneficial response comprises preventing, alleviating, arresting, or curing the disease or condition, or symptom of the disease or condition. The beneficial response can be measured by detecting a measurable improvement in the presence, level, or activity, of biomarkers, transcriptomic risk profile, or intestinal microbiome in the subject. An “improvement,” as used herein refers to shift in the presence, level, or activity towards a presence, level, or activity, observed in normal individuals (e.g. individuals who do not suffer from the disease or condition). In instances wherein the therapeutic rAAV composition is not therapeutically effective or is not providing a sufficient alleviation of the disease or condition, or symptom of the disease or condition, then the dosage amount and/or route of administration can be changed, or an additional agent can be administered to the subject, along with the therapeutic rAAV composition. As a patient is started on a regimen of a therapeutic rAAV composition, the patient can also be weaned off (e.g., step-wise decrease in dose) a second treatment regimen.

Pharmaceutical compositions in accordance with the present disclosure can be administered at dosage levels sufficient to deliver from about 0.0001 mg/kg to about 100 mg/kg, from about 0.001 mg/kg to about 0.05 mg/kg, from about 0.005 mg/kg to about 0.05 mg/kg, from about 0.001 mg/kg to about 0.005 mg/kg, from about 0.05 mg/kg to about 0.5 mg/kg, from about 0.01 mg/kg to about 50 mg/kg, from about 0.1 mg/kg to about 40 mg/kg, from about 0.5 mg/kg to about 30 mg/kg, from about 0.01 mg/kg to about 10 mg/kg, from about 0.1 mg/kg to about 10 mg/kg, or from about 1 mg/kg to about 25 mg/kg, of subject body weight per day, one or more times a day, to obtain the desired therapeutic, diagnostic, or prophylactic effect. It will be understood that the above dosing concentrations may be converted to vg or viral genomes per kg or into total viral genomes administered by one of skill in the art.

A dose of the pharmaceutical composition can comprise a concentration of infectious particles of at least or about 10⁷, 10⁸, 10⁹, 10¹⁰, 10¹¹, 10¹², 10¹³, 10¹⁴, 10¹⁵, 10¹⁶, or 10¹⁷. In some cases, the concentration of infectious particles is 2×10⁷, 2×10⁸, 2×10⁹, 2×10¹⁰, 2×10¹¹, 2×10¹², 2×10¹³, 2×10¹⁴, 2×10¹⁵, 2×10¹⁶, 2×10¹⁷, or a range between any two of these values. In some cases, the concentration of the infectious particles is 3×10⁷, 3×10⁸, 3×10⁹, 3×10¹⁰, 3×10¹¹, 3×10¹², 3×10¹³, 3×10¹⁴, 3×10¹⁵, 3×10¹⁶, 3×10¹⁷, or a range between any two of these values. In some cases the concentration of the infectious particles is 4×10⁷, 4×10⁸, 4×10⁹, 4×10¹⁰, 4×10¹¹, 4×10¹², 4×10¹³, 4×10¹⁴, 4×10¹⁵, 4×10¹⁶, 4×10¹⁷, or a range between any two of these values. In some cases, the concentration of the infectious particles is 5×10⁷, 5×10⁸, 5×10⁹, 5×10¹⁰, 5×10¹¹, 5×10¹², 5×10¹³, 5×10¹⁴, 5×10¹⁵, 5×10¹⁶, 5×10¹⁷, or a range between any two of these values. In some cases, the concentration of the infectious particles is 6×10⁷, 6×10⁸, 6×10⁹, 6×10¹⁰, 6×10¹¹, 6×10¹², 6×10¹³, 6×10¹⁴, 6×10¹⁵, 6×10¹⁶, 6×10¹⁷, or a range between any two of these values. In some cases, the concentration of the infectious particles is 7×10⁷, 7×10⁸, 7×10⁹, 7×10¹⁰, 7×10¹¹, 7×10¹², 7×10¹³, 7×10¹⁴, 7×10¹⁵, 7×10¹⁶, 7×10¹⁷, or a range between any two of these values. In some cases, the concentration of the infectious particles is 8×10⁷, 8×10⁸, 8×10⁹, 8×10¹⁰, 8×10¹¹, 8×10¹², 8×10¹³, 8×10¹⁴, 8×10¹⁵, 8×10¹⁶, 8×10¹⁷, or a range between any two of these values. In some cases, the concentration of the infectious particles is 9×10⁷, 9×10⁸, 9×10⁹, 9×10¹⁰, 9×10¹¹, 9×10¹², 9×10¹³, 9×10¹⁴, 9×10¹⁵, 9×10¹⁶, 9×10¹⁷, or a range between any two of these values.

The recombinant viruses disclosed herein can be administered to a subject (e.g., a human) in need thereof. The route of the administration is not particularly limited. For example, a therapeutically effective amount of the recombinant viruses can be administered to the subject by via routes standard in the art. The composition can be for intravenous administration. The composition can be for systemic administration.

Non-limiting examples of the route include intramuscular, intravaginal, intravenous, intraperitoneal, subcutaneous, epicutaneous, intradermal, rectal, intraocular, pulmonary, intracranial, intraosseous, oral, buccal, systematic, or nasal. The recombinant virus can be administered to the subject by systematic transduction. The recombinant virus can be administered to the subject by intramuscular injection. The rAAV can be administered to the subject by the parenteral route (e.g., by intravenous, intramuscular or subcutaneous injection), by surface scarification or by inoculation into a body cavity of the subject. Route(s) of administration and serotype(s) of AAV components of the rAAV virus can be readily determined by one skilled in the art taking into account the infection and/or disease state being treated and the target cells/tissue(s) that are to express the protein of interest. It can be advantageous to administer the rAAV via intravenous administration. The variant AAV provided herein can advantageously provide for intravenous administration of vectors with enhanced tropisms for CNS, PNS and lung.

The agent can be delivered to the nervous system or the lung of the subject at least 1.5-fold more efficiently (e.g., 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to other organs or tissues. The agent can be delivered to the nervous system or the lung of the subject with an enrichment score relative to other organs or tissues of at least 0.01 (e.g., 0.01, 0.05, 0.1, 0.15, 0.2, 0.25, 0.3, 0.4, 0.45, 0.5, 0.6, 0.7, 0.8, 0.9, 1, 1.5, 2, 3, 4, 5, 6, 7, 8, 9, 10 or a number or a range between any of these values).

The nervous system can be the CNS and the PNS. The agent can be delivered to a neuron or an astrocyte of the nervous system of the subject at least 1.5-fold more efficiently (e.g., 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to other cells of the organs in which the PNS can be embedded. The agent can be delivered to a neuron or an astrocyte of the nervous system of the subject with an enrichment score relative to other cells in which the PNS is embedded of least 0.01 (e.g., 0.01, 0.05, 0.1, 0.15, 0.2, 0.25, 0.3, 0.4, 0.45, 0.5, 0.6, 0.7, 0.8, 0.9, 1, 1.5, 2, 3, 4, 5, 6, 7, 8, 9, 10 or a number or a range between any of these values). The agent can be delivered to the PNS in the gastrointestinal tract of the subject at least 1.5-fold more efficiently (e.g., 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to the nervous system or the lung than other organs or tissues. The agent can be delivered to the PNS in the gastrointestinal tract of the subject with an enrichment score relative to other organs or tissues of at least 0.01 (e.g., 0.01, 0.05, 0.1, 0.15, 0.2, 0.25, 0.3, 0.4, 0.45, 0.5, 0.6, 0.7, 0.8, 0.9, 1, 1.5, 2, 3, 4, 5, 6, 7, 8, 9, 10 or a number or a range between any of these values).

Disclosed herein include methods of delivering an agent to a PNS of a subject. The method can comprise: providing an AAV vector comprising an AAV capsid protein, and administering the AAV vector to the subject. The AAV capsid protein can comprise an AAV targeting peptide that can comprise at least 4 contiguous amino acids from the sequence of PNASVNS (SEQ ID NO: 387). The AAV vector can further comprise an agent to be delivered to the PNS. The agent can be delivered to a neuron or an astrocyte of the PNS of the subject at least 1.5-fold more efficiently (e.g., 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to the cells other than the neurons and the astrocytes in the organs in which the PNS is embedded. The agent can be delivered to a neuron or an astrocyte of the PNS of the subject with an enrichment score relative to cells other than the neurons and the astrocytes in the organs in which the PNS is embedded of at least 0.01 (e.g., 0.01, 0.05, 0.1, 0.15, 0.2, 0.25, 0.3, 0.4, 0.45, 0.5, 0.6, 0.7, 0.8, 0.9, 1, 1.5, 2, 3, 4, 5, 6, 7, 8, 9, 10 or a number or a range between any of these values).

Disclosed herein include methods of delivering an agent to a lung of a subject. The method can comprise: providing an AAV vector comprising an AAV capsid protein; and administering the AAV vector to the subject. The AAV capsid protein can comprise an AAV targeting peptide that can comprise at least 4 contiguous amino acids from the sequence of LNTIRNV (SEQ ID NO: 388). The AAV vector can further comprise an agent to be delivered to the lung. The agent can be delivered to alveolar cells in the lung of the subject at least 1.5-fold more efficiently (e.g., 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values) than the delivery of the agent to any cells in other organs or tissues. The agent can be delivered to the lung of the subject with an enrichment score relative to other organs or tissues of at least 0.01 (e.g., 0.01, 0.05, 0.1, 0.15, 0.2, 0.25, 0.3, 0.4, 0.45, 0.5, 0.6, 0.7, 0.8, 0.9, 1, 1.5, 2, 3, 4, 5, 6, 7, 8, 9, 10 or a number or a range between any of these values).

The AAVs disclosed herein can increase transduction efficiency of AAVs to a target environment (e.g., the CNS, the PNS, and the lung) in the subject as compared to a non-variant AAV (e.g., AAV9). For example, the inclusion of one or more of the targeting peptides disclosed herein in an rAAV can result in an increase in transduction efficiency by, or by at least, 10%, 20%, 30%, 40%, 50%, 60%, 70%, 80%, 90%, 100%, 1.5-fold, 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 10-fold, 20-fold, 30-fold, 40-fold, 50-fold, 60-fold, 70-fold, 80-fold, 90-fold, 100-fold, 150-fold, 200-fold or a number or a range between any two of these values, as compared to a non-variant AAV (e.g., AAV9). The increase can be at least 1.5-fold. The increase can be a 40-150 fold increase. The transduction efficiency can be increased for transducing the variant AAV to the CNS, the PNS, or both. The transduction efficiency can be increased for transducing the variant AAV to the lung. The transduction efficiency can be increased for transducing neurons and/or glia in the PNS and/or the CNS.

The agent can be delivered to the nervous system or lung of the subject at least 1.5-fold, 2-fold, 5-fold, 10-fold, 20-fold, 30-fold, 40-fold, 50-fold, 100-fold, 150-fold, 200-fold or a number or a range between any two of these values, more efficiently than the agent is delivered to the nervous system or lung of the subject by an AAV vector that does not comprise the targeting peptide (e.g., AAV9). The agent can be delivered to a neuron or an astrocyte of the nervous system of the subject at least 1.5-fold, 2-fold, 5-fold, 10-fold, 20-fold, 30-fold, 40-fold, 50-fold, 100-fold, 150-fold, 200-fold or a number or a range between any two of these values, more efficiently than the agent is delivered to a neuron or an astrocyte of the nervous system of the subject by an AAV vector that does not comprise the targeting peptide (e.g., AAV9). The agent can be delivered to the PNS in the gastrointestinal tract of the subject at least 1.5-fold, 2-fold, 5-fold, 10-fold, 20-fold, 30-fold, 40-fold, 50-fold, 100-fold, 150-fold, 200-fold or a number or a range between any two of these values, more efficiently than the agent is delivered to the PNS in the gastrointestinal tract of the subject by an AAV vector that does not comprise the targeting peptide (e.g., AAV9). The agent can be delivered to the lung of the subject at least 1.5-fold (e.g., 1.5-fold, 2-fold, 3-fold, 4-fold, 5-fold, 6-fold, 7-fold, 8-fold, 9-fold, 10-fold, 20-fold, 30-fold, 40-fold, 50-fold, 60-fold, 70-fold, 80-fold, 90-fold, 100-fold, or a number or a range between any of these values), more efficiently than the agent is delivered to the lung of the subject by an AAV vector that does not comprise the targeting peptide (e.g., AAV9).

Disclosed herein include methods of delivering an agent to a cell. The method can comprise: contacting an AAV vector comprising an AAV capsid protein provided herein with the cell. The AAV vector can further comprise an agent to be delivered to a nervous system, a lung, or a combination thereof. The cell can be a neuron, or an astrocyte in the nervous system, any cell in the lung or a combination thereof.

The AAV capsid protein can comprise an AAV targeting peptide that can comprise at least 4 contiguous amino acids from the sequence of PNASVNS (SEQ ID NO: 387). The AAV vector can further comprise an agent to be delivered to the PNS. The cell can be a neuron, an astrocyte, or a combination thereof. The AAV capsid protein can comprise an AAV targeting peptide that can comprise at least 4 contiguous amino acids from the sequence of LNTIRNV (SEQ ID NO: 388). The AAV vector can further comprise an agent to be delivered to a lung. The cell can be any cell in the lung. Contacting the AAV vector with the cell can occur in vitro, in vivo or ex vivo. The cell can be present in a tissue, an organ, or a subject. The agent to be delivered can comprise a nucleic acid, a peptide, a small molecule, an aptamer or a combination thereof. The nucleic acid can encode a therapeutic protein.

The nucleic acid can comprise one or more of: a) a DNA sequence that encodes a trophic factor, a growth factor, or other soluble factors capable of being released from the transduced cells and affect the survival or function of that cell and/or surrounding cells; b) a cDNA that restores protein function to humans or animals harboring a genetic mutation(s) in that gene; c) a cDNA that encodes a protein that can be used to control or alter the activity or state of a cell; d) a cDNA that encodes a protein or a nucleic acid that can be used for assessing the state of a cell; e) a cDNA that encodes a protein for gene editing, or a guide RNA; f) a DNA sequence for genome editing via homologous recombination; g) a DNA sequence encoding a therapeutic RNA; h) an shRNA or an artificial miRNA delivery system; and i) a DNA sequence that influences the splicing of an endogenous gene.

The AAV vector can be an AAV9 vector, or a variant thereof. The AAV vector can be derived from AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, or a variant thereof. The serotype of the AAV vector can be different from the serotype of the AAV capsid.

The variant AAV capsid can comprise tropism for a target tissue or a target cell. The target tissue or the target cell can comprise a tissue or a cell of a CNS, a PNS, or lung. The target cell can be a neuronal cell, a neural stem cell, glial cells, astrocytes, alveolar cells or a tumor cell. The target cell can be located in a brain, spinal cord, nerve fibre, dorsal root ganglia, nodose ganglia, or enteric ganglia. The target cell can comprise, a dendritic cell, a neural cell, a brain cell, an astrocyte, a microglial cell, and a neuron.

Actual administration of the rAAV can be accomplished by using any physical method that will transport the rAAV into the target tissue of the subject. For example, the rAAV disclosed herein can advantageously be administered intravenously for delivery to the CNS, the PNS or the lung. As disclosed herein, capsid proteins of the rAAV can be modified so that the rAAV is targeted to a particular target environment of interest such as central nervous system, and to enhance tropism to the target environment of interest (e.g., CNS, PNS or lung tropism). Pharmaceutical compositions can be prepared, for example, as injectable formulations.

The recombinant virus to be used can be utilized in liquid or freeze-dried form (in combination with one or more suitable preservatives and/or protective agents to protect the virus during the freeze-drying process). For gene therapy (e.g., of neurological disorders which may be ameliorated by a specific gene product) a therapeutically effective dose of the recombinant virus expressing the therapeutic protein is administered to a host in need of such treatment. The use of the recombinant virus disclosed herein in the manufacture of a medicament for inducing immunity in, or providing gene therapy to, a host is within the scope of the present application.

In instances where human dosages for the rAAV have been established for at least some condition, those same dosages, or dosages that are between about 0.1% and 500%, more preferably between about 25% and 250% of the established human dosage can be used. Where no human dosage is established, as will be the case for newly-discovered pharmaceutical compositions, a suitable human dosage can be inferred from ED₅₀ or ID₅₀ values, or other appropriate values derived from in vitro or in vivo studies, as qualified by toxicity studies and efficacy studies in animals.

A therapeutically effective amount of the rAAV can be administered to a subject at various points of time. For example, the rAAV can be administered to the subject prior to, during, or after the subject has developed a disease or disorder. The rAAV can also be administered to the subject prior to, during, or after the occurrence of a disease or disorder (e.g., Huntington's disease (HD), Alzheimer's disease, Parkinson's disease, Amyotrophic lateral sclerosis, spinal muscular atrophy, types I and II, Friedreich's Ataxia, Spinocerebellar ataxia and any of the lysosomal storage disorders that involve cells with CNS, which includes but is not limited to Krabbe disease, Sandhoff disease, Tay-Sachs, Gaucher disease (Type I, II, or 111), Niemann-Pick disease (NPC1 or NPC2 deficiency), Hurler syndrome, Pompe disease, Batten disease, or any combination thereof), chronic pain, bronchiectasis, bronchitis, bronchopulmonary dysplasia, interstitial lung disease, occupational lung disease, cystic fibrosis, acute respiratory distress syndrome (ARDS), severe acute respiratory syndrome (SARS), intermittent asthma, mild persistent asthma, moderate persistent asthma, severe persistent asthma, chronic bronchitis, chronic obstructive pulmonary disease (COPD), interstitial lung disease, sarcoidosis, asbestosis, aspergilloma, aspergillosis, lobar pneumonia, multilobar pneumonia, bronchial pneumonia, interstitial pneumonia, pulmonary fibrosis, pulmonary tuberculosis, rheumatoid lung disease, pulmonary embolism, and non-small-cell lung carcinoma, adenocarcinoma, squamous-cell lung carcinoma, large-cell lung carcinoma, or small-cell lung carcinoma or a combination thereof. The rAAV can be administered to the subject during remission of the disease or disorder. The rAAV can be administered prior to the onset of the disease or disorder in the subject. The rAAV can be administered to a subject at a risk of developing the disease or disorder.

The disease or disorder can comprise a neurological disease or disorder. For example, the neurological disease or disorder can comprise epilepsy, Dravet Syndrome, Lennox Gastaut Syndrome, myocolonic seizures, juvenile myocolonic epilepsy, refractory epilepsy, schizophrenia, juvenile spasms, West syndrome, infantile spasms, refractory infantile spasms, Alzheimer's disease, Creutzfeld-Jakob's syndrome/disease, bovine spongiform encephalopathy (BSE), prion related infections, diseases involving mitochondrial dysfunction, diseases involving β-amyloid and/or tauopathy, Down's syndrome, hepatic encephalopathy, Huntington's disease, motor neuron diseases, amyotrophic lateral sclerosis (ALS), olivoponto-cerebellar atrophy, post-operative cognitive deficit (POCD), systemic lupus erythematosus, systemic sclerosis, Sjogren's syndrome, Neuronal Ceroid Lipofuscinosis, neurodegenerative cerebellar ataxias, Parkinson's disease, Parkinson's dementia, mild cognitive impairment, cognitive deficits in various forms of mild cognitive impairment, cognitive deficits in various forms of dementia, dementia pugilistica, vascular and frontal lobe dementia, cognitive impairment, learning impairment, eye injuries, eye diseases, eye disorders, glaucoma, retinopathy, macular degeneration, head or brain or spinal cord injuries, head or brain or spinal cord trauma, convulsions, epileptic convulsions, epilepsy, temporal lobe epilepsy, myoclonic epilepsy, tinnitus, dyskinesias, chorea, Huntington's chorea, athetosis, dystonia, stereotypy, ballism, tardive dyskinesias, tic disorder, torticollis spasmodicus, blepharospasm, focal and generalized dystonia, nystagmus, hereditary cerebellar ataxias, corticobasal degeneration, tremor, essential tremor, addiction, anxiety disorders, panic disorders, social anxiety disorder (SAD), attention deficit hyperactivity disorder (ADHD), attention deficit syndrome (ADS), restless leg syndrome (RLS), hyperactivity in children, autism, dementia, dementia in Alzheimer's disease, dementia in Korsakoff syndrome, Korsakoff syndrome, vascular dementia, dementia related to HIV infections, HIV-1 encephalopathy, AIDS encephalopathy, AIDS dementia complex, AIDS-related dementia, major depressive disorder, major depression, depression, memory loss, stress, bipolar manic-depressive disorder, drug tolerance, drug tolerance to opioids, movement disorders, fragile-X syndrome, irritable bowel syndrome (IBS), migraine, multiple sclerosis (MS), muscle spasms, pain, chronic pain, acute pain, inflammatory pain, neuropathic pain, posttraumatic stress disorder (PTSD), schizophrenia, spasticity, Tourette's syndrome, eating disorders, food addiction, binge eating disorders, agoraphobia, generalized anxiety disorder, obsessive-compulsive disorder, panic disorder, social phobia, phobic disorders, substance-induced anxiety disorder, delusional disorder, schizoaffective disorder, schizophreniform disorder, substance-induced psychotic disorder, hypertension, emphysema, lung reperfusion injury, ischemia-reperfusion injury of the lung, or ventilator-induced lung injury or any combination thereof.

Disclosed herein can be formulations of pharmaceutically-acceptable excipients and carrier solutions suitable for delivery of the rAAV compositions described herein, as well as suitable dosing and treatment regimens for using the particular compositions described herein in a variety of treatment regimens. The amount of therapeutic gene expression product in each therapeutically-useful composition can be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. The rAAV compositions can be suitably formulated pharmaceutical compositions disclosed herein, to be delivered either intraocularly, intravitreally, parenterally, subcutaneously, intravenously, intracerebro-ventricularly, intramuscularly, intrathecally, orally, intraperitoneally, by oral or nasal inhalation, or by direct injection to one or more cells, tissues, or organs by direct injection. The rAAV disclosed herein can advantageously be administered intravenously for delivery to the CNS, the PNS or the lung.

The pharmaceutical forms of the AAV-based viral compositions suitable for injectable use can include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity can be maintained, for example, by the use of a coating, such as lecithin, by the maintenance of the required particle size in the case of dispersion and by the use of surfactants. The prevention of the action of microorganisms can be brought about by various antibacterial ad antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution can be suitably buffered, if necessary, and the liquid diluent first rendered isotonic with sufficient saline or glucose. These particular aqueous solutions are especially suitable for intravenous, intramuscular, subcutaneous and intraperitoneal administration. Some variation in dosage will necessarily occur depending on the condition of the subject being treated. The person responsible for administration will, in any event, determine the appropriate dose for the individual subject. Moreover, for human administration, preparations should meet sterility, pyrogenicity, and the general safety and purity standards as required by FDA Office of Biologics standards.

Disclosed herein are sterile injectable solutions comprising the rAAV compositions disclosed herein, which are prepared by incorporating the rAAV compositions disclosed herein in the required amount in the appropriate solvent with several of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Injectable solutions can be advantageous for systemic administration, for example by intravenous administration.

Also provided herein are formulations in a neutral or salt form. Pharmaceutically-acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

Formulations for intranasal administration can comprise a coarse powder comprising the active ingredient and having an average particle size from about 0.2 m to 500 m. Such formulations are administered in the manner in which snuff is taken, e.g. by rapid inhalation through the nasal passage from a container of the powder held close to the nose. Formulations suitable for nasal administration can, for example, comprise from about as little as 0.1% (w/w) and as much as 100% (w/w) of active ingredient, and can comprise one or more of the additional ingredients described herein. A pharmaceutical composition can be prepared, packaged, and/or sold in a formulation suitable for buccal administration. Such formulations can, for example, be in the form of tablets and/or lozenges made using conventional methods, and can, for example, comprise 0.1% to 20% (w/w) active ingredient, the balance comprising an orally dissolvable and/or degradable composition and, optionally, one or more of the additional ingredients described herein. Alternately, formulations suitable for buccal administration can comprise powders and/or an aerosolized and/or atomized solutions and/or suspensions comprising active ingredients. Such powdered, aerosolized, and/or aerosolized formulations, when dispersed, can comprise average particle and/or droplet sizes in the range of from about 0.1 nm to about 200 nm, and can further comprise one or more of any additional ingredients described herein.

Suitable dose and dosage administrated to a subject is determined by factors including, but not limited to, the particular therapeutic rAAV composition, disease condition and its severity, the identity (e.g., weight, sex, age) of the subject in need of treatment, and can be determined according to the particular circumstances surrounding the case, including, e.g., the specific agent being administered, the route of administration, the condition being treated, and the subject or host being treated.

The amount of AAV compositions and time of administration of such compositions will be within the purview of the skilled artisan having benefit of the present teachings. However, the administration of therapeutically-effective amounts of the disclosed compositions can be achieved by a single administration, such as for example, a single injection of sufficient numbers of infectious particles to provide therapeutic benefit to the patient undergoing such treatment. This is made possible, at least in part, by the fact that certain target cells (e.g., neurons) do not divide, obviating the need for multiple or chronic dosing.

It is advantageous to provide multiple, or successive administrations of the AAV vector compositions, either over a relatively short, or a relatively prolonged period of time, as can be determined by the medical practitioner overseeing the administration of such compositions. For example, the number of infectious particles administered to a mammal can be on the order of about 10⁷, 10⁸, 10⁹, 10¹⁰, 10¹¹, 10¹², 10¹³, or even higher, infectious particles/ml given either as a single dose, or divided into two or more administrations as can be required to achieve therapy of the particular disease or disorder being treated. In fact, it can be desirable to administer two or more different AAV vector compositions, either alone, or in combination with one or more other therapeutic drugs to achieve the desired effects of a particular therapy regimen. The daily and unit dosages can be altered depending on a number of variables including, but not limited to, the activity of the therapeutic rAAV composition used, the disease or condition to be treated, the mode of administration, the requirements of the individual subject, the severity of the disease or condition being treated, and the judgment of the practitioner.

The targeting peptides described herein can be used to generate rAAVs with enhanced CNS tropisms with capsid proteins derived from different AAV serotypes (e.g., AAV9 and AAV1). This can advantageously provide for administration of two or more different AAV vector compositions without inducing immune response in the subject.

The dosing frequency of the rAAV virus can vary. For example, the rAAV virus can be administered to the subject about once every week, about once every two weeks, about once every month, about one every six months, about once every year, about once every two years, about once every three years, about once every four years, about once every five years, about once every six years, about once every seven years, about once every eight years, about once every nine years, about once every ten years, or about once every fifteen years. The rAAV virus can be administered to the subject at most about once every week, at most about once every two weeks, at most about once every month, at most about one every six months, at most about once every year, at most about once every two years, at most about once every three years, at most about once every four years, at most about once every five years, at most about once every six years, at most about once every seven years, at most about once every eight years, at most about once every nine years, at most about once every ten years, or at most about once every fifteen years.

Disclosed herein are kits comprising compositions disclosed herein. Also disclosed herein are kits for the treatment or prevention of a disease or conditions of the CNS, PNS, lung or target organ or environment (e.g., CNS, PNS, and lung). The disease or condition can be cancer, a pathogen infection, neurological disease, muscular disease, or an immune disorder. A kit can include a therapeutic or prophylactic composition containing an effective amount of a composition of a rAAV particle encapsidating a heterologous nucleic acid provided herein and a rAAV capsid protein of the present disclosure. A kit can include a therapeutic or prophylactic composition containing an effective amount of cells modified by the rAAV described herein (“modified cell”), in unit dosage form that express therapeutic nucleic acid. A kit can comprise a sterile container which can contain a therapeutic composition; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

The rAAV can be provided together with instructions for administering the rAAV to a subject having or at risk of developing the disease or condition. Instructions can generally include information about the use of the composition for the treatment or prevention of the disease or condition.

The kit can include allogenic cells. The kit can include cells that can comprise a genomic modification. The kit can comprise “off-the-shelf” cells. The kit can include cells that can be expanded for clinical use. The kit can contain contents for a research purpose.

The instructions can include at least one of the following: description of the therapeutic rAAV composition; dosage schedule and administration for treatment or prevention of the disease or condition disclosed herein; precautions; warnings; indications; counter-indications; overdosage information; adverse reactions; animal pharmacology; clinical studies; and/or references. The instructions can be printed directly on the container (when present), or as a label applied to the container, or as a separate sheet, pamphlet, card, or folder supplied in or with the container. The instructions can provide procedures for administering the rAAV to the subject alone. The instructions provide can procedures for administering the rAAV to the subject at least about 1 hour (hr), 2 hrs, 3 hrs, 4 hrs, 5 hrs, 6 hrs, 7 hrs, 8 hrs, 9 hrs, 10 hrs, 11 hrs, 12 hrs, 13 hrs, 14 hrs, 15 hrs, 16 hrs, 17 hrs, 18 hrs, 19 hrs, 20 hrs, 21 hrs, 22 hrs, 23 hrs, 24 hrs, 25 hrs, 26 hrs, 27 hrs, 28 hrs, 29 hrs, 30 hrs, or up to 2 days, 3 days, 4 days, 5 days, 6 days, or 7 days after or before administering an additional therapeutic agent disclosed herein. The instructions can provide that the rAAV is formulated for intravenous injection and/or intranasal administration.

### EXAMPLES

Some aspects of the embodiments discussed above are disclosed in further detail in the following examples, which are not in any way intended to limit the scope of the present disclosure.

### Materials and Methods

The following experimental materials and methods were used for Example 1 described below.

### Experimental Model and Subject Details

All animal procedures in mice that were carried out in the present disclosure were approved by the California Institute of Technology Institutional Animal Care and Use Committee (IACUC), and Harvard Medical School Institutional Animal Care and Use Committee (IACUC). C57BL/6J (000664), Tek-Cre (008863), ChAT-IRES-Cre (006410), Nestin-Cre (003771), and TRPV1-Cre (017769) mice were purchased from the Jackson Laboratory (JAX). TH-Cre mice were obtained from the European Mouse Mutant Archive (EM::00254) and crossed with wild-type C57BL/6N mice. Heterozygous TH-Cre mice were used. For capsid selection experiments, 6-8 week old male and female mice were used. For in vivo validation studies of AAV capsid variants, 6-8 weeks old male mice were used.

All procedures performed on rats in the present disclosure were approved by the Animal Ethics Committee of the University of Melbourne (Ethics Number 1814639) and complied with the Australian Code for the Care and Use of Animals for Scientific Purposes (National Health and Medical Research Council of Australia). Six male Sprague-Dawley rats (Biomedical Sciences Animal Facility, University of Melbourne) aged 7 weeks were used in this study. Rats were housed in groups of 3 with environmental enrichment under a 12-hour (h) light-dark cycle with ad libitum access to food and water. Six male animals were used in this study and received tail vein injections of AAVs.

All experimental procedures performed on marmosets were approved by the University of California, San Diego, Institutional Animal Care and Use Committee (IACUC) and in accordance with National Institutes of Health and the American Veterinary Medical Association guidelines. 2 female animals and 1 male animal were used in this study and received intravenous injections of AAVs.

All experimental procedures performed on rhesus macaques were approved by the Institutional Animal Care and Use Committee at the University of California, Davis and the California National Primate Research Center (CNPRC). Two infant female animals were used in this study and received intravenous injections of AAVs.

For all the experiments performed in the present disclosure, the animals were randomly assigned, and the experimenters were not blinded while performing the experiments in the present disclosure unless indicated otherwise.

### Library Plasmid Preparation

Briefly, plasmid rAAV-ΔCap-in-cis-Lox2 (FIG. 1A) was used for building the heptamer insertion (7-mer-i) AAV library. Plasmid pCRII-9Cap-XE was used as a PCR template for the DNA library generation. Plasmid AAV2/9-REP-AAP-ΔCap was used to supplement the AAV library during virus production.

### Plasmid Preparation for Capsid Characterization

The AAV capsid variants such as AAV-MaCPNS1 (Addgene plasmid #185136) and AAV-MaCPNS2 (Addgene plasmid #185137) capsids were built by inserting 7-mer peptides between AAs 588-589 of the AAV9 cap gene in the pUCmini-iCAP-PHP.B backbone (Addgene plasmid #103002). The AAV-PHP.S capsid was described previously (Addgene plasmid #103006).

For in vivo validation of AAV capsids, the vectors were packaged with a single-stranded (ss) rAAV genome: pAAV:CAG-2xNLS-EGFP (available from Caltech CLOVER Center upon request, a similar version with 1xNLS is in Addgene, plasmid #104061), pAAV:CAG-EGFP, pAAV:hSyn1-tdTomato (Addgene plasmid #51506), pAAV:CAG-tdTomato (Addgene plasmid #59462), pAAV:hSyn-DIO-hM3D(Gq)-mCherry (Addgene plasmid #44361). To make the pAAV:CAG-jGCaMP8s plasmid, jGCaMP8s was synthesized as a gBlocks Gene Fragment (IDT) based on the sequence in the plasmid pGP-AAV-CAG-FLEX-jGCaMP8s-WPRE (Addgene plasmid #162380) and subcloned into the plasmid pAAV:CAG-EGFP by replacing the EGFP gene.

### AAV Capsid Library Generation

Briefly, the R1 library involved a randomized 21-nucleotide (7×NNK mutagenesis) insertion between AAs 588-589 of AAV9 capsid. The R2 library was built using a synthetic pool method. The R2 library was composed of an equimolar ratio of ˜9000 variants that were recovered from the tissues of interest in R1 (DRG, heart, small and large intestine). The Spike-in variants as part of the synthetic pool library consisted of previously validated variants such as AAV-PHP.B, AAV-PHP.B4, AAV-PHP.C1 (CNS variants), AAV-PHP.S (PNS variant) and AAV9 parent.

### In Vivo Selection and Capsid Library Recovery

For capsid selection in vivo, the virus library was intravenously administered to male and female mice of various Cre transgenic lines (n=2-3 per Cre line) at 2×10¹¹ vg per mouse in R1 selection, and at 1×10¹² vg per mouse in R2 selection. Two weeks post injection, mice were euthanized, and the organs of interest were harvested and snap-frozen on dry ice. The tissues were stored at −80° C. for long-term. To recover capsids from the tissue, the tissues were processed using Trizol, and the rAAV genomes were recovered by Cre-dependent PCR or Cre-independent PCR as previously described. The AAV DNA library, virus library and the libraries recovered from tissue post in vivo selection were processed for NGS as also described previously.

### AAV Vector Production for In Vivo Characterization

The AAV vectors were produced using an optimized vector production protocol. The average yield was ˜1×10¹² vg per plate.

### AAV Vector Administration and Tissue Harvest in Mice

For the intravenous injection procedures in mice, the AAV vectors were injected intravenously via the retro-orbital route into 6-8 week old adult mice at a dose of 0.1-1×10¹² vg per mouse. The retro-orbital injections were performed as described previously. The expression times were ˜3 weeks from the time of injection. The dosage and expression time were kept consistent across different experimental groups unless noted otherwise.

For the intraperitoneal injection procedures in mice, neonatal pups at postnatal stage 1 (P1) were intraperitoneally injected with the AAV vectors at a dose of 3×10¹¹ or 1×10¹² vg per mouse. Six weeks after AAV administration, tissue collections were performed.

To harvest the tissues of interest, the mice were anesthetized with Euthasol (pentobarbital sodium and phenytoin sodium solution, Virbac AH) and transcardially perfused using 30-50 mL of 0.1 M phosphate buffered saline (PBS) (pH 7.4), followed by 30-50 mL of 4% paraformaldehyde (PFA) in 0.1 M PBS. The organs were collected and post-fixed 24-48 h in 4% PFA at 4° C. Following this, the tissues were washed with 0.1 M PBS twice and stored in fresh PBS-azide (0.1 M PBS containing 0.05% sodium azide) at 4° C.

### AAV Vector Administration and Tissue Harvest in Rat

Six 7-week-old male Sprague Dawley rats received lateral tail vein injections of either MaCPNS1 or MaCPNS2 (2×10¹³ vg/kg-3 rats/group). After a 3-week incubation period, animals were transcardially perfused with saline followed by 4% PFA, as per a published protocol. Tissues were then dissected out and post-fixed in 4% PFA for 1 h before being washed in 0.1 M PBS (3×30 min). Tissues were stored in PBS-azide until processing. In one rat (MaCPNS2 injection), the perfusion fixation process was unsuccessful, so tissues were fixed only by immersion (18-24 h), prior to washing and storage as described above.

### AAV Vector Administration and Tissue Harvest in Marmoset

Marmoset monkeys were anesthetized using an intramuscular Ketamine (20 mg/kg) and Acepromazine (0.5 mg/kg) injection. An intravenous catheter was placed in the saphenous vein of the hind leg and flushed with ˜2 mL of LRS (Lactated Ringer's solution) for 2 min. Viruses were pooled together in a single syringe (˜500-900 μL) and infused at a rate of 200 μL/min into the catheter. Following the infusion, the catheter was flushed with ˜3 mL of LRS for 2 min and removed. The animal was then returned to a recovery cage.

Following an incubation period of 4-6 weeks post viral injection, the animals were euthanized by injecting pentobarbital intraperitoneally. Two researchers worked in parallel to harvest the tissue as quickly as possible to limit degradation. Each organ—brain, lungs, kidneys, etc.—was removed and separated into two parts. One half of the tissue was flash-frozen in 2-methylbutane that was chilled with dry ice to preserve mRNA and DNA in the harvested tissues. The other half of the tissue was fixed in 4% PFA solution for estimation of protein expression. Flash-frozen tissue samples were transferred to a −80° C. freezer, while PFA-fixed tissue samples were stored in a 4° C. fridge.

### AAV Vector Administration and Tissue Harvest in Rhesus Macaque

Two female rhesus macaques were injected within 10 days of birth. Prior to injection, animals were anesthetized with ketamine (0.1 mL) and the skin over the saphenous vein was shaved and sanitized. AAVs (2.5×10¹³ vg/kg) were slowly infused into the saphenous vein for ˜1 min in <0.75 mL of 0.1 M PBS. Animals were monitored while they recovered from anesthesia in their home enclosure, and daily for the remainder of the study. Monkeys were individually housed within sight and sound of conspecifics.

Tissues were collected 4 weeks post AAV administration. Animals were deeply anesthetized and euthanized using sodium pentobarbital in accordance with guidelines for humane euthanasia of animals at the CNPRC. The whole body was perfused with ice-cold RNase-free 0.1M PBS. Brains were removed from the skull and blocked into 4 mm thick slabs in the coronal plane. Brain slabs and organs were subsequently post-fixed in 4% PFA for 48 h. One hemisphere of each animal was cryoprotected in 10%, 15%, and 30% sucrose in 0.1 M PBS.

### Immunohistochemistry on Tissues

In mice and NHP experiments, tissue sections, typically 100-μm thick, were first incubated in blocking buffer (10% normal donkey serum (NDS), 0.1% Triton X-100, and 0.01% sodium azide in 0.1 M PBS, pH 7.4) with primary antibodies (rabbit anti-NeuN (Abcam ab177487, 1:500), chicken anti-PGP9.5 (Invitrogen PA1-10011, 1:500), rabbit anti-S100 beta (Abcam ab52642, 1:200), rabbit anti-Parvalbumin (Abcam 181086, 1:200), rabbit anti-Olig2 (Abcam ab109186, 1:200), rabbit anti-GLUT1 (Millipore 07-1401, 1:200)) at appropriate dilutions for 24 h at room temperature (RT) on a rocker. After primary antibody incubation, the tissues were washed 1-3 times with wash buffer 1 (0.1% Triton X-100 in 0.1 M PBS buffer, pH 7.4) over a period of 5-6 h in total. The tissues were then incubated in blocking buffer with secondary antibodies (goat anti-chicken Alexa647 (Invitrogen, A32933, 1:1000), donkey anti-rabbit Alexa555 (Invitrogen, A21432, 1:1000), goat anti-rabbit Alexa647 (Invitrogen, A21245, 1:1000) at appropriate dilutions for 12-24h at RT and then washed 3 times in 0.1 M PBS over a total duration of 5-6 h. When performing DNA staining, the tissues were incubated with 4′,6-Diamidine-2′-phenylindole dihydrochloride (DAPI) (Sigma Aldrich, 10236276001, 1:1000) in 0.1 M PBS for 15 min followed by a single wash for 10 min in 0.1 M PBS. The DAPI and/or antibody-stained tissue sections were mounted with ProLong Diamond Antifade Mountant (ThermoFisher Scientific, P36970) before imaging under the microscope.

For immunostaining of DRG in mice neonates and the pain induction experiment, DRGs from spinal cord segments: thoracic levels 11-13 and lumbar levels 1-5 or 1-6 were dissected, fixed for 2 h in 4% PFA (diluted in 0.1 M PBS) at 4° C., incubated overnight at 4° C. in 30% sucrose solution (diluted in 0.1 M PBS), and embedded in OCT (Tissue-Tek®). Sections of 14 μm thickness were cut and blocked with blocking buffer-1 (10% NDS, in PBST solution containing 0.3% Triton X-100 and 0.1 M PBS, pH 7.4) for 2 h at room temperature. Tissues were stained with primary antibodies (rabbit anti-CGRP (Millipore PC205L, 1:500), rabbit anti-Neurofilament 200 (Sigma N4142, 1:500), mouse anti-Tuj1 (Abcam ab7751, 1:1,000)) that were diluted in blocking buffer-2 (2% NDS in PBST solution) and incubated overnight at 4° C. Sections were washed in PBST buffer, then stained with secondary antibodies (donkey anti-rabbit DyLight488 (Abcam ab96919, 1:1000), donkey anti-mouse Alexa647 (Abcam ab150107, 1:500)) diluted in blocking buffer-2 for 2 h at RT. After washing with 0.1 M PBS, sections were mounted in VectaShield (Vector Labs), and imaged in an Olympus Fluoview 1000 confocal microscope with 20× magnification at HMS Microscopy Resources on the North Quad (MicRoN) Core. Five DRG images per mouse were used for quantification.

To immune-stain rat organs and sensory ganglia, cryosections (14 μm) mounted onto gelatinized slides were washed in PBS (1×10 min) followed by 1 h incubation in blocking solution (PBS containing 0.1% Triton X-100 and 10% horse serum). Sections were incubated with primary antibody (hypertonic PBS, 18-24 h in a humid chamber), washed in PBS (1×15 min) and incubated with secondary antibody (2 h). Sections were again washed in PBS (1×15 min) and then coverslipped using buffered glycerol. This method is previously described in detail. Briefly, DRG and trigeminal tissue were labeled for DsRed and each of the 3 markers (NF-200, CGRP, TRPV1) while liver tissue was labeled for DsRed and DAPI. Primary antibodies were: rabbit anti-dsRed (Takara Bio, 632496, 1:5000); mouse anti-neurofilament 200 (Sigma-Aldrich, N0142, 1:4000); goat anti-calcitonin gene-related peptide, Bio-Rad, 1720-9007, 1:5000); goat anti-TRPV1 (Neuromics, GT15129, 1:1000). Secondary antibodies were: donkey anti-rabbit AF594 (Jackson ImmunoResearch Laboratories, 711-585-152, 1:1000); donkey anti-mouse AF488 (Jackson, 715-545-150, 1:2000); donkey anti-goat AF488 (Jackson, 705-545-147, 1:1000).

To immune-stained rat CNS, cryosections (40 μm) of brain (coronal) and L4-L5 spinal cord (transverse) were collected in a 1 in 4 series and stored in PBS-azide until use. Sections were washed in PBS (3×10 min) followed by 2 h incubation in blocking solution (PBS containing 0.5% Triton X-100 and 10% horse serum), washed in PBS (3×10 min) then incubated for 48 h with primary antibody (0.1% PBS-azide containing 0.5% Triton X-100 and 2% horse serum). Tissues were then washed in PBS (3×10 min) followed by 4 h incubation with secondary antibody. Tissues were washed in PBS (3×10 min) and then mounted and coverslipped using buffered glycerol. This method is previously described in detail. Primary antibodies used were: rabbit anti-dsRed (Takara Bio, 632496, 1:5000) and mouse anti-NeuN (Chemicon, MAB377, 1:2000). Secondary antibodies are described above.

To immune-stained whole mounts of autonomic ganglia (IMG, MPG, sympathetic chain) and intestine, tissues were washed in PBS (3×30 min) followed by 2 h incubation in blocking solution (PBS containing 0.5% Triton X-100 and 10% horse serum). Tissues were again washed in PBS (3×30 min) before 72 h incubation with primary antibody (0.1% PBS-Azide containing 0.5% Triton X-100 and 2% horse serum). Tissues were then washed in PBS (3×30 min) followed by 24 h incubation with secondary antibody. Tissues were washed in PBS (3×30 min) before being cleared using ethyl cinnamate. Briefly, tissues were washed in methanol (100%, 3×30 min) followed by dichloromethane (1×30 min) and then ethyl cinnamate (2×30 min). Tissues were then mounted on glass slides and coverslipped using ethyl cinnamate.

### Optical Clearing for Thick Tissues

For imaging tissues >100 microns in thickness such as gut, optical clearing was carried out by incubating small pieces (0.5-1 cm length) of tissue in 1-2 mL of ScaleS4(0) solution overnight at RT with gentle agitation. The tissues were then mounted in fresh ScaleS4(0) solution with spacers (0.1-1 mm thick) on glass slides and imaged under the microscope.

### Tissue Imaging and Image Processing

The images used in this study were acquired with a Zeiss LSM 880 confocal microscope using the following objectives: Plan-Apochromat 10×0.45 M27 (working distance 2.0 mm), and Plan-Apochromat 25×0.8 Imm Corr DIC M27 multi-immersion. The liver images were acquired with a Keyence BZ-X700 microscope using a 20× objective. The images were then processed in the following image processing software: Zen Black 2.3 SP1 (for Zeiss confocal images) and BZ-X Analyzer (for Keyence images).

Image collection and analysis for cryosections of rat sensory ganglia and organs was performed using wide-field fluorescence microscopy with an ApoTome attachment (Zeiss AxioImager M2). Quantification of AAV+ and AAV+/Marker+ neurons was performed for DRG and trigeminal ganglia on 4 non-sequential sections with only nucleated neuronal profiles counted. Counts were performed manually while viewing sections under the microscope. For CNS sections and whole mounts of intestine and ganglia, image collection and analysis were performed using wide-field fluorescence microscopy with an ApoTome attachment (Zeiss AxioImager M2).

### Nodose Calcium Imaging Experiment

In vivo vagal ganglion imaging was conducted as previously described. Briefly, animals were anesthetized with pentobarbital at a dose of 100 mg/kg body weight, injected intraperitoneally (i.p.). A skin incision was made to expose the abdominal region. For intestinal glucose infusion and distension, an input tubing (HelixMark Silicone Tubing 60-011-04) was inserted in the jejunum and the output tubing (HelixMark Silicone Tubing 60-011-09) was set at the ileum. Surgical thread was used to fasten and secure tubing sites. During the abdominal surgery, isotonic saline was applied occasionally to prevent tissue drying. After suturing the abdominal region, a midline neck incision was made. Muscles were separated and retracted laterally to expose the trachea and vagus trunk. To free the vagal ganglion, the carotid artery was retracted aside, and the vagus nerve was transected superior to the jugular ganglion. The vagal ganglion was then placed on a custom 5-mm diameter glass coverslip (72290-01, Electron Microscopy Sciences), and immediately immersed in silicon adhesive (KWIK-SIL, World Precision Instruments). Imaging was conducted with a Leica SP8 confocal microscope, with a frame rate of 1 Hz. For glucose stimulus, 300 mM glucose solution was infused at a flow rate of 800 L/min for 2 min. Intestinal distension stimulus was achieved by closing the exit port for 1 min while infusing saline.

### Pain Behavior Experiment

Trpv1-Cre neonatal pups at postnatal stage 1 (P1) were intraperitoneally injected with 10 μL of MaCPNS1:Syn-DIO-hM3D(Gq)-mCherry virus (3×10¹³ vg/ml). Six weeks after injection, mice were subjected to a pain test in an infrared Behavior Observation Box (iBOB) (Harvard Apparatus) which allowed video recording of mice in the dark and independent of an observer. Prior to Clozapine-N-oxide (CNO) injection, mice were habituated for 30 min in individual chambers of the iBOB. Mice were then given intraplantar injection of 10 μL of CNO or vehicle (5% Dimethyl sulfoxide (DMSO) in 0.1 M PBS). Injections were performed under light restraint without anesthesia. Mice were immediately placed in the iBOB and recorded. Total bouts of lifting/licking of the footpad and total time spent licking/lifting the footpad for the first 15 minutes after CNO injection were quantified.

### Analysis of Pain Behavior

The 20-min videos recorded in the pain-induction experiment were scored by a blinded observer to quantify the total bouts and time spent licking and shaking the injected paw within 15 min. GraphPad Prism was used for statistical comparison and plotting.

### Quantification of AAV Transduction In Vivo

The quantification of AAV transduction across NG, DRG and GI tract was carried out by manually counting fluorescent expression resulting from the AAV genome. The Adobe Photoshop CC 2018 Count Tool was used for this purpose. To quantify expression in the liver, Keyence Analyzer automated cell count software was used. The efficiency was determined by the percentage of cells expressing EGFP or tdTomato relative to a specific cell marker, namely, NeuN, PGP9.5, DAPI, CGRP, NF200, TRPV1, Tuj1, GLUT1, Olig2 or S100β.

### NGS Data Alignment, Processing and Analysis

The raw fastq DNA files were aligned to the AAV9 capsid template using custom alignment software as described previously. The NGS data analysis was carried out using a custom data-processing pipeline with scripts written in Python and plotting software such as Plotly, Seaborn, and GraphPad PRISM 7.05. The AAV9 capsid structure model was produced with PyMOL.

The enrichment score for a variant was determined using the following formula:

$$Enrichment score of variant “ x ” = log 10 [ ( Variant “ x ” RC in tissue library 1 / Sum of variants N RC in library 1 ) / ( Variant 1 RC in virus library / Sum of variants N RC in virus library ) ]$$

Where N is the total number of variants in a library.

$$\\ The fold ‐ change of a variant “ x ” to AAV 9 = ( The enrichment of “ x ” - The enrichment of AAV 9 ) / \vert The enrichment of AAV 9 \vert .$$

### UMAP Clustering Analysis

For profiling, the overall tropism of the AAV library post R2 selection, a non-linear algorithm, UMAP, was used for dimension reduction and visualization with a script adapted from GitHub. After the M-CREATE data-processing pipeline, the R2 capsid library yielded enrichment scores across 22 targets (tissues, FIG. 1B). These enrichment scores served as 22 dimensions that were fed into the UMAP algorithm, and the parameters considered were n_neighbors (15), n_components (2), random_state (42), distance_metric (‘correlation’), verbose (3).

### Data Analysis for Calcium Imaging

Imaging data was analyzed using CaImAn-MATLAB with modified MATLAB code adapted from GitHub. Basically, imaging frames from the same animal were first registered to correct for motion. A non-negative matrix factorization (CNMF) algorithm was applied to recognize individual cells and to extract fluorescence activities. A 30 s window before the stimulus onset was used as baseline signal. To quantify signals, the mean (μ) and standard deviation (σ) of F0(t) over the baseline period were computed as F(t)=(F0(t)−μ)/σ. Cells were defined as responsive if the average ΔF/F(σ) value during the stimulus period was more than 3 s.d. above the baseline mean activity.

### Key Resources

The key resources used in this example included Rabbit anti-NeuN antibody, Chicken anti-PGP9.5 antibody, Goat anti-chicken Alexa647 antibody, Donkey anti-rabbit Alexa555 antibody, Rabbit anti-CGRP antibody, Rabbit anti-Neurofilament 200 antibody, Mouse anti-Tuj1 antibody, Donkey anti-rabbit DyLight488 antibody, Donkey anti-mouse Alexa647 antibody, Rabbit anti-dsRed antibody, Mouse anti-Neurofilament 200 antibody, Goat anti-calcitonin gene-related peptide antibody, Goat anti-TRPV1 antibody, Donkey anti-rabbit AF594 antibody, Donkey anti-mouse AF488 antibody, Donkey anti-goat AF488 antibody, Mouse anti-NeuN antibody, Rabbit anti-S100 beta antibody, Rabbit anti-Parvalbumin antibody, Rabbit anti-Olig2 antibody, Rabbit anti-GLUT1 antibody, Goat anti-rabbit Alexa647 antibody, Mouse: C57BL/6J, Mouse: Tek-Cre, Mouse: ChAT-IRES-Cre, Mouse: Nestin-Cre, Mouse: TRPV1-Cre, Mouse: TH-Cre, Sprague-Dawley rat, Marmoset, Rhesus Macaque, pUCmini-iCAP-AAV.MaCPNS, pUCmini-CAP-AAV.MaCPNS, pAAV:hSyn1-tdTomato plasmid, pAAV:CAG-tdTomato plasmid, pAAV:hSyn-DIO-hM3D(Gq)-mCherry plasmid, and pGP-AAV-CAG-FLEX-jGCaMP8s-WPRE plasmid.

### Example 1

### Engineered AAVs for Non-Invasive Gene Delivery to Rodent and Non-Human Primate Nervous Systems

AAV Capsid Selection in Mice Identifies Two AAV Variants with PNS Specificity

As a starting point for capsid engineering, AAV9 was chosen due to its broad tropism when delivered systemically, including for the nervous system (both CNS and PNS). The AAV9 capsid was diversified by inserting a randomized 7-mer peptide between positions 588 and 589 (FIG. 1A), and intravenously injected the resulting virus library into adult mice of three Cre-transgenic lines: SNAP-Cre (for neurons), GFAP-Cre (for glia), and Tek-Cre (for endothelial cells forming the blood-organ barrier). Two weeks post-injection, peripheral tissues (such as heart, small and large intestines, and DRG) were processed and viral genomes were selectively extracted from Cre+ cells using Cre-dependent PCR (step (2) in FIG. 1A).

After round-1 (R1) selection, a total of ˜9,000 variants were recovered from the Cre lines in the PNS tissues of interest: heart, DRG, small and large intestine. Of these, ˜10% overlapped with the CNS libraries (step (3) in FIG. 1A). Then, these variants were synthesized in an equimolar ratio to create a synthetic oligopool library for round-2 (R2) PNS selection. This synthetic pool also included a spike-in library of previously-validated internal controls. The R2 virus library was intravenously injected into different Cre lines: Nestin-Cre (for neurons), CHAT-Cre (for cholinergic neurons), TRPV1-Cre (for primary afferent thermosensitive neurons), and Tek-Cre (for endothelial cells, providing a negative selection). Two weeks post-injection, capsids were selectively recovered by Cre-dependent PCR from tissues of interest (DRG, heart, small and large intestine, brain, and spinal cord (SC)), and other targets (spleen, liver, lung, kidney, testis and muscle) by performing Cre-independent PCR (step (4) in FIG. 1A, FIG. 1B).

After two rounds of in vivo selection, among all the variants that were included in the R2 library, 6,300 variants showed a bias towards one or more of the PNS tissues (FIG. 1B, FIG. 16A). The recovered variants were further classified based on distinct tropisms. The Uniform Manifold Approximation and Projection (UMAP) algorithm was used, which takes into account differences among variants' enrichments across organs, to identify clusters of variants representing distinct tropisms (the first panel of FIG. 1C). Members of Cluster-1 (1,846 variants), which includes AAV9, show relatively higher enrichment in off-target tissues such as the liver (the second panel of FIG. 1C). Cluster-2 members (7,148 variants), including a previously engineered variant PHP.S, exhibit relatively higher enrichment in the PNS and lower enrichment in off-target tissues (the third panel of FIG. 1C). Cluster-3 members (5 variants), including variants from the internal control, were highly enriched in the CNS.

Based on this analysis, Cluster-2 contained variants distinct from the parental AAV9, which were observed by comparing their tropism directly to that of AAV9 (FIG. 1D). Two new capsids were identified from Cluster-2 that exhibited low off-target transduction compared to AAV9 and PHP.S (FIG. 1E) and that could package viral genomes with similar efficiency to AAV9 (FIG. 1B). The first capsid, with a 7-mer peptide insertion of PHEGSSR between the 588-89 residues of the AAV9 parent, will be henceforth referred to as AAV-MaCPNS1. The second, with an insertion of PNASVNS, will be referred to as AAV-MaCPNS2 (FIG. 1F).

IV-Delivered AAV-MaCPNS1/2 Efficiently Transduces Sensory and Enteric Ganglia in Mice, with Low Liver Transduction

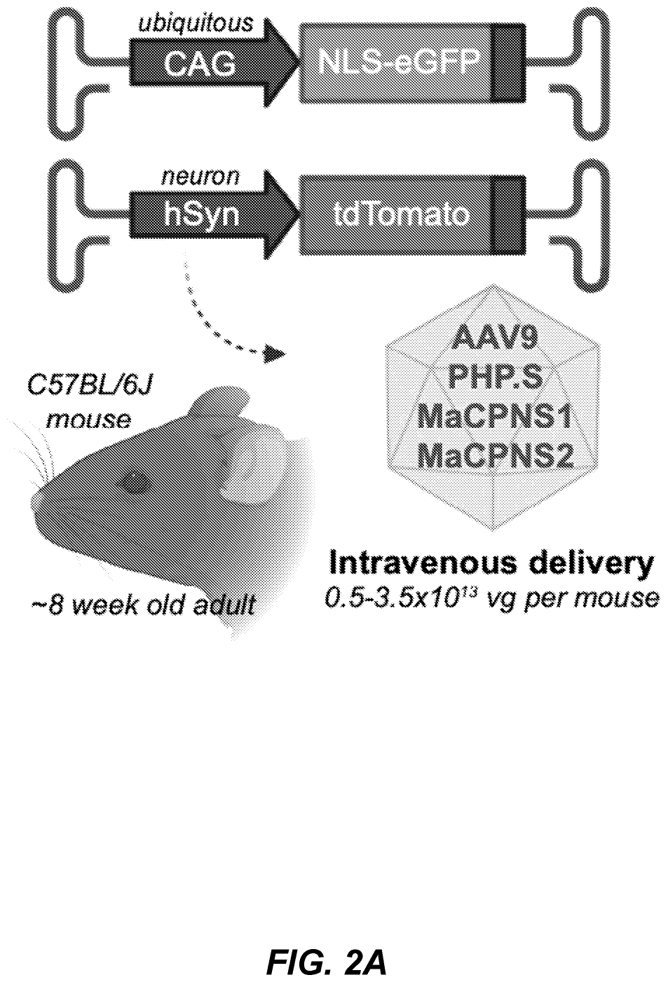

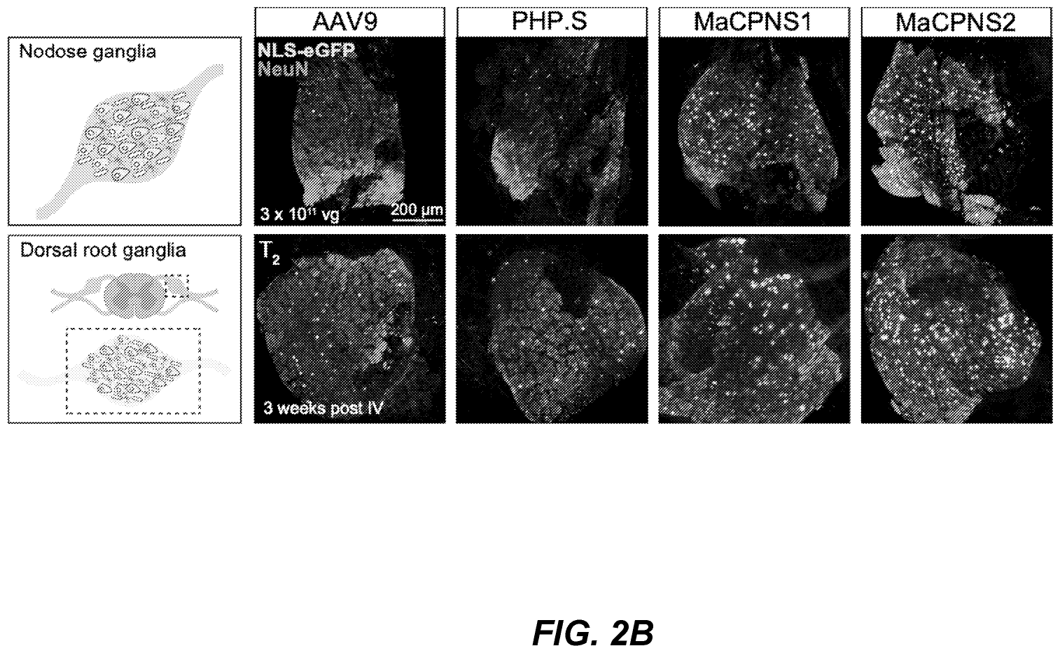

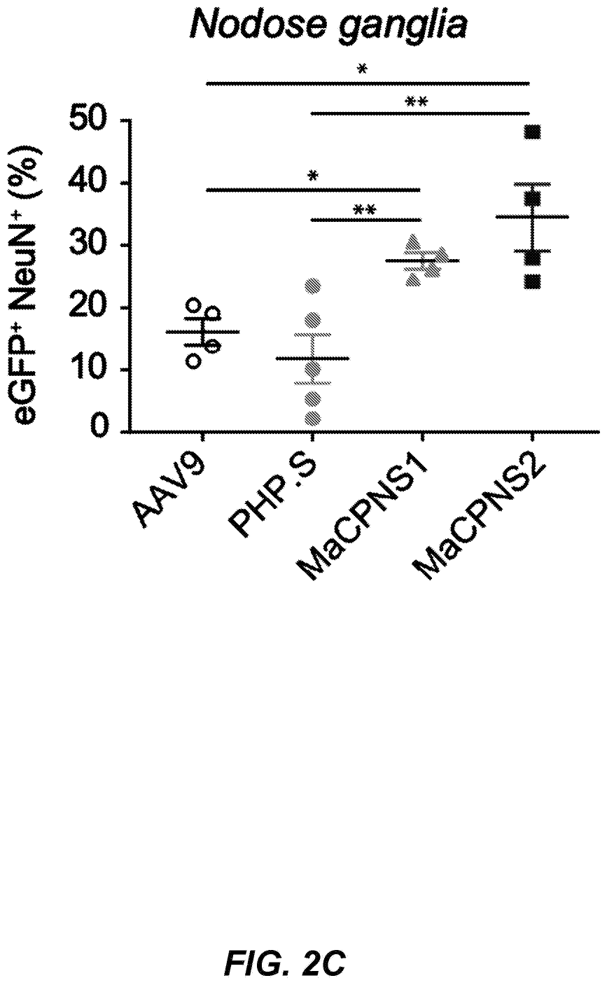

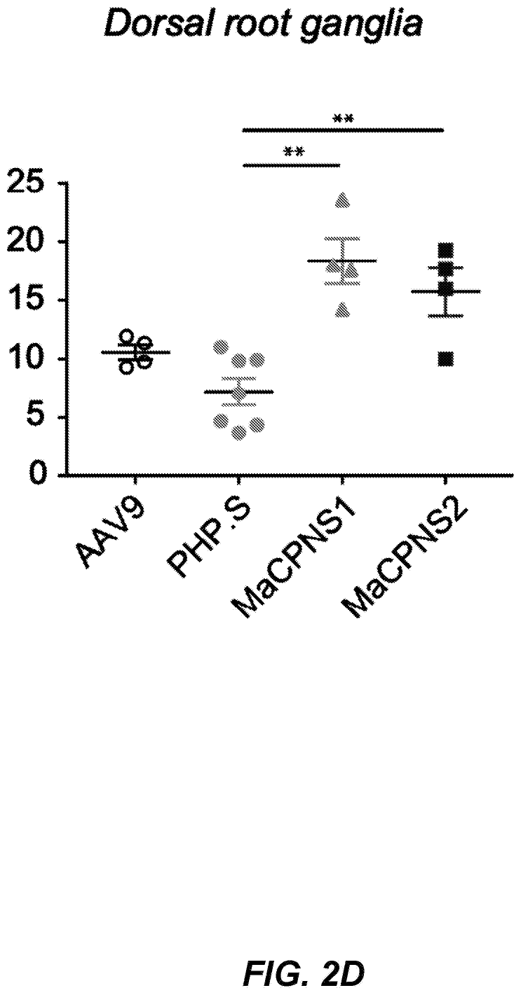

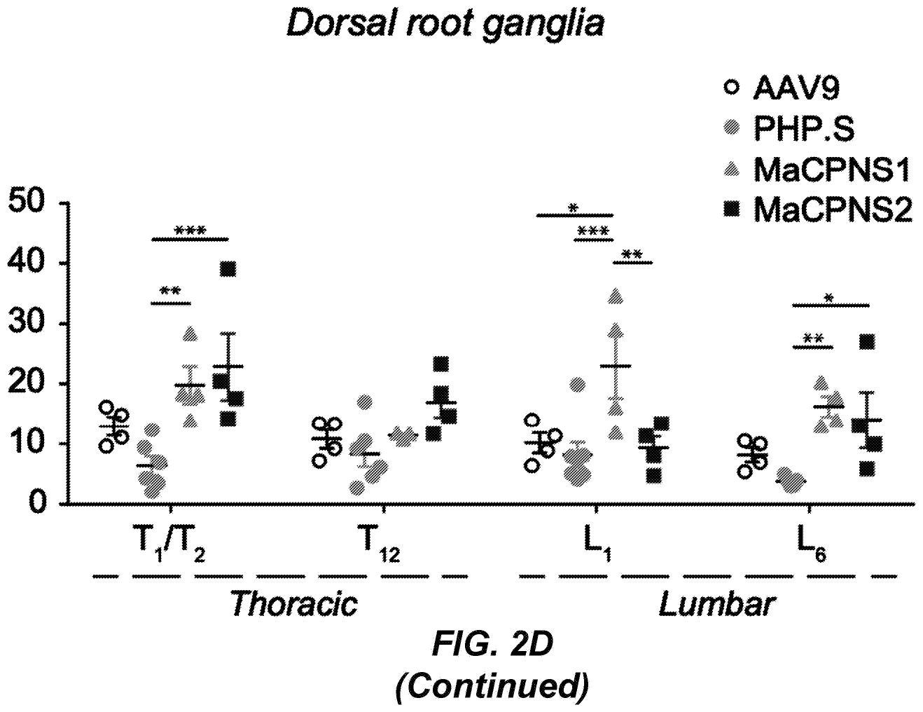

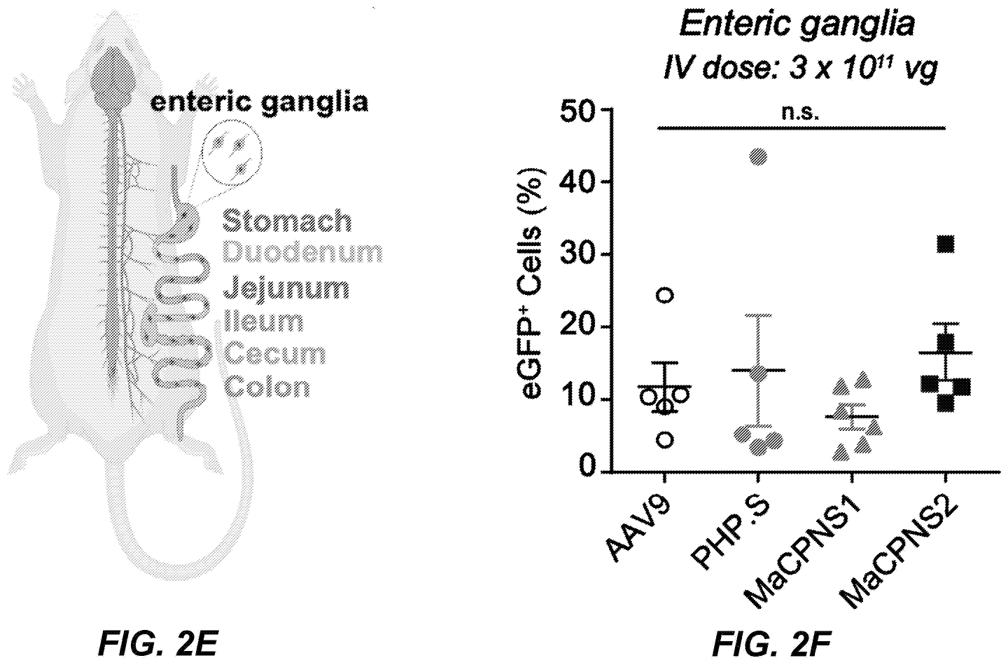

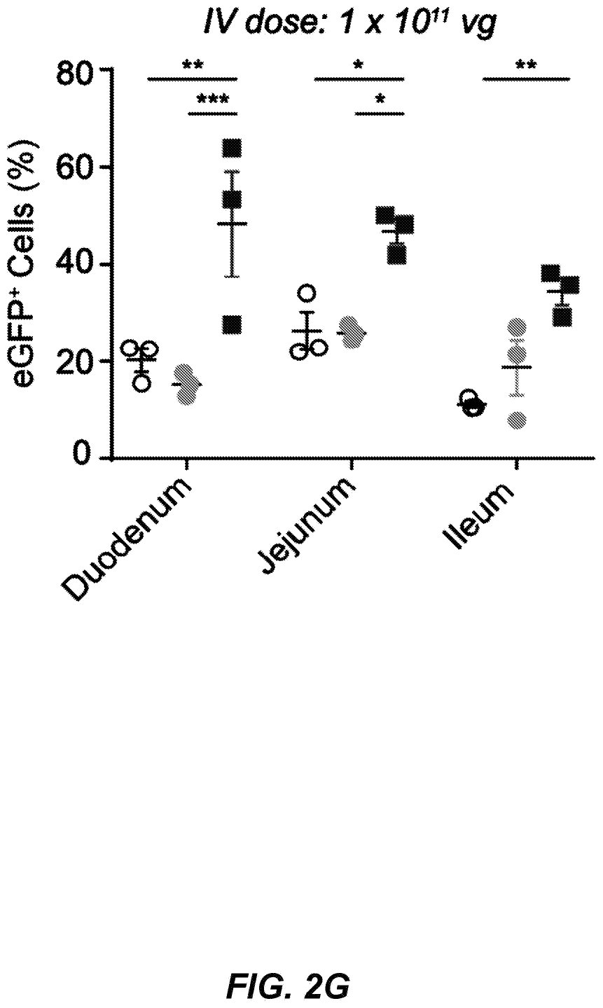

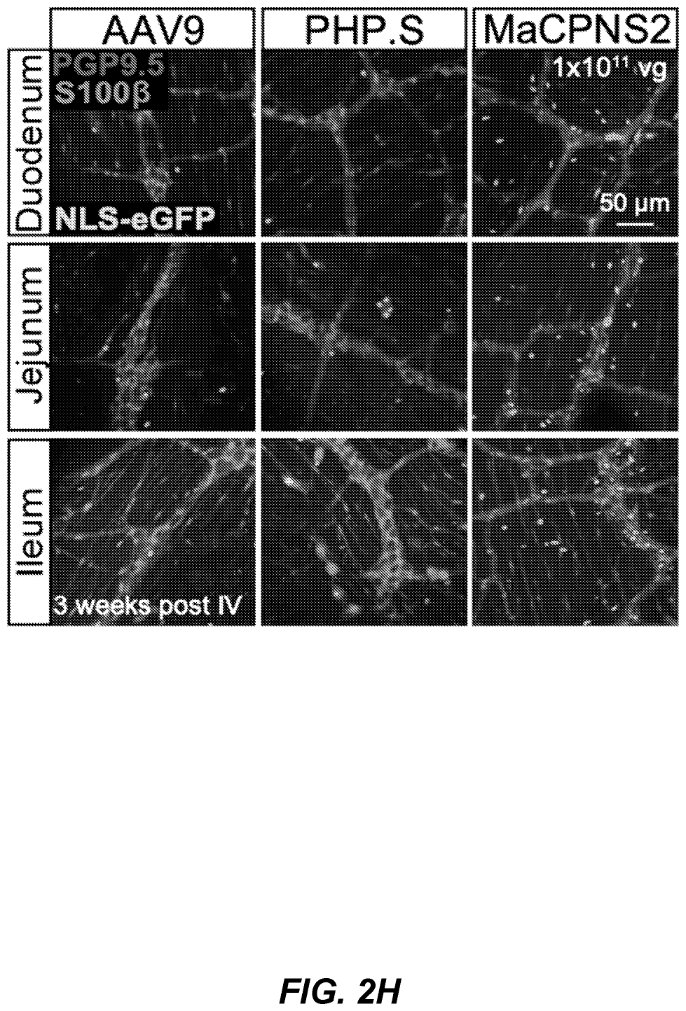

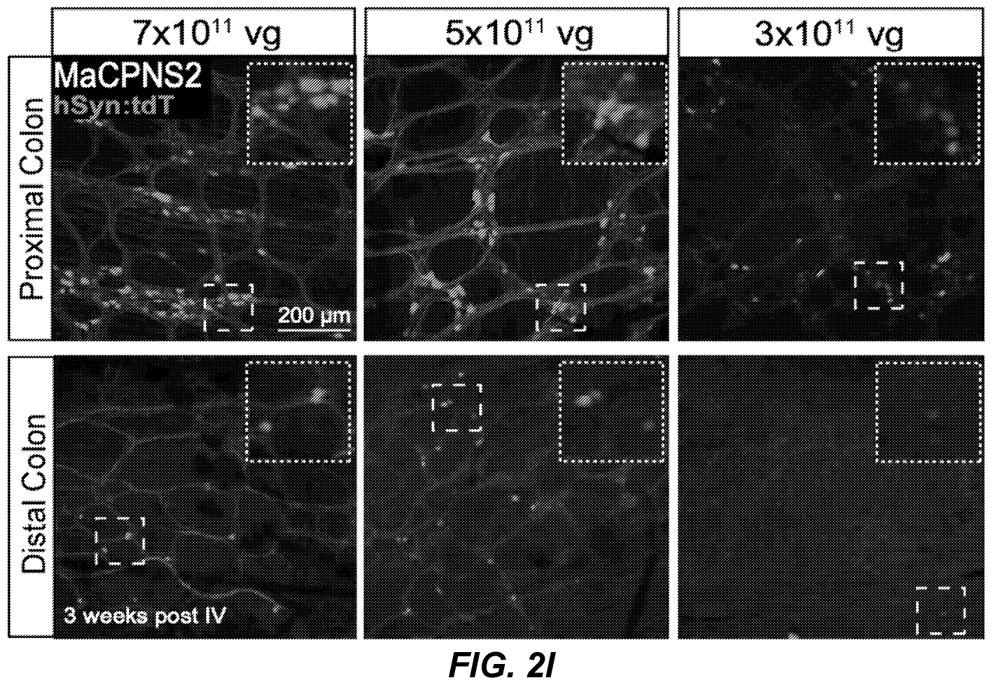

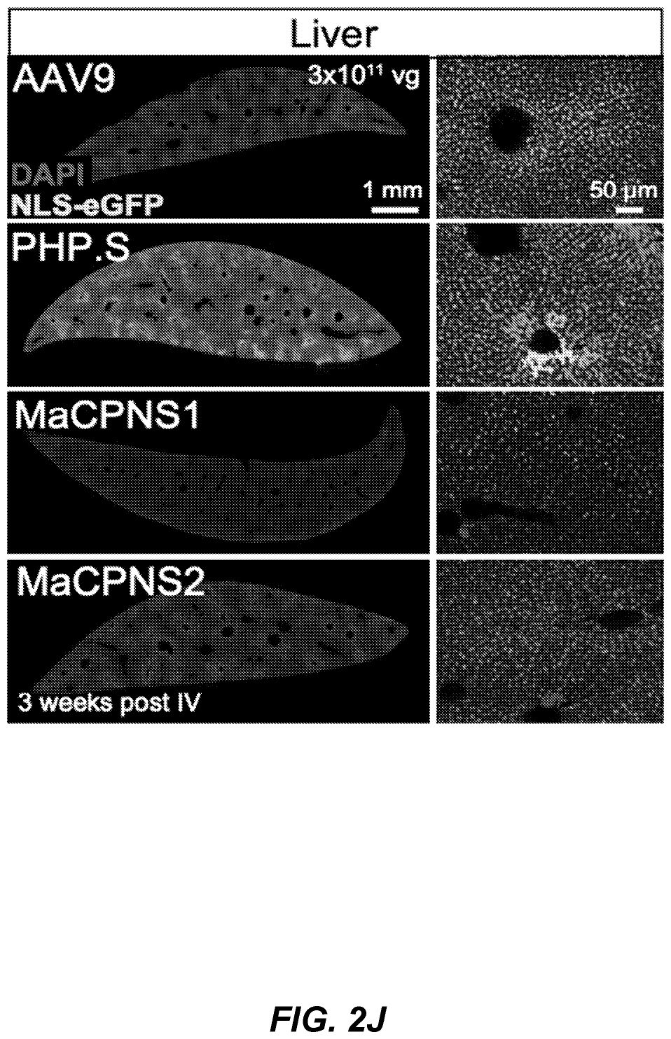

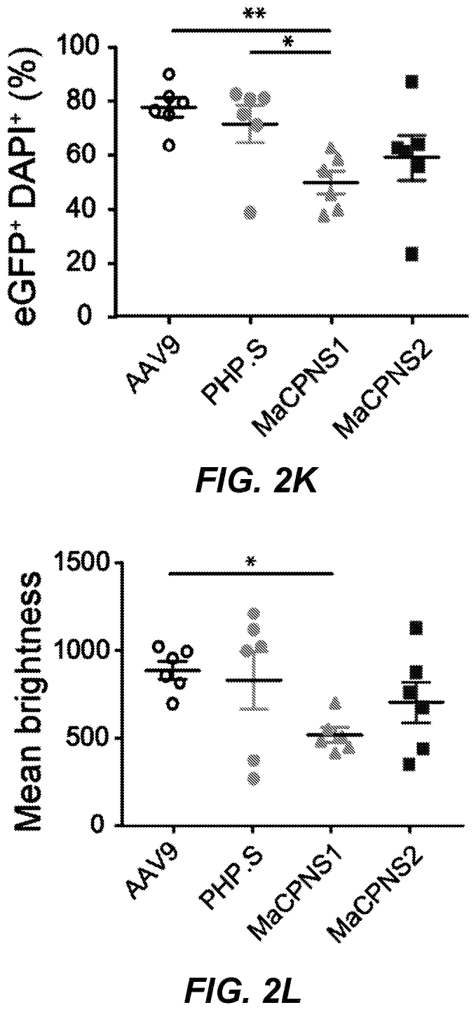

To characterize the transduction capability of AAV-MaCPNS1 and AAV-MaCPNS2 variants in vivo, these variants were packaged with a single-stranded (ss) AAV genome carrying a strong ubiquitous promoter, CAG, driving expression of nuclear-localized eGFP reporter and the variants were intravenously injected into adult mice at 3×10¹¹ vg per animal (FIG. 2A-FIG. 211, FIG. 2J-FIG. 2L). By quantifying expression in nodose ganglia (NG) that overlapped with the NeuN neuronal marker, MaCPNS1 and MaCPNS2 had mean transduction efficiencies of ˜28% and ˜35%, respectively. By comparison, the efficiency of AAV9 was ˜16%, and AAV-PHP.S ˜12% (FIG. 2B-FIG. 2C). Thus, MaCPNS1/2 capsids exhibit about 2-fold higher transduction of NG than previously available vectors.

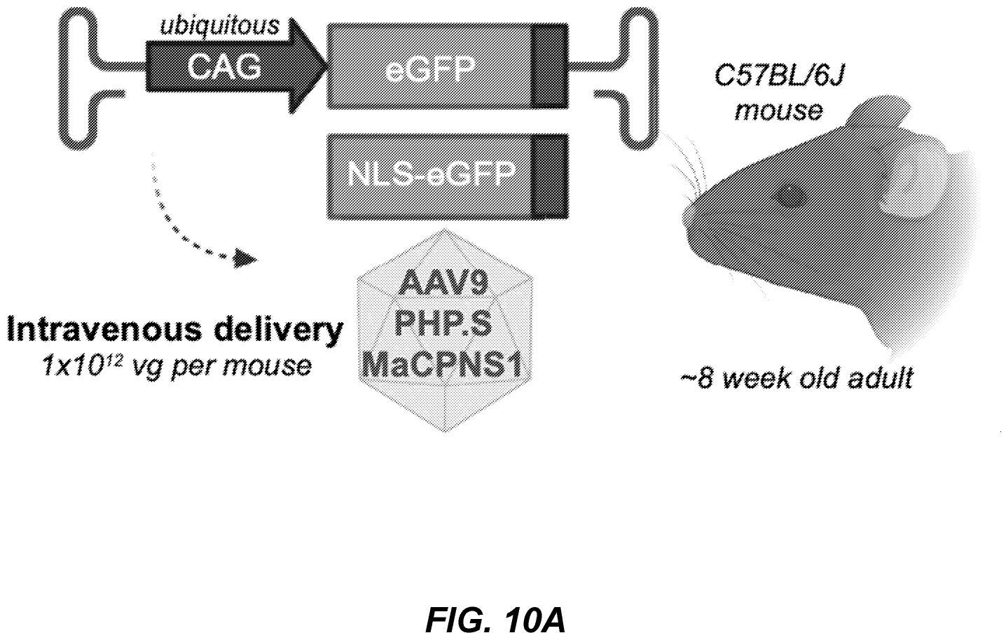

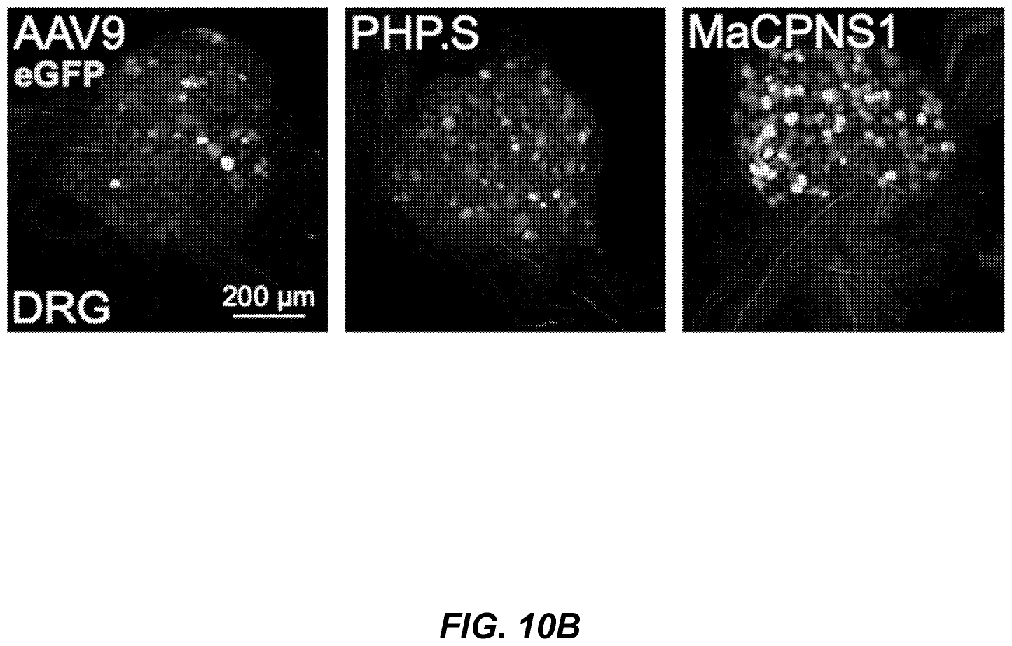

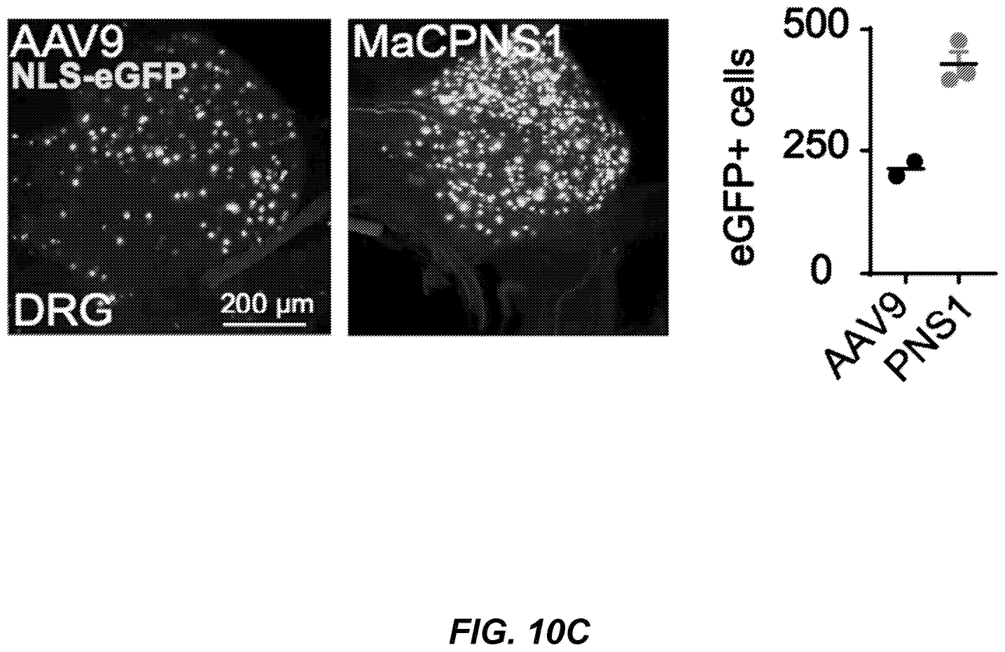

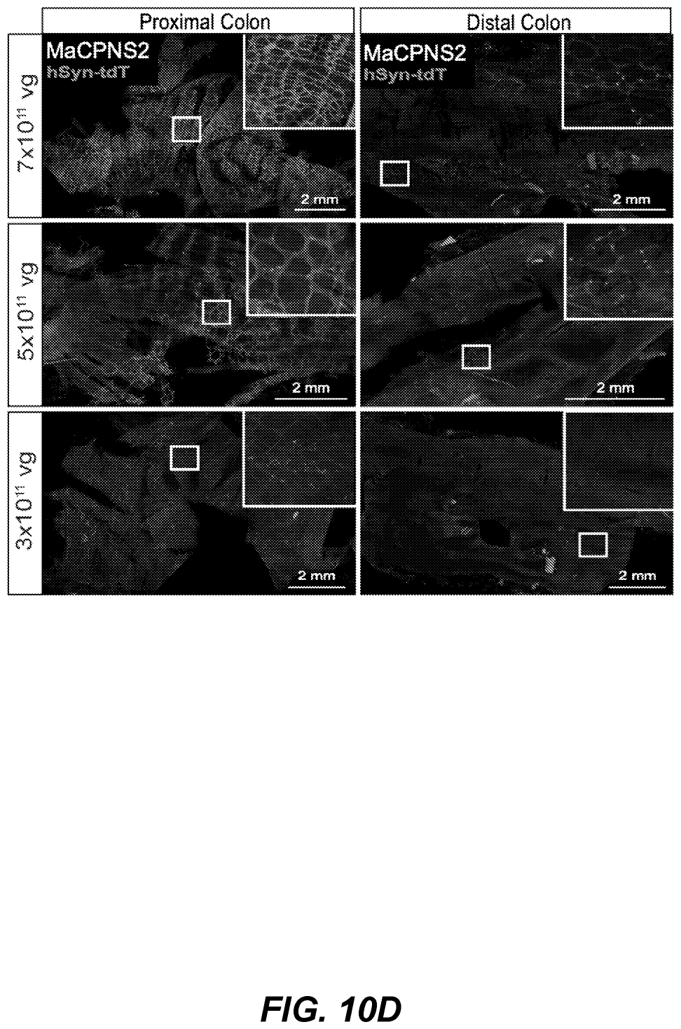

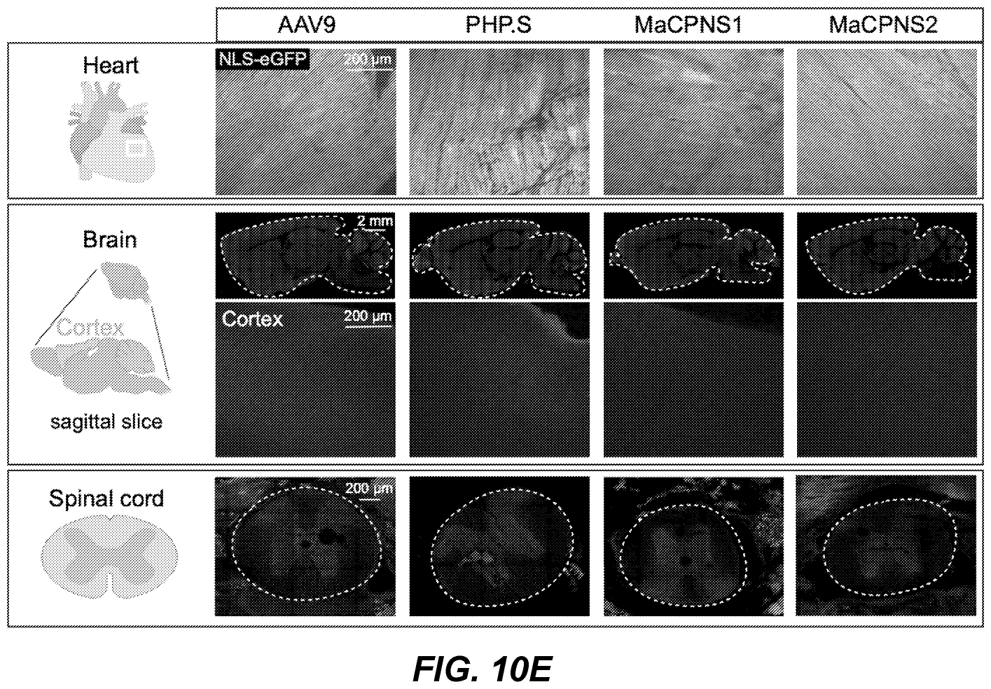

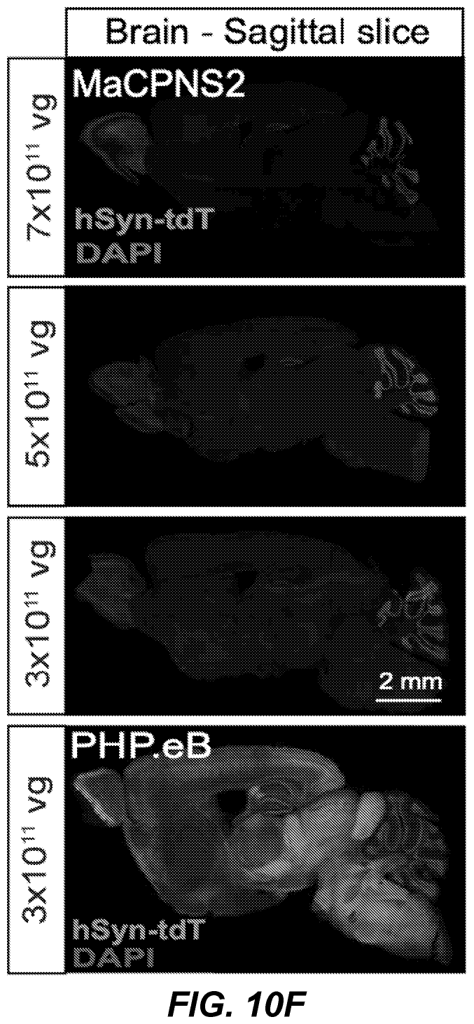

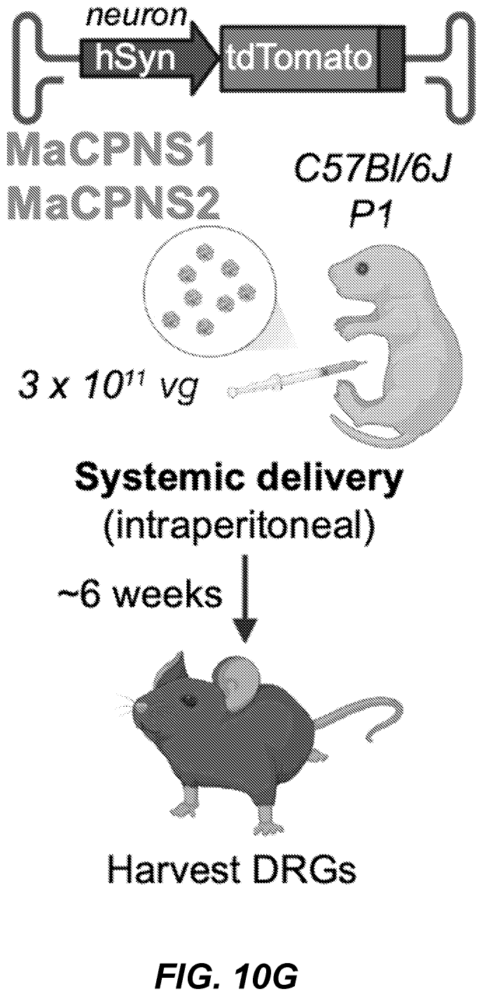

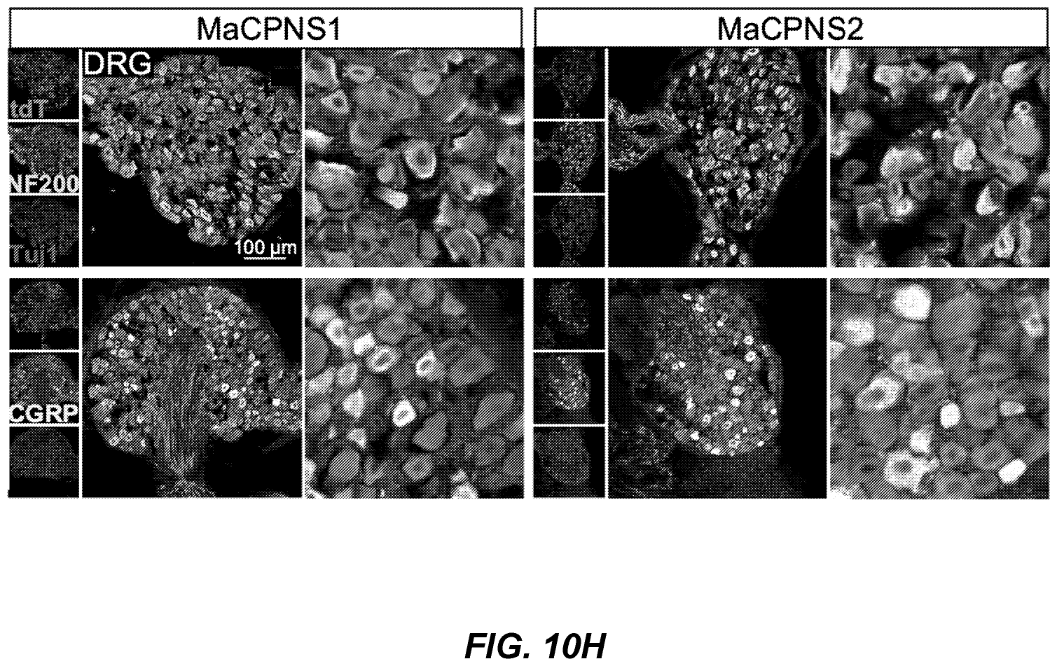

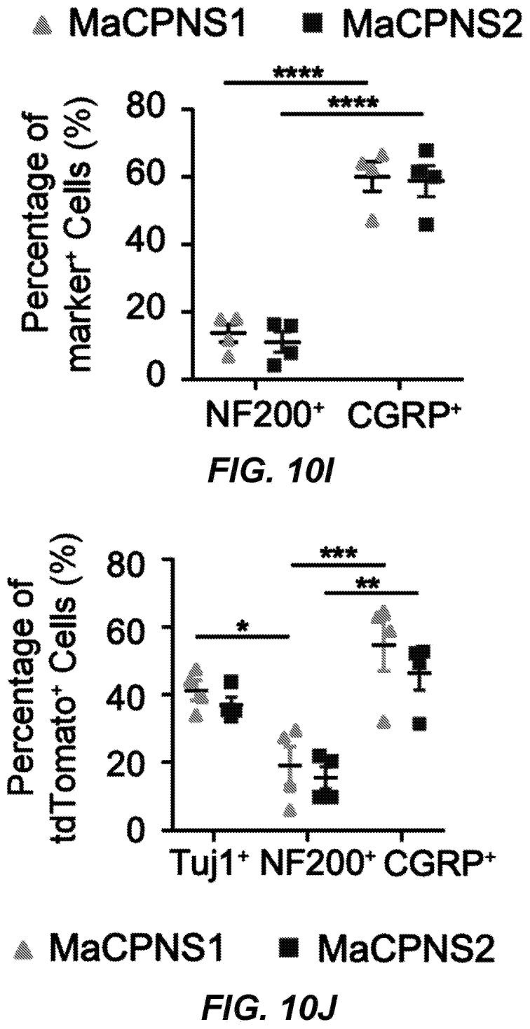

Next, the efficiency of DRG transduction was investigated from selected spinal levels (thoracic and lumbar). The MaCPNS1 vector demonstrated a mean transduction efficiency of ˜18%, and MaCPNS2 ˜16%, compared to ˜11% for AAV9 and ˜7% for AAV-PHP.S (FIG. 2B bottom panel, the first panel of FIG. 2D), with some variability in transduction across different segments of the SC (FIG. 2D (Continued)). Thus, compared to known capsids, MaCPNS1/2 can achieve about 2-fold higher transduction of DRG via IV delivery. With vector dose increased to 1×10¹² vg, PHP.S had higher transduction in DRG compared to AAV9 while MaCPNS1 exhibited even higher transduction (FIG. 10A-FIG. 10C), further confirming that MaCPNS1/2 capsids transduce DRG in adult mice more efficiently than either AAV9 or PHP.S.

To investigate the transduction efficiency of the new vectors in the enteric nervous system (ENS), AAV-mediated eGFP expression was assessed in the enteric ganglia of the myenteric plexus across different areas of the GI tract—stomach, duodenum, jejunum, ileum, proximal colon and cecum (FIG. 2E-FIG. 2F). Mean transduction efficiency of ˜8% by MaCPNS1, ˜17% by MaCPNS2, ˜13% by PHP.S and ˜12% by AAV9 were observed (FIG. 2F). From the analysis of individual GI segments, variability in transduction of enteric ganglia was observed, with a bias of MaCPNS2 transduction towards small intestinal (SI) segments (duodenum, jejunum and ileum) over other areas, indicating a modest improvement in SI ENS transduction by MaCPNS2 compared to other vectors. To more closely examine this SI bias, the vector dose was slightly lowered to 1×10¹¹ vg and a 2-fold increase was observed in transduction of the SI by MaCPNS2 compared to AAV9 (FIG. 2G-FIG. 2H). Next, it was asked if the transduction efficiency across large intestinal (LI) segments could be improved by a higher dose (FIG. 2I, FIG. 10D). Compared to the initial dose of 3×10¹¹ vg, a MaCPNS2 dose of either 5×10¹¹ vg or 7×10¹¹ vg achieved robust and uniform transduction of the enteric ganglia of the myenteric plexus in the proximal colon as well as sparser transduction in the distal colon. Thus, intravenously-administered MaCPNS2 offers improved transduction of enteric ganglia in the SI, with the option of additional targeting of LI segments at higher doses.

To investigate the liver transduction of MaCPNS1/2 capsids, eGFP expression in vivo was assessed (FIG. 2J-FIG. 2L). MaCPNS1 exhibited 1.5-fold lower transduction compared to the same dose of AAV9 or PHP.S (FIG. 2K). Quantifying the mean brightness of the eGFP fluorescence signal revealed that the new vectors also exhibited 1.3- to 2.1-fold lower signal compared to AAV9 or PHP.S (FIG. 2L), suggesting reduced transgene expression per cell. Collectively these data suggest that MaCPNS1 exhibits lower transduction of hepatocytes and lower transgene expression per liver cell compared to other vectors used for PNS. In addition to the liver, other tissues, including the CNS, were also investigated given AAV9's ability to cross the BBB. At a modest IV dose of 3×10¹¹ vg, CNS transduction was low for all vectors tested, including AAV9 (FIG. 10E). Even when the dose of MaCPNS2 was increased to 5-7×10¹¹ vg, CNS transduction in mice was not observed (FIG. 10F). In addition, MaCPNS1/2 transduction of cardiac muscle was similar to that of AAV9 (FIG. 10E).

All of the above experiments were conducted in adult mice. However, many potential applications require early age intervention. Therefore, MaCPNS1/2 transduction in DRG in neonates was investigated using an alternative delivery route. MaCPNS1/2 capsids packaged with ssAAV:hSyn-tdTomato was introduced into postnatal stage 1 (P1) mice via intraperitoneal (IP) injection, with a dose of 3×10¹¹ vg per mouse. After 6 weeks of expression, MaCPNS1 transduced ˜39% of neurons (marked with Tuj1) in DRG and MaCPNS2 transduced ˜35%, including ˜55% of all CGRP+ neurons and ˜19% of all NF200+ neurons (FIG. 10G-FIG. 10I). With regard to targeting preference, the capsids were predominantly targeting two types of neurons: about 60% of transduced neurons were CGRP+ and ˜11% NF200+(FIG. 10J).

In summary, systemic delivery of MaCPNS1 and MaCPNS2 vectors in mice can efficiently transduce sensory ganglia (such as NG and DRG) compared to AAV9, with MaCPNS2 distinguished from MaCPNS1 by enhanced transduction of the ENS. Further, MaCPNS1 and MaCPNS2 exhibited improved specificity for the PNS, with relatively lower transduction of AAV9's primary target, the liver.

### Systemic MaCPNS1-Mediated Sensor and Actuator Expression Enable Functional Characterization of Neurons in Nodose Ganglia and Dorsal Root Ganglia.

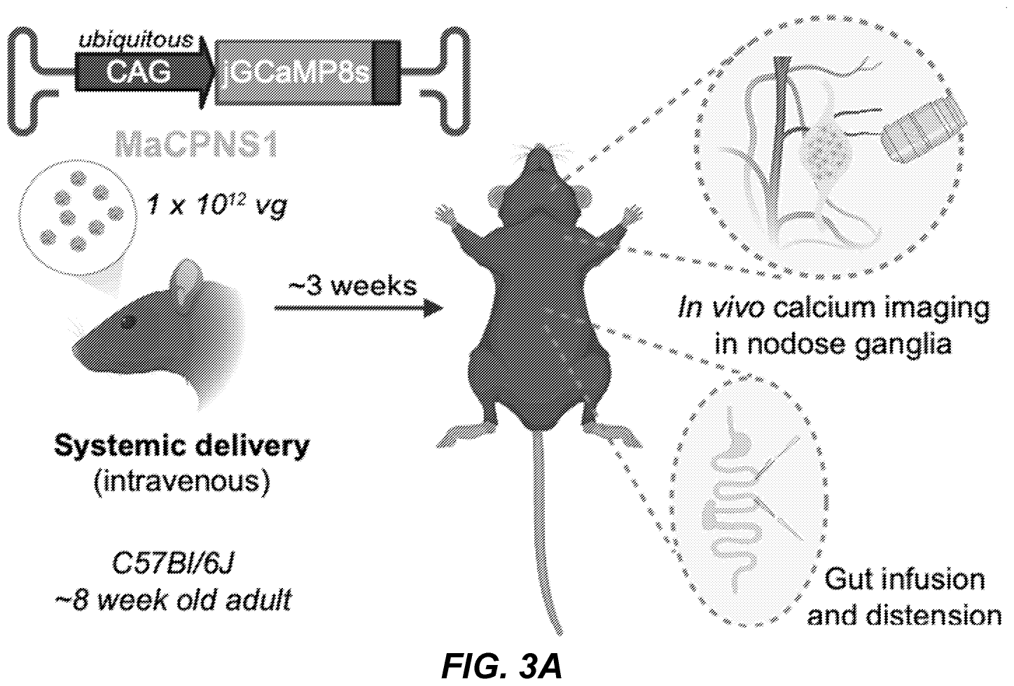

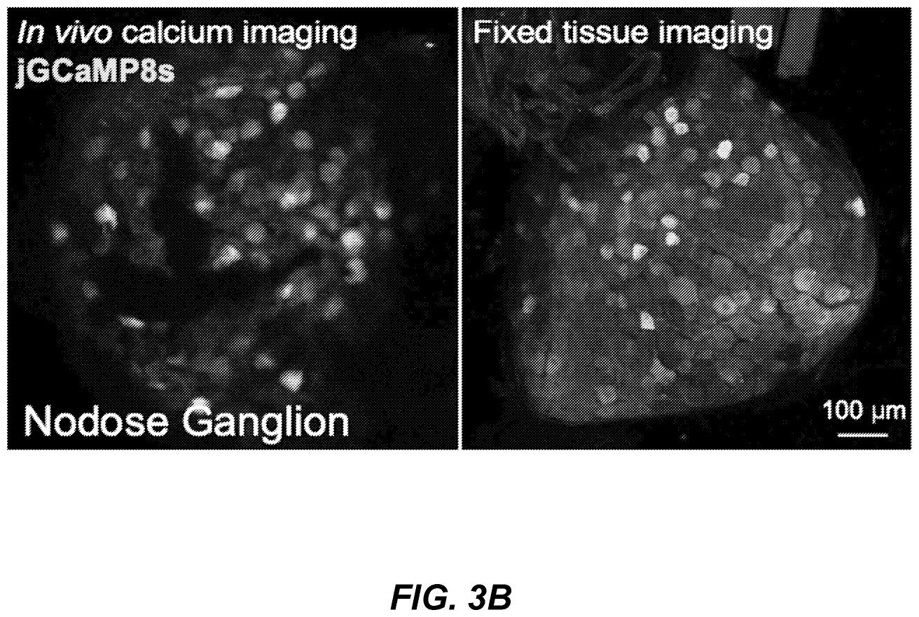

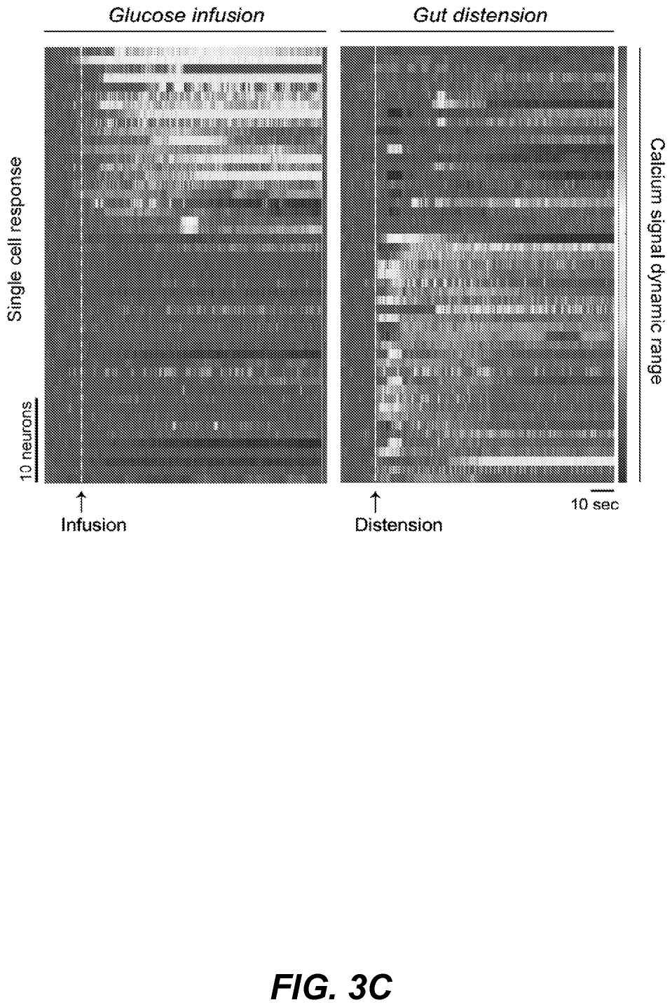

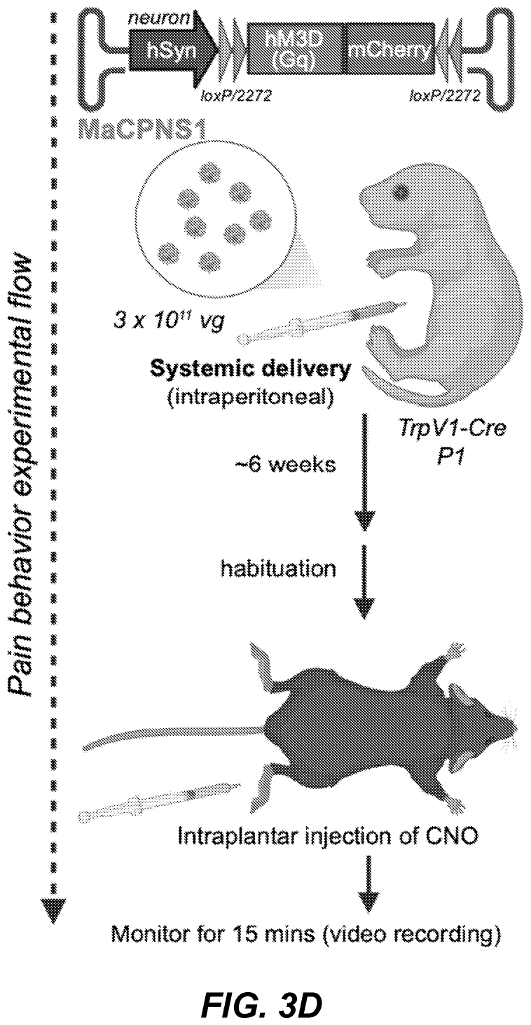

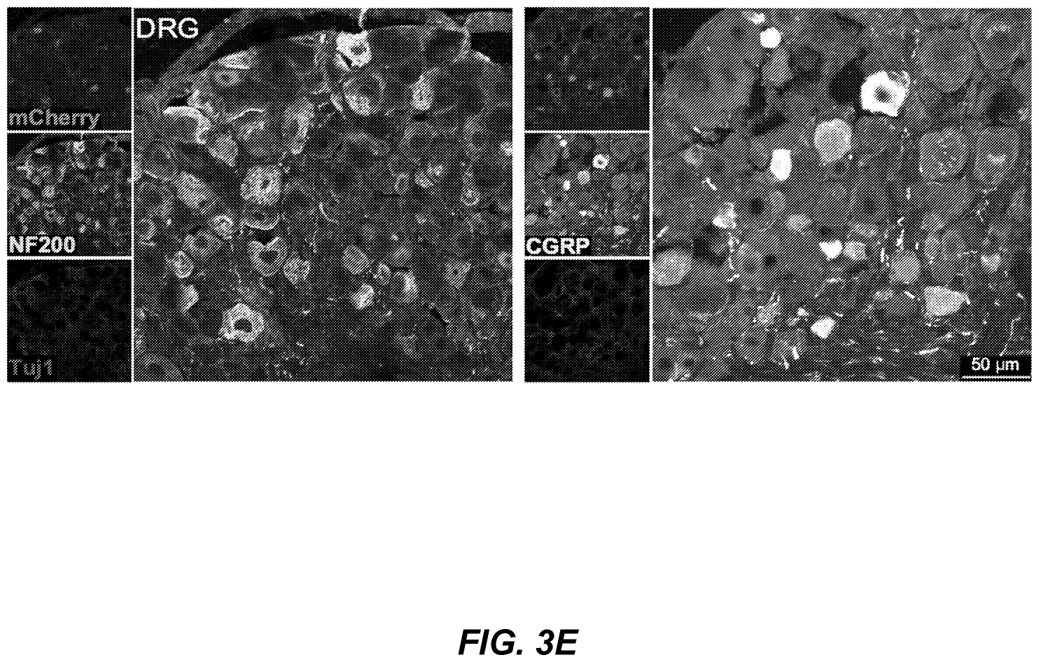

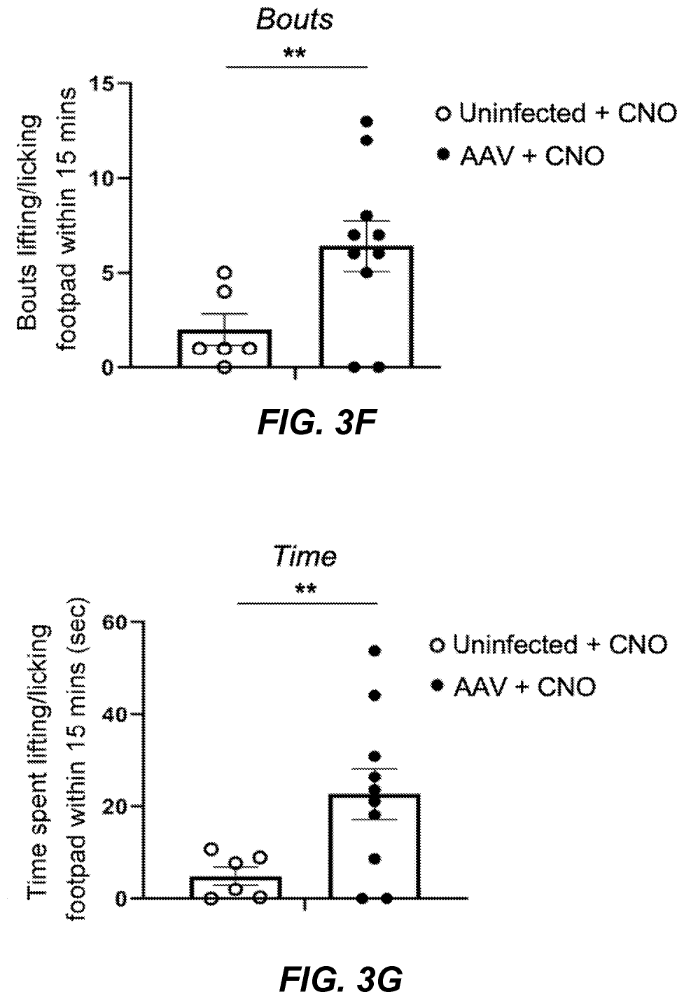

Functional readout of PNS activity during physiological conditions is key to understanding the interaction between the brain and peripheral system. However, commonly-used imaging with genetically encoded calcium indicators (GECIs) in the CNS has been challenging in the PNS due to the low efficiency of delivering GECI to PNS targets. To test the applicability of MaCPNS1/2 capsids to this problem, MaCPNS1 capsid packaged with a recently-developed GECI, jGCaMP8s, was intravenously delivered to adult mice. After three weeks of expression, calcium signals in vivo were recorded during procedures on the gut (FIG. 3A-FIG. 3C). The gut was infused with either glucose solution or saline (to induce distension) while recording neuronal activity in the NG of the anesthetized mice and observed distinguishable neuronal responses in NG (FIG. 3A-FIG. 3C). No significant expression was observed in the CNS or liver of these mice.

After verifying the new vectors' potential for enabling functional readout of the PNS, the further step of seeing whether functional modulation could be achieved with the vectors was taken. A mouse pain-induction system was constructed with improved temporal control, a critical tool for understanding and potentially managing pain. To activate DRG TrpV1 neurons, Cre-dependent excitatory Designer Receptors Exclusively Activated by Designer Drugs (DREADD) was packaged into MaCPNS1 and intraperitoneally injected the vector into P1 TRPV1-Cre pups (FIG. 3D). After 6 weeks of expression (FIG. 3E), mice were given an intraplantar injection of the DREADD agonist clozapine N-oxide (CNO) and evaluated resulting pain-like behaviors. Increases in the number of bouts and overall time spent lifting or licking the injected footpad were observed in the experimental AAV-administered group compared to the control group, indicating nocifensive pain-like behavior (FIG. 3F-FIG. 3G).

Together, these results provide a proof of concept for the application of these new vectors to a wide range of studies involving the monitoring and modulation of sensory processes, including pain.

### IV-Administered AAV-MaCPNS1/2 Efficiently Transduces the PNS in Adult Rats.

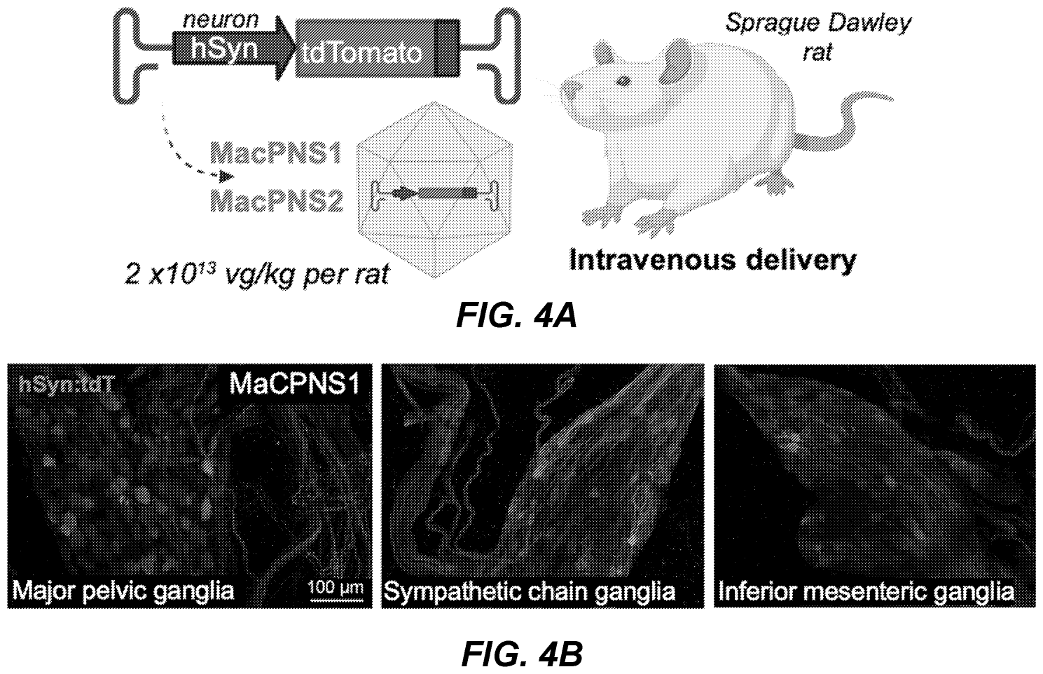

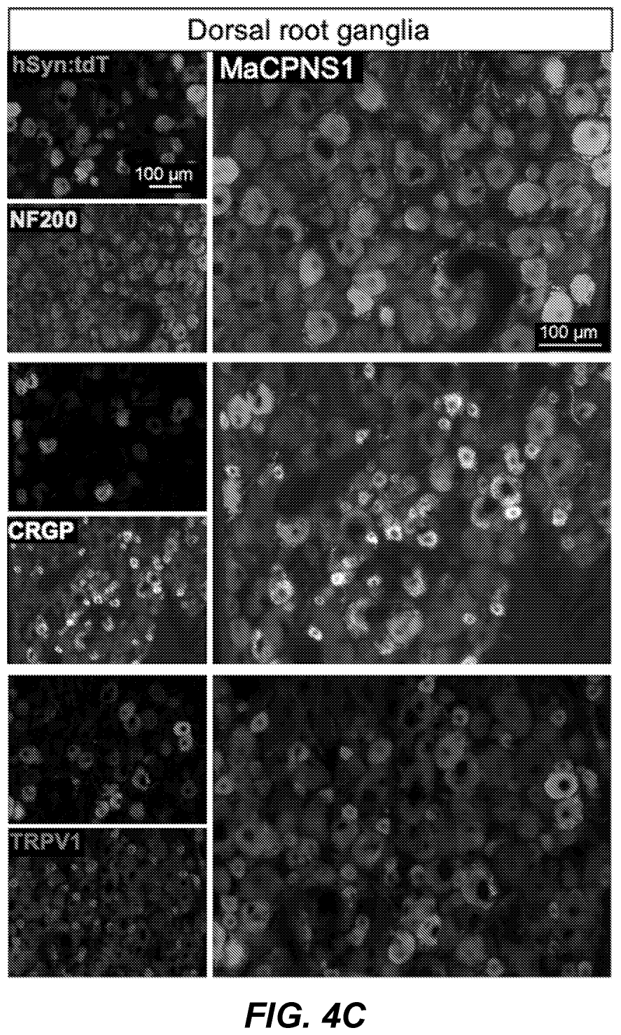

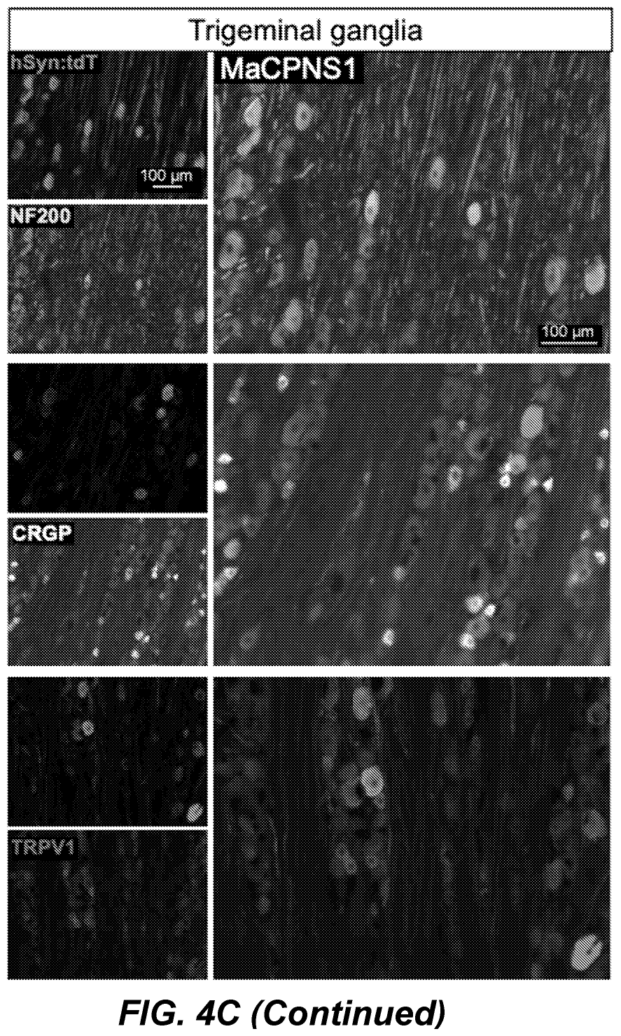

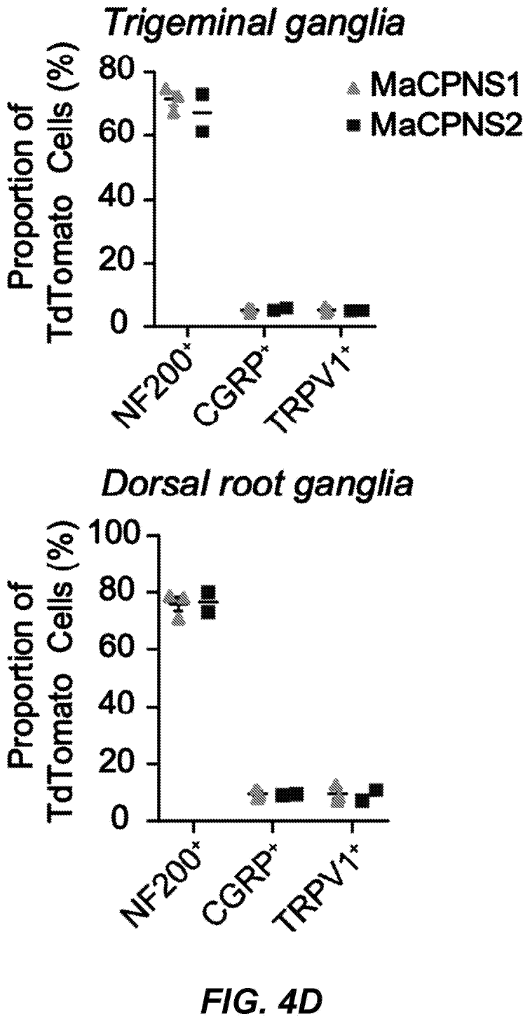

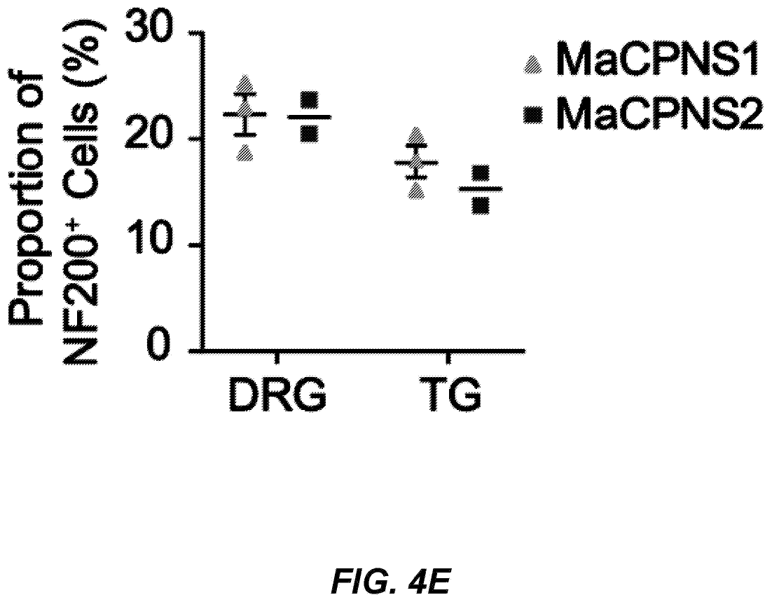

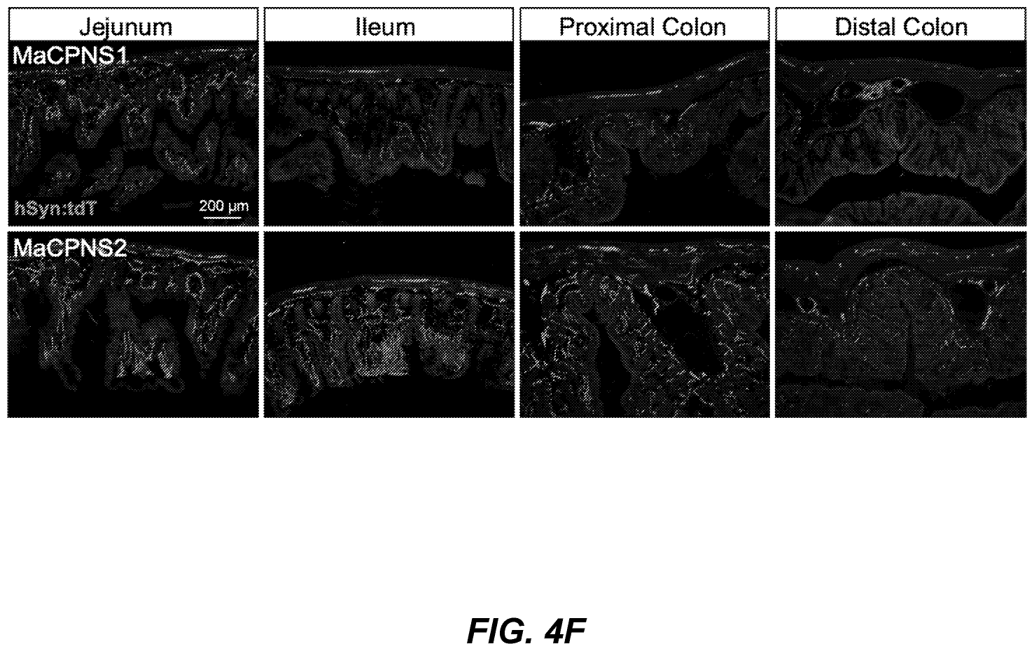

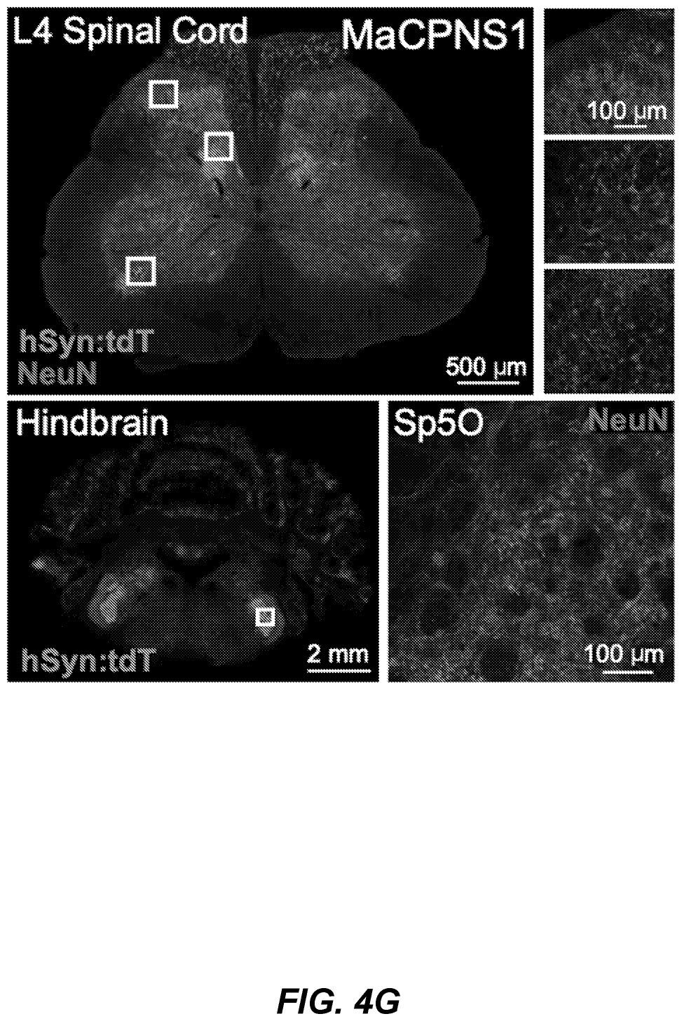

Having validated the new variants' transduction profiles in mouse models, their efficacy in another common research model system, rats, was investigated. Systemic delivery of MaCPNS1/2 capsids packaged with ssAAV:hSyn-tdTomato in Sprague Dawley adults (FIG. 1A) transduced sensory ganglia (DRG and trigeminal ganglia (TG)), sympathetic chain ganglia (SCG), parasympathetic ganglia (inferior mesenteric ganglia (IMG)), mixed sympathetic-parasympathetic ganglia (the major pelvic ganglion (MPG)), and enteric ganglia across the SI and LI (FIG. 4A-FIG. 4F, FIG. 11A-FIG. 11B). The majority of cells transduced in both DRG and TG were NF200+(about 70-75%), with only 5% either CGRP+ or TRPV1+(FIG. 4C-FIG. 4D). In terms of efficiency, both capsids transduced ˜22% of the NF200+ cells in DRG, and 15%-17% of the NF200+ cells in TG (FIG. 4E). Interestingly, this cell type bias contrasts with the observation in neonatal mice, where the vectors showed a bias towards CGRP+ neurons (FIG. 10I-FIG. 10J). Without being bound by any particular theory, this profile could result from differences in species or age/time of injection, as well as the different systemic routes of vector delivery (similar tropism shifts have been reported elsewhere. The tropism diversity highlights the necessity of considering all relevant experimental variables to achieve the desired transduction profile.

The vectors' efficient labeling of sensory ganglia led to further investigation into the ENS (FIG. 4F, FIG. 11C). Shown by the analysis of segments of the SI and LI, MaCPNS1 and MaCPNS2 were comparable at transducing the myenteric plexus and the submucosal plexus, including the vascular and periglandular plexuses. Next, projections to the SC and brainstem were investigated (FIG. 4G, FIG. 11D). Sensory nerve fibers entering the dorsal horn of the SC were densely labeled, and the ascending afferent tracts in the dorsal column were also strongly transduced by both virus vectors. In the brainstem, fibers were densely labeled in the spinal trigeminal nucleus oralis (Sp5O), which can be projected from the TG. In addition to the transduction in the PNS and the nerve projections labeling in the CNS, some labeling of neuronal cell bodies was also observed across regions in the brain (FIG. 11D). Furthermore, no expression in the liver was observed (FIG. 11E).

Together, these results show that the potent PNS tropism of the MaCPNS1/2 vectors is conserved across rodent models tested.

IV-Delivered AAV-MaCPNS1/2 Transduces the Adult Marmoset CNS and PNS More Efficiently than AAV9.

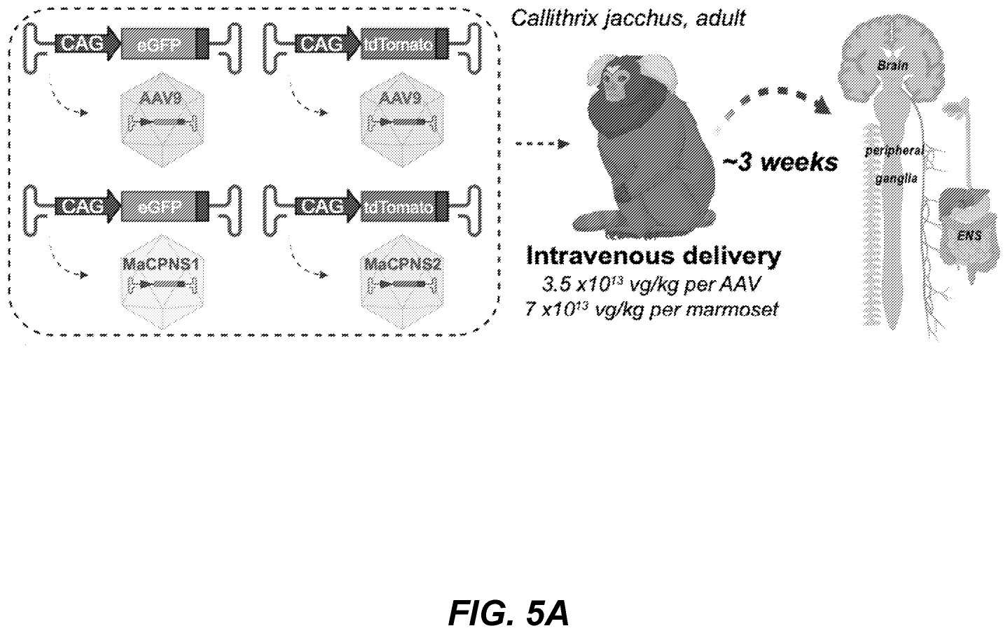

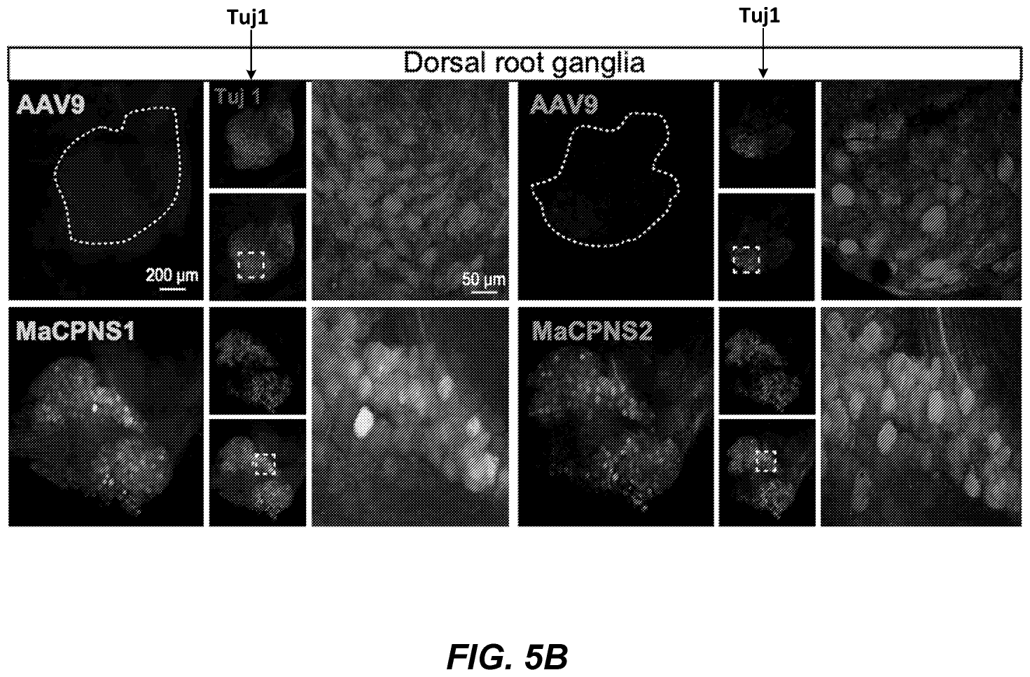

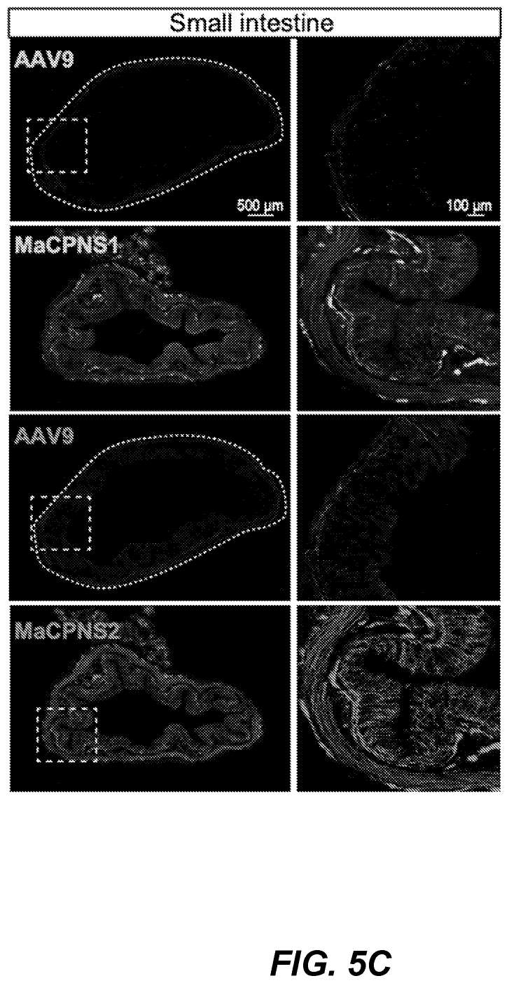

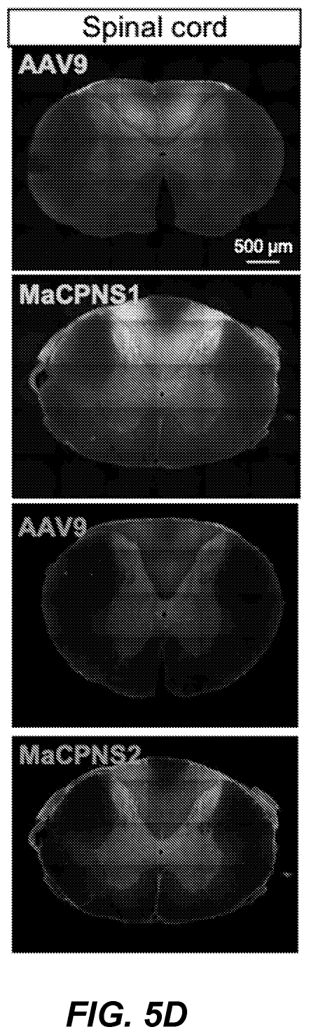

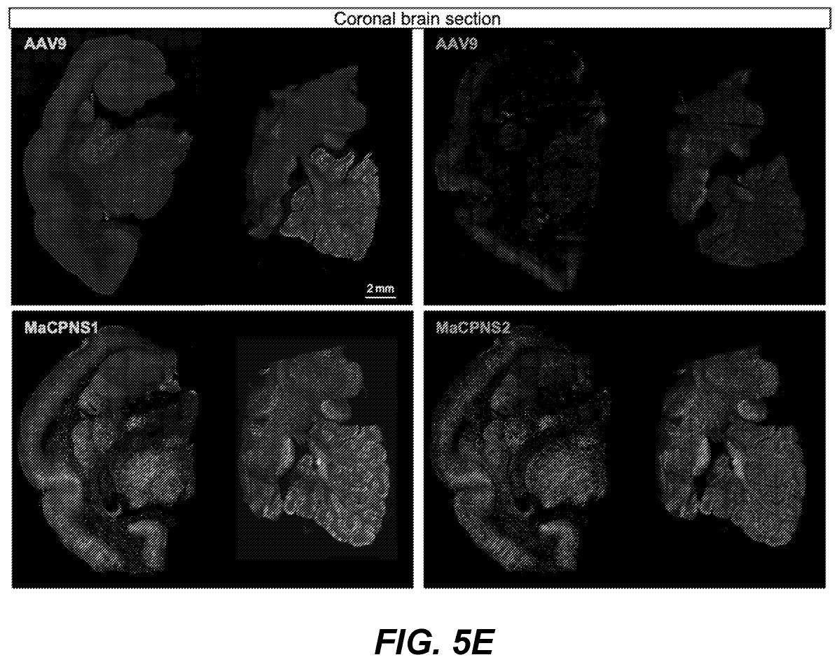

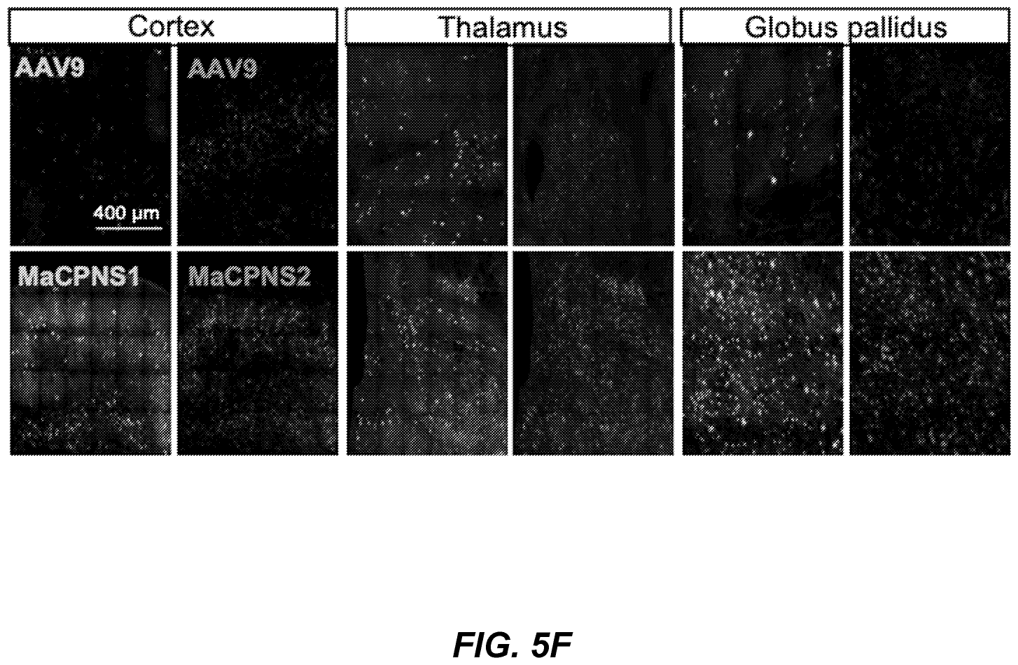

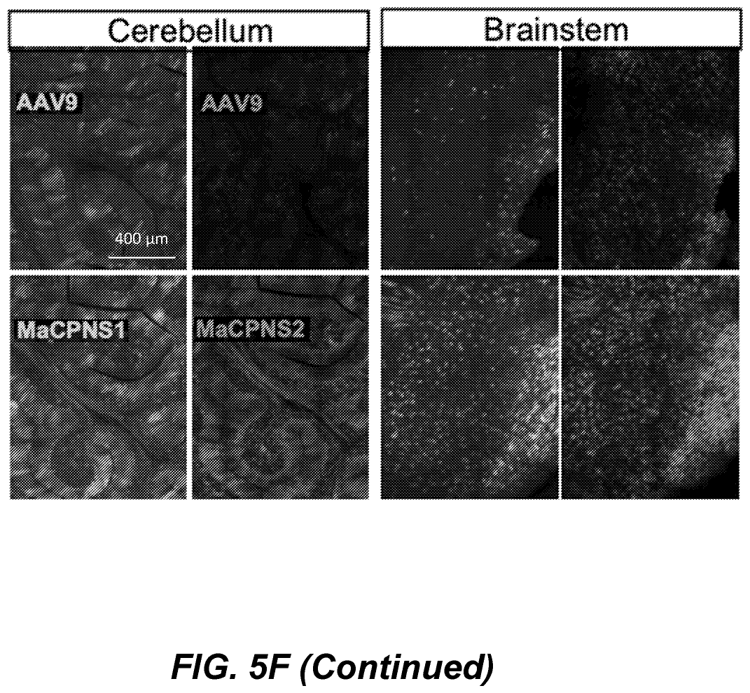

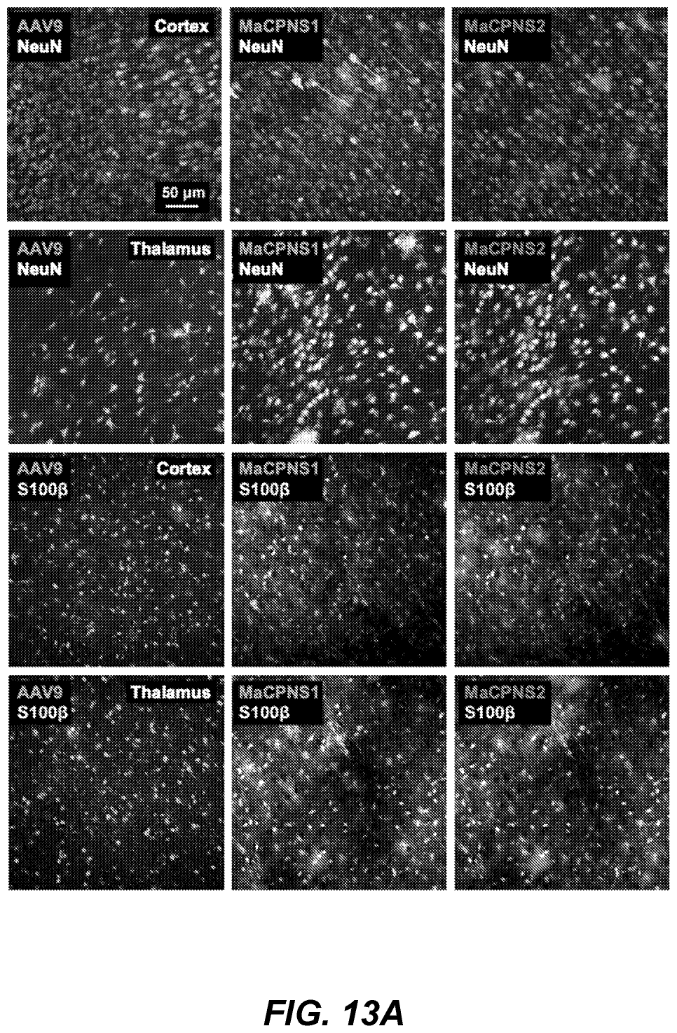

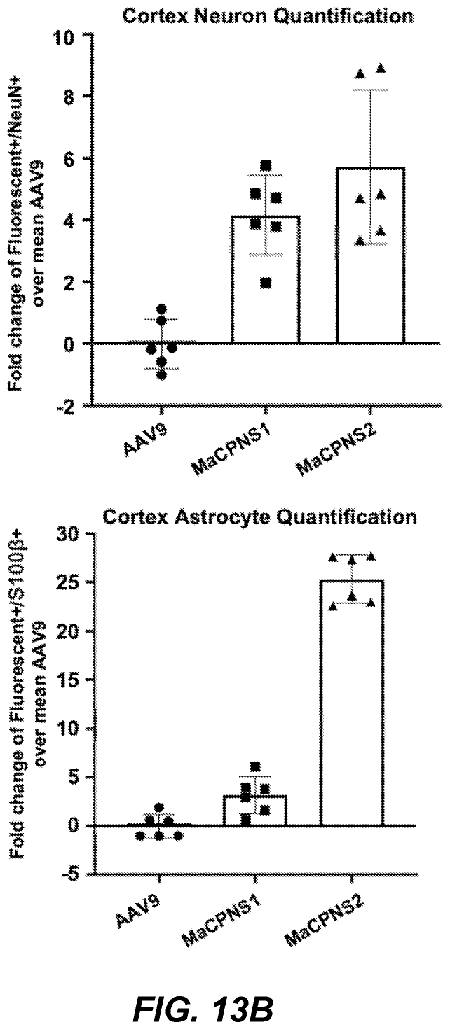

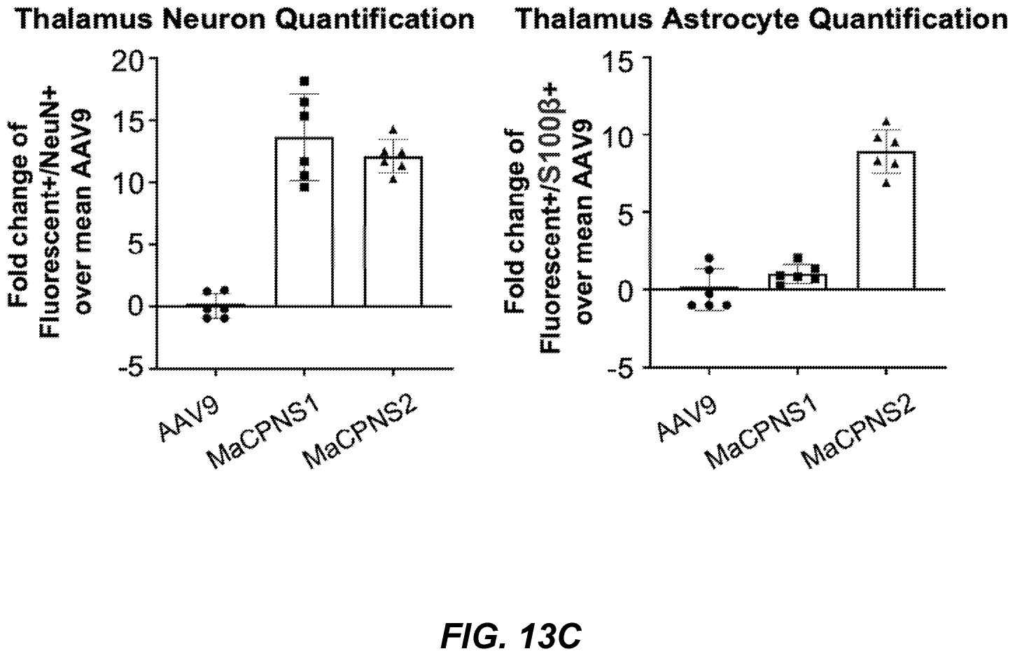

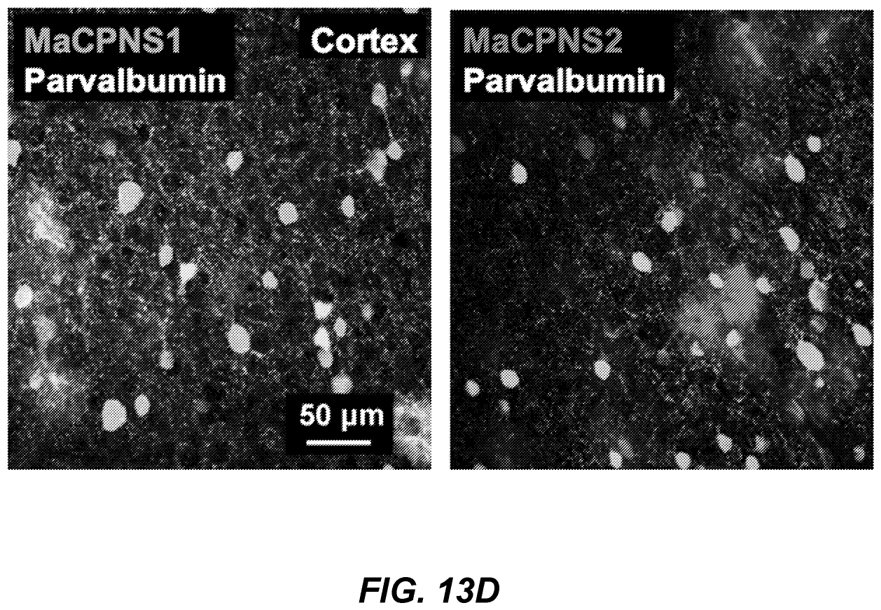

Novel capsids selected in mice do not always translate to NHPs. After validating the MaCPNS1/2 capsids in mice and rats, their performance in NHPs was assessed. The marmoset, a New World monkey and an emerging animal model, was first chosen for translational research. Owing to the limited availability of these animals, in each adult animal, two viral capsids (AAV9, MaCPNS1 or MaCPNS2) packaging different fluorescent reporters (either ssAAV:CAG-eGFP or ssAAV:CAG-tdTomato) were tested (FIG. 5A). Compared to the parent AAV9, intravenously-delivered MaCPNS1/2 capsids carrying either ssAAV:CAG-eGFP or ssAAV:CAG-tdTomato genome showed robust transduction of the PNS and the CNS. In the PNS, enhanced transduction of DRG, the SI and the ascending fiber tracts in the dorsal column of the SC (FIG. 5B-FIG. 5D), was observed similar to what was observed in rodents. In the CNS, robust brain-wide transduction by MaCPNS1/2 capsids, but not AAV9, was evident in regions including the cortex, thalamus, globus pallidus, cerebellum and brainstem (FIG. 5E-FIG. 5F). Further antibody staining and quantification revealed that capsids mainly transduce neurons and astrocytes in the marmoset brain. In the marmoset cortex, MaCPNS2 displayed a ˜5.5-fold increase in neuronal transduction over AAV9 while MaCPNS1 displayed a ˜4-fold increase over AAV9 (FIG. 13A-FIG. 13C). Among the neurons transduced, few overlapped with the PV marker, indicating the vectors' potential bias to excitatory population (FIG. 13D). For astrocyte transduction, MaCPNS2 displayed a ˜25-fold increase over AAV9 in cortex (FIG. 13B). Similar improvement in both neuronal and astrocytic transduction was also observed in the thalamus (FIG. 13C).

These results demonstrate that the MaCPNS1/2 capsids can efficiently cross the BBB in adult marmosets, while still exhibiting the enhanced PNS tropism observed in rodents.

IV-Delivered AAV-MaCPNS1/2 Transduce the Infant Rhesus Macaque CNS and PNS More Efficiently than AAV9.

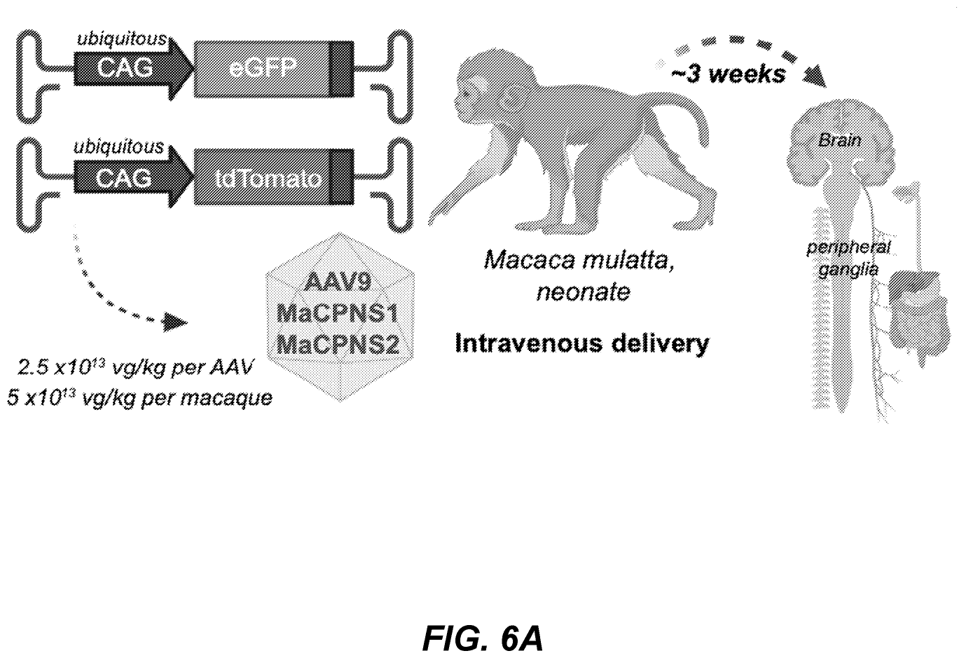

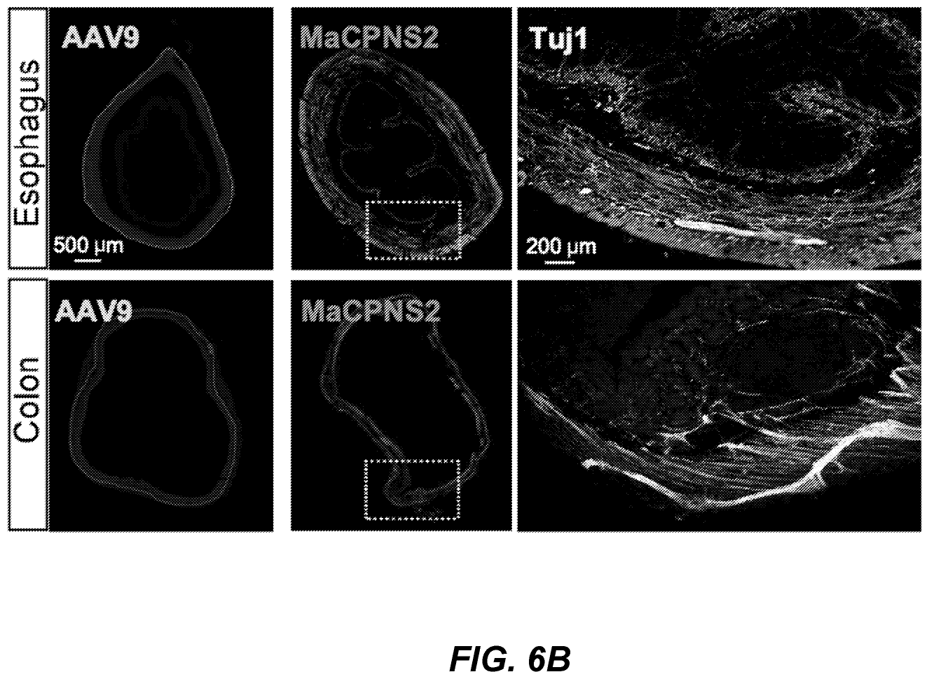

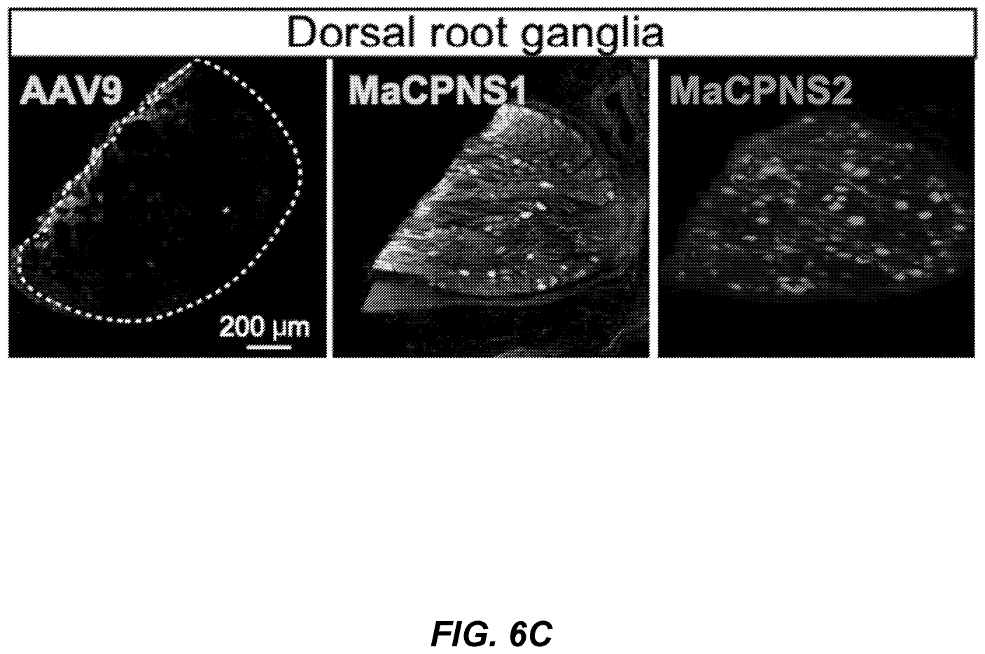

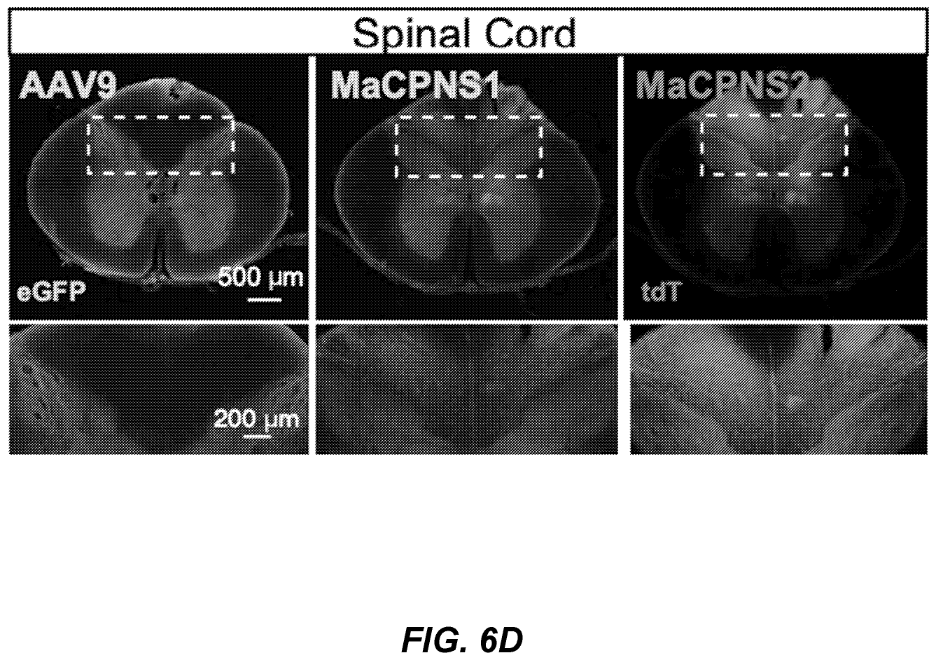

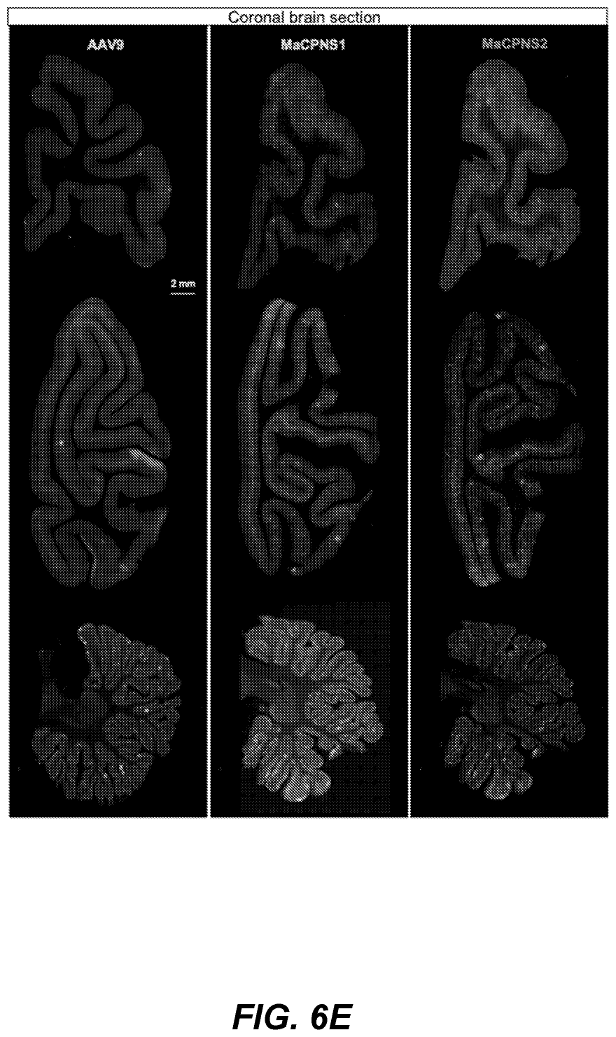

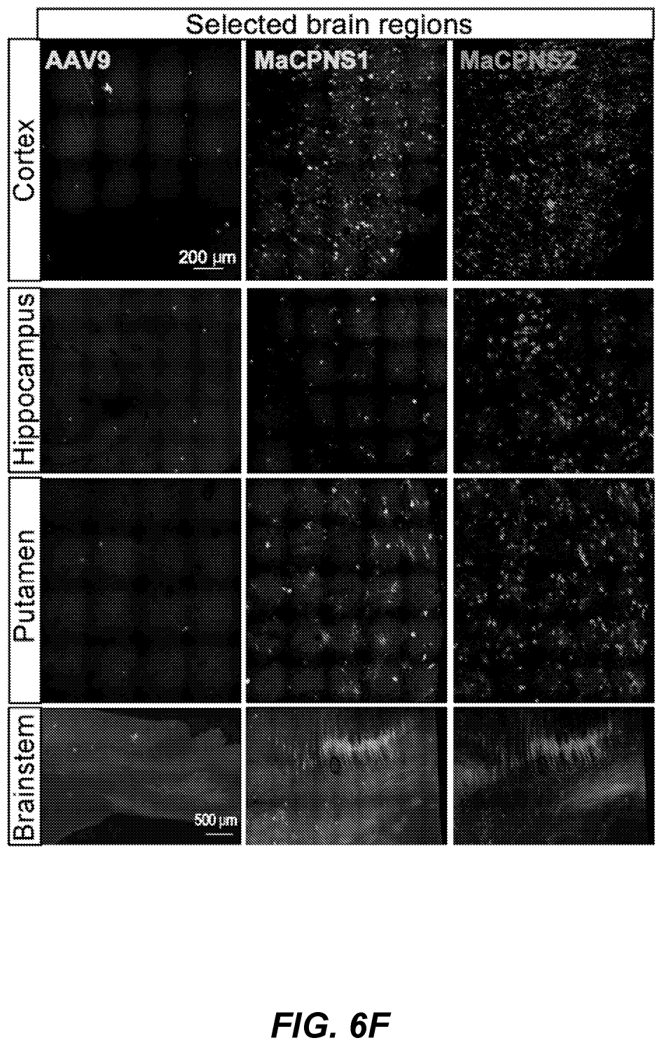

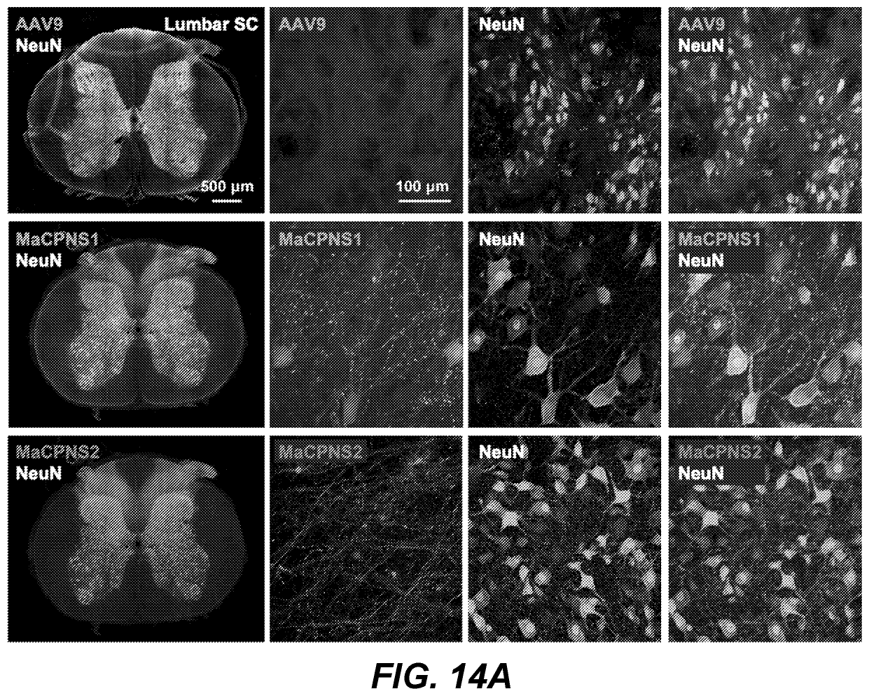

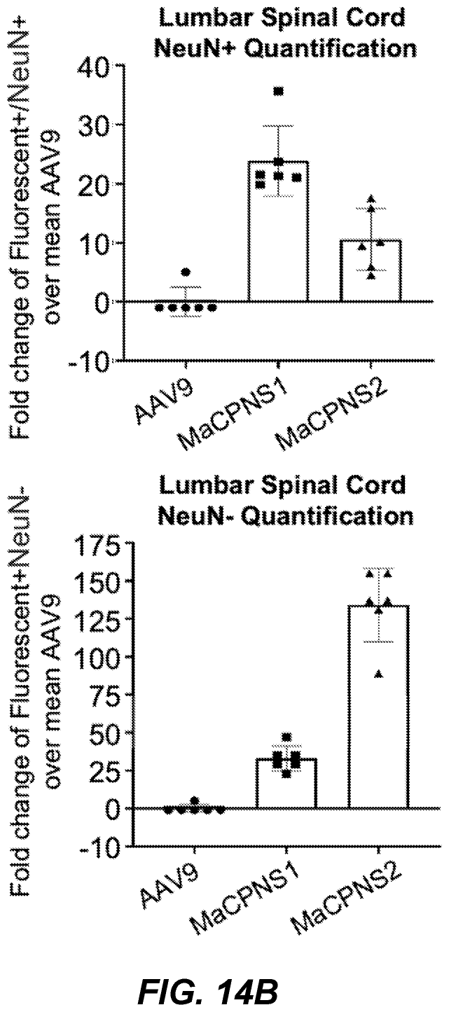

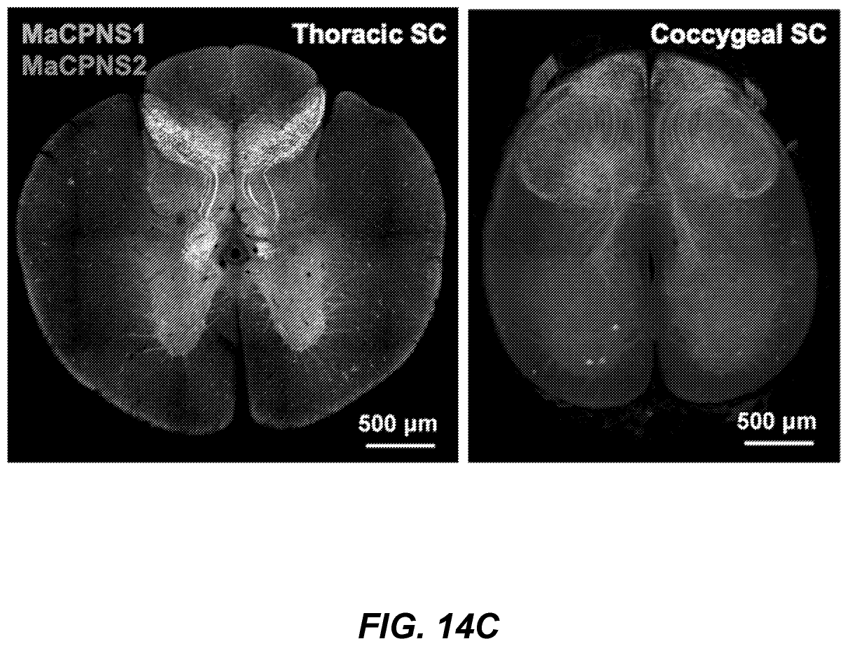

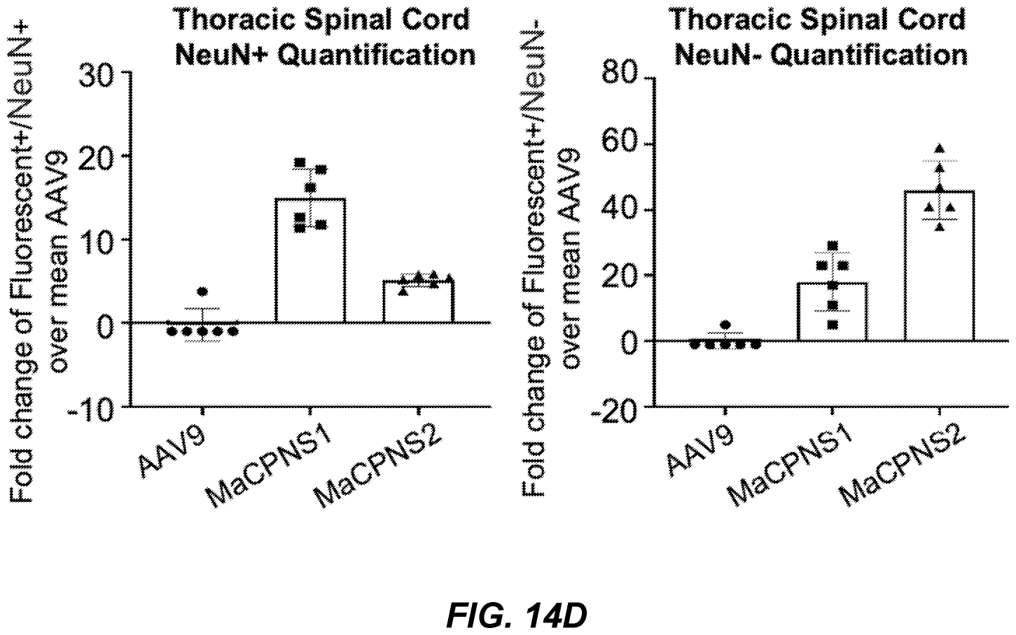

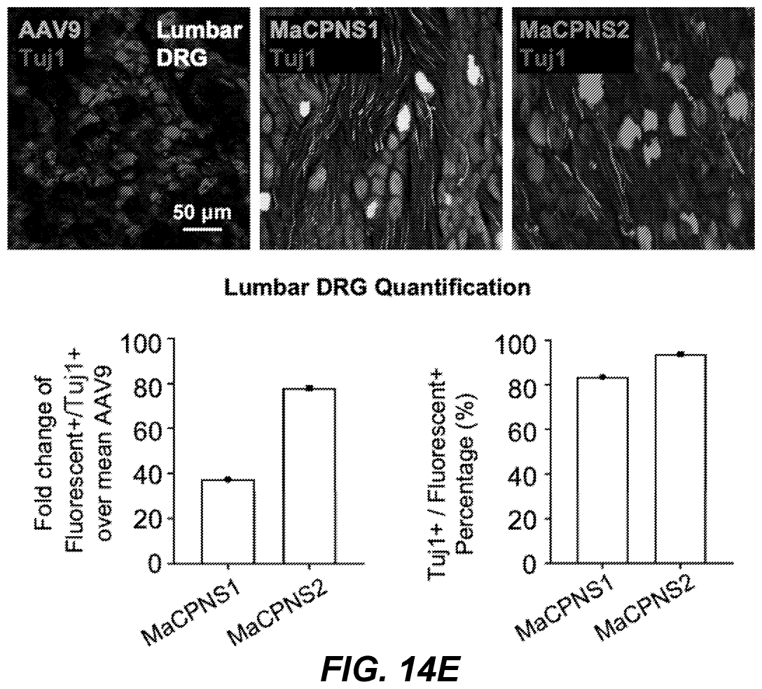

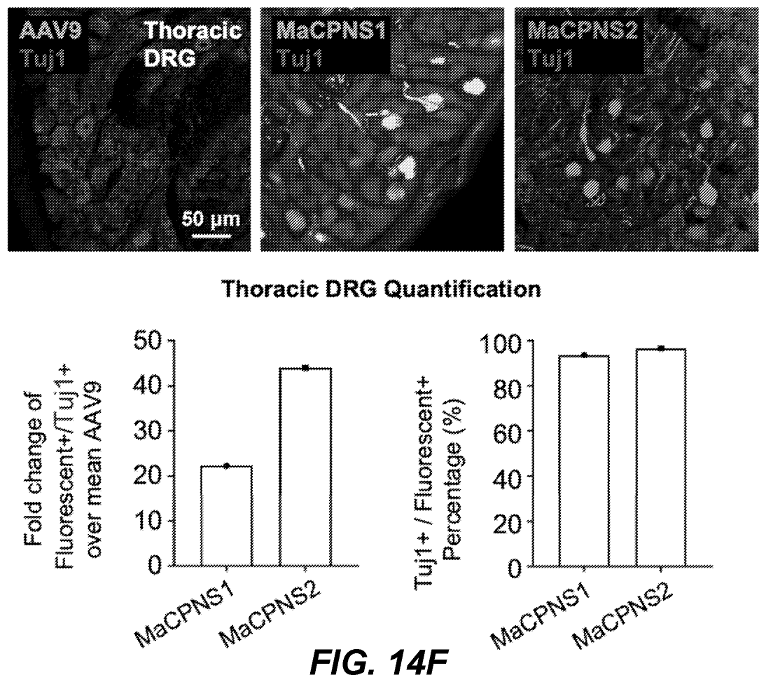

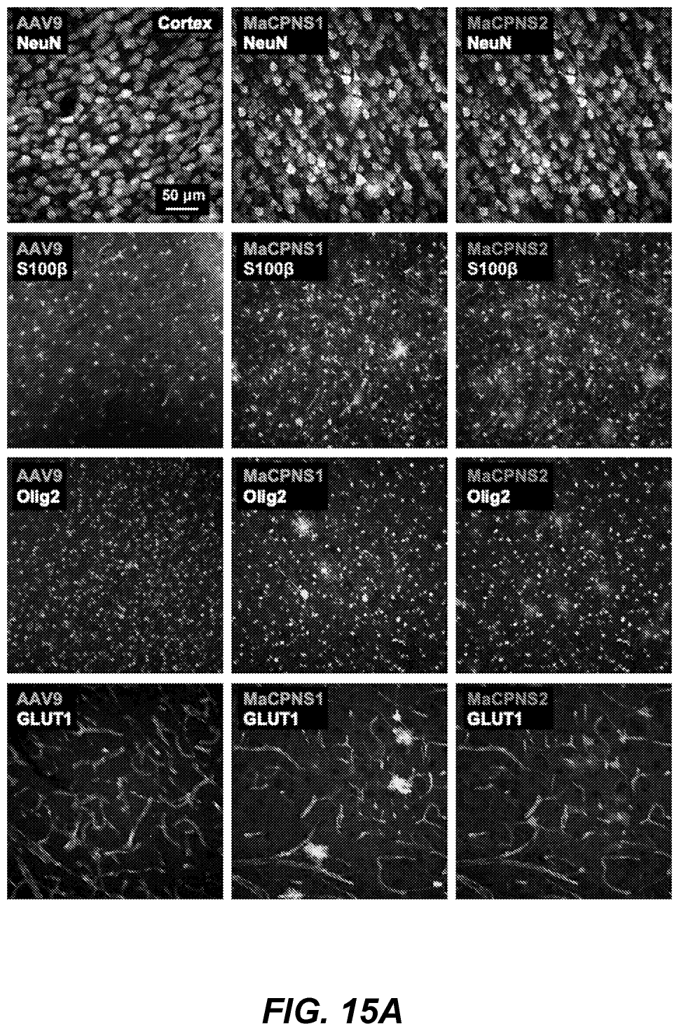

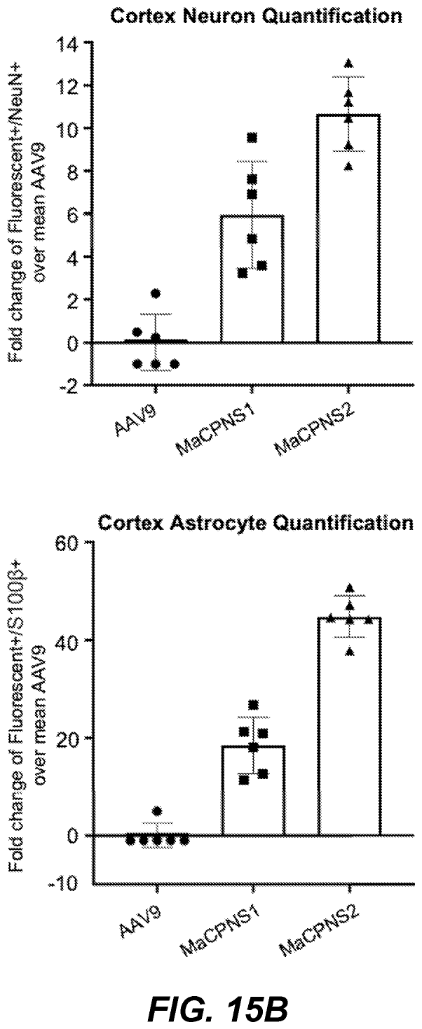

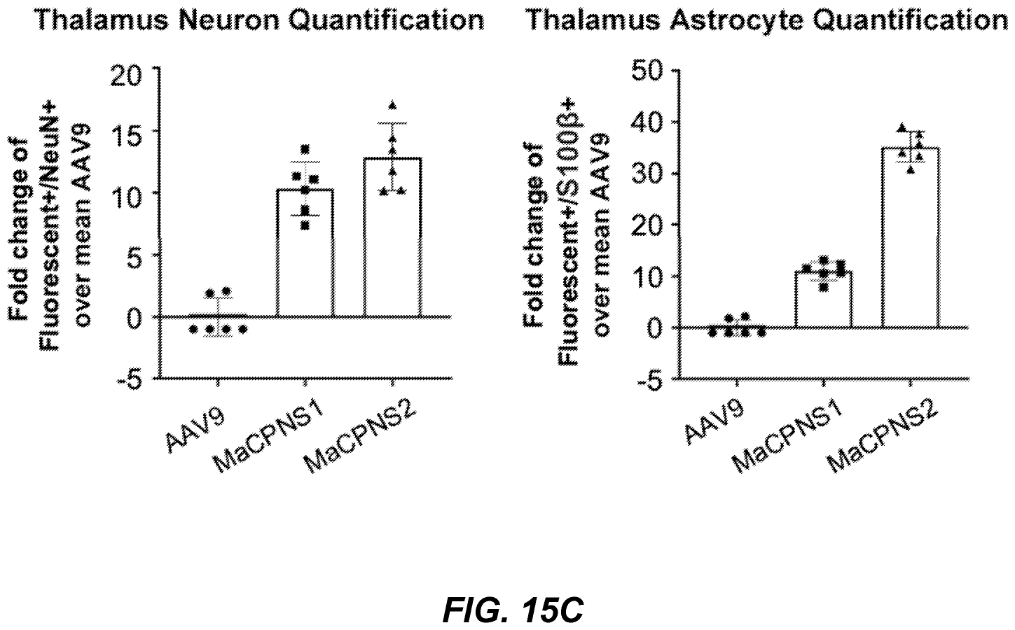

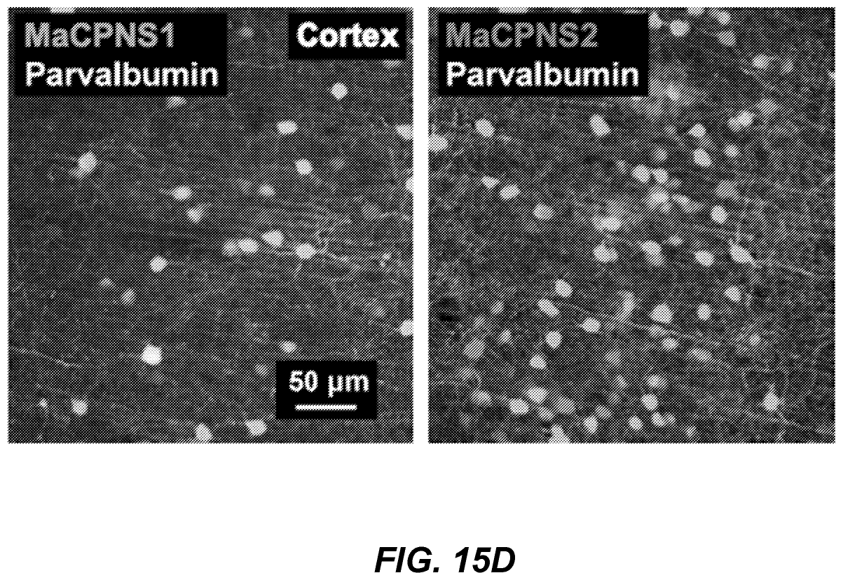

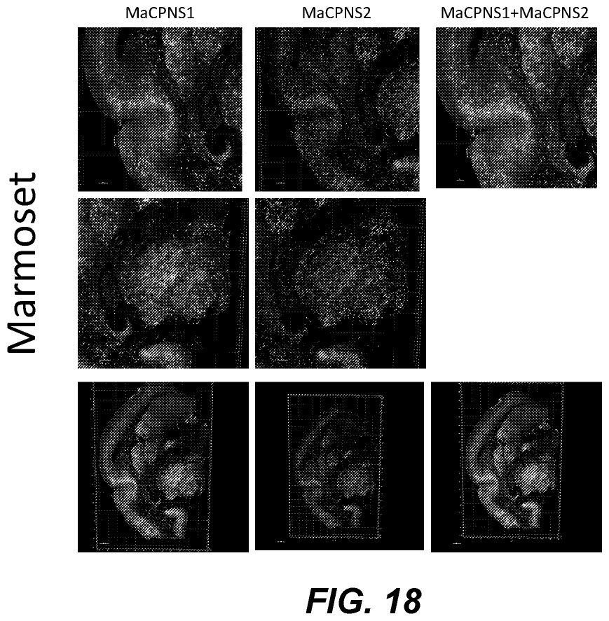

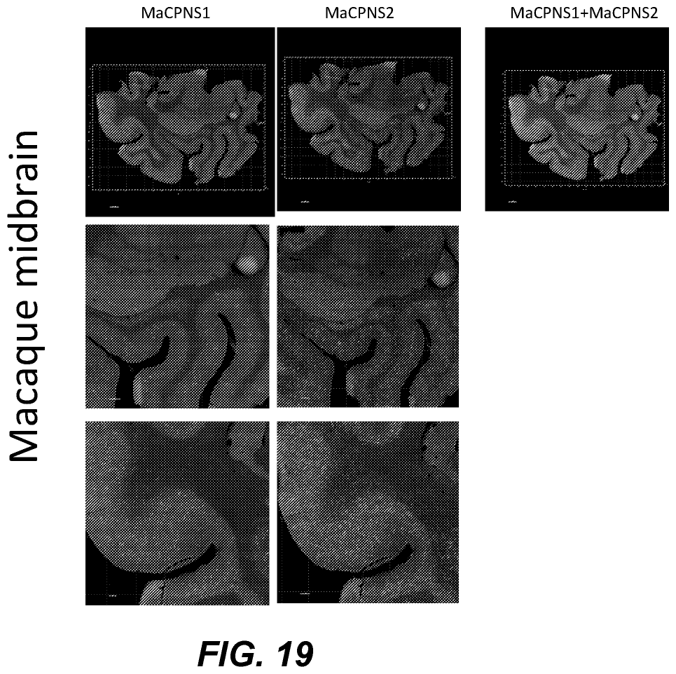

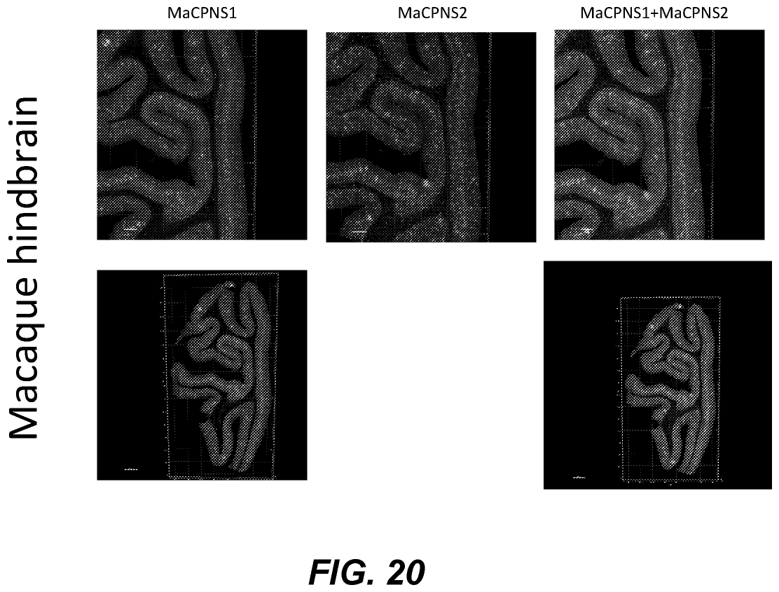

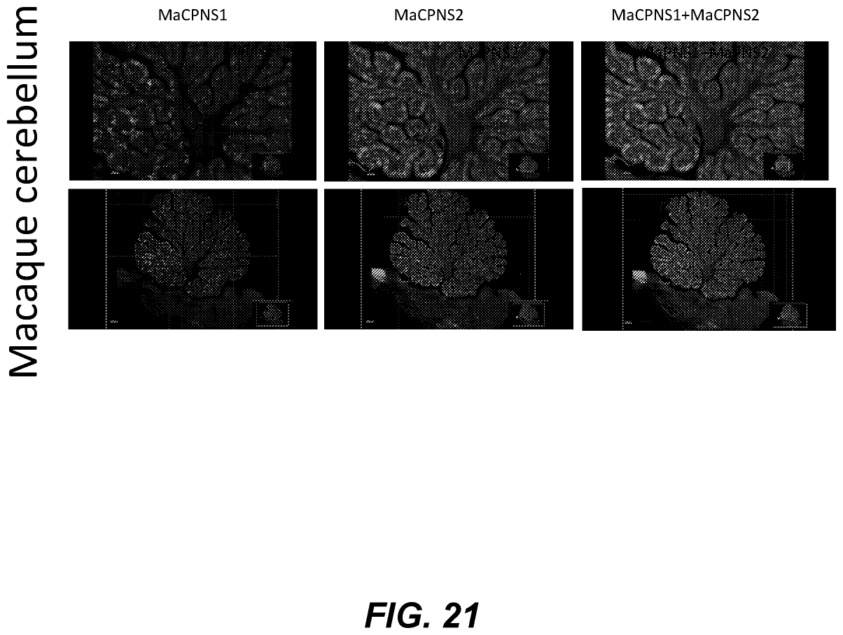

Encouraged by the tropism of MaCPNS1/2 capsids in marmosets, their transduction capability was further assessed in another NHP, the rhesus macaque, an Old World monkey and a common pre-clinical research model for AAV gene therapy. The same strategy was employed as in marmosets, intravenously administering AAV9, MaCPNS1 and MaCPNS2 capsids packaging either ssAAV:CAG-eGFP or ssAAV:CAG-tdTomato to an infant rhesus macaque (FIG. 6A). Similar to the results in marmosets, MaCPNS1/2 efficiently targeted both the CNS and PNS compared to the parent AAV9 after three weeks of expression. Enhanced transduction in the SC, DRG and GI tract, including the esophagus, colon and SI was observed (FIG. 6B-FIG. 6D), as observed in rodents and marmosets. In the SC, there was strong labeling of both ascending fiber tracts in the dorsal column as well as the peripheral fiber tracks coming into the dorsal horn. Within the grey matter of the lumbar SC, MaCPNS1 displayed a ˜25-fold increase in neuronal transduction over AAV9. For non-neuronal cells, MaCPNS2 displayed a ˜130-fold increase in transduction over AAV9 (FIG. 14A-FIG. 14B). Similar improvement in transduction was also observed in the thoracic SC (FIG. 14C-FIG. 14D). In the lumbar and thoracic DRG, MaCPNS2 displayed ˜77-fold and ˜44-fold increases in neuronal transduction over AAV9, respectively. MaCPNS1 also showed ˜37-fold and ˜22-fold increases in neuronal targeting in lumbar and thoracic DRG. Within lumbar DRG (FIG. 14E), MaCPNS2 was highly specific to neurons, with ˜94% of all transduced cells being neurons. In thoracic DRG, both vectors displayed high specificity in targeting neurons (FIG. 14F). In the CNS, MaCPNS1/2 capsids mediated enhanced brain-wide transduction including in areas such as the cortex, hippocampus, putamen and brainstem (FIG. 6E-FIG. 6F, FIG. 12, FIG. 18-FIG. 21), similar to the observations in marmosets. Both vectors transduced neurons and astrocytes but not oligodendrocytes or endothelial cells (FIG. 15A-FIG. 15B). In the macaque cortex, neuron transduction by MaCPNS1 and MaCPNS2 was ˜6-fold and ˜11-fold higher, respectively, than AAV9 (FIG. 15B). Most neurons transduced by both vectors were PV-, indicating a potential bias to excitatory populations (FIG. 15D). MaCPNS2 also displayed a ˜44-fold increase in astrocyte transduction over AAV9 in cortex (FIG. 15B). A similar improvement in neuronal and astrocytic transduction was also observed in the thalamus (FIG. 15C). Consistent with the observations in marmoset, MaCPNS1's transduction was more biased towards neurons while MaCPNS2 showed increased transduction of both neurons and astrocytes. No significant difference in liver transduction was observed between MaCPNS1, MaCPNS2 and AAV9 (FIG. 12).

These experiments demonstrate that the new capsids, MaCPNS1/2, can efficiently transduce the PNS and CNS in both New and Old World monkeys, making them useful vectors for translational research across the nervous system.

Systemic AAVs described herein can address the pressing need for efficient gene delivery vectors to target the nervous system across species. By in vivo selection and data analysis, a library of capsids with divergent tropism compared to their parent, AAV9, were identified. Two variants, MaCPNS1 and MaCPNS2, were noteworthy for their potent neurotropic behavior in the mouse model, in which they were selected. In contrast to the previously-engineered PNS-targeting variant, AAV-PHP.S, which requires a high dose to be potent, intravenous delivery of a modest dose of the new variants in adult mice showed about 2-fold improvement in the transduction of NG and DRG compared to AAV9. In addition to improved sensory ganglia transduction, the MaCPNS2 capsid showed improved transduction of the SI of the ENS. These MaCPNS1/2 capsids also stood out from AAV9 and PHP.S in their specificity for the PNS, with lower transduction in the liver. In addition to their performance in adult mice, the new vectors efficiently transduced DRG when delivered at the P1 neonatal stage in mice, via a technically easy intraperitoneal injection. In neonatal mice, both vectors showed a significant bias towards transducing CGRP+ neurons.

Experiments involving functional readout or modulation in the sensory system require a high copy-number of functional proteins. This can be challenging to achieve with systemic delivery, and is thought to require a combination of both an efficient vector and an engineered genetic indicator/probe. In the present disclosure, the new MaCPNS1 capsid can be used systemically for both monitoring and modulating neuronal function. By systemically delivering a recently engineered GECI, jGCaMP8s, vagal neuron calcium dynamics can be visualized in response to gut glucose infusion and distension. By demonstrating the use of viral vector-mediated GECI delivery for imaging in wild-type mice, similar imaging studies in species, where transgenic models cannot be available, were performed. Following the success with GECI sensor delivery, the application of the MaCPNS1 vector was extended to systemic delivery of a DREADD actuator to a TrpV1-Cre neonate, enabling pain induction by chemogenetic modulation of TrpV1+ neurons in DRG, which mediate thermosensation and pain. These proof-of-concept experiments demonstrate the application of MaCPNS1/2 for modulating different sensory modalities with higher temporal resolution. With the rapid development of ultra-sensitive opsins and a wireless light source, these newly-developed AAV variants can open the door to less invasive modulation of hard-to-access peripheral ganglia with precise temporal control.

In some embodiments, MaCPNS1 and MaCPNS2 also show promise in rats, another commonly used research model for PNS applications. Both capsids translated their potent PNS tropism across rodents, showing efficient transduction of sensory ganglia, sympathetic ganglia, parasympathetic ganglia, and enteric neurons. Detailed cell-type characterization in DRG and TG of adult rats showed that the vectors were biased towards transducing NF200+ neurons, in contrast to the bias towards CGRP+ neurons observed in DRG in mice injected intraperitoneally at the neonate P1 stage. Without being bound by any particular theory, such tropism shifts have been previously noted with other AAV serotypes, and emphasize the importance of considering the roles various experimental conditions play in determining a vector's tropism in a given animal model.

The conservation of the vectors' potent PNS tropism across rodents led to the test of their performance in NHPs. In one embodiment, the new AAVs were tested in a New World monkey, the marmoset, which has been gaining attention in the neuroscience community as a promising animal model for biomedical research. Intravenous delivery of MaCPNS1 and MaCPNS2 to adult marmosets showed potent PNS tropism. In some embodiments, the vectors also efficiently crossed the BBB to transduce the CNS, making them potent vectors across the nervous system. In another embodiment, the vectors' tropism was validated in an Old World monkey, the rhesus macaque, which is more closely related to humans and is widely used as an animal model for pre-clinical research, including gene therapy. As in marmosets, intravenously delivered MaCPNS1 and MaCPNS2 efficiently transduced both the PNS and CNS in an infant rhesus macaque. Without being bound by any particular theory, the enhanced CNS tropism observed in NHPs can be explained by the heterogeneity of the BBB across species.

The conservation of these AAV variants' potent PNS tropism across species validates the usefulness of selecting capsids in mouse models, a preferred model among capsid engineers due to the relatively fewer challenges implementing iterations of in vivo selection or capsid evolution given animal availability. In one embodiment, the translatability for CNS tropism was further investigated. Prior CNS-specific selections have yielded capsids, which may or may not be translatable across species or whose potential has yet to be tested.

In some embodiments, new AAVs were provided to address some significant challenges in the field of gene delivery vectors for the nervous system (Table 1). Non-invasive delivery of transgenes across the nervous system can be transformative for many applications, including basic science, as demonstrated with previously-engineered AAV vectors. With several therapeutic candidates now in the pipeline for various neurological disorders, the new systemic AAV vectors described in the present disclosure, e.g., AAV-MaCPNS1 and AAV-MaCPNS2, can be applied to accelerate translational research as well.

In at least some of the previously described embodiments, one or more elements used in an embodiment can interchangeably be used in another embodiment unless such a replacement is not technically feasible. It will be appreciated by those skilled in the art that various other omissions, additions and modifications may be made to the methods and structures described above without departing from the scope of the claimed subject matter. All such modifications and changes are intended to fall within the scope of the subject matter, as defined by the appended claims.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. As used in this specification and the appended claims, the singular forms “a,” “an,” and “the” include plural references unless the context clearly dictates otherwise. Any reference to “or” herein is intended to encompass “and/or” unless otherwise stated.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms.

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible sub-ranges and combinations of sub-ranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” “greater than,” “less than,” and the like include the number recited and refer to ranges which can be subsequently broken down into sub-ranges as discussed above. Finally, as will be understood by one skilled in the art, a range includes each individual member. Thus, for example, a group having 1-3 articles refers to groups having 1, 2, or 3 articles. Similarly, a group having 1-5 articles refers to groups having 1, 2, 3, 4, or 5 articles, and so forth.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

What is claimed is:

1. An adeno-associated virus (AAV) targeting peptide comprising an amino acid sequence that comprises at least 4 contiguous amino acids from a sequence selected from the group consisting of PHEGSSR (SEQ ID NO: 169), LNNTKTT (SEQ ID NO: 237), SNLARNV (SEQ ID NO: 274) and TNNTKPL (SEQ ID NO: 390).

2. The AAV targeting peptide of claim 1, wherein the targeting peptide comprises at least 5 contiguous amino acids from the sequence of PHEGSSR (SEQ ID NO: 169).

3. The AAV targeting peptide of claim 1, wherein the targeting peptide comprises at least 6 contiguous amino acids from the sequence of PHEGSSR (SEQ ID NO: 169).

4. The AAV targeting peptide of claim 1, wherein the targeting peptide comprises PHEGSSR (SEQ ID NO: 169).

5. The AAV targeting peptide of claim 1, wherein the targeting peptide comprises at least 5 contiguous amino acids from the sequence of LNNTKTT (SEQ ID NO: 237).

6. The AAV targeting peptide of claim 1, wherein the targeting peptide comprises at least 6 contiguous amino acids from the sequence of LNNTKTT (SEQ ID NO: 237).

7. The AAV targeting peptide of claim 1, wherein the targeting peptide comprises GNNTRDT LNNTKTT (SEQ ID NO: 237).

8. The AAV targeting peptide of claim 1, wherein the targeting peptide comprises at least 5 contiguous amino acids from the sequence of SNLARNV (SEQ ID NO: 274).

9. The AAV targeting peptide of claim 1, wherein the targeting peptide comprises at least 6 contiguous amino acids from the sequence of SNLARNV (SEQ ID NO: 274).

10. The AAV targeting peptide of claim 1, wherein the targeting peptide comprises SNLARNV (SEQ ID NO: 274).

11. The AAV targeting peptide of claim 1, wherein the targeting peptide comprises at least 5 contiguous amino acids from the sequence of TNNTKPL (SEQ ID NO: 390).

12. The AAV targeting peptide of claim 1, wherein the targeting peptide comprises at least 6 contiguous amino acids from the sequence of TNNTKPL (SEQ ID NO: 390).

13. The AAV targeting peptide of claim 1, wherein the targeting peptide comprises TNNTKPL (SEQ ID NO: 390).

14. The AAV targeting peptide of any one of claims 1-13, wherein the targeting peptide is a central nervous system (CNS) targeting peptide, a peripheral nervous system (PNS) targeting peptide, and/or a lung targeting peptide.

15. The AAV targeting peptide of any one of claims 1-14, wherein the targeting AAV peptide is part of an AAV.

16. The AAV targeting peptide of claim 15, wherein the targeting peptide is part of a capsid protein of the AAV.

17. The AAV targeting peptide of any one of claims 1-16, wherein the targeting peptide is conjugated to a nanoparticle, a second molecule, a viral capsid protein, or a combination thereof.

18. An adeno-associated virus (AAV) capsid protein comprising an AAV targeting peptide of any one of claims 1-17.

19. The AAV capsid protein of claim 18, wherein the AAV capsid is derived from an AAV selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, and rhesus isolate rh.10.

20. A nucleic acid, comprising a sequence encoding the AAV targeting peptide of any one of claims 1-17.

21. A nucleic acid, comprising a sequence encoding the AAV capsid protein of any one of claims 18-20.

22. A recombinant adeno-associated virus (rAAV), comprising an AAV targeting peptide of any one of claims 1-17, or an AAV capsid protein of any one of claims 18-20.

23. A recombinant adeno-associated virus (rAAV), comprising an AAV capsid protein which comprises the AAV targeting peptide of any one of claims 1-17.

24. The rAAV of claim 23, wherein the AAV capsid protein comprises the AAV targeting peptide of any one of claims 1-17 inserted between two adjacent amino acids in AA586-592 or functional equivalents thereof of the AAV capsid protein.

25. The rAAV of claim 24, wherein the two adjacent amino acids are AA588 and AA589.

26. The rAAV of any one of claims 22-25, wherein the AAV capsid protein comprises, or consists thereof, SEQ ID NO: 391.

27. The rAAV of any one of claims 22-26, wherein the rAAV comprises an rAAV vector genome.

28. A composition, comprising
an AAV targeting peptide of any one of claims 1-17,
an AAV capsid protein of any one of claims 18-19,
a nucleic acid of any one of claims 20-21,
an rAAV of any one of claims 22-27, or a combination thereof.

29. The composition of claim 28, wherein the composition is a pharmaceutical composition comprising one or more pharmaceutically acceptable carriers.

30. A composition for use in the delivery of an agent to a target environment of a subject in need, comprising an AAV comprising (1) an AAV capsid protein of any one of claims 18-19 and (2) an agent to be delivered to the target environment of the subject, wherein the target environment is the nervous system, the lung or a combination thereof.

31. The composition for use of claim 30, wherein the nervous system is the central nervous system (CNS), the peripheral nervous system (PNS), or a combination thereof.

32. The composition for use of any one of claims 30-31, wherein the target environment is a neuron, a glial cell, an oligodendrocyte, an ependymal cell, an astrocyte, a Schwann cell, a satellite cell, or an enteric glial cell in the CNS, a neuron or an astrocyte in the PNS, an endothelial cell in the lung, or a combination thereof.

33. The composition for use of any one of claims 30-32, wherein the agent is delivered to neural tissue in the CNS, ganglia or nerve fibre in the PNS, or epithelial lining of the lung or a combination thereof of the subject.

34. A composition for use in the delivery of an agent to a target environment of a subject in need, comprising an AAV comprising (1) an AAV capsid protein comprising an AAV targeting peptide that comprises at least 4 contiguous amino acids from the sequence of PNASVNS (SEQ ID NO: 387) and (2) an agent to be delivered to the target environment of the subject, wherein the target environment is the peripheral nervous system (PNS).

35. The composition for use of claim 34, wherein the target environment is a neuron or a glial cell in the PNS, or a combination thereof, and optionally the glial cell is an astrocyte.

36. The composition for use of any one of claims 34-35, wherein the agent is delivered to ganglia or nerve fibre in the PNS, or a combination thereof of the subject.

37. The composition for use of any one of claims 34-36, wherein the agent is delivered to dorsal root ganglia, nodose ganglia, or enteric ganglia in the peripheral nervous system (PNS), or a combination thereof of the subject.

38. A composition for use in the delivery of an agent to a target environment of a subject in need, comprising an AAV comprising (1) an AAV capsid protein comprising an AAV targeting peptide that comprises at least 4 contiguous amino acids from the sequence of LNTIRNV (SEQ ID NO: 388) and (2) an agent to be delivered to the target environment of the subject, wherein the target environment is the lung.

39. The composition for use of claim 38, wherein the target environment is an alveolar cell.

40. The composition for use of any one of claims 30-39, wherein the composition is a pharmaceutical composition comprising one or more pharmaceutically acceptable carriers.

41. The composition for use of any one of claims 30-40, wherein the agent to be delivered comprises a nucleic acid, a peptide, a small molecule, an aptamer, or a combination thereof.

42. The composition for use of claim 41, wherein the nucleic acid comprises one or more of:
a) a DNA sequence that encodes a trophic factor, a growth factor, or a soluble protein;
b) a cDNA that restores protein function to humans or animals harboring a genetic mutation(s) in that gene;
c) a cDNA that encodes a protein that can be used to control or alter the activity or state of a cell;
d) a cDNA that encodes a protein or a nucleic acid that can be used for assessing the state of a cell;
e) a cDNA that encodes a protein for gene editing, or a guide RNA;
f) a DNA sequence for genome editing via homologous recombination;
g) a DNA sequence encoding a therapeutic RNA;
h) an shRNA or an artificial miRNA delivery system; and
i) a DNA sequence that influences the splicing of an endogenous gene.

43. The composition for use of any one of claims 30-42, wherein the subject in need is a subject suffering from or at a risk to develop one or more of chronic pain, Friedreich's ataxia, Huntington's disease (HD), Alzheimer's disease (AD), Parkinson's disease (PD), Amyotrophic lateral sclerosis (ALS), spinal muscular atrophy types I and II (SMA I and II), Friedreich's Ataxia (FA), Spinocerebellar ataxia, multiple sclerosis (MS), chronic traumatic encephalopathy (CTE), HIV-1 associated dementia, lysosomal storage disorders that involve cells within the CNS, bronchiectasis, bronchitis, bronchopulmonary dysplasia, interstitial lung disease, occupational lung disease, cystic fibrosis, acute respiratory distress syndrome (ARDS), severe acute respiratory syndrome (SARS), intermittent asthma, mild persistent asthma, moderate persistent asthma, severe persistent asthma, chronic bronchitis, chronic obstructive pulmonary disease (COPD), interstitial lung disease, sarcoidosis, asbestosis, aspergilloma, aspergillosis, lobar pneumonia, multilobar pneumonia, bronchial pneumonia, interstitial pneumonia, pulmonary fibrosis, pulmonary tuberculosis, rheumatoid lung disease, pulmonary embolism, and non-small-cell lung carcinoma, adenocarcinoma, squamous-cell lung carcinoma, large-cell lung carcinoma, or small-cell lung carcinoma.

44. The composition for use of claim 43, wherein the lysosomal storage disorder is Krabbe disease, Sandhoff disease, Tay-Sachs, Gaucher disease (Type I, II or III), Niemann-Pick disease (NPC1 or NPC2 deficiency), Hurler syndrome, Pompe Disease, or Batten disease.

45. The composition for use of any one of claims 30-42, wherein the subject in need is a subject suffering from, at risk to develop, or has suffered from a stroke, traumatic brain injury, epilepsy, spinal cord injury, emphysema, lung reperfusion injury, ischemia-reperfusion injury of the lung, or ventilator-induced lung injury

46. The composition for use of any one of claims 30-45, wherein the composition is for intravenous administration and/or systemic administration.

47. The composition for use of any one of claims 30-46, wherein the subject is an adult animal.

48. A method of delivering an agent to a nervous system or a lung of a subject, the method comprising:
providing an AAV vector comprising an AAV capsid protein of any one of claims 18-19, wherein the AAV vector further comprises an agent to be delivered to the nervous system, the lung, or a combination thereof, and
administering the AAV vector to the subject.

49. The method of claim 48, wherein the agent is delivered to the nervous system or the lung of the subject at least 1.5-fold more efficiently than the delivery of the agent to other organs or tissues.

50. The method of claim 48, wherein the agent is delivered to the nervous system or the lung of the subject with an enrichment score relative to other organs or tissues of at least 0.1.

51. The method of claim 48, wherein the agent is delivered to the nervous system or lung of the subject at least 1.5-fold more efficiently than the agent is delivered to the nervous system or lung of the subject by an AAV vector that does not comprise the targeting peptide.

52. The method of any one of claims 48-51, wherein the nervous system is the central nervous system (CNS) and the peripheral nervous system (PNS).

53. The method of claim 52, wherein the agent is delivered to a neuron or an astrocyte of the nervous system of the subject at least 1.5-fold more efficiently than the delivery of the agent to other cells of the organs in which the PNS is embedded.

54. The method of claim 52, wherein the agent is delivered to a neuron or an astrocyte of the nervous system of the subject with an enrichment score relative to other cells of the organs in which the PNS is embedded of at least 0.1.

55. The method of claim 52, wherein the agent is delivered to a neuron or an astrocyte of the nervous system of the subject at least 1.5-fold more efficiently than the agent is delivered to a neuron or an astrocyte of the nervous system of the subject by an AAV vector that does not comprise the targeting peptide.

56. The method of claim 52, wherein the agent is delivered to the PNS in the gastrointestinal tract of the subject at least 1.5-fold more efficiently than the delivery of the agent to the nervous system or the lung than other organs or tissues.

57. The method of claim 52, wherein the agent is delivered to the peripheral nervous system (PNS) in the gastrointestinal tract of the subject with an enrichment score relative to other organs or tissues of at least 0.1.

58. The method of claim 52, wherein the agent is delivered to the PNS in the gastrointestinal tract of the subject at least 1.5-fold more efficiently than the agent is delivered to the PNS in the gastrointestinal tract of the subject by an AAV vector that does not comprise the targeting peptide.

59. A method of delivering an agent to a peripheral nervous system (PNS) of a subject, the method comprising:
providing an AAV vector comprising an AAV capsid protein, wherein the AAV capsid protein comprises an AAV targeting peptide that comprises at least 4 contiguous amino acids from the sequence of PNASVNS (SEQ ID NO: 387), wherein the AAV vector further comprises an agent to be delivered to the PNS; and
administering the AAV vector to the subject.

60. The method of claim 59, wherein the agent is delivered to a neuron or an astrocyte of the PNS of the subject at least 1.5-fold more efficiently than the delivery of the agent to the cells other than the neurons and the astrocytes in the organs in which the PNS is embedded.

61. The method of claim 59, wherein the agent is delivered to a neuron or an astrocyte of the PNS of the subject with an enrichment score relative to cells other than the neurons and the astrocytes in the organs in which the PNS is embedded of at least 0.1.

62. The method of claim 59, wherein the agent is delivered to the PNS in the gastrointestinal tract of the subject at least 1.5-fold more efficiently than the delivery of the agent to the nervous system or the lung than other organs or tissues.

63. The method of claim 59, wherein the agent is delivered to the PNS in the gastrointestinal tract of the subject with an enrichment score relative to other organs or tissues of at least 0.1.

64. The method of claim 59, wherein the agent is delivered to the PNS in the gastrointestinal tract of the subject at least 1.5-fold more efficiently than the agent is delivered to the PNS in the gastrointestinal tract of the subject by an AAV vector that does not comprise the targeting peptide.

65. A method of delivering an agent to a lung of a subject, the method comprising:
providing an AAV vector comprising an AAV capsid protein, wherein the AAV capsid protein comprises an AAV targeting peptide that comprises at least 4 contiguous amino acids from the sequence of LNTIRNV (SEQ ID NO: 388), wherein the AAV vector further comprises an agent to be delivered to the lung; and
administering the AAV vector to the subject.

66. The method of claim 65, wherein the agent is delivered to alveolar cells in the lung of the subject at least 1.5-fold more efficiently than the delivery of the agent to any cells in other organs or tissues.

67. The method of claim 65, wherein the agent is delivered to the lung of the subject with an enrichment score relative to other organs or tissues of at least 0.1.

68. The method of claim 65, wherein the agent is delivered to the lung of the subject at least 1.5-fold more efficiently than the agent is delivered to the lung of the subject by an AAV vector that does not comprise the targeting peptide.

69. The method of any one of claims 48-68, wherein the subject is a primate.

70. The method of any one of claims 48-69, wherein the administration is a systemic administration and/or an intravenous administration.

71. A method of delivering an agent to a cell, the method comprising: contacting an AAV vector comprising an AAV capsid protein of any one of claims 18-19 with the cell, wherein the AAV vector further comprises an agent to be delivered to a nervous system, a lung, or a combination thereof.

72. The method of claim 71, wherein the cell is a neuron, or an astrocyte in the nervous system, any cell in the lung or a combination thereof.

73. A method of delivering an agent to a cell, the method comprising: contacting an AAV vector comprising an AAV capsid protein with the cell, wherein the AAV capsid protein comprises an AAV targeting peptide that comprises at least 4 contiguous amino acids from the sequence of PNASVNS (SEQ ID NO: 387), wherein the AAV vector further comprises an agent to be delivered to a peripheral nervous system (PNS).

74. The method of claim 73, wherein the cell is a neuron, an astrocyte, or a combination thereof.

75. A method of delivering an agent to a cell, the method comprising: contacting an AAV vector comprising an AAV capsid protein with the cell, wherein the AAV capsid protein comprises an AAV targeting peptide that comprises at least 4 contiguous amino acids from the sequence of LNTIRNV (SEQ ID NO: 388), wherein the AAV vector further comprises an agent to be delivered to a lung.

76. The method of claim 75, wherein the cell is any cell in the lung.

77. The method of any one of claims 71-76, wherein contacting the AAV vector with the cell occurs in vitro, in vivo or ex vivo.

78. The method of any one of claims 71-77, wherein the cell is present in a tissue, an organ, or a subject.

79. The method of any one of claims 48-78, wherein the agent to be delivered comprises a nucleic acid, a peptide, a small molecule, an aptamer or a combination thereof.

80. The method of claim 79, wherein the nucleic acid encodes a therapeutic protein.

81. The method of claim 79, wherein the nucleic acid comprises one or more of:
a) a DNA sequence that encodes a trophic factor, a growth factor, or other soluble factors capable of being released from the transduced cells and affect the survival or function of that cell and/or surrounding cells;
b) a cDNA that restores protein function to humans or animals harboring a genetic mutation(s) in that gene;
c) a cDNA that encodes a protein that can be used to control or alter the activity or state of a cell;
d) a cDNA that encodes a protein or a nucleic acid that can be used for assessing the state of a cell;
e) a cDNA that encodes a protein for gene editing, or a guide RNA;
f) a DNA sequence for genome editing via homologous recombination;
g) a DNA sequence encoding a therapeutic RNA;
h) an shRNA or an artificial miRNA delivery system; and
i) a DNA sequence that influences the splicing of an endogenous gene.

82. The method of any one of claims 48-81, wherein the AAV vector is a vector selected from the group consisting of AAV1, AAV2, AAV3, AAV3b, AAV4, AAV5, AAV6, AAV7, AAV8, AAV9, AAV10, human isolate hu.31, human isolate hu.32, rhesus isolate rh.8, rhesus isolate rh.10, and variants thereof.

83. The method of any one of claims 48-82, wherein the serotype of the AAV vector is different from the serotype of the AAV capsid.

84. A adeno-associated virus (AAV) targeting peptide comprising an amino acid sequence of 7 contiguous amino acids X₁X₂X₃X₄X₅X₆X₇, wherein
X₁ is G;
X₂ is N;
X₃ is A, C, D, E, F, G, H, I, K, L, M, P, Q, S, T, V, W, or Y;
X₄ is A, C, D, E, F, G, H, I, K, L, M, N, P, Q, R, S, T, V, W, or Y;
X₅ is A, C, D, E, F, G, H, I, K, L, M, N, P, Q, R, S, T, V, W, or Y;
X₆ is R; and
X₇ is A, C, D, E, F, G, H, I, K, L, M, N, P, Q, R, S, T, V, W, or Y.

85. A adeno-associated virus (AAV) targeting peptide comprising an amino acid sequence of 7 contiguous amino acids X₁X₂X₃X₄X₅X₆X₇, wherein
X₁ is A, C, D, E, F, G, H, I, K, L, M, N, P, Q, R, T, V, W, or Y;
X₂ is N;
X₃ is R;
X₄ is A, C, D, E, F, G, H, I, K, L, M, N, P, Q, R, S, T, V, W, or Y;
X₅ is A, C, D, E, F, G, H, I, K, L, M, N, P, Q, R, S, T, V, W, or Y;
X₆ is A, C, D, E F, G, H, I, K, L, M, N, P, Q, R, S, T, V, W, or Y; and
X₇ is T.

86. A adeno-associated virus (AAV) targeting peptide comprising an amino acid sequence of 7 contiguous amino acids X₁X₂X₃X₄X₅X₆X₇, wherein
X₁ is A, C, D, E, F, G, H, I, K, L, M, N, P, Q, R, S, T, V, W, or Y;
X₂ is N;
X₃ is R;
X₄ is A, C, D, E, F, G, H, I, K, L, M, N, P, Q, R, S, T, V, W, or Y;
X₅ is A, C, D, E, F, G, H, I, K, L, M, N, P, Q, R, S, T, V, W, or Y;
X₆ is D; and
X₇ is A, C, D, E, F, G, H, I, K, L, M, N, P, Q, R, S, T, V, W, or Y.

87. A adeno-associated virus (AAV) targeting peptide comprising an amino acid sequence of 7 contiguous amino acids X₁X₂X₃X₄X₅X₆X₇, wherein
X₁ is S;
X₂ is N;
X₃ is R;
X₄ is A, C, D, E, F, G, H, I, K, L, M, N, P, Q, R, S, T, V, W, or Y;
X₅ is A, C, D, E, F, G, H, I, K, L, M, N, P, Q, R, S, T, V, W, or Y;
X₆ is A, C, D, E, F, G, H, I, K, L, M, N, P, Q, R, S, T, V, W, or Y; and
X₇ is A, C, D, E, F, G, H, I, K, L, M, N, P, Q, R, S, T, V, W, or Y.

88. A adeno-associated virus (AAV) targeting peptide comprising an amino acid sequence of 7 contiguous amino acids X₁X₂X₃X₄X₅X₆X₇, wherein
X₁ is K, L, S, H, I, Y, or N;
X₂ is N;
X₃ is R;
X₄ is K, A, T, D, M, H, or V;
X₅ is A, R, I, M, D, Y, or K;
X₆ is D; and
X₇ is A, G, R, S, V, D, or M.

89. A adeno-associated virus (AAV) targeting peptide comprising an amino acid sequence of 7 contiguous amino acids X₁X₂X₃X₄X₅X₆X₇, wherein
X₁ is S;
X₂ is N;
X₃ is R;
X₄ is R, V, P, T, E, F, or I;
X₅ is A, T, P, G, S, R, or V;
X₆ is F, H, P, I, L, T, or D; and
X₇ is A, E, I, Q, Y, V, or M.

90. A adeno-associated virus (AAV) targeting peptide comprising an amino acid sequence of 7 contiguous amino acids X₁X₂X₃X₄X₅X₆X₇, wherein
X₁ is L, M, A, K, G, T, or E;
X₂ is N;
X₃ is R;
X₄ is S, I, Q, N, A, G, or Y;
X₅ is V, K, S, E, D, A, or N;
X₆ is V, N, P, S, R, Q, or Y; and
X₇ is T.

91. A adeno-associated virus (AAV) targeting peptide comprising an amino acid sequence of 7 contiguous amino acids X₁X₂X₃X₄X₅X₆X₇, wherein
X₁ is G;
X₂ is N;
X₃ is Q, Y, L, T, A, I, or G;
X₄ is T, D, H, P, A, K, or Y;
X₅ is P, A, I, E, N, S, or Q;
X₆ is R; and
X₇ is K, G, M, S, A, P, or H.


SEQUENCE LISTING (452 sequences)

SEQ ID NO: 1 - protein, 7 aa, synthetic construct
TPGNRTP

SEQ ID NO: 2 - protein, 7 aa, synthetic construct
QIGSGGD

SEQ ID NO: 3 - protein, 7 aa, synthetic construct
SYTDGPK

SEQ ID NO: 4 - protein, 7 aa, synthetic construct
MHNNPAA

SEQ ID NO: 5 - protein, 7 aa, synthetic construct
AVTLHTE

SEQ ID NO: 6 - protein, 7 aa, synthetic construct
QLTNNLG

SEQ ID NO: 7 - protein, 7 aa, synthetic construct
RVNDPIL

SEQ ID NO: 8 - protein, 7 aa, synthetic construct
NYGTMNE

SEQ ID NO: 9 - protein, 7 aa, synthetic construct
HGTSITN

SEQ ID NO: 10 - protein, 7 aa, synthetic construct
WSAAAQE

SEQ ID NO: 11 - protein, 7 aa, synthetic construct
TSSQNVE

SEQ ID NO: 12 - protein, 7 aa, synthetic construct
NQPAVTA

SEQ ID NO: 13 - protein, 7 aa, synthetic construct
TMLASGE

SEQ ID NO: 14 - protein, 7 aa, synthetic construct
HDSNISS

SEQ ID NO: 15 - protein, 7 aa, synthetic construct
GDIIYTK

SEQ ID NO: 16 - protein, 7 aa, synthetic construct
LPETSNV

SEQ ID NO: 17 - protein, 7 aa, synthetic construct
DNTFLST

SEQ ID NO: 18 - protein, 7 aa, synthetic construct
YLEKMSA

SEQ ID NO: 19 - protein, 7 aa, synthetic construct
YDASQLA

SEQ ID NO: 20 - protein, 7 aa, synthetic construct
LDATYGS

SEQ ID NO: 21 - protein, 7 aa, synthetic construct
TGHNQLA

SEQ ID NO: 22 - protein, 7 aa, synthetic construct
STAAPRL

SEQ ID NO: 23 - protein, 7 aa, synthetic construct
NITAIPN

SEQ ID NO: 24 - protein, 7 aa, synthetic construct
TGTSIHP

SEQ ID NO: 25 - protein, 7 aa, synthetic construct
MSVASQH

SEQ ID NO: 26 - protein, 7 aa, synthetic construct
LPKPVIS

SEQ ID NO: 27 - protein, 7 aa, synthetic construct
DPRTSLD

SEQ ID NO: 28 - protein, 7 aa, synthetic construct
ANINKEN

SEQ ID NO: 29 - protein, 7 aa, synthetic construct
ASNSPAL

SEQ ID NO: 30 - protein, 7 aa, synthetic construct
SNLMSDF

SEQ ID NO: 31 - protein, 7 aa, synthetic construct
VAASVSS

SEQ ID NO: 32 - protein, 7 aa, synthetic construct
QRIEFSS

SEQ ID NO: 33 - protein, 7 aa, synthetic construct
SMTPTSV

SEQ ID NO: 34 - protein, 7 aa, synthetic construct
TTSTVTS

SEQ ID NO: 35 - protein, 7 aa, synthetic construct
HDNFGSA

SEQ ID NO: 36 - protein, 7 aa, synthetic construct
AEAKHPP

SEQ ID NO: 37 - protein, 7 aa, synthetic construct
LKAQLPD

SEQ ID NO: 38 - protein, 7 aa, synthetic construct
DINPTYS

SEQ ID NO: 39 - protein, 7 aa, synthetic construct
SSASLLA

SEQ ID NO: 40 - protein, 7 aa, synthetic construct
GNQTPSV

SEQ ID NO: 41 - protein, 7 aa, synthetic construct
IGPTVRM

SEQ ID NO: 42 - protein, 7 aa, synthetic construct
GIPTRFS

SEQ ID NO: 43 - protein, 7 aa, synthetic construct
RESLPGH

SEQ ID NO: 44 - protein, 7 aa, synthetic construct
GDTLREC

SEQ ID NO: 45 - protein, 7 aa, synthetic construct
VPSMTVE

SEQ ID NO: 46 - protein, 7 aa, synthetic construct
TDQAFRS

SEQ ID NO: 47 - protein, 7 aa, synthetic construct
GVNDHVH

SEQ ID NO: 48 - protein, 7 aa, synthetic construct
LPANQGT

SEQ ID NO: 49 - protein, 7 aa, synthetic construct
TLLPRSN

SEQ ID NO: 50 - protein, 7 aa, synthetic construct
GSGLAGN

SEQ ID NO: 51 - protein, 7 aa, synthetic construct
HMPSANY

SEQ ID NO: 52 - protein, 7 aa, synthetic construct
LSGSGSA

SEQ ID NO: 53 - protein, 7 aa, synthetic construct
VFDLSRT

SEQ ID NO: 54 - protein, 7 aa, synthetic construct
TALTGPT

SEQ ID NO: 55 - protein, 7 aa, synthetic construct
TLNPKSP

SEQ ID NO: 56 - protein, 7 aa, synthetic construct
TGPTMSL

SEQ ID NO: 57 - protein, 7 aa, synthetic construct
RAKILAS

SEQ ID NO: 58 - protein, 7 aa, synthetic construct
LAIASAP

SEQ ID NO: 59 - protein, 7 aa, synthetic construct
SKNDAVA

SEQ ID NO: 60 - protein, 7 aa, synthetic construct
VPATDDK

SEQ ID NO: 61 - protein, 7 aa, synthetic construct
LSISLGD

SEQ ID NO: 62 - protein, 7 aa, synthetic construct
QASNLEM

SEQ ID NO: 63 - protein, 7 aa, synthetic construct
DLLSNIA

SEQ ID NO: 64 - protein, 7 aa, synthetic construct
LSSALPK

SEQ ID NO: 65 - protein, 7 aa, synthetic construct
HTTLGIN

SEQ ID NO: 66 - protein, 7 aa, synthetic construct
LHVNSTN

SEQ ID NO: 67 - protein, 7 aa, synthetic construct
PVTQLSR

SEQ ID NO: 68 - protein, 7 aa, synthetic construct
IALNSEP

SEQ ID NO: 69 - protein, 7 aa, synthetic construct
QEIPRLN

SEQ ID NO: 70 - protein, 7 aa, synthetic construct
INSTLQF

SEQ ID NO: 71 - protein, 7 aa, synthetic construct
AVPAKER

SEQ ID NO: 72 - protein, 7 aa, synthetic construct
GQNKEPG

SEQ ID NO: 73 - protein, 7 aa, synthetic construct
DPQNSGL

SEQ ID NO: 74 - protein, 7 aa, synthetic construct
DELQSRS

SEQ ID NO: 75 - protein, 7 aa, synthetic construct
MQTTERE

SEQ ID NO: 76 - protein, 7 aa, synthetic construct
ELSHQLA

SEQ ID NO: 77 - protein, 7 aa, synthetic construct
TSAQGTQ

SEQ ID NO: 78 - protein, 7 aa, synthetic construct
LNKESSW

SEQ ID NO: 79 - protein, 7 aa, synthetic construct
ESSPHRT

SEQ ID NO: 80 - protein, 7 aa, synthetic construct
RSLSDFS

SEQ ID NO: 81 - protein, 7 aa, synthetic construct
TLIDSAA

SEQ ID NO: 82 - protein, 7 aa, synthetic construct
ADTMSFR

SEQ ID NO: 83 - protein, 7 aa, synthetic construct
DVAGRGF

SEQ ID NO: 84 - protein, 7 aa, synthetic construct
LLGAEPP

SEQ ID NO: 85 - protein, 7 aa, synthetic construct
QLSDAMA

SEQ ID NO: 86 - protein, 7 aa, synthetic construct
GSEIYRG

SEQ ID NO: 87 - protein, 7 aa, synthetic construct
DGVSHTL

SEQ ID NO: 88 - protein, 7 aa, synthetic construct
VVHEASL

SEQ ID NO: 89 - protein, 7 aa, synthetic construct
ILQSHPL

SEQ ID NO: 90 - protein, 7 aa, synthetic construct
ATYVTST

SEQ ID NO: 91 - protein, 7 aa, synthetic construct
TAMQLGH

SEQ ID NO: 92 - protein, 7 aa, synthetic construct
PVNSTGA

SEQ ID NO: 93 - protein, 7 aa, synthetic construct
RPVSPRA

SEQ ID NO: 94 - protein, 7 aa, synthetic construct
IPASPFT

SEQ ID NO: 95 - protein, 7 aa, synthetic construct
ANDYRYD

SEQ ID NO: 96 - protein, 7 aa, synthetic construct
YNSEPRI

SEQ ID NO: 97 - protein, 7 aa, synthetic construct
AIETRNV

SEQ ID NO: 98 - protein, 7 aa, synthetic construct
PASQGLS

SEQ ID NO: 99 - protein, 7 aa, synthetic construct
RDSDALR

SEQ ID NO: 100 - protein, 7 aa, synthetic construct
CQAPKAD

SEQ ID NO: 101 - protein, 7 aa, synthetic construct
ALSGAVT

SEQ ID NO: 102 - protein, 7 aa, synthetic construct
VQPIFPL

SEQ ID NO: 103 - protein, 7 aa, synthetic construct
SVLGNAS

SEQ ID NO: 104 - protein, 7 aa, synthetic construct
APRLDAA

SEQ ID NO: 105 - protein, 7 aa, synthetic construct
SASSIVN

SEQ ID NO: 106 - protein, 7 aa, synthetic construct
GVHVKPN

SEQ ID NO: 107 - protein, 7 aa, synthetic construct
LQVSEPR

SEQ ID NO: 108 - protein, 7 aa, synthetic construct
AGAHVPS

SEQ ID NO: 109 - protein, 7 aa, synthetic construct
PLMGTAS

SEQ ID NO: 110 - protein, 7 aa, synthetic construct
QVPTWNS

SEQ ID NO: 111 - protein, 7 aa, synthetic construct
DHRTTYK

SEQ ID NO: 112 - protein, 7 aa, synthetic construct
HPEKEAA

SEQ ID NO: 113 - protein, 7 aa, synthetic construct
AGPGLGL

SEQ ID NO: 114 - protein, 7 aa, synthetic construct
ITPTPSS

SEQ ID NO: 115 - protein, 7 aa, synthetic construct
TGMSVVH

SEQ ID NO: 116 - protein, 7 aa, synthetic construct
LMDASGT

SEQ ID NO: 117 - protein, 7 aa, synthetic construct
SPYPNTS

SEQ ID NO: 118 - protein, 7 aa, synthetic construct
SNHAPVS

SEQ ID NO: 119 - protein, 7 aa, synthetic construct
ASGLALA

SEQ ID NO: 120 - protein, 7 aa, synthetic construct
PTAATGT

SEQ ID NO: 121 - protein, 7 aa, synthetic construct
DFVHVAN

SEQ ID NO: 122 - protein, 7 aa, synthetic construct
LTNSRFM

SEQ ID NO: 123 - protein, 7 aa, synthetic construct
LIIPSLP

SEQ ID NO: 124 - protein, 7 aa, synthetic construct
GTLTMQT

SEQ ID NO: 125 - protein, 7 aa, synthetic construct
GTDHYTA

SEQ ID NO: 126 - protein, 7 aa, synthetic construct
ALPKGTT

SEQ ID NO: 127 - protein, 7 aa, synthetic construct
FLAPVQS

SEQ ID NO: 128 - protein, 7 aa, synthetic construct
HDAPSIS

SEQ ID NO: 129 - protein, 7 aa, synthetic construct
VTIGPQR

SEQ ID NO: 130 - protein, 7 aa, synthetic construct
VDHGTMA

SEQ ID NO: 131 - protein, 7 aa, synthetic construct
TDSVSTT

SEQ ID NO: 132 - protein, 7 aa, synthetic construct
STVAIVP

SEQ ID NO: 133 - protein, 7 aa, synthetic construct
ALQADRA

SEQ ID NO: 134 - protein, 7 aa, synthetic construct
DPRLSVE

SEQ ID NO: 135 - protein, 7 aa, synthetic construct
NTQTPAS

SEQ ID NO: 136 - protein, 7 aa, synthetic construct
SGNGGMH

SEQ ID NO: 137 - protein, 7 aa, synthetic construct
KNLLESI

SEQ ID NO: 138 - protein, 7 aa, synthetic construct
NSSDSAY

SEQ ID NO: 139 - protein, 7 aa, synthetic construct
LGYATAE

SEQ ID NO: 140 - protein, 7 aa, synthetic construct
VLDHSAD

SEQ ID NO: 141 - protein, 7 aa, synthetic construct
LLGALAP

SEQ ID NO: 142 - protein, 7 aa, synthetic construct
HIPDRQH

SEQ ID NO: 143 - protein, 7 aa, synthetic construct
LPASSGL

SEQ ID NO: 144 - protein, 7 aa, synthetic construct
PVTTSTS

SEQ ID NO: 145 - protein, 7 aa, synthetic construct
AHNANMA

SEQ ID NO: 146 - protein, 7 aa, synthetic construct
LLQNLSL

SEQ ID NO: 147 - protein, 7 aa, synthetic construct
TVTMPAS

SEQ ID NO: 148 - protein, 7 aa, synthetic construct
LSLSHNQ

SEQ ID NO: 149 - protein, 7 aa, synthetic construct
SHPPSLP

SEQ ID NO: 150 - protein, 7 aa, synthetic construct
PEPNGTP

SEQ ID NO: 151 - protein, 7 aa, synthetic construct
TDQVMLY

SEQ ID NO: 152 - protein, 7 aa, synthetic construct
EKMLVGA

SEQ ID NO: 153 - protein, 7 aa, synthetic construct
PQSHSRQ

SEQ ID NO: 154 - protein, 7 aa, synthetic construct
LQAISLS

SEQ ID NO: 155 - protein, 7 aa, synthetic construct
IGPTDRM

SEQ ID NO: 156 - protein, 7 aa, synthetic construct
IVNDSVG

SEQ ID NO: 157 - protein, 7 aa, synthetic construct
TNGAAGW

SEQ ID NO: 158 - protein, 7 aa, synthetic construct
TGIAVQS

SEQ ID NO: 159 - protein, 7 aa, synthetic construct
SSGTPSW

SEQ ID NO: 160 - protein, 7 aa, synthetic construct
LLTLAPS

SEQ ID NO: 161 - protein, 7 aa, synthetic construct
SWSERPV

SEQ ID NO: 162 - protein, 7 aa, synthetic construct
NCPMKSQ

SEQ ID NO: 163 - protein, 7 aa, synthetic construct
KLSSGLD

SEQ ID NO: 164 - protein, 7 aa, synthetic construct
TTGMIST

SEQ ID NO: 165 - protein, 7 aa, synthetic construct
MAHDTQS

SEQ ID NO: 166 - protein, 7 aa, synthetic construct
ILSLAES

SEQ ID NO: 167 - protein, 7 aa, synthetic construct
GGDVLTL

SEQ ID NO: 168 - protein, 7 aa, synthetic construct
LSQGTQT

SEQ ID NO: 169 - protein, 7 aa, synthetic construct
PHEGSSR

SEQ ID NO: 170 - protein, 7 aa, synthetic construct
QIVNNTY

SEQ ID NO: 171 - protein, 7 aa, synthetic construct
GLLATNA

SEQ ID NO: 172 - protein, 7 aa, synthetic construct
QGDTSGL

SEQ ID NO: 173 - protein, 7 aa, synthetic construct
KVSTPLR

SEQ ID NO: 174 - protein, 7 aa, synthetic construct
SNAMSST

SEQ ID NO: 175 - protein, 7 aa, synthetic construct
LTALEMM

SEQ ID NO: 176 - protein, 7 aa, synthetic construct
ILSIDQS

SEQ ID NO: 177 - protein, 7 aa, synthetic construct
PLTLAPK

SEQ ID NO: 178 - protein, 7 aa, synthetic construct
YVAEAAV

SEQ ID NO: 179 - protein, 7 aa, synthetic construct
GKQSWAD

SEQ ID NO: 180 - protein, 7 aa, synthetic construct
NLEDKST

SEQ ID NO: 181 - protein, 7 aa, synthetic construct
YSNSQHH

SEQ ID NO: 182 - protein, 7 aa, synthetic construct
KVTPSTP

SEQ ID NO: 183 - protein, 7 aa, synthetic construct
KTNVPGD

SEQ ID NO: 184 - protein, 7 aa, synthetic construct
NRTTVDN

SEQ ID NO: 185 - protein, 7 aa, synthetic construct
LIPEPPK

SEQ ID NO: 186 - protein, 7 aa, synthetic construct
HSPMAIN

SEQ ID NO: 187 - protein, 7 aa, synthetic construct
YAPQMPF

SEQ ID NO: 188 - protein, 7 aa, synthetic construct
DTLQYPR

SEQ ID NO: 189 - protein, 7 aa, synthetic construct
IIVSECS

SEQ ID NO: 190 - protein, 7 aa, synthetic construct
WLDKLSK

SEQ ID NO: 191 - protein, 7 aa, synthetic construct
PVQHVSN

SEQ ID NO: 192 - protein, 7 aa, synthetic construct
AVENTTP

SEQ ID NO: 193 - protein, 7 aa, synthetic construct
IDTTSCC

SEQ ID NO: 194 - protein, 7 aa, synthetic construct
GQLVTNL

SEQ ID NO: 195 - protein, 7 aa, synthetic construct
NGTGSTT

SEQ ID NO: 196 - protein, 7 aa, synthetic construct
STPTVEP

SEQ ID NO: 197 - protein, 7 aa, synthetic construct
MNGQLTC

SEQ ID NO: 198 - protein, 7 aa, synthetic construct
GNFLRAD

SEQ ID NO: 199 - protein, 7 aa, synthetic construct
TGLPHNE

SEQ ID NO: 200 - protein, 7 aa, synthetic construct
TSGEDAL

SEQ ID NO: 201 - protein, 7 aa, synthetic construct
AAVSSIP

SEQ ID NO: 202 - protein, 7 aa, synthetic construct
ANYTVGK

SEQ ID NO: 203 - protein, 7 aa, synthetic construct
AQNSTRA

SEQ ID NO: 204 - protein, 7 aa, synthetic construct
ASARVNV

SEQ ID NO: 205 - protein, 7 aa, synthetic construct
DNRSKMI

SEQ ID NO: 206 - protein, 7 aa, synthetic construct
DNTTVMR

SEQ ID NO: 207 - protein, 7 aa, synthetic construct
DNTVRHN

SEQ ID NO: 208 - protein, 7 aa, synthetic construct
DTAPTRS

SEQ ID NO: 209 - protein, 7 aa, synthetic construct
EGTKSMS

SEQ ID NO: 210 - protein, 7 aa, synthetic construct
EIRSSSY

SEQ ID NO: 211 - protein, 7 aa, synthetic construct
ELRRSAN

SEQ ID NO: 212 - protein, 7 aa, synthetic construct
FRTTGNA

SEQ ID NO: 213 - protein, 7 aa, synthetic construct
GADLRNP

SEQ ID NO: 214 - protein, 7 aa, synthetic construct
GFHTTSG

SEQ ID NO: 215 - protein, 7 aa, synthetic construct
GGTGSRI

SEQ ID NO: 216 - protein, 7 aa, synthetic construct
GLKTSGL

SEQ ID NO: 217 - protein, 7 aa, synthetic construct
GMTSKAT

SEQ ID NO: 218 - protein, 7 aa, synthetic construct
GNSVFPD

SEQ ID NO: 219 - protein, 7 aa, synthetic construct
GNVNNRV

SEQ ID NO: 220 - protein, 7 aa, synthetic construct
GQGHTSP

SEQ ID NO: 221 - protein, 7 aa, synthetic construct
GQNPLRQ

SEQ ID NO: 222 - protein, 7 aa, synthetic construct
GTNSRPN

SEQ ID NO: 223 - protein, 7 aa, synthetic construct
HENFTRS

SEQ ID NO: 224 - protein, 7 aa, synthetic construct
HSASGAA

SEQ ID NO: 225 - protein, 7 aa, synthetic construct
IATIGSK

SEQ ID NO: 226 - protein, 7 aa, synthetic construct
IGGSASQ

SEQ ID NO: 227 - protein, 7 aa, synthetic construct
IGPTGRM

SEQ ID NO: 228 - protein, 7 aa, synthetic construct
INSARDT

SEQ ID NO: 229 - protein, 7 aa, synthetic construct
IRSLERD

SEQ ID NO: 230 - protein, 7 aa, synthetic construct
KMASTQS

SEQ ID NO: 231 - protein, 7 aa, synthetic construct
KNEAITC

SEQ ID NO: 232 - protein, 7 aa, synthetic construct
KNGLAQN

SEQ ID NO: 233 - protein, 7 aa, synthetic construct
KQLTASA

SEQ ID NO: 234 - protein, 7 aa, synthetic construct
KTPSVAS

SEQ ID NO: 235 - protein, 7 aa, synthetic construct
KVTLTSN

SEQ ID NO: 236 - protein, 7 aa, synthetic construct
LNGVSSR

SEQ ID NO: 237 - protein, 7 aa, synthetic construct
LNNTKTT

SEQ ID NO: 238 - protein, 7 aa, synthetic construct
LNSVRRD

SEQ ID NO: 239 - protein, 7 aa, synthetic construct
LQSKLNS

SEQ ID NO: 240 - protein, 7 aa, synthetic construct
LRTGELT

SEQ ID NO: 241 - protein, 7 aa, synthetic construct
LSTRQTT

SEQ ID NO: 242 - protein, 7 aa, synthetic construct
LTGVGSQ

SEQ ID NO: 243 - protein, 7 aa, synthetic construct
LTKTVSA

SEQ ID NO: 244 - protein, 7 aa, synthetic construct
LTNGSNY

SEQ ID NO: 245 - protein, 7 aa, synthetic construct
LTYATVG

SEQ ID NO: 246 - protein, 7 aa, synthetic construct
LVQNNTM

SEQ ID NO: 247 - protein, 7 aa, synthetic construct
MNRTLST

SEQ ID NO: 248 - protein, 7 aa, synthetic construct
MNVRQSA

SEQ ID NO: 249 - protein, 7 aa, synthetic construct
MRLERAG

SEQ ID NO: 250 - protein, 7 aa, synthetic construct
MTRSSGY

SEQ ID NO: 251 - protein, 7 aa, synthetic construct
NKTSNTP

SEQ ID NO: 252 - protein, 7 aa, synthetic construct
NLKTGAT

SEQ ID NO: 253 - protein, 7 aa, synthetic construct
NSHQNTI

SEQ ID NO: 254 - protein, 7 aa, synthetic construct
NSLKNVA

SEQ ID NO: 255 - protein, 7 aa, synthetic construct
NSVKNVL

SEQ ID NO: 256 - protein, 7 aa, synthetic construct
NSVSRDI

SEQ ID NO: 257 - protein, 7 aa, synthetic construct
NTKNSSG

SEQ ID NO: 258 - protein, 7 aa, synthetic construct
NVGKLQP

SEQ ID NO: 259 - protein, 7 aa, synthetic construct
PAPGRVS

SEQ ID NO: 260 - protein, 7 aa, synthetic construct
PNGYATV

SEQ ID NO: 261 - protein, 7 aa, synthetic construct
PNTQVRA

SEQ ID NO: 262 - protein, 7 aa, synthetic construct
PPSGQAL

SEQ ID NO: 263 - protein, 7 aa, synthetic construct
PRTDKGS

SEQ ID NO: 264 - protein, 7 aa, synthetic construct
PSPQLRS

SEQ ID NO: 265 - protein, 7 aa, synthetic construct
PVNTQRV

SEQ ID NO: 266 - protein, 7 aa, synthetic construct
QKSQQHG

SEQ ID NO: 267 - protein, 7 aa, synthetic construct
QRESVKV

SEQ ID NO: 268 - protein, 7 aa, synthetic construct
RHTVNPS

SEQ ID NO: 269 - protein, 7 aa, synthetic construct
RSQHTTS

SEQ ID NO: 270 - protein, 7 aa, synthetic construct
SAVTIKA

SEQ ID NO: 271 - protein, 7 aa, synthetic construct
SGKTPHT

SEQ ID NO: 272 - protein, 7 aa, synthetic construct
SLGKVEN

SEQ ID NO: 273 - protein, 7 aa, synthetic construct
SNATHNT

SEQ ID NO: 274 - protein, 7 aa, synthetic construct
SNLARNV

SEQ ID NO: 275 - protein, 7 aa, synthetic construct
SRLNTPG

SEQ ID NO: 276 - protein, 7 aa, synthetic construct
SSNVARG

SEQ ID NO: 277 - protein, 7 aa, synthetic construct
SSRSAQA

SEQ ID NO: 278 - protein, 7 aa, synthetic construct
STISTRP

SEQ ID NO: 279 - protein, 7 aa, synthetic construct
SYLASAS

SEQ ID NO: 280 - protein, 7 aa, synthetic construct
TFSQTRE

SEQ ID NO: 281 - protein, 7 aa, synthetic construct
TGHNQMA

SEQ ID NO: 282 - protein, 7 aa, synthetic construct
TGNSSGT

SEQ ID NO: 283 - protein, 7 aa, synthetic construct
TKISPGS

SEQ ID NO: 284 - protein, 7 aa, synthetic construct
TMNLAKA

SEQ ID NO: 285 - protein, 7 aa, synthetic construct
TQRLPST

SEQ ID NO: 286 - protein, 7 aa, synthetic construct
TQRSNYE

SEQ ID NO: 287 - protein, 7 aa, synthetic construct
TRGSYPP

SEQ ID NO: 288 - protein, 7 aa, synthetic construct
TRMDRGQ

SEQ ID NO: 289 - protein, 7 aa, synthetic construct
TRSSLSG

SEQ ID NO: 290 - protein, 7 aa, synthetic construct
TRTVTSA

SEQ ID NO: 291 - protein, 7 aa, synthetic construct
TSQSLIG

SEQ ID NO: 292 - protein, 7 aa, synthetic construct
TSTRQST

SEQ ID NO: 293 - protein, 7 aa, synthetic construct
VNHVARP

SEQ ID NO: 294 - protein, 7 aa, synthetic construct
VNTAKPF

SEQ ID NO: 295 - protein, 7 aa, synthetic construct
VRTSQSS

SEQ ID NO: 296 - protein, 7 aa, synthetic construct
VVITGRS

SEQ ID NO: 297 - protein, 7 aa, synthetic construct
VVTKSLA

SEQ ID NO: 298 - protein, 7 aa, synthetic construct
WSGSSLS

SEQ ID NO: 299 - protein, 7 aa, synthetic construct
YNSASRN

SEQ ID NO: 300 - protein, 7 aa, synthetic construct
YNTTRER

SEQ ID NO: 301 - protein, 7 aa, synthetic construct
YRLSNDA

SEQ ID NO: 302 - protein, 7 aa, synthetic construct
ANNSRPF

SEQ ID NO: 303 - protein, 7 aa, synthetic construct
ANRIRDN

SEQ ID NO: 304 - protein, 7 aa, synthetic construct
ANRTKDY

SEQ ID NO: 305 - protein, 7 aa, synthetic construct
ANTIRNV

SEQ ID NO: 306 - protein, 7 aa, synthetic construct
DNHVMAR

SEQ ID NO: 307 - protein, 7 aa, synthetic construct
DNTTRLK

SEQ ID NO: 308 - protein, 7 aa, synthetic construct
DRNTTVR

SEQ ID NO: 309 - protein, 7 aa, synthetic construct
ENRTVPT

SEQ ID NO: 310 - protein, 7 aa, synthetic construct
ENRVKSA

SEQ ID NO: 311 - protein, 7 aa, synthetic construct
ENTTRIA

SEQ ID NO: 312 - protein, 7 aa, synthetic construct
GNATPRS

SEQ ID NO: 313 - protein, 7 aa, synthetic construct
GNAVRST

SEQ ID NO: 314 - protein, 7 aa, synthetic construct
GNDIRSR

SEQ ID NO: 315 - protein, 7 aa, synthetic construct
GNHARNL

SEQ ID NO: 316 - protein, 7 aa, synthetic construct
GNMIKVA

SEQ ID NO: 317 - protein, 7 aa, synthetic construct
GNMTKFT

SEQ ID NO: 318 - protein, 7 aa, synthetic construct
GNNSRNM

SEQ ID NO: 319 - protein, 7 aa, synthetic construct
GNNTAKL

SEQ ID NO: 320 - protein, 7 aa, synthetic construct
GNQILRL

SEQ ID NO: 321 - protein, 7 aa, synthetic construct
GNQTNRS

SEQ ID NO: 322 - protein, 7 aa, synthetic construct
GNRISNT

SEQ ID NO: 323 - protein, 7 aa, synthetic construct
GNRTTSS

SEQ ID NO: 324 - protein, 7 aa, synthetic construct
GNSVRLI

SEQ ID NO: 325 - protein, 7 aa, synthetic construct
GNWVKDI

SEQ ID NO: 326 - protein, 7 aa, synthetic construct
GNYVSRP

SEQ ID NO: 327 - protein, 7 aa, synthetic construct
HNLTRNL

SEQ ID NO: 328 - protein, 7 aa, synthetic construct
LNAVKNV

SEQ ID NO: 329 - protein, 7 aa, synthetic construct
LNSTRNT

SEQ ID NO: 330 - protein, 7 aa, synthetic construct
LYSNKSN

SEQ ID NO: 331 - protein, 7 aa, synthetic construct
MNSTKNV

SEQ ID NO: 332 - protein, 7 aa, synthetic construct
NHIRAMQ

SEQ ID NO: 333 - protein, 7 aa, synthetic construct
NHTRMNE

SEQ ID NO: 334 - protein, 7 aa, synthetic construct
NNATRYT

SEQ ID NO: 335 - protein, 7 aa, synthetic construct
NNLRPVY

SEQ ID NO: 336 - protein, 7 aa, synthetic construct
NNLTRVT

SEQ ID NO: 337 - protein, 7 aa, synthetic construct
NNNTKMN

SEQ ID NO: 338 - protein, 7 aa, synthetic construct
NNRTTNN

SEQ ID NO: 339 - protein, 7 aa, synthetic construct
NNSLRSN

SEQ ID NO: 340 - protein, 7 aa, synthetic construct
NNTARSV

SEQ ID NO: 341 - protein, 7 aa, synthetic construct
NNTVMRS

SEQ ID NO: 342 - protein, 7 aa, synthetic construct
NSTRIIV

SEQ ID NO: 343 - protein, 7 aa, synthetic construct
NSVRQSL

SEQ ID NO: 344 - protein, 7 aa, synthetic construct
NTVRSST

SEQ ID NO: 345 - protein, 7 aa, synthetic construct
QNLTKGQ

SEQ ID NO: 346 - protein, 7 aa, synthetic construct
QNQSRFT

SEQ ID NO: 347 - protein, 7 aa, synthetic construct
SANATKL

SEQ ID NO: 348 - protein, 7 aa, synthetic construct
SNAIRPV

SEQ ID NO: 349 - protein, 7 aa, synthetic construct
SNATMRF

SEQ ID NO: 350 - protein, 7 aa, synthetic construct
SNHINRV

SEQ ID NO: 351 - protein, 7 aa, synthetic construct
SNHTRSY

SEQ ID NO: 352 - protein, 7 aa, synthetic construct
SNNSRPN

SEQ ID NO: 353 - protein, 7 aa, synthetic construct
SNNTRPT

SEQ ID NO: 354 - protein, 7 aa, synthetic construct
SNQIRDS

SEQ ID NO: 355 - protein, 7 aa, synthetic construct
SNRIVPV

SEQ ID NO: 356 - protein, 7 aa, synthetic construct
SNRKIDD

SEQ ID NO: 357 - protein, 7 aa, synthetic construct
SNRTVRE

SEQ ID NO: 358 - protein, 7 aa, synthetic construct
SNRVRDL

SEQ ID NO: 359 - protein, 7 aa, synthetic construct
SNSTKAI

SEQ ID NO: 360 - protein, 7 aa, synthetic construct
SNSTKPN

SEQ ID NO: 361 - protein, 7 aa, synthetic construct
SNSTSRA

SEQ ID NO: 362 - protein, 7 aa, synthetic construct
SNYTKII

SEQ ID NO: 363 - protein, 7 aa, synthetic construct
TNFTKSG

SEQ ID NO: 364 - protein, 7 aa, synthetic construct
TNHTRNT

SEQ ID NO: 365 - protein, 7 aa, synthetic construct
TNHVKFT

SEQ ID NO: 366 - protein, 7 aa, synthetic construct
TNNTRNT

SEQ ID NO: 367 - protein, 7 aa, synthetic construct
TNQSKNF

SEQ ID NO: 368 - protein, 7 aa, synthetic construct
TNRTIQT

SEQ ID NO: 369 - protein, 7 aa, synthetic construct
TNRTQAH

SEQ ID NO: 370 - protein, 7 aa, synthetic construct
TNRTQLY

SEQ ID NO: 371 - protein, 7 aa, synthetic construct
TNSTLRQ

SEQ ID NO: 372 - protein, 7 aa, synthetic construct
TNSTTKS

SEQ ID NO: 373 - protein, 7 aa, synthetic construct
TNTTRAM

SEQ ID NO: 374 - protein, 7 aa, synthetic construct
TNVTRST

SEQ ID NO: 375 - protein, 7 aa, synthetic construct
VNKVRDV

SEQ ID NO: 376 - protein, 7 aa, synthetic construct
VNSVRMT

SEQ ID NO: 377 - protein, 7 aa, synthetic construct
VNTTRFS

SEQ ID NO: 378 - protein, 7 aa, synthetic construct
VQSNRTN

SEQ ID NO: 379 - protein, 7 aa, synthetic construct
WGNRIQI

SEQ ID NO: 380 - protein, 11 aa, synthetic construct
AQGNNTRPTAH

SEQ ID NO: 381 - protein, 11 aa, synthetic construct
AQGNRTTSSEQ

SEQ ID NO: 382 - protein, 11 aa, synthetic construct
AQSNAVRANSQ

SEQ ID NO: 383 - protein, 11 aa, synthetic construct
AQTNDTRRISQ

SEQ ID NO: 384 - protein, 11 aa, synthetic construct
AQTNNTKPLAH

SEQ ID NO: 385 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQPHEGSSRAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 386 - protein, 743 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSRTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQTNNTKPLAHAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 387 - protein, 7 aa, synthetic construct
PNASVNS

SEQ ID NO: 388 - protein, 7 aa, synthetic construct
LNTIRNV

SEQ ID NO: 389 - protein, 7 aa, synthetic construct
QAVRTSL

SEQ ID NO: 390 - protein, 7 aa, synthetic construct
TNNTKPL

SEQ ID NO: 391 - protein, 736 aa, synthetic construct
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 392 - protein, 4 aa, synthetic construct
PHEG

SEQ ID NO: 393 - protein, 4 aa, synthetic construct
HEGS

SEQ ID NO: 394 - protein, 4 aa, synthetic construct
EGSS

SEQ ID NO: 395 - protein, 4 aa, synthetic construct
GSSR

SEQ ID NO: 396 - protein, 5 aa, synthetic construct
PHEGS

SEQ ID NO: 397 - protein, 5 aa, synthetic construct
HEGSS

SEQ ID NO: 398 - protein, 5 aa, synthetic construct
EGSSR

SEQ ID NO: 399 - protein, 6 aa, synthetic construct
PHEGSS

SEQ ID NO: 400 - protein, 6 aa, synthetic construct
HEGSSR

SEQ ID NO: 401 - protein, 4 aa, synthetic construct
LNNT

SEQ ID NO: 402 - protein, 4 aa, synthetic construct
NNTK

SEQ ID NO: 403 - protein, 4 aa, synthetic construct
NTKT

SEQ ID NO: 404 - protein, 4 aa, synthetic construct
TKTT

SEQ ID NO: 405 - protein, 5 aa, synthetic construct
LNNTK

SEQ ID NO: 406 - protein, 5 aa, synthetic construct
NNTKT

SEQ ID NO: 407 - protein, 5 aa, synthetic construct
NTKTT

SEQ ID NO: 408 - protein, 6 aa, synthetic construct
LNNTKT

SEQ ID NO: 409 - protein, 6 aa, synthetic construct
NNTKTT

SEQ ID NO: 410 - protein, 4 aa, synthetic construct
SNLA

SEQ ID NO: 411 - protein, 4 aa, synthetic construct
NLAR

SEQ ID NO: 412 - protein, 4 aa, synthetic construct
LARN

SEQ ID NO: 413 - protein, 4 aa, synthetic construct
ARNV

SEQ ID NO: 414 - protein, 5 aa, synthetic construct
SNLAR

SEQ ID NO: 415 - protein, 5 aa, synthetic construct
NLARN

SEQ ID NO: 416 - protein, 5 aa, synthetic construct
LARNV

SEQ ID NO: 417 - protein, 6 aa, synthetic construct
SNLARN

SEQ ID NO: 418 - protein, 6 aa, synthetic construct
NLARNV

SEQ ID NO: 419 - protein, 4 aa, synthetic construct
TNNT

SEQ ID NO: 420 - protein, 4 aa, synthetic construct
NNTK

SEQ ID NO: 421 - protein, 4 aa, synthetic construct
NTKP

SEQ ID NO: 422 - protein, 4 aa, synthetic construct
TKPL

SEQ ID NO: 423 - protein, 5 aa, synthetic construct
TNNTK

SEQ ID NO: 424 - protein, 5 aa, synthetic construct
NNTKP

SEQ ID NO: 425 - protein, 5 aa, synthetic construct
NTKPL

SEQ ID NO: 426 - protein, 6 aa, synthetic construct
TNNTKP

SEQ ID NO: 427 - protein, 6 aa, synthetic construct
NNTKPL

SEQ ID NO: 428 - protein, 4 aa, synthetic construct
PNAS

SEQ ID NO: 429 - protein, 4 aa, synthetic construct
NASV

SEQ ID NO: 430 - protein, 4 aa, synthetic construct
ASVN

SEQ ID NO: 431 - protein, 4 aa, synthetic construct
SVNS

SEQ ID NO: 432 - protein, 5 aa, synthetic construct
PNASV

SEQ ID NO: 433 - protein, 5 aa, synthetic construct
NASVN

SEQ ID NO: 434 - protein, 5 aa, synthetic construct
ASVNS

SEQ ID NO: 435 - protein, 6 aa, synthetic construct
PNASVN

SEQ ID NO: 436 - protein, 6 aa, synthetic construct
NASVNS

SEQ ID NO: 437 - protein, 4 aa, synthetic construct
LNTI

SEQ ID NO: 438 - protein, 4 aa, synthetic construct
NTIR

SEQ ID NO: 439 - protein, 4 aa, synthetic construct
TIRN

SEQ ID NO: 440 - protein, 4 aa, synthetic construct
IRNV

SEQ ID NO: 441 - protein, 5 aa, synthetic construct
LNTIR

SEQ ID NO: 442 - protein, 5 aa, synthetic construct
NTIRN

SEQ ID NO: 443 - protein, 5 aa, synthetic construct
TIRNV

SEQ ID NO: 444 - protein, 6 aa, synthetic construct
LNTIRN

SEQ ID NO: 445 - protein, 6 aa, synthetic construct
NTIRNV

SEQ ID NO: 446 - protein, 4 aa, synthetic construct
AQAQ

SEQ ID NO: 447 - protein, 11 aa, synthetic construct
AQQAVRTSLAQ

SEQ ID NO: 448 - protein, 11 aa, synthetic construct
AQPHEGSSRAQ

SEQ ID NO: 449 - protein, 11 aa, synthetic construct
AQPNASVNSAQ

SEQ ID NO: 450 - protein, 11 aa, synthetic construct
AQLNNTKTTAQ

SEQ ID NO: 451 - protein, 11 aa, synthetic construct
AQSNLARNVAQ

SEQ ID NO: 452 - protein, 11 aa, synthetic construct
AQLNTIRNVAQ